(19) United States
(12) Patent Application Publication
(10) Pub. No.: US 2014/0275143 A1
(43) Pub. Date: Sep. 18, 2014

(54) Compositions Comprising An Opioid And An Additional Active Pharmaceutical Ingredient For Rapid Onset And Extended Duration Of Analgesia That May Be Administered Without Regard To Food

(71) Applicant: MALLINCKRODT LLC, Hazelwood, MO (US)

(72) Inventors: Krishna R. Devarakonda, Saint Louis, MO (US); Michael J. Giuliani, Creve Coeur, MO (US); Vishal K. Gupta, Hillsborough, NJ (US); Ralph A. Heasley, Webster Groves, MO (US); Susan Shelby, Town and Country, MO (US)

(73) Assignee: MALLINCKRODT LLC, Hazelwood, MO (US)

(21) Appl. No.: 14/188,582

(22) Filed: Feb. 24, 2014

Related U.S. Application Data
(60) Provisional application No. 61/794,848, filed on Mar. 15, 2013.
(60) Provisional application No. 61/798,525, filed on Mar. 15, 2013.
(60) Provisional application No. 61/871,956, filed on Aug. 30, 2013.
(60) Provisional application No. 61/871,690, filed on Aug. 29, 2013.
(60) Provisional application No. 61/926,027, filed on Jan. 10, 2014.
(60) Provisional application No. 61/928,509, filed on Jan. 17, 2014.

(51) Int. Cl. A61K 31/485; A61K 9/24; A61K 9/20; A61K 31/167

(52) U.S. Cl. CPC A61K 31/485; A61K 31/167; A61K 9/209; A61K 9/2031
USPC 514/282

(57) ABSTRACT

The present disclosure provides pharmaceutical compositions comprising an opioid and an additional active pharmaceutical ingredient, wherein the composition exhibits gastric retentive properties which are achieved by a combination of a physical property of the composition and release of the opioid, wherein upon administration to a subject, the composition has at least one pharmacokinetic parameter that differs by less than about 30% when the subject is in a fasted state as compared to a fed state. The present disclosure further provides pharmaceutical composition comprising oxycodone and acetaminophen that provides a rapid onset of analgesia, and reduced levels of acetaminophen near the end of the dosing interval. Also provided are an extended release pharmaceutical composition comprising oxycodone and acetaminophen that provides reduced abuse potential.

### RELATED CASES

This application claims priority to U.S. Provisional Application Nos. 61/794,848 and 61/798,525 filed on Mar. 15, 2013, U.S. Provisional Application No. 61/871,956 filed on Aug. 30, 2013, U.S. Provisional Application No. 61/871,690 U.S. filed on Aug. 29, 2013, Provisional Application No. 61/926,027 filed on Jan. 10, 2014, and U.S. Provisional Application No. 61/928,509 filed on Jan. 17, 2014, which are incorporated herein by reference in their entirety to the full extent permitted by law.

### FIELD OF THE INVENTION

The present disclosure relates to pharmaceutical compositions comprising an opioid and an additional active pharmaceutical ingredient wherein the compositions may be administered under fed or fasted conditions. The present disclosure further relates to extended release pharmaceutical compositions comprising oxycodone and acetaminophen that provide a rapid onset of analgesia, followed by an extended duration of analgesia of about 12 hours.

### BACKGROUND OF THE INVENTION

Oral drug administration remains the route of choice for the majority of clinical applications. Modified release (MR) dosage forms that are administered once or twice daily offer advantages over their immediate release (IR) counterparts because they reduce the magnitude of peaks and troughs of drug plasma concentration, provide longer dosing intervals, sustained analgesic effect, and increased patient compliance. These modified release formulations may be referred to as controlled release (CR), sustained release (SR) and/or extended release (ER) etc. For certain types of patients, such as those suffering from pain, these MR products may permit the patient to sleep through the night without having to wake up during the night to take the next dose. Thus, these dosage forms can significantly increase the quality of life for such patients.

Gastroretentive (GR) dosage formulations have demonstrated successful delivery of drugs for extended durations of action. One way to improve drug absorption is to hold a drug delivery system above the preferred absorption site or window (proximal small intestine), and maintain the drug release at an appropriate rate. For example, one strategy is to retain the formulation in the stomach (gastroretention). Over the last few decades, several gastroretentive drug delivery approaches have been designed and developed, including: high density (sinking) systems, which are retained in the bottom of the stomach, low density systems that float in gastric fluid due to buoyancy, mucoadhesive systems that release drugs following bio-adhesion to the gastric mucosa, superporous hydrogel systems, magnetic systems, and extendible or swellable systems that expand in the presence of water (gastric fluid) and fail to pass through the pyloric sphincter of stomach.

Parameters controlling the gastric retention of oral dosage forms include: density, size and shape of the dosage form, food intake and its nature (particularly fat content), total caloric content and frequency of intake, posture, gender, age, sex, sleep, body mass index, physical activity, disease states of the individual (e.g., diabetes), and administration of drugs with impact on gastrointestinal transit time, for example, drugs acting as anticholinergic agents (e.g., atropine, propantheline), opiates (e.g., codeine) and prokinetic agents (e.g., metoclopramide, cisapride).

Food intake (i.e., viscosity of food, food volume, caloric value, and frequency of feeding) may have a profound effect on the gastric retention of dosage forms. The presence or absence of food in the gastrointestinal tract (GIT) influences the gastric retention time (GRT) of the dosage form. Usually the presence of food in the gastrointestinal tract (GIT) improves the GRT of the dosage form and, thus, absorption increases because the drug stays at the preferred absorption site for a longer period of time. Indeed, GR formulations of the prior art should be administered with food in order to achieve the desired bioavailability.

There is a need, therefore, for extended release GR compositions comprising an opioid and a second active agent, wherein bioavailability of such composition is independent of food intake, thereby increasing the flexibility and ease of the administration of the composition.

Researchers have also combined various classes of pain drugs to provide better analgesia to patients. For example, a combination of acetaminophen-oxycodone hydrochloride is commercially available as Percocet and acetaminophen-hydrocodone bitartrate as Vicodin. In randomized controlled trials, it was shown that the combination product Percocet was statistically superior to MR oxycodone in various outcome measures of pain relief. Other combination products such as Acetaminophen-Hydrocodone and Acetaminophen-Tramadol are either available or described in the literature. It is postulated that the combination of two analgesic drugs with complementary mechanisms of action results in enhanced analgesia due to an additive effect, an “opioid-sparing” effect, and an improved side effect and safety profile. The improved safety profile results from the use of reduced doses of two analgesics with different side-effects rather than an equieffective dose of a single agent.

Acetaminophen is absorbed from the small intestine and primarily metabolized by conjugation, like glucuronidation and sulfation, in the liver to nontoxic, water-soluble compounds that are eliminated in the urine. When the maximum daily dose is exceeded over a prolonged period, metabolism by conjugation becomes saturated, and excess acetaminophen is oxidatively metabolized by cytochrome P450 (CYP) enzymes (e.g., CYP2E1, 1A2, 2A6, 3A4) to a reactive metabolite, N-acetyl-p-benzoquinone-imine (NAPQI). NAPQI is a reactive free radical with an extremely short half-life that is rapidly inactivated by conjugation with glutathione, which is acting as a sulfhydryl donor. Once the pool of available glutathione is exhausted, the cysteines of cellular proteins become sulfhydryl donors to NAPQI, binding covalently and initiating a cascade of oxidative and cellular damage, resulting in necrosis and, ultimately, liver failure. Thus, avoiding excessive NAPQI formation is an important strategy when using acetaminophen, although to date acetaminophen-sparing has not been an approach any manufacturers have chosen to take. However, due to the prevalence of acetaminophen in many over-the-counter products, it is prudent to consider acetaminophen-sparing precautions when considering combination therapy lasting more than a few days to avoid an inadvertent reduction in glutathione stores.

Thus, various options for pain management are available that are both IR and MR, and contain either a single drug or a combination of analgesics. While these combination products provide the benefits associated with combining two analgesics as described above, both IR and MR, in itself, have a significant disadvantage. IR combination products lack the advantages of MR products described previously. MR combination products lack a significant benefit associated with IR products—rapid onset of analgesia—that is extremely desirable for pain management. Because MR products retard the rate of drug release to sustain the drug effect over prolonged period, release of drug is slow resulting in significant time before effective analgesic drug concentration is attained in the bloodstream. There exists a clinical need for pain management that combines the desirable features of IR and MR in combination pain products.

### SUMMARY OF THE INVENTION

Among the various aspects of the present disclosure is pharmaceutical composition comprising at least one extended release portion comprising an opioid, an additional active pharmaceutical ingredient, or a combination thereof, and at least one extended release component. At least one extended release portion comprises from about 60% to about 80% of the total amount of the opioid in the composition, and the composition has gastric retentive properties that are achieved by a combination of a physical property of the composition and release of the opioid. Moreover, when the composition is orally administered to a subject, the opioid or the additional active pharmaceutical ingredient produces a plasma profile characterized by at least one pharmacokinetic parameter that differs by less than about 30% when the subject is in a fasted state as compared to a fed.

A further aspect of the disclosure encompasses an extended release pharmaceutical composition comprising (a) at least one immediate release portion comprising an opioid, an additional active pharmaceutical ingredient, or a combination thereof, and (b) at least one extended release portion comprising an extended release component and an opioid, an additional active pharmaceutical ingredient, or a combination thereof. At least one immediate release portion comprises from about 20% to about 40% of the total amount of the opioid in the composition, and the composition has gastric retentive properties that are achieved by a combination of a physical property of the composition and release of the opioid. Additionally, when the composition is orally administered to a subject, the opioid or the additional active pharmaceutical ingredient in the composition produce a plasma profile characterized by at least one pharmacokinetic parameter that differs by less than about 30% when the subject is in a fasted state as compared to a fed state.

Still another aspect of the disclosure provides a method for administering a gastric retentive pharmaceutical composition comprising an opioid to a subject in need thereof. The method comprises orally administering an effective amount of the gastric retentive composition to the subject, the subject being in a fasted state, wherein the opioid in the composition produces a plasma profile characterized by at least one pharmacokinetic parameter that differs by less than about 30% when the subject is in a fasted state as compared to a fed state.

Still another aspect of the disclosure provides a method for administering a gastric retentive pharmaceutical composition comprising an opioid to a subject in need thereof. The method comprises orally administering an effective amount of the gastric retentive composition to the subject, the subject being in a fasted state, wherein the opioid in the composition produces a plasma profile characterized by at least one pharmacokinetic parameter that differs by less than about 30% when the subject is in a fasted state as compared to a fed state.

Yet another aspect of the disclosure encompasses a method for treating pain in a subject in need thereof. The method comprises orally administering an effective amount of a gastric retentive pharmaceutical composition comprising an opioid to the subject in a fasted state, wherein the opioid in the composition produces a plasma profile characterized by at least one pharmacokinetic parameter that differs by less than about 30% when the subject is in a fasted state as compared to a fed state.

Yet another aspect of the disclosure provides a pharmaceutical composition for oral administration in the treatment of pain, comprising (a) at least one immediate release portion comprising acetaminophen and oxycodone or a pharmaceutically acceptable salt thereof; and (b) at least one extended release portion comprising acetaminophen and oxycodone or salt thereof, and an extended release component, wherein the total amount of acetaminophen in the composition is about 325 mg to about 650 mg, and the total amount of oxycodone or salt in the composition is about 7.5 mg to about 15 mg, and wherein upon placement of the composition in an in vitro dissolution test comprising USP Paddle Method at a paddle speed of about 100 rpm in 900 ml of 0.1 N HCl using a USP type II apparatus at a constant temperature of 37° C., about 30%, by weight, of the oxycodone or salt thereof is released at about 15 minutes in the test and at least about 90%, by weight, of the acetaminophen is released at about 8 hours in the test. Further, upon oral administration of a single dose of the composition to a subject in need of analgesia, the composition provides a Cₘₐₓ for oxycodone from about 0.9 ng/mL/mg to about 1.6 ng/mL/mg, a Cₘₐₓ for acetaminophen from about 4.0 ng/mL/mg to about 11.0 ng/mL/mg, a Tₘₐₓ for oxycodone from about 2 hours to about 7 hours, and a Tₘₐₓ for acetaminophen from about 0.5 hour to about 6 hours.

In a further aspect of the disclosure provides a pharmaceutical composition for oral administration in the treatment of pain, comprising (a) at least one immediate release portion comprising acetaminophen and oxycodone or a pharmaceutically acceptable salt thereof, and (b) at least one extended release portion comprising acetaminophen and oxycodone or salt thereof, and an extended release component; wherein the total amount of acetaminophen in the composition is about 325 mg to about 650 mg, and the total amount of oxycodone or salt in the composition is about 7.5 mg to about 15 mg. Moreover, upon placement of the composition in an in vitro dissolution test comprising USP Paddle Method at a paddle speed of about 150 rpm in 900 ml of 0.1 N HCl using a USP type II apparatus at a constant temperature of 37° C., no more than about 65%, by weight, of the total amount of the oxycodone or salt is released and no more than about 75%, by weight, of the total amount of the acetaminophen is released after 2 hours; from about 65% to about 85%, by weight, of the total amount of the oxycodone or salt is released and from about 70% to about 90%, by weight, of the total amount of the acetaminophen is released after 4 hours; from about 85% to about 100%, by weight, of the total amount of the oxycodone or salt is released and from about 85% to about 100%, by weight, of the total amount of the acetaminophen is released after 8 hours; and from about 95% to about 100%, by weight, of the total amount of the oxycodone or salt is released and from about 90% to about 100%, by weight, of the total amount of the acetaminophen is released after 12 hours.

An additional aspect of the disclosure provides for a pharmaceutical composition for oral administration in the treatment of pain, comprising (a) at least one immediate release portion comprising acetaminophen and oxycodone or a pharmaceutically acceptable salt thereof; and (b) at least one extended release portion comprising acetaminophen and oxycodone or salt thereof, and an extended release component; wherein the total amount of acetaminophen in the composition is about 325 mg to about 650 mg, and the total amount of oxycodone or salt in the composition is about 7.5 mg to about 15 mg. And upon oral administration of the composition in an amount of about 15 mg oxycodone or salt and about 650 mg acetaminophen, the composition provides an AUC_{0-1.7h} for acetaminophen of about 5.0 ng·h/mL/mg to about 13.0 ng·h/mL/mg; an AUC_{1.7-48h} for acetaminophen of about 25.0 ng·h/mL/mg to about 75.0 ng·h/mL/mg; an AUC_{0-2.8h} for oxycodone or salt of about 1.0 ng·h/mL/mg to about 3.0 ng·h/mL/mg; and AUC_{2.8-48h} of about 7.5 ng·h/mL/mg to about 15.0 ng·h/mL/mg.

Another aspect of the present disclosure is an extended release pharmaceutical composition comprising at least one extended release portion comprising oxycodone, acetaminophen, or a combination thereof, and at least one extended release component. At least one extended release portion comprises from about 60% to about 80% of the total amount of the oxycodone in the composition, and the composition has gastric retentive properties that are achieved by a combination of a physical property of the composition and release of the oxycodone. Moreover, when the composition is orally administered to a subject, the oxycodone or the acetaminophen produces a plasma profile characterized by at least one pharmacokinetic parameter that differs by less than about 30% when the subject is in a fasted state as compared to a fed state.

An additional aspect of the present disclosure provides a dosage form comprising from about 7.5 mg to about 30 mg of oxycodone and from about 325 mg to about 650 mg of acetaminophen. The dosage form comprises (a) at least one immediate release portion comprising about 25% of the total amount of oxycodone in the composition and about 50% of the total amount of acetaminophen in the composition; and (b) at least one extended release portion comprising about 75% of the total amount of oxycodone in the composition, about 50% of the total amount of acetaminophen in the composition, and about 35% to about 45%, by weight of the at least one extended release portion, of an extended release polymer comprising a polyethylene oxide.

A further aspect of the disclosure encompasses an extended release pharmaceutical composition comprising (a) at least one immediate release portion comprising oxycodone, acetaminophen, or a combination thereof, and (b) at least one extended release portion comprising an extended release component and oxycodone, acetaminophen, or a combination thereof. At least one immediate release portion comprises from about 20% to about 40% of the total amount of the oxycodone in the composition, and the composition has gastric retentive properties that are achieved by a combination of a physical property of the composition and release of the oxycodone. Additionally, when the composition is orally administered to a subject, the oxycodone or the acetaminophen in the composition produce a plasma profile characterized by at least one pharmacokinetic parameter that differs by less than about 30% when the subject is in a fasted state as compared to a fed state.

A further aspect of the disclosure provides a method for reducing the risk of acetaminophen-induced hepatic damage in a subject being treated for pain with a dosage regimen that comprises administering to the subject at least two consecutive doses of a pharmaceutical composition comprising oxycodone and acetaminophen. The method comprises (a) administering a first dose of the pharmaceutical composition comprising at least one extended release portion comprising acetaminophen, oxycodone or a combination thereof, and an extended release component to the subject, wherein the composition maintains a therapeutic blood plasma concentration of oxycodone of at least 5 ng/mL from about 0.75 hours to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration; and (b) administering a second dose of the pharmaceutical composition to the subject at about 12 hours after administration of the first dose.

Yet another aspect of the disclosure encompasses a method for treating pain in a subject in need thereof with a pharmaceutical composition that comprises oxycodone and acetaminophen. The method comprises orally administering to the subject an effective amount of pharmaceutical composition comprising least one extended release portion comprising oxycodone, acetaminophen or a combination thereof, and an extended release component, wherein the composition maintains a therapeutic plasma concentration of oxycodone of at least about 5 ng/mL from about 0.75 hour to about 10 hours after administration of the composition, and wherein at least about 90% of the acetaminophen is released from the composition by about 8 hours after administration of the composition such that, by about 10 hours after administration of the composition, the blood plasma concentration of acetaminophen is less than about 30% of acetaminophen's maximum plasma concentration.

A further aspect of the disclosure encompasses a pharmaceutical composition for extended release of oxycodone and acetaminophen comprising (a) at least one immediate release portion oxycodone, acetaminophen or a combination thereof, and (b) at least one extended release portion comprising oxycodone, acetaminophen or a combination thereof, and an extended release component wherein about 30% of the oxycodone in the pharmaceutical composition is released within about 15 minutes of administration and at least about 90% of the acetaminophen in the pharmaceutical composition is released in about 8 hours when measured in 900 ml of 0.1N HCl using a USP type II apparatus at a paddle speed of about 100 rpm and a constant temperature of 37° C.

Still another aspect of the disclosure provides a dosage form comprising (a) an immediate release portion comprising acetaminophen and oxycodone, wherein the immediate release portion comprises, by weight of the immediate release portion, from about 70% to about 80% of acetaminophen and from about 0.5% to about 1% of oxycodone; and (b) an extended release portion comprising acetaminophen, oxycodone, and an extended release polymer, wherein the extended release portion comprises, by weight of the extended release portion, from about 20% to about 40% of acetaminophen, from about 0.5% to about 2% of oxycodone, and from about 30% to about 50% of the extended release polymer.

Other features and aspects of the disclosure are described in detail below.

### REFERENCE TO COLOR FIGURES

This application file contains at least one drawing executed in color. Copies of this patent application publication with color drawings will be provided by the Office upon request and payment of the necessary fee.

### BRIEF DESCRIPTION OF THE DRAWINGS

FIG. 1 presents the in vitro release profile of oxycodone from oxycodone-acetaminophen bilayer tablets comprising either 15 or 30 mg of oxycodone, 500 mg of acetaminophen (APAP), and either 35% (w/w) POLYOX® 1105, 45% (w/w) POLYOX® 1105, or 45% (w/w) POLYOX® N60K, as indicated.

FIG. 2 shows the in vitro release profile of acetaminophen from oxycodone-acetaminophen bilayer tablets comprising either 15 or 30 mg of oxycodone, 500 mg of acetaminophen (APAP), and either 35% (w/w) POLYOX® 1105, 45% (w/w) POLYOX® 1105, or 45% (w/w) POLYOX® N60K, as indicated.

FIG. 3 presents the in vitro release profile of oxycodone from bilayer tablets comprising 7.5 mg of oxycodone and 325 mg of acetaminophen, and bilayer tablets comprising 15 mg of oxycodone and 650 mg of acetaminophen, as indicated.

FIG. 4 presents the in vitro release profile of acetaminophen from bilayer tablets comprising 7.5 mg of oxycodone and 325 mg of acetaminophen, and bilayer tablets comprising 15 mg of oxycodone and 650 mg of acetaminophen, as indicated.

FIG. 5 is a graphical representation of the mean plasma oxycodone concentrations as a function of time after administration of a single dose of bilayer tablet comprising 15 mg oxycodone/500 mg acetaminophen and having fast, medium, or slow release properties as compared to an immediate release 7.5 oxycodone/325 acetaminophen tablet administered twice at a 6 hr interval.

FIG. 6 is a graphical representation of the mean plasma acetaminophen concentrations as a function of time after administration of a single dose of bilayer tablet comprising 15 mg oxycodone/500 mg acetaminophen and having fast, medium, or slow release properties as compared to an immediate release 7.5 oxycodone/325 acetaminophen tablet administered twice at a 6 hr interval. The immediate release 7.5 oxycodone/325 acetaminophen tablet dose was normalized.

FIG. 7 is a graphical representation of the mean plasma oxycodone concentrations as a function of time after administration of a single dose of bilayer tablet comprising 30 mg oxycodone/500 mg acetaminophen and having fast, medium, or slow release properties as compared to an immediate release 7.5 oxycodone/325 acetaminophen tablet administered twice at a 6 hr interval. The immediate release 7.5 oxycodone/325 acetaminophen tablet dose was normalized.

FIG. 8 is a graphical representation of the mean plasma acetaminophen concentrations as a function of time after administration of a single dose of bilayer tablet comprising 30 mg oxycodone/500 mg acetaminophen and having fast, medium, or slow release properties as compared to an immediate release 7.5 oxycodone/325 acetaminophen tablet administered twice at a 6 hr interval. The immediate release 7.5 oxycodone/325 acetaminophen tablet dose was normalized.

FIG. 9 shows the mean plasma concentrations of oxycodone versus time by treatment. Treatment A was one tablet of 15 mg oxycodone/650 mg acetaminophen administered orally under fed conditions. Treatment B was two tablets of 15 mg oxycodone/650 mg acetaminophen administered orally one at a time under fed conditions. Treatment C was one tablet of an immediate release 7.5 oxycodone/325 acetaminophen tablet administered orally every 6 hours for 2 doses under fed conditions.

FIG. 10 presents the mean plasma concentrations of acetaminophen versus time by treatment. Treatment A was one tablet of 15 mg oxycodone/650 mg acetaminophen administered orally under fed conditions. Treatment B was two tablets of 15 mg oxycodone/650 mg acetaminophen administered orally one at a time under fed conditions. Treatment C was one tablet of an immediate release 7.5 oxycodone/325 acetaminophen tablet administered orally every 6 hours for 2 doses under fed conditions.

FIG. 11 shows the mean plasma concentrations of oxycodone versus time by treatment. Treatment A was one tablet of 15 mg oxycodone/650 mg acetaminophen administered orally every 12 hours for 4.5 days (9 doses) under fed conditions. Treatment B was two tablets of 15 mg oxycodone/650 mg acetaminophen administered orally one at a time every 12 hours for 4.5 days (9 doses) under fed conditions. Treatment C was two tablets of an immediate release 7.5 oxycodone/325 acetaminophen tablet administered orally every 6 hours for 4.5 days (18 doses) under fed conditions.

FIG. 12 shows the mean plasma concentrations of acetaminophen versus time by treatment. Treatment A was one tablet of 15 mg oxycodone/650 mg acetaminophen administered orally every 12 hours for 4.5 days (9 doses) under fed conditions. Treatment B was two tablets of 15 mg oxycodone/650 mg acetaminophen administered orally one at a time every 12 hours for 4.5 days (9 doses) under fed conditions. Treatment C was two tablets of an immediate release 7.5 oxycodone/325 acetaminophen tablet administered orally every 6 hours for 4.5 days (18 doses) under fed conditions.

FIG. 13 presents the mean plasma concentrations of oxycodone versus time by treatment following oral administration of one tablet of 15 mg oxycodone/650 mg acetaminophen. Treatment A was under fed conditions. Treatment B was under fasted conditions.

FIG. 14 shows the mean plasma concentrations of oxycodone versus time by treatment following oral administration of two tablets of 15 mg oxycodone/650 mg acetaminophen. Treatment A was under fed conditions. Treatment B was under fasted conditions.

FIG. 15 presents the mean plasma concentrations of acetaminophen versus time by treatment following oral administration of one tablet of 15 mg oxycodone/650 mg acetaminophen. Treatment A was under fed conditions. Treatment B was under fasted conditions.

FIG. 16 shows mean plasma concentrations of acetaminophen versus time by treatment following oral administration of two tablets of 15 mg oxycodone/650 mg acetaminophen. Treatment A was under fed conditions. Treatment B was under fasted conditions.

FIG. 17 illustrates the in vitro release of oxycodone from a bilayer tablet comprising 7.5 mg of oxycodone/325 mg of acetaminophen tested in 0.1 N HCl at a paddle speed of 150 rpm containing 0%, 5%, 20%, or 40% ethanol. Plotted is the percent of oxycodone released over a period of 2 hours.

FIG. 18 presents the in vitro release of acetaminophen from a bilayer tablet comprising 7.5 mg of oxycodone/325 mg of acetaminophen tested in 0.1 N HCl at a paddle speed of 150 rpm containing 0%, 5%, 20%, or 40% ethanol. Plotted is the percent of acetaminophen released over a 2 hour period.

FIG. 19 shows the mean plasma concentrations of oxycodone as a function of time by treatment following oral administration of two tablets of 7.5 mg of oxycodone/325 mg of acetaminophen. Treatment A was under fed (high fat) conditions. Treatment B was under fed (low fat) conditions. Treatment C was under fasted conditions.

FIG. 20 presents the mean plasma concentrations of acetaminophen as a function of time by treatment following oral administration of two tablets of 7.5 mg of oxycodone/325 mg of acetaminophen. Treatment A was under fed (high fat) conditions. Treatment B was under fed (low fat) conditions. Treatment C was under fasted conditions.

FIG. 21 shows the mean plasma concentrations of oxycodone versus time by treatment. Treatment A was one tablet of 7.5 mg oxycodone/325 mg acetaminophen administered orally under fasted conditions. Treatment B was two tablets of 7.5 mg oxycodone/325 mg acetaminophen administered orally under fasted conditions. Treatment C was one tablet of an immediate release 7.5 oxycodone/325 acetaminophen tablet administered orally every 6 hours for 2 doses under fasted conditions. Treatment D was two tablets of an immediate release 7.5 oxycodone/325 acetaminophen tablet administered orally every 6 hours for 2 doses under fasted conditions.

FIG. 22 presents the mean plasma concentrations of acetaminophen versus time by treatment. Treatment A was one tablet of 7.5 mg oxycodone/325 mg acetaminophen administered orally under fasted conditions. Treatment B was two tablets of 7.5 mg oxycodone/325 mg acetaminophen administered orally under fasted conditions. Treatment C was one tablet of an immediate release 7.5 oxycodone/325 acetaminophen tablet administered orally every 6 hours for 2 doses under fasted conditions. Treatment D was two tablets an immediate release 7.5 oxycodone/325 acetaminophen tablet administered orally every 6 hours for 2 doses under fasted conditions.

FIG. 23 shows a deconvolution plot of the biphasic absorption of oxycodone from tablets of the 7.5 mg oxycodone/325 mg acetaminophen formulation. The cumulative amount of oxycodone is plotted versus time. Circles represent one tablet of 7.5 mg oxycodone/325 mg acetaminophen; squares represent two tablets of 7.5 mg oxycodone/325 mg acetaminophen; and the immediate release 7.5 oxycodone/325 acetaminophen tablet is shown in a solid line with no symbols.

FIG. 24 presents a deconvolution plot of the biphasic absorption of acetaminophen from tablets of the 7.5 mg oxycodone/325 mg acetaminophen formulation. The cumulative amount of acetaminophen is plotted versus time. Circles represent one tablet of 7.5 mg oxycodone/325 mg acetaminophen; triangles represent two tablets of 7.5 mg oxycodone/325 mg acetaminophen; and squares represent the immediate release 7.5 oxycodone/325 acetaminophen product.

FIG. 25 shows the mean plasma concentrations of oxycodone versus time by treatment. Treatment A was one tablet of 7.5 mg oxycodone/325 mg acetaminophen administered orally every 12 hours for 4.5 days (9 doses) under fasted conditions. Treatment B was two tablets of 7.5 mg oxycodone/325 mg acetaminophen administered orally every 12 hours for 4.5 days (9 doses) under fasted conditions. Treatment C was one tablet of an immediate release 7.5 oxycodone/325 acetaminophen tablet administered orally every 6 hours for 4.5 days (18 doses) under fasted conditions.

FIG. 26 presents the mean plasma concentrations of acetaminophen versus time by treatment. Treatment A was one tablet of 7.5 mg oxycodone/325 mg acetaminophen administered orally every 12 hours for 4.5 days (9 doses) under fasted conditions. Treatment B was two tablets of 7.5 mg oxycodone/325 mg acetaminophen administered orally every 12 hours for 4.5 days (9 doses) under fasted conditions. Treatment C was one tablet of an immediate release 7.5 oxycodone/325 acetaminophen tablet administered orally every 6 hours for 4.5 days (18 doses) under fasted conditions.

FIG. 27A is a bar graph depicting the simulated fractional absorption of acetaminophen in the upper GIT of a human subject after treatment of a 7.5 mg oxycodone/325 mg acetaminophen immediate release formulation.

FIG. 27B is a bar graph depicting the simulated fractional absorption of acetaminophen in the upper GIT of a human subject after treatment of a 7.5 mg oxycodone/325 mg acetaminophen immediate release formulation, wherein the formulation's transit time from the stomach through ileum 3 has been doubled.

FIG. 27C is a bar graph depicting the simulated fractional absorption of acetaminophen in the upper GIT of a human subject after treatment of a 7.5 mg oxycodone/325 mg acetaminophen immediate release formulation, wherein the formulation's transit time in the stomach has been increased by two hours.

FIG. 28A is a bar graph depicting the simulated fractional absorption of oxycodone in the upper GIT of a human subject after treatment of a 7.5 mg oxycodone/325 mg acetaminophen immediate release formulation.

FIG. 28B is a bar graph depicting the simulated fractional absorption of oxycodone in the upper GIT of a human subject after treatment of a 7.5 mg oxycodone/325 mg acetaminophen immediate release formulation, wherein the formulation's transit time from the stomach through ileum 3 has been doubled.

FIG. 28C is a bar graph depicting the simulated fractional absorption of oxycodone in the upper GIT of a human subject after treatment of a 7.5 mg oxycodone/325 mg acetaminophen immediate release formulation, wherein the formulation's transit time in the stomach has been increased by two hours.

FIG. 29A presents the mean plasma concentrations and Partial AUCs of acetaminophen (e.g., AUC_{0-1.7h} and AUC_{1.7-48h}) versus time by treatment: (1) Treatment B of Example 10, (2) Treatment C of Example 9, and (3) Treatment D of Example 10.

FIG. 29B presents the mean plasma concentrations and Partial AUCs of oxycodone (e.g., AUC0-2.8 h and AUC2.8-48 h) versus time treatment: (1) Treatment B of Example 10, (2) Treatment C of Example 9, and (3) Treatment D of Example 10.

FIG. 30A presents the mean plasma concentrations and Partial AUCs of oxycodone versus time for Treatment A of Example 4, Treatment A of Example 6, and Treatment C of Example 4.

FIG. 30B presents the mean plasma concentrations and Partial AUCs of acetaminophen versus time for Treatment A of Example 4, Treatment A of Example 6, and Treatment C of Example 4.

FIG. 31 presents oxycodone dissolution data from crushed and intact immediate release tablets containing 7.5 mg oxycodone and 325 mg acetaminophen.

FIGS. 32A and 32B present acetaminophen dissolution data from crushed and intact pharmaceutical formulations described herein containing a total of 7.5 mg oxycodone and a total of 325 mg acetaminophen per tablet.

FIGS. 33A and 33B present oxycodone HCl dissolution data from crushed and intact pharmaceutical formulations described herein containing a total of 7.5 mg oxycodone and a total of 325 mg acetaminophen per tablet.

FIG. 34 presents acetaminophen dissolution data for three pharmaceutical formulations described herein. The dissolution data represents an extended release tablet with the immediate release data theoretically added. For each formulation, the tablet contained a total of 9 mg oxycodone HCl and a total of 250 mg acetaminophen. The three pharmaceutical formulations contained 25% by weight POLYOX® 205, 1105, and N-60K, respectively.

FIG. 35 presents oxycodone HCl dissolution data for the three pharmaceutical formulations described in FIG. 34.

FIG. 36 presents acetaminophen dissolution data for three pharmaceutical formulations described herein. The dissolution data represents an extended release tablet with the immediate release data theoretically added. For each formulation, the tablet contained a total of 9 mg oxycodone HCl and a total of 250 mg acetaminophen. The three pharmaceutical formulations contained 45% by weight POLYOX® 205, 1105, and N-60K, respectively.

FIG. 37 presents oxycodone HCl dissolution data for the three pharmaceutical formulations described in FIG. 36.

FIG. 38 presents acetaminophen dissolution data for four pharmaceutical formulations described herein. The dissolution data represents an extended release tablet with the immediate release data theoretically added. For each formulation, the tablet contained a total of 9 mg oxycodone HCl and a total of 250 mg acetaminophen. The four pharmaceutical compositions contained 25% by weight, 35% by weight, 45% by weight, and 55% by weight POLYOX® 1105, respectively.

FIG. 39 presents oxycodone HCl dissolution data for the three pharmaceutical formulations described in FIG. 38.

FIG. 40 presents the in vitro release of oxycodone from a bilayer tablet comprising 7.5 mg of oxycodone/325 mg of acetaminophen tested in 0.1 N HCl at a paddle speed of 100 rpm containing 0%, 5%, 20%, or 40% ethanol. Plotted is the percent of oxycodone released over a period of 8 hours.

FIG. 41 presents the in vitro release of acetaminophen from a bilayer tablet comprising 7.5 mg of oxycodone/325 mg of acetaminophen tested in 0.1 N HCl at a paddle speed of 100 rpm containing 0%, 5%, 20%, or 40% ethanol. Plotted is the percent of acetaminophen released over a 8 hour period.

FIG. 42 presents the mean plasma concentrations of oxycodone versus time for A-F of Example 29.

FIG. 43 presents the mean plasma concentrations of acetaminophen versus time for Groups A-F of Example 29.

FIG. 44 presents the mean drug liking scores over time for Groups A-G of Example 29.

FIG. 45 presents the mean drug high scores over time for Groups A-G of Example 29.

FIG. 46 presents the mean good drug effects scores over time for Groups A-G of Example 29.

FIG. 47 presents the baseline adjusted pupillometry scores versus time for the subjects who completed the study set out in Example 29.

FIG. 48 presents the mean plasma concentrations of oxycodone versus time for Treatments A, B, and D of Example 30.

FIG. 49 presents the mean plasma concentrations of acetaminophen versus time for Treatments A, C, and D of Example 30.

FIG. 50 presents the mean plasma concentrations of oxycodone versus time for Treatments A, B, and D of Example 31.

FIG. 51 presents the mean plasma concentrations of acetaminophen versus time for Treatments A, C, and D of Example 31.

FIG. 52 presents the plasma oxycodone concentration over time, as well as half-value duration (HVD) and Cₘₐₓ in a single dose study in which patients received either a single dose of immediate-release oxycodone/acetaminophen (two total tablets, 7.5 mg oxycodone/325 mg acetaminophen per tablet administered at 0 h and 6 h) or a single dose of controlled-release oxycodone/acetaminophen (two total tablets, 7.5 mg oxycodone/325 mg acetaminophen per tablet, both administered at 0 h).

FIG. 53 presents the plasma oxycodone concentration over time, as well as half-value duration (HVD) and mean Cₘₐₓ, during day 1 of a multi-dose study in which patients received either immediate-release oxycodone/acetaminophen (7.5 mg oxycodone/325 mg acetaminophen per tablet administered as one tablet every 6 hours for 4.5 days) or controlled-release oxycodone/acetaminophen (two tablets of 7.5 mg oxycodone/325 mg acetaminophen per tablet (15 mg/650 mg total per dose) administered every 12 hours for 4.5 days).

FIG. 54 presents the plasma oxycodone concentration over time, as well as half-value duration (HVD) and mean Cₘₐₓ, during steady state (day 5) of a multi-dose study in which patients received either immediate-release oxycodone/acetaminophen (7.5 mg oxycodone/325 mg acetaminophen per tablet administered as one tablet every 6 hours for 4.5 days) or controlled-release oxycodone/acetaminophen (two tablets of 7.5 mg oxycodone/325 mg acetaminophen per tablet (15 mg/650 mg total per dose) administered every 12 hours for 4.5 days).

FIG. 55 presents the plasma acetaminophen concentration following single-dose of administration of controlled release oxycodone/acetaminophen. Patients received either 1 tablet (7.5 mg oxycodone/325 mg acetaminophen), 2 tablets (15 mg oxycodone/650 mg acetaminophen), or 4 tablets (30 mg oxycodone/1300 mg acetaminophen) as a single dose.

FIG. 56 presents the plasma acetaminophen concentration over days 1 to 4 during a multi-dose study of controlled-release oxycodone/acetaminophen. Patients received either 1 tablet (7.5 mg oxycodone/325 mg acetaminophen) every 12 hours or 2 tablets (15 mg oxycodone/650 mg acetaminophen) every 12 hours for 4.5 days (9 total doses).

FIG. 57 presents the steady-state plasma acetaminophen concentration (hours 96 to 144 on day 5) during a multi-dose study of controlled-release oxycodone/acetaminophen. Patients received either 1 tablet (7.5 mg oxycodone/325 mg acetaminophen) every 12 hours or 2 tablets (15 mg oxycodone/650 mg acetaminophen) every 12 hours for 4.5 days (9 total doses).

FIG. 58 presents the plasma oxycodone concentration following single-dose of administration of controlled release oxycodone/acetaminophen. Patients received either 1 tablet (7.5 mg oxycodone/325 mg acetaminophen), 2 tablets (15 mg oxycodone/650 mg acetaminophen), or 4 tablets (30 mg oxycodone/1300 mg acetaminophen) as a single dose.

FIG. 59 presents the plasma oxycodone concentration over days 1 to 4 during a multi-dose study of controlled-release oxycodone/acetaminophen. Patients received either 1 tablet (7.5 mg oxycodone/325 mg acetaminophen) every 12 hours or 2 tablets (15 mg oxycodone/650 mg acetaminophen) every 12 hours for 4.5 days (9 total doses).

FIG. 60 presents the steady-state plasma oxycodone concentration (hours 96 to 144 on day 5) during a multi-dose study of controlled-release oxycodone/acetaminophen. Patients received either 1 tablet (7.5 mg oxycodone/325 mg acetaminophen) every 12 hours or 2 tablets (15 mg oxycodone/650 mg acetaminophen) every 12 hours for 4.5 days (9 total doses).

FIG. 61 presents the participant-reported visual analog scale (VAS) scores for drug liking over time for patients receiving either low-dose intact controlled-release oxycodone/acetaminophen (2 tablets of 7.5 mg/325 mg, total dose of 15 mg/650 mg) or low-dose intact immediate-release oxycodone/acetaminophen (2 tablets of 7.5 mg/325 mg, total dose of 15 mg/650 mg).

FIG. 62 presents the participant-reported visual analog scale (VAS) scores for drug liking over time for patients receiving either high-dose intact controlled-release oxycodone/acetaminophen (4 tablets of 7.5 mg/325 mg, total dose of 30 mg/1300 mg) or high-dose intact immediate-release oxycodone/acetaminophen (4 tablets of 7.5 mg/325 mg, total dose of 30 mg/1300 mg).

FIG. 63 presents the participant-reported visual analog scale (VAS) scores for drug liking over time for patients receiving either high-dose intact controlled-release oxycodone/acetaminophen (4 tablets of 7.5 mg/325 mg, total dose of 30 mg/1300 mg) or high-dose crushed controlled-release oxycodone/acetaminophen (4 tablets of 7.5 mg/325 mg crushed and encapsulated, total dose of 30 mg/1300 mg).

FIG. 64 presents the participant-reported visual analog scale (VAS) scores for drug liking over time for patients receiving either high-dose crushed controlled-release oxycodone/acetaminophen (4 tablets of 7.5 mg/325 mg crushed and encapsulated, total dose of 30 mg/1300 mg) or high-dose crushed immediate-release oxycodone/acetaminophen (4 tablets of 7.5 mg/325 mg crushed and encapsulated, total dose of 30 mg/1300 mg).

FIG. 65 presents the participant-reported visual analog scale (VAS) scores for drug high over time for patients receiving either low-dose intact controlled-release oxycodone/acetaminophen (2 tablets of 7.5 mg/325 mg, total dose of 15 mg/650 mg) or low-dose intact immediate-release oxycodone/acetaminophen (2 tablets of 7.5 mg/325 mg, total dose of 15 mg/650 mg).

FIG. 66 presents the participant-reported visual analog scale (VAS) scores for drug high over time for patients receiving either high-dose intact controlled-release oxycodone/acetaminophen (4 tablets of 7.5 mg/325 mg, total dose of 30 mg/1300 mg) or high-dose intact immediate-release oxycodone/acetaminophen (4 tablets of 7.5 mg/325 mg, total dose of 30 mg/1300 mg).

FIG. 67 presents the participant-reported visual analog scale (VAS) scores for drug high over time for patients receiving either high-dose intact controlled-release oxycodone/acetaminophen (4 tablets of 7.5 mg/325 mg, total dose of 30 mg/1300 mg) or high-dose crushed controlled-release oxycodone/acetaminophen (4 tablets of 7.5 mg/325 mg crushed and encapsulated, total dose of 30 mg/1300 mg).

FIG. 68 presents the participant-reported visual analog scale (VAS) scores for drug high over time for patients receiving either high-dose crushed controlled-release oxycodone/acetaminophen (4 tablets of 7.5 mg/325 mg crushed and encapsulated, total dose of 30 mg/1300 mg) or high-dose crushed immediate-release oxycodone/acetaminophen (4 tablets of 7.5 mg/325 mg crushed and encapsulated, total dose of 30 mg/1300 mg).

FIG. 69 presents the least-squares mean Eₘₐₓ for drug liking for patients receiving either high-dose intact controlled-release oxycodone/acetaminophen (4 tablets of 7.5 mg/325 mg, total dose of 30 mg/1300 mg) or high-dose intact immediate-release oxycodone/acetaminophen (4 tablets of 7.5 mg/325 mg, total dose of 30 mg/1300 mg).

FIG. 70 presents the least-squares mean Eₘₐₓ for drug high and good drug effects for patients receiving either high-dose intact controlled-release oxycodone/acetaminophen (4 tablets of 7.5 mg/325 mg, total dose of 30 mg/1300 mg) or high-dose intact immediate-release oxycodone/acetaminophen (4 tablets of 7.5 mg/325 mg, total dose of 30 mg/1300 mg).

FIG. 71 presents the least-squares mean TEₘₐₓ for drug liking, drug high, and good drug effects for patients receiving either high-dose intact controlled-release oxycodone/acetaminophen (4 tablets of 7.5 mg/325 mg, total dose of 30 mg/1300 mg) or high-dose intact immediate-release oxycodone/acetaminophen (4 tablets of 7.5 mg/325 mg, total dose of 30 mg/1300 mg).

FIG. 72 presents the mean pupillometry scores over time for patients receiving the following: low-dose intact controlled-release oxycodone/acetaminophen (2 tablets of 7.5 mg/325 mg, total dose of 15 mg/650 mg); low-dose intact immediate-release oxycodone/acetaminophen (2 tablets of 7.5 mg/325 mg, total dose of 15 mg/650 mg); high-dose intact controlled-release oxycodone/acetaminophen (4 tablets of 7.5 mg/325 mg, total dose of 30 mg/1300 mg); high-dose intact immediate-release oxycodone/acetaminophen (4 tablets of 7.5 mg/325 mg, total dose of 30 mg/1300 mg); high-dose crushed controlled-release oxycodone/acetaminophen (4 tablets of 7.5 mg/325 mg crushed and encapsulated, total dose of 30 mg/1300 mg); or high-dose crushed immediate-release oxycodone/acetaminophen (4 tablets of 7.5 mg/325 mg crushed and encapsulated, total dose of 30 mg/1300 mg).

FIG. 73 presents simulated oxycodone plasma levels over time during multidose administration of either controlled-release oxycodone/acetaminophen or immediate-release oxycodone/acetaminophen. The solid line represents the simulated plasma levels for patients receiving two tablets of controlled-release 7.5 mg oxycodone/325 mg acetaminophen (total dose of 15 mg/650 mg) every 12 hours for 4.5 days (18 tablets total). The dashed line represents the simulated plasma levels for patients receiving one tablet of immediate-release 7.5 mg oxycodone/325 mg acetaminophen every 6 hours for 4.5 days (18 tablets total).

FIG. 74 presents the mean steady-state plasma concentration of oxycodone beginning at the point in time when patients in the study receiving controlled-release oxycodone/acetaminophen were administered their last dose. Patients in the study were administered oxycodone/acetaminophen according to one of the following three dosing regimens: Treatment A administered 1 tablet of controlled-release oxycodone/acetaminophen (7.5 mg oxycodone/325 mg acetaminophen) every 12 hours for 4.5 days; Treatment B administered 2 tablets of controlled-release oxycodone/acetaminophen (15 mg oxycodone/650 mg acetaminophen total per dose) every 12 hours for 4.5 days; Treatment C administered 1 tablet of immediate-release oxycodone/acetaminophen (7.5 mg oxycodone/325 mg acetaminophen) every 6 hours for 4.5 days.

FIG. 75 presents the mean steady-state plasma concentration of acetaminophen beginning at the point in time when patients in the study receiving controlled-release oxycodone/acetaminophen were administered their last dose. Patients in the study were administered oxycodone/acetaminophen according to one of the following three dosing regimens: Treatment A administered 1 tablet of controlled-release oxycodone/acetaminophen (7.5 mg oxycodone/325 mg acetaminophen) every 12 hours for 4.5 days; Treatment B administered 2 tablets of controlled-release oxycodone/acetaminophen (15 mg oxycodone/650 mg acetaminophen total per dose) every 12 hours for 4.5 days; Treatment C administered 1 tablet of immediate-release oxycodone/acetaminophen (7.5 mg oxycodone/325 mg acetaminophen) every 6 hours for 4.5 days.

FIG. 76 presents the mean plasma concentration of oxycodone over time at steady state for patients completing all study periods of the study described in Example 37. Patients in the study received each of the following treatments separately, in one of four different sequences, with a washout period between them: 2 tablets of controlled-release 7.5 mg oxycodone/325 mg acetaminophen (15 mg oxycodone/650 mg acetaminophen total per dose) every 12 hours for 4.5 days; 1 tablet of commercially available oxycodone (15 mg) every 6 hours for 4.5 days; 1 tablet of immediate-release 37.5 mg tramadol/325 mg acetaminophen every 6 hours for 4.5 days; and 1 tablet of immediate-release 7.5 mg oxycodone/325 mg acetaminophen every 6 hours for 4.5 days. Only data for the three treatments with oxycodone are included in FIG. 76.

FIG. 77 presents the mean plasma concentration of acetaminophen over time at steady state for patients completing all study periods of the study described in Example 37. Patients in the study received each of the following treatments separately, in one of four different sequences, with a washout period between them: 2 tablets of controlled-release 7.5 mg oxycodone/325 mg acetaminophen (15 mg oxycodone/650 mg acetaminophen total per dose) every 12 hours for 4.5 days; 1 tablet of commercially available oxycodone (15 mg) every 6 hours for 4.5 days; 1 tablet of immediate-release 37.5 mg tramadol/325 mg acetaminophen every 6 hours for 4.5 days; and 1 tablet of immediate-release 7.5 mg oxycodone/325 mg acetaminophen every 6 hours for 4.5 days. Only data for the three treatments with acetaminophen are included in FIG. 77.

FIG. 78 presents the mean plasma concentration of oxycodone over time for patients completing all study periods of the study described in Example 38. Patients in the study received each of the following treatments separately, in one of four different sequences, with a washout period between them: 2 tablets of controlled-release 7.5 mg oxycodone/325 mg acetaminophen (15 mg oxycodone/650 mg acetaminophen total per dose) administered once; 1 tablet of commercially available oxycodone (15 mg) every 6 hours for two doses; 1 tablet of immediate-release 37.5 mg tramadol/325 mg acetaminophen every 6 hours for two doses; and 1 tablet of immediate-release 7.5 mg oxycodone/325 mg acetaminophen every 6 hours for two doses. Only data for the three treatments with oxycodone are included in FIG. 78.

FIG. 79 presents the mean plasma concentration of acetaminophen over time for patients completing all study periods of the study described in Example 38. Patients in the study received each of the following treatments separately, in one of four different sequences, with a washout period between them: 2 tablets of controlled-release 7.5 mg oxycodone/325 mg acetaminophen (15 mg oxycodone/650 mg acetaminophen total per dose) administered once; 1 tablet of commercially available oxycodone (15 mg) every 6 hours for two doses; 1 tablet of immediate-release 37.5 mg tramadol/325 mg acetaminophen every 6 hours for two doses; and 1 tablet of immediate-release 7.5 mg oxycodone/325 mg acetaminophen every 6 hours for two doses. Only data for the three treatments with acetaminophen are included in FIG. 79.

FIG. 80 presents the plasma acetaminophen concentration over time during the first 12 hours after dosing, as well as half-value duration (HVD) and Cₘₐₓ, in a single dose study in which patients received either a single dose of immediate-release oxycodone/acetaminophen (two total tablets, 7.5 mg oxycodone/325 mg acetaminophen per tablet, 1 tablet administered at 0 h and 1 tablet at 6 h) or a single dose of controlled-release oxycodone/acetaminophen (two total tablets, 7.5 mg oxycodone/325 mg acetaminophen per tablet, both administered at 0 h).

FIG. 81 presents the plasma acetaminophen concentration over time during the first 12 hours after initial dosing, as well as half-value duration (HVD) and Cₘₐₓ, in a multi-dose study in which patients received either 1 tablet of immediate-release 7.5 mg oxycodone/325 mg acetaminophen every 6 hours for 4.5 days, or 2 tablets of controlled-release 7.5 mg oxycodone/325 mg acetaminophen (15 mg/650 mg total per dose) every 12 hours for 4.5 days.

FIG. 82 presents the steady-state (day 5) plasma acetaminophen concentration over time, as well as half-value duration (HVD) and Cₘₐₓ, in a multi-dose study in which patients received either 1 tablet of immediate-release 7.5 mg oxycodone/325 mg acetaminophen every 6 hours for 4.5 days, or 2 tablets of controlled-release 7.5 mg oxycodone/325 mg acetaminophen (15 mg/650 mg total per dose) every 12 hours for 4.5 days.

FIG. 83 presents the plasma oxycodone concentrations and patient-reported visual analog scale (VAS) scores for drug liking over the first 12 hours after dosing in patients receiving either low-dose intact controlled-release oxycodone/acetaminophen (two tablets of 7.5 mg oxycodone/325 mg acetaminophen, 15 mg/650 mg total) or low-dose intact immediate-release oxycodone/acetaminophen (two tablets of 7.5 mg oxycodone/325 mg acetaminophen, 15 mg/650 mg total).

FIG. 84 presents the plasma oxycodone concentrations and patient-reported visual analog scale (VAS) scores for drug liking over the first 12 hours after dosing in patients receiving either high-dose intact controlled-release oxycodone/acetaminophen (four tablets of 7.5 mg oxycodone/325 mg acetaminophen, 30 mg/1300 mg total) or high-dose intact immediate-release oxycodone/acetaminophen (four tablets of 7.5 mg oxycodone/325 mg acetaminophen, 30 mg/1300 mg total).

FIG. 85 presents the plasma oxycodone concentrations and patient-reported visual analog scale (VAS) scores for drug liking over the first 12 hours after dosing in patients receiving either high-dose crushed controlled-release oxycodone/acetaminophen (four tablets of 7.5 mg oxycodone/325 mg acetaminophen, 30 mg/1300 mg total, crushed and encapsulated) or high-dose crushed immediate-release oxycodone/acetaminophen (four tablets of 7.5 mg oxycodone/325 mg acetaminophen, 30 mg/1300 mg total, crushed and encapsulated).

FIG. 86 presents the plasma oxycodone concentrations and patient-reported visual analog scale (VAS) scores for drug high over the first 12 hours after dosing in patients receiving either low-dose intact controlled-release oxycodone/acetaminophen (two tablets of 7.5 mg oxycodone/325 mg acetaminophen, 15 mg/650 mg total) or low-dose intact immediate-release oxycodone/acetaminophen (two tablets of 7.5 mg oxycodone/325 mg acetaminophen, 15 mg/650 mg total).

FIG. 87 presents the plasma oxycodone concentrations and patient-reported visual analog scale (VAS) scores for drug high over the first 12 hours after dosing in patients receiving either high-dose intact controlled-release oxycodone/acetaminophen (four tablets of 7.5 mg oxycodone/325 mg acetaminophen, 30 mg/1300 mg total) or high-dose intact immediate-release oxycodone/acetaminophen (four tablets of 7.5 mg oxycodone/325 mg acetaminophen, 30 mg/1300 mg total).

FIG. 88 presents the plasma oxycodone concentrations and patient-reported visual analog scale (VAS) scores for drug high over the first 12 hours after dosing in patients receiving either high-dose crushed controlled-release oxycodone/acetaminophen (four tablets of 7.5 mg oxycodone/325 mg acetaminophen, 30 mg/1300 mg total, crushed and encapsulated) or high-dose crushed immediate-release oxycodone/acetaminophen (four tablets of 7.5 mg oxycodone/325 mg acetaminophen, 30 mg/1300 mg total, crushed and encapsulated).

FIG. 89 presents a correlation plot for peak drug effects (Eₘₐₓ) for drug liking versus Cₘₐₓ. The correlation plot includes data from patients receiving the following forms of oxycodone/acetaminophen: low-dose intact controlled-release oxycodone/acetaminophen (two tablets of 7.5 mg oxycodone/325 mg acetaminophen, 15 mg/650 mg total); low-dose intact immediate-release oxycodone/acetaminophen (two tablets of 7.5 mg oxycodone/325 mg acetaminophen, 15 mg/650 mg total); high-dose intact controlled-release oxycodone/acetaminophen (four tablets of 7.5 mg oxycodone/325 mg acetaminophen, 30 mg/1300 mg total); high-dose intact immediate-release oxycodone/acetaminophen (four tablets of 7.5 mg oxycodone/325 mg acetaminophen, 30 mg/1300 mg total); high-dose crushed controlled-release oxycodone/acetaminophen (four tablets of 7.5 mg oxycodone/325 mg acetaminophen, 30 mg/1300 mg total, crushed and encapsulated); or high-dose crushed immediate-release oxycodone/acetaminophen (four tablets of 7.5 mg oxycodone/325 mg acetaminophen, 30 mg/1300 mg total, crushed and encapsulated).

FIG. 90 presents a correlation plot for peak drug effects (Eₘₐₓ) for drug high versus Cₘₐₓ. The correlation plot includes data from patients receiving the following forms of oxycodone/acetaminophen: low-dose intact controlled-release oxycodone/acetaminophen (two tablets of 7.5 mg oxycodone/325 mg acetaminophen, 15 mg/650 mg total); low-dose intact immediate-release oxycodone/acetaminophen (two tablets of 7.5 mg oxycodone/325 mg acetaminophen, 15 mg/650 mg total); high-dose intact controlled-release oxycodone/acetaminophen (four tablets of 7.5 mg oxycodone/325 mg acetaminophen, 30 mg/1300 mg total); high-dose intact immediate-release oxycodone/acetaminophen (four tablets of 7.5 mg oxycodone/325 mg acetaminophen, 30 mg/1300 mg total); high-dose crushed controlled-release oxycodone/acetaminophen (four tablets of 7.5 mg oxycodone/325 mg acetaminophen, 30 mg/1300 mg total, crushed and encapsulated); or high-dose crushed immediate-release oxycodone/acetaminophen (four tablets of 7.5 mg oxycodone/325 mg acetaminophen, 30 mg/1300 mg total, crushed and encapsulated).

FIG. 91 presents a correlation plot for area under the drug effects curve (AUE) for drug liking versus area under the concentration-time curve (AUC) for oxycodone. The correlation plot includes data from patients receiving the following forms of oxycodone/acetaminophen: low-dose intact controlled-release oxycodone/acetaminophen (two tablets of 7.5 mg oxycodone/325 mg acetaminophen, 15 mg/650 mg total); low-dose intact immediate-release oxycodone/acetaminophen (two tablets of 7.5 mg oxycodone/325 mg acetaminophen, 15 mg/650 mg total); high-dose intact controlled-release oxycodone/acetaminophen (four tablets of 7.5 mg oxycodone/325 mg acetaminophen, 30 mg/1300 mg total); high-dose intact immediate-release oxycodone/acetaminophen (four tablets of 7.5 mg oxycodone/325 mg acetaminophen, 30 mg/1300 mg total); high-dose crushed controlled-release oxycodone/acetaminophen (four tablets of 7.5 mg oxycodone/325 mg acetaminophen, 30 mg/1300 mg total, crushed and encapsulated); or high-dose crushed immediate-release oxycodone/acetaminophen (four tablets of 7.5 mg oxycodone/325 mg acetaminophen, 30 mg/1300 mg total, crushed and encapsulated).

FIG. 92 presents a correlation plot for area under the drug effects curve (AUE) for drug high versus area under the concentration-time curve (AUC) for oxycodone. The correlation plot includes data from patients receiving the following forms of oxycodone/acetaminophen: low-dose intact controlled-release oxycodone/acetaminophen (two tablets of 7.5 mg oxycodone/325 mg acetaminophen, 15 mg/650 mg total); low-dose intact immediate-release oxycodone/acetaminophen (two tablets of 7.5 mg oxycodone/325 mg acetaminophen, 15 mg/650 mg total); high-dose intact controlled-release oxycodone/acetaminophen (four tablets of 7.5 mg oxycodone/325 mg acetaminophen, 30 mg/1300 mg total); high-dose intact immediate-release oxycodone/acetaminophen (four tablets of 7.5 mg oxycodone/325 mg acetaminophen, 30 mg/1300 mg total); high-dose crushed controlled-release oxycodone/acetaminophen (four tablets of 7.5 mg oxycodone/325 mg acetaminophen, 30 mg/1300 mg total, crushed and encapsulated); or high-dose crushed immediate-release oxycodone/acetaminophen (four tablets of 7.5 mg oxycodone/325 mg acetaminophen, 30 mg/1300 mg total, crushed and encapsulated).

FIG. 93 presents a summary of the study design for the study described in Example 12, which was a randomized, double-blind, placebo-controlled, phase 3 study was conducted to evaluate the safety and efficacy of controlled-release oxycodone/acetaminophen in patients with moderate to severe acute pain.

FIG. 94 presents the mean pain intensity scores during the first two hours after first metatarsal bunionectomy for patients receiving either placebo or controlled-release oxycodone/acetaminophen (two tablets of 7.5 mg oxycodone/325 mg acetaminophen, total of 15 mg/650 mg per dose) every 12 hours.

FIG. 95 presents the mean pain intensity scores during hours 0 to 48 after first metatarsal bunionectomy for patients receiving either placebo or controlled-release oxycodone/acetaminophen (two tablets of 7.5 mg oxycodone/325 mg acetaminophen, total of 15 mg/650 mg per dose) every 12 hours.

FIG. 96 presents the proportion of patients with ≧30% reduction in pain intensity score at different times during the first 2 hours of treatment of the study described in Example 12.

FIG. 97 presents the proportion of patients “satisfied” or “very satisfied” with placebo or controlled-release oxycodone/acetaminophen, respectively, according to several measures of the Global Assessment of Satisfaction as depicted, after 48 hours of the study described in Example 12, during which patients received either placebo or two tablets of controlled-release oxycodone/acetaminophen (total 15 mg oxycodone/650 mg acetaminophen per dose) every 12 hours after a first metatarsal bunionectomy.

FIG. 98 presents the proportion of patients “satisfied” or “very satisfied” with controlled-release oxycodone/acetaminophen, according to several measures as depicted, after 7 or 14 days of open-label phase treatment, during which patients received two tablets of controlled-release oxycodone/acetaminophen (total 15 mg oxycodone/650 mg acetaminophen per dose) every 12 hours.

FIG. 99 presents a summary of the study design for Example 13, which was a multicenter, phase 3, open-label study conducted to (1) evaluate the safety and tolerability of controlled-release oxycodone/acetaminophen with up to 35 days of use in patients who were receiving only nonopioid analgesics but with pain sufficient to warrant escalation of treatment to opioid therapy and (2) evaluate the efficacy of controlled-release oxycodone/acetaminophen using changes from baseline in pain intensity, pain-related quality of life, and disease-specific quality of life.

FIG. 100 presents a summary of patient disposition for the patients participating in the study described in Example 13.

FIG. 101 presents a summary of the pain intensity score (brief pain inventory)—both at baseline and at the end of treatment—for patients receiving two tablets of controlled-release oxycodone/acetaminophen (total 15 mg oxycodone/650 mg acetaminophen per dose) every 12 hours for up to 35 days. As summarized in Example 13, patients participating in the study had either osteoarthritis of the knee or hip, or chronic low back pain.

FIG. 102 presents a summary of the Western Ontario and McMaster Universaties Arthritis Index (WOMAC) domain for “pain”—both at baseline and at the end of treatment—for patients with osteoarthritis who received two tablets of controlled-release oxycodone/acetaminophen (total 15 mg oxycodone/650 mg acetaminophen per dose) every 12 hours for up to 35 days.

FIG. 103 presents a summary of the Western Ontario and McMaster Universaties Arthritis Index (WOMAC) domain for “stiffness”—both at baseline and at the end of treatment—for patients with osteoarthritis who received two tablets of controlled-release oxycodone/acetaminophen (total 15 mg oxycodone/650 mg acetaminophen per dose) every 12 hours for up to 35 days.

FIG. 104 presents a summary of the Western Ontario and McMaster Universaties Arthritis Index (WOMAC) domain for “physical function”—both at baseline and at the end of treatment—for patients with osteoarthritis who received two tablets of controlled-release oxycodone/acetaminophen (total 15 mg oxycodone/650 mg acetaminophen per dose) every 12 hours for up to 35 days.

FIG. 105 presents a summary of the Western Ontario and McMaster Universaties Arthritis Index (WOMAC) total across all domains—both at baseline and at the end of treatment—for patients with osteoarthritis who received two tablets of controlled-release oxycodone/acetaminophen (total 15 mg oxycodone/650 mg acetaminophen per dose) every 12 hours for up to 35 days.

FIG. 106 presents stimulated human oxycodone pharmacokinetic profiles for oxycodone/acetaminophen formulations based on canine data.

FIG. 107 presents the pharmacokinetic profiles for oxycodone and acetaminophen achieved by an embodiment of the present invention.

### DETAILED DESCRIPTION OF THE INVENTION

Disclosed herein is a combination product of oxycodone and acetaminophen that has the desirable attributes of both IR and MR products. The extended release pharmaceutical composition disclosed herein comprises at least one extended release portion and, optionally, at least one immediate release portion. The extended release and immediate release portions may comprise oxycodone, acetaminophen, or combinations thereof. The at least one immediate release portion releases acetaminophen (APAP) and/or oxycodone instantly in an immediate release fashion that provides rapid onset for the attainment of therapeutically effective plasma concentrations within about the first 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition. The at least one extended release portion releases acetaminophen and/or oxycodone in an extended release fashion to maintain plasma concentrations above the minimum effective concentration for about 8-12 hours. In addition, two other features of this composition are: 1) to allow the plasma concentrations of oxycodone to fall as rapidly as an immediate release formulation to provide the same rate of termination of drug effects as the immediate release product, and 2) to allow the concentrations of APAP to fall even quicker towards the later part of the dosing interval and bring down the levels of APAP lower than those of the immediate release product. The concentrations of APAP in the last quarter of the dosing interval are comparable to the pre-dose concentrations in a multiple dose setting, allowing for the glutathione synthase enzyme cycle to replenish the body's levels of glutathione to avoid the formation of toxic intermediates with subsequent doses of APAP. Moreover, the concentrations of APAP in the later part of the dosing interval are lower than those present when administered a conventional extended release formulation. This feature has been deliberately introduced to reduce the hepatic injury due to APAP and is termed “APAP time-off”.

Abuse potential is a concern with any opioid product. The addition of APAP to the opioid, however, is likely to reduce the amount of abuse by illicit routes of administration, particularly intravenous or intranasal administration. This deterrence is likely due to the bulk (grams) that the APAP provides as well as the relative aqueous insolubility compared to freely soluble opioid salts. Further, APAP is known to be irritating to nasal passages and to make drug abusers sneeze violently when they are trying to snort it. In addition, embodiments disclosed herein may be tamper resistant in that the compositions are difficult to crush for administration intravenously or intranasally; difficult to extract with water or alcohol because the mixture becomes too viscous for injecting or snorting; and resistant to dose dumping in alcohol.

In one embodiment, the pharmaceutical composition disclosed herein, therefore, provides: 1) rapid onset of analgesia within about 15, 30, 45, or 60 minutes after administration of the composition mediated by both oxycodone and APAP, with APAP providing maximal contribution during the early phase; 2) prolonged analgesia for the entire 12 hours period, mainly contributed by oxycodone, with minimal fluctuations during this period; 3) relatively low levels of APAP toward end of dosing interval to allow for recovery of the depleted hepatic glutathione system; 4) low abuse quotient; and 5) abuse deterrence.

In a further embodiment, gastric retentive extended release pharmaceutical compositions are disclosed comprising at least one opioid wherein gastric retention of the composition is achieved by a combination of a physical property of the composition and release of the opioid. In particular, the opioid is released at a rate that is sufficient to delay gastric emptying but insufficient to cause serious adverse gastrointestinal effects. Because gastric retention of the composition is aided by release of the opioid, oral administration of the composition is independent of food intake. That is, the composition may be administered to a subject in either a fed state or a fasted state. It was discovered that, upon oral administration to a subject, the composition produces a plasma profile characterized by at least one pharmacokinetic parameter that differs by less than about 30% when the subject is in a fasted state as compared to a fed state. The food independence of this gastric retentive composition increases the convenience of administration of the composition in that it may be administered with or without food. Moreover, this property of the composition increases patient/subject compliance. The present disclosure also provides methods for administering the gastric retentive extended release composition disclosed herein, wherein the composition may be administered to a subject without regard to meals.

Headings included herein are simply for ease of reference, and are not intended to limit the disclosure in any way.

### I. DEFINITIONS

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

When introducing elements of the various embodiment(s) of the present disclosure, the articles “a”, “an”, “the” and “said” are intended to mean that there are one or more of the elements. The terms “comprising”, “including” and “having” are intended to be inclusive and mean that there may be additional elements other than the listed elements.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

The term “abuse quotient” for a pharmaceutical composition as used herein is the numerical value obtained via dividing the Cₘₐₓ for a drug by the Tₘₐₓ for the same drug. Generally speaking, the abuse quotient provides a means for predicting the degree of addictiveness of a given pharmaceutical composition. Pharmaceutical compositions with lower abuse quotients typically are less addictive compared to pharmaceutical compositions with higher abuse quotients.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “bioequivalent,” as used herein, refers to two compositions, products or methods where the 90% Confidence Intervals (CI) for AUC, partial AUC and/or Cmax are between 0.80 to 1.25.

The term “bulk density,” as used herein, refers to a property of powders and is defined as the mass of many particles of the material divided by the total volume they occupy. The total volume includes particle volume, inter-particle void volume and internal pore volume.

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

The term “friability,” as used herein, refers to the ease with which a tablet will break or fracture. The test for friability is a standard test known to one skilled in the art. Friability is measured under standardized conditions by weighing out a certain number of tablets (generally 20 tablets or less), placing them in a rotating Plexiglas drum in which they are lifted during replicate revolutions by a radial lever, and then dropped approximately 8 inches. After replicate revolutions (typically 100 revolutions at 25 rpm), the tablets are reweighed and the percentage of composition abraded or chipped is calculated.

The term “ER” as used herein refers to extended release. The phrases “extended release layer,” “ER layer,” “ER portion,” and “extended release portion” are used interchangeable in this document. Further, as used herein the “extended release layer,” “ER layer,” “ER portion,” and “extended release portion” can be either (i) a discrete part(s) of the pharmaceutical composition, (ii) integrated within the pharmaceutical composition, or (iii) a combination thereof.

The term “IR” as used herein refers to immediate release. The phrases “immediate release layer,” “IR layer,” “IR portion” and “immediate release portion” are used interchangeable in this document. In addition, as used herein the “immediate release layer,” “IR layer,” “IR portion” and “immediate release portion” can be either (i) a discrete part(s) of the pharmaceutical composition, (ii) integrated within the pharmaceutical composition, or (iii) a combination thereof.

The term “half life” as used herein refers to the time required for a drug's blood or plasma concentration to decrease by one half. This decrease in drug concentration is a reflection of its metabolism plus excretion or elimination after absorption is complete and distribution has reached an equilibrium or quasi equilibrium state. The half life of a drug in the blood may be determined graphically off of a pharmacokinetic plot of a drug's blood-concentration time plot, typically after intravenous administration to a sample population. The half life can also be determined using mathematical calculations that are well known in the art. Further, as used herein the term “half life” also includes the “apparent half-life” of a drug. The apparent half life may be a composite number that accounts for contributions from other processes besides elimination, such as absorption, reuptake, or enterohepatic recycling.

“Optional” or “optionally” means that the subsequently described element, component or circumstance may or may not occur, so that the description includes instances where the element, component, or circumstance occurs and instances where it does not.

“Partial AUC” means an area under the drug concentration-time curve (AUC) calculated using linear trapezoidal summation for a specified interval of time, for example, AUC(0-1 hr) AUC(0-2 hr), AUC(0-4 hr), AUC(0-6 hr), AUC(0-8 hr), AUC(0-(Tmax of IR product+2SD)), AUC(0-(x)hr), AUC(x-yhr), AUC(Tmax-t), AUC(0-(t)hr), AUC(Tmax of IR product+2SD)-t), or AUC(0-∞).

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid. The specific results of dissolution tests claimed herein are performed on dosage forms or pharmaceutical compositions immersed in 900 mL of 0.1 N HCl using a USP Type II apparatus at a paddle speed of either about 100 rpm or about 150 rpm and a constant temperature of about 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals.

The terms “subject” or “patient” are used interchangeably herein and refer to a vertebrate, preferably a mammal. Mammals include, but are not limited to, humans.

The term “tap density” or “tapped density,” as used herein, refers to a measure of the density of a powder. The tapped density of a pharmaceutical powder is determined using a tapped density tester, which is set to tap the powder at a fixed impact force and frequency. Tapped density by the USP method is determined by a linear progression of the number of taps.

### II. PHARMACEUTICAL COMPOSITIONS COMPRISING AN OPIOID AND AN ADDITIONAL ACTIVE PHARMACEUTICAL INGREDIENT

The present disclosure provides pharmaceutical compositions comprising at least one opioid (e.g., oxycodone) and its pharmaceutical salts and at least one other active pharmaceutical ingredient (API) (e.g., acetaminophen). It would be understood that when present in a pharmaceutical composition, the opioid would be in its salt form. For example, the pharmaceutical composition comprises at least one extended release portion comprising oxycodone, acetaminophen or a combination thereof, and an extended release component. The pharmaceutical composition may also comprise at least one immediate release portion comprising oxycodone, acetaminophen, or a combination thereof. The compositions disclosed herein are formulated to deliver therapeutic concentrations of oxycodone and acetaminophen within about the first hour after oral administration and to maintain therapeutic concentrations of oxycodone and acetaminophen for an extended period of time (e.g., 10-12 hours).

The present disclosure further provides for gastric retentive, extended release compositions comprising at least one opioid (e.g., oxycodone) and at least one other (API) (e.g., acetaminophen) that is preferably absorbed in the upper gastrointestinal tract. In general, the gastric retentive, extended release composition comprises at least one extended release portion. The extended release portion(s) may comprise at least one opioid, at least one API, or combinations thereof. The gastric retentive, extended release composition disclosed herein may further comprise at least one immediate release portion. The immediate release portion(s) may comprise at least one opioid (e.g., oxycodone), at least one other API (e.g., acetaminophen), or combinations thereof.

### (a) Active Agents

The composition disclosed herein comprises at least one opioid and at least one additional API, each of which is discussed in more detail below. In one embodiment, the same opioid or combination of opioids is present in both the at least one immediate release portion and the at least one extended release portion of the composition; and the same API or combination of APIs is present in both the at least one immediate release portion and the at least one extended release portion of the composition.

(i) Opioids

The opioid(s) useful in the present invention include adulmine, alfentanil, allocryptopine, allylprodine, alphaprodine, anileridine, aporphine, benzylmorphine, berberine, bicuculine, bicucine, bezitramide, buprenorphine, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, tramadol, and pharmaceutical salts of any of the foregoing.

In various embodiments, the extended release dosage form may comprise one, two, three, four, or more than four opioids. In another embodiment, the opioid is selected from the group consisting of oxycodone, hydrocodone, tramadol, codeine, and pharmaceutical salts of any of the foregoing. In yet another embodiment, opioid is selected from the group consisting of adulmine, alfentanil, allocryptopine, allylprodine, alphaprodine, anileridine, aporphine, benzylmorphine, berberine, bicuculine, bicucine, bezitramide, buprenorphine, bulbocaprine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, and pharmaceutical salts of any of the foregoing. In one embodiment, the extended release dosage form comprises one opioid. In a further embodiment, the dosage form comprises oxycodone.

In one embodiment, the composition may comprise from about 1.0 mg to about 500 mg of the opioid. In another embodiment, the composition may comprise from about 1.4 mg to about 400 mg of the opioid. In yet another embodiment, the amount of opioid in the composition may range from about 5 mg to about 300 mg. In still another embodiment, the amount of opioid in the composition may range from about 4 mg to about 30 mg. In another embodiment, the amount of opioid in the composition may range from about 30 mg to about 60 mg. In yet another embodiment, the amount of opioid in the composition may range from about 60 mg to about 120 mg. In an alternate embodiment, the amount of opioid in the composition may range from about 120 mg to about 300 mg. In various embodiments, the amount of opioid in the composition may be about 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, 60 mg, 62 mg, 64 mg, 66 mg, 68 mg, 70 mg, 80 mg, 90 mg, 100 mg, 110 mg, 120 mg, 130 mg, 140 mg, 150 mg, 160 mg, 170 mg, 180 mg, 190 mg, 200 mg, 220 mg, 240 mg, 260 mg, 280 mg, 300 mg, 320 mg, 340 mg, 360 mg, 380 mg, or 400 mg. In one embodiment, the amount of opioid in the composition may range from about 7.5 mg to about 30 mg. In another embodiment, the amount of opioid in the composition may range from about 7.5 mg to about 15 mg. In still another embodiment, the amount of opioid in the composition may range from about 15 mg to about 30 mg.

In additional embodiments, the dosage form comprises oxycodone, and the total amount of oxycodone present in the pharmaceutical composition can and will vary. In some embodiments, the total amount of oxycodone present in the pharmaceutical composition may range from about 2 mg to about 160 mg, about 5 mg to about 75 mg, about 5 mg to about 40 mg, or about 10 mg to about 30 mg. In another embodiment, the total amount of oxycodone in the pharmaceutical composition may range from about 5 mg to about 30 mg. In additional embodiments, the total amount of oxycodone present in the pharmaceutical composition may be about 5 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 45 mg, 50 mg, 60 mg, 70 mg, 80 mg, 100 mg, 110 mg, 120 mg, 130 mg, 140 mg, 150 mg, or 160 mg. In one embodiment, the total amount of oxycodone in the pharmaceutical composition may be about 30 mg. In another embodiment, the total amount of oxycodone in the pharmaceutical composition may be about 15 mg. In still another embodiment, the total amount of oxycodone in the pharmaceutical composition may be about 7.5 mg.

(ii) Other API

The composition disclosed herein may also comprise at least one other API. In general, the other API is preferentially absorbed in the upper gastrointestinal tract (GIT). Accordingly, optimal absorption of the API may occur in the upper GIT (i.e., duodenum, jejunum, and ileum of the small intestine), with little or no absorption in the lower GIT (i.e., cecum and colon of the large intestine).

In some embodiments, the other API may be a non-opioid analgesic. Suitable non-opioid analgesics include acetaminophen (also known as paracetamol), acetylsalicylic acid, diclofenac, diflunisol, ibuprofen, indomethacin, ketoprofen, ketorolac, naproxen, mefamanic acid, phenacetin, piroxicam, sulindac, and tolmetin. In other embodiments, the other API may be a steroidal anti-inflammatory agent such as celecoxib, deracoxib, ketoprofen, lumiracoxib, meloxicam, parecoxib, rofecoxib, or valdecoxib. In a further embodiment, the other API may be a steroidal anti-inflammatory agent such as alclometasone, dexamethasone, fluocinonide, hydrocortisone, methylprednisolone, prednisone, prednisolone, or triamcinolone. In further embodiments, the other API may be a norepinephrine transporter modulator such as tapentadol, a tricyclic antidepressant such as amitriptyline, an alpha-2 adrenergic agonist such as clonidine, a calcium channel blocker such as nimodipine, a GABA B agonist such as baclofen, a cannabinoid, a NMDA receptor antagonist, a CCK receptor antagonist, a beta blocker, or a serotonin receptor antagonist. Any of the aforementioned APIs may be in the form of a pharmaceutically acceptable salt. In various embodiments, the at least one extended release portion may comprise one, two, three, four, or more APIs. In one embodiment, one extended release portion may comprise one of the other APIs.

The amount of the other API in the gastric retentive, extended release composition can and will vary. In one embodiment, the composition may comprise from about 1.0 mg to about 1500 mg of the API. In another embodiment, the amount of API in the composition may range from about 100 mg to about 1000 mg. In still another embodiment, the amount of API in the composition may range from about 50 mg to about 500 mg. In another embodiment, the amount of API in the composition may range from about 10 mg to about 100 mg. In yet another embodiment, the amount of API in the composition may range from about 1.0 mg to about 10 mg. In one embodiment, the amount of API in the composition may range from about 250 mg to about 1300 mg. In another embodiment, the amount of API in the composition may range from about 325 mg to about 650 mg. In still another embodiment, the amount of API in the composition may range from about 650 mg to about 1300 mg.

In additional embodiments, the dosage form comprises acetaminophen, and the total amount of acetaminophen present in the pharmaceutical composition can and will vary. In one embodiment, the total amount of acetaminophen present in the pharmaceutical composition may range from about 80 mg to about 1600 mg. In another embodiment, the total amount of acetaminophen present in the pharmaceutical composition may be about 250 mg to about 1300 mg. In a further embodiment, the total amount of acetaminophen present in the pharmaceutical composition may be about 300 mg to about 600 mg. In yet another embodiment, the total amount of acetaminophen present in the pharmaceutical composition may be about 325 mg to about 650 mg. In another embodiment, the total amount of acetaminophen present in the pharmaceutical composition may be about 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 600 mg, 650 mg, 700 mg, 750 mg, 1000 mg, or 1300 mg. In one embodiment, the total amount of acetaminophen in the pharmaceutical composition may be about 650 mg. In another embodiment, the total amount of acetaminophen in the pharmaceutical composition may be about 500 mg. In yet another embodiment, the total amount of acetaminophen in the pharmaceutical composition may be about 325 mg.

### (b) Immediate Release Portion

The pharmaceutical composition disclosed herein may comprise at least one immediate release portion. In one embodiment, the at least one immediate release portion may comprise oxycodone. In another embodiment, the at least one immediate release portion may comprise acetaminophen. In a further embodiment, the at least one immediate release portion may comprise oxycodone and acetaminophen.

The at least one immediate release portion of the pharmaceutical composition is designed to release more than 80%, more than 90%, or essentially all of the opioid(s) and/or the other API(s) in the at least one immediate release portion(s) within about one hour. In one embodiment, more than 80%, more than 90%, or essentially all of the opioid(s) and/or the other API(s) in the at least one immediate release portion(s) may be released in less than about 45 minutes. In another embodiment, more than 80%, more than 90%, or essentially all of the opioid(s) and/or the other API(s) in the at least one immediate release portion(s) may be released in less that about 30 minutes. In a further embodiment, more than 80%, more than 90%, or essentially all of the opioid(s) and/or the other API(s) in the at least one immediate release portion(s) may be released in less than about 20 minutes. In yet another embodiment, more than 80%, more than 90%, or essentially all of the opioid(s) and/or the other API(s) in the at least one immediate release portion(s) may be released in less that about 15 minutes. In an alternate embodiment, more than 80%, more than 90%, or essentially all of the opioid(s) and/or the other API(s) in the at least one immediate release portion(s) may be released in less that about 10 minutes. In yet another embodiment, more than 80%, more than 90%, or essentially all of the opioid(s) and/or the other API(s) in the at least one immediate release portion may be released in less that about 5 minutes.

In some embodiments, the immediate release portion may be part of or homogeneously mixed with the extended release portion.

(i) Opioid(s)

At least one immediate release portion of the composition may comprise at least one opioid. Suitable opioids are detailed above in Section (II)(a)(i). In one embodiment, the opioid may be codeine or a salt thereof. In another embodiment, the opioid may be hydrocodone or a salt thereof. In yet another embodiment, the opioid may be hydromorphone or a salt thereof. In still another embodiment, the opioid may be morphine or a salt thereof. In a further embodiment, the opioid may be oxymorphone or a salt thereof. In an alternate embodiment, the opioid may be tramadol or a salt thereof. In another embodiment, the opioid may be oxycodone or a salt thereof.

The amount of opioid present in the at least one immediate release portion of the pharmaceutical composition can and will vary. In one embodiment, the amount of opioid in the at least one immediate release portion may range from about 0.4 mg to about 100 mg. In another embodiment, the amount of opioid in the at least one immediate release portion may range from about 1.25 mg to about 75 mg. In another embodiment, the amount of opioid in the at least one immediate release portion may range from about 1 mg to about 20 mg. In still another embodiment, the amount of opioid in the at least one immediate release portion may range from about 0.5 mg to about 10 mg. In another embodiment, the amount of opioid in the at least one immediate release portion may range from about 7.5 mg to about 15 mg. In yet another embodiment, the amount of opioid in the at least one immediate release portion may range from about 15 mg to about 30 mg. In an alternate embodiment, the amount of opioid in the at least one immediate release portion may range from about 30 mg to about 75 mg. In various embodiments, the amount of opioid in the at least one immediate release portion may be about 1.25 mg, 1.3 mg, 1.325 mg, 1.35 mg, 1.375 mg, 1.4 mg, 1.425 mg, 1.45 mg, 1.475 mg, 1.5 mg, 1.525 mg, 1.55 mg, 1.575 mg, 1.6 mg, 1.625 mg, 1.65 mg, 1.675 mg, 1.7 mg, 1.725 mg, 1.75 mg, 1.775 mg, 1.8 mg, 1.825 mg, 1.85 mg, 1.875 mg, 1.9 mg, 1.925 mg, 1.95 mg, 1.975 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6.0 mg, 6.25 mg, 6.5 mg, 6.75 mg, 7.0 mg, 7.25 mg, 7.5 mg, 7.75 mg, 8.0 mg, 8.25 mg, 8.5 mg, 8.75 mg, 9.0 mg, 9.25 mg, 9.5 mg, 9.75 mg, 10.0 mg, 0 mg, 12.0 mg, 13.0 mg, 14.0 mg, 15.0 mg, 20.0 mg, 25 mg, 30 mg, 35 mg or 40.0 mg. In one embodiment, the amount of opioid in the at least one immediate release portion may range from about 1.0 mg and about 2.0 mg, for example, about 1.25 mg, or in another example, about 1.875 mg. In yet another embodiment, the amount of opioid in the at least one immediate release portion may range from about 2.0 mg and about 3.0 mg, for example, about 2.25 mg, or in a further example, about 2.5 mg. In an additional embodiment, the amount of opioid in the at least one immediate release portion may range from 3 mg and about 4.0 mg, for example, about 3.75 mg. In another embodiment, the amount of opioid in the at least one immediate release portion may range from 7.0 mg and about 8.0 mg, for example, about 7.5 mg. In a further embodiment, the amount of opioid in the at least one immediate release portion may range from about 1.0 mg and about 5.0 mg. In yet another embodiment, the amount of opioid in the at least one immediate release portion may range from about 1.0 mg and about 4.5 mg. In another embodiment, the amount of opioid in the at least one immediate release portion may range from about 1.0 mg and about 4.0 mg. In still another embodiment, the amount of opioid in the at least one immediate release portion may range from about 1.0 mg and about 3.75 mg. In yet another embodiment, the amount of opioid in the at least one immediate release portion may range from about 1.0 mg and about 3.5 mg.

The amount of opioid present in the at least one immediate release portion(s) may be expressed as a percentage (w/w) of the total amount of opioid in the pharmaceutical composition. In one embodiment, the at least one immediate release portion may comprise from about 20% to about 40% (w/w) of the total amount of opioid present in the pharmaceutical composition. In certain embodiments, the percentage of opioid present in the at least one immediate release portion of the pharmaceutical composition may be about 20%, 21%, 22%, 23%, 24%, 25%, 26%, 27%, 28%, 29%, 30%, 31%, 32%, 33%, 34%, 35%, 36%, 37%, 38%, 39%, or 40% (w/w) of the total amount of opioid present in the composition. In one embodiment, the percentage of opioid present in the at least one immediate release portion may range from about 20% to about 30% (w/w) of the total amount of opioid present in the composition. In another embodiment, the percentage of opioid present in the at least one immediate release portion of the pharmaceutical composition may be about 25% (w/w) of the total amount of opioid present in the pharmaceutical composition.

The amount of opioid in the at least one immediate release portion also may be expressed as a percentage (w/w) of the total weight of the immediate release portion(s) of the pharmaceutical composition. In one embodiment, the amount of opioid in an immediate release portion may range from about 0.2% (w/w) to about 20% (w/w) of the total weight of such immediate release portion of the pharmaceutical composition. In another embodiment, the amount of opioid in an immediate release portion may range from about 0.5% (w/w) to about 5% (w/w) of the total weight of such immediate release portion. In various embodiments, an immediate release portion may comprise an amount of opioid that is approximately 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, 10%, 11%, 12%, 13%, 14%, 15%, 16%, 17%, 18%, 19%, or 20% (w/w) of the total weight of such immediate release portion of the pharmaceutical composition. In yet another embodiment, the amount of opioid in an immediate release portion may be about 0.5% (w/w) to about 1.0% (w/w) of the total weight of such immediate release portion of the pharmaceutical composition.

In some embodiments, the opioid in the at least one immediate release portion(s) of the pharmaceutical composition may be in the form of particles comprising opioid and at least one excipient. The at least one immediate release portion, therefore, may comprise particles of opioid(s) that are admixed with other API(s) and optional excipient(s). Suitable oxycodone particles are described in co-pending application U.S. application Ser. No. 13/166,770, filed Jun. 22, 2011, which is incorporated herein by reference in its entirety. The opioid particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

In one embodiment, the opioid found in the at least one immediate release portion of the pharmaceutical composition is oxycodone. The amount of oxycodone in the at least one immediate release portion of the pharmaceutical composition can and will vary. In one embodiment, the amount of oxycodone in the at least one immediate release portion may range from about 0.4 mg to about 100 mg. In an additional embodiment, the amount of oxycodone in the at least one immediate release portion may range from about 1 mg to about 40 mg. In a further embodiment, the amount of oxycodone in the at least one immediate release portion of the pharmaceutical composition may range from about 1 mg to about 7.5 mg. In another embodiment, the amount of oxycodone in the at least one immediate release portion may range from about 7.5 mg to about 15 mg. In yet another embodiment, the amount of oxycodone in the at least one immediate release portion may range from about 15 mg to about 40 mg. In various embodiments, the amount of oxycodone in the at least one immediate release portion may be about 1.25 mg, 1.3 mg, 1.325 mg, 1.35 mg, 1.375 mg, 1.4 mg, 1.425 mg, 1.45 mg, 1.475 mg, 1.5 mg, 1.525 mg, 1.55 mg, 1.575 mg, 1.6 mg, 1.625 mg, 1.65 mg, 1.675 mg, 1.7 mg, 1.725 mg, 1.75 mg, 1.775 mg, 1.8 mg, 1.825 mg, 1.85 mg, 1.875 mg, 1.9 mg, 1.925 mg, 1.95 mg, 1.975 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6.0 mg, 6.25 mg, 6.5 mg, 6.75 mg, 7.0 mg, 7.25 mg, 7.5 mg, 7.75 mg, 8.0 mg, 8.25 mg, 8.5 mg, 8.75 mg, 9.0 mg, 9.25 mg, 9.5 mg, 9.75 mg, 10.0 mg, 11.0 mg, 12.0 mg, 12.5 mg, 13.0 mg, 14.0 mg, 15.0 mg, 17.5 mg, 20.0 mg, 22.5 mg, 25 mg, 27.5 mg, 30 mg, 40.0 mg, 75 mg, or 100 mg. In one embodiment, the amount of oxycodone in the at least one immediate release portion may range from about 7.0 mg and about 8.0 mg, for example, about 7.5 mg. In another embodiment, the amount of oxycodone in the at least one immediate release portion may be between about 3.0 mg and about 4.0 mg, for example, about 3.75 mg. In still another embodiment, the amount of opioid in the at least one immediate release portion may range from about 1.0 mg and about 2.0 mg, for example, about 1.875 mg. In a further embodiment, the amount of oxycodone in the at least one immediate release portion may range from about 1.0 mg and about 5.0 mg. In yet another embodiment, the amount of oxycodone in the at least one immediate release portion may range from about 1.0 mg and about 4.5 mg. In another embodiment, the amount of oxycodone in the at least one immediate release portion may range from about 1.0 mg and about 4.0 mg. In still another embodiment, the amount of oxycodone in the at least one immediate release portion may range from about 1.0 mg and about 3.5 mg. In yet another embodiment, the amount of oxycodone in the at least one immediate release portion may range from about 1.0 mg and about 3.0 mg.

The amount of oxycodone present in the at least one immediate release portion(s) may be expressed as a percentage (w/w) of the total amount of oxycodone in the pharmaceutical composition. In one embodiment, the at least one immediate release portion may comprise from about 20% to about 40% (w/w) of the total amount of oxycodone present in the pharmaceutical composition. In certain embodiments, the percentage of oxycodone present in the at least one immediate release portion of the pharmaceutical composition may be about 20%, 21%, 22%, 23%, 24%, 25%, 26%, 27%, 28%, 29%, 30%, 31%, 32%, 33%, 34%, 35%, 36%, 37%, 38%, 39%, or 40% (w/w) of the total amount of oxycodone. In another embodiment, the percentage of oxycodone present in the at least one immediate release portion of the pharmaceutical composition may be about 25% (w/w) of the total amount of oxycodone present in the pharmaceutical composition.

The amount of oxycodone in the at least one immediate release portion also may be expressed as a percentage (w/w) of the total weight of the immediate release portion(s) of the pharmaceutical composition. In one embodiment, the amount of oxycodone in an immediate release portion may range from about 0.2% (w/w) to about 20% (w/w) of the total weight of such immediate release portion of the pharmaceutical composition. In another embodiment, the amount of oxycodone in an immediate release portion may range from about 0.5% (w/w) to about 5% (w/w) of the total weight of such immediate release portion. In various embodiments, an immediate release portion may comprise an amount of oxycodone that is approximately 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, 10%, 11%, 12%, 13%, 14%, 15%, 16%, 17%, 18%, 19%, or 20% (w/w) of the total weight of such immediate release portion of the pharmaceutical composition. In yet another embodiment, the amount of oxycodone in an immediate release portion may be about 0.5% (w/w) to about 1.0% (w/w) of the total weight of such immediate release portion of the pharmaceutical composition.

In some embodiments, the oxycodone of the at least one immediate release portion(s) of the pharmaceutical composition may be in the form of particles comprising oxycodone and at least one excipient. The at least one immediate release portion, therefore, may comprise particles of oxycodone that are admixed with other API(s), such as acetaminophen and optional excipient(s). Suitable oxycodone particles are described in co-pending application U.S. application Ser. No. 13/166,770, filed Jun. 22, 2011, which is incorporated herein by reference in its entirety. The oxycodone particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

(ii) Other API(s)

At least one immediate release portion of the composition may comprise at least one other API. Examples of suitable APIs that may be included in the at least one immediate release portion are presented above in Section (II)(a)(ii). In one embodiment, the other API may be acetylsalicylic acid or a salt thereof. In another embodiment, the other API may be diclofenac or a salt thereof. In yet another embodiment, the other API may be ibuprofen or a salt thereof. In still another embodiment, the other API may be indomethacin or a salt thereof. In a further embodiment, the other API may be ketoprofen or a salt thereof. In an alternate embodiment, the other API may be naproxen or a salt thereof. In another embodiment, the other API may be piroxicam or a salt thereof. In still another embodiment, the other API may be prednisolone or a salt thereof. In one embodiment, the other API may be acetaminophen or salt thereof.

The amount of the other API in the at least one immediate release portion can and will vary. In one embodiment, the immediate release portion may comprise from about 0.5 mg to about 750 mg of the API. In another embodiment, the amount of API in the at least one immediate release portion may range from about 50 mg to about 500 mg. In another embodiment, the amount of API in the at least one immediate release portion may range from about 25 mg to about 250 mg. In another embodiment, the amount of API in the at least one immediate release portion may range from about 150 mg to about 500 mg. In yet another embodiment, the amount of API in the at least one immediate release portion may range from about 0.5 mg to about 5 mg. In one embodiment, the amount of API in the at least one immediate release portion may range from about 125 mg to about 650 mg. In another embodiment, the amount of API in the at least one immediate release portion may range from about 162.5 mg to about 325 mg. In still another embodiment, the amount of API in the at least one immediate release portion may range from about 325 mg to about 650 mg. In an additional embodiment, the amount of API in the at least one immediate release portion may range from about 100 mg to about 400 mg. In still another embodiment, the amount of API in the at least one immediate release portion may range from about 125 mg to about 325 mg.

The amount of other API in the at least one immediate release portion of the pharmaceutical composition can and will vary. In general, the amount of other API present in the at least one immediate release portion may range from about 30% to about 70% (w/w) of the total amount of other API in the composition. In one embodiment, the amount of other API present in the at least one immediate release portion ranges from about 40% to about 60% (w/w) of the total amount of API in the composition. In various embodiments, the at least one immediate release portion of the composition may comprise about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% (w/w) of the total amount of API in the composition.

The amount of other API in an immediate release portion of the composition may range from about 15% to about 95% (w/w) of the total weight of such immediate release portion of the composition. In various embodiments, the amount of other API(s) in an immediate release portion may be about 15%, 20%, 25%, 30%, 35%, 40%, 45%, 50%, 55%, 60%, 65%, 68%, 69%, 70%, 71%, 72%, 73%, 74%, 75%, 76%, 77%, 78%, 79%, 80%, 82%, 84%, 86%, 88%, 90%, 92%, or 95% (w/w) of the total weight of such immediate release portion.

In embodiments in which the other API is acetaminophen, the amount of acetaminophen in the at least one immediate release may range from about 40 mg to about 800 mg. In still another embodiment, the at least one immediate release portion of the pharmaceutical composition may comprise from about 100 mg to about 600 mg of acetaminophen. In another embodiment, the at least one immediate release portion may comprise from about 150 mg to about 400 mg of acetaminophen. In a further embodiment, the amount of acetaminophen in the at least one immediate release portion may range from about 160 mg to about 325 mg. In an additional embodiment, the amount of acetaminophen in the at least one immediate release portion may range from about 100 mg to about 400 mg. In still another embodiment, the amount of acetaminophen in the at least one immediate release portion may range from about 125 mg to about 325 mg. In yet another embodiment, the amount of acetaminophen in the at least one immediate release portion may range from about 125 mg to about 400 mg.

In yet another embodiment, the amount of acetaminophen in the at least one immediate release portion may be about 120 mg, 125 mg, 130 mg, 135 mg, 140 mg, 145 mg, 150 mg, 155 mg, 160 mg, 162.5 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 500 mg, 520 mg, 600 mg, 650 mg, 700 mg, 750 mg, or 780 mg. In one embodiment, the at least one immediate release portion may comprise about 325 mg of acetaminophen. In another embodiment, the amount of acetaminophen in the at least one immediate release portion may be about 250 mg. In yet another embodiment, the amount of acetaminophen in the at least one immediate release portion may be about 162.5 mg. In still another embodiment, the amount of acetaminophen in the at least one immediate release portion may be about 125 mg.

The at least one immediate release portion(s) of the pharmaceutical composition may comprise from about 40% to about 60% (w/w) of the total amount of acetaminophen present in the pharmaceutical composition. The amount of acetaminophen in the at least one immediate release portion may be about 40%, 41%, 42%, 43%, 44%, 45%, 46%, 47%, 48%, 49%, 50%, 51%, 52%, 53%, 54%, 55%, 56%, 57%, 58%, 59%, or 60% (w/w) of the total amount of acetaminophen present in the pharmaceutical composition. In one embodiment, the percentage of acetaminophen present in the at least one immediate release portion may be about 50% (w/w) of the total amount of acetaminophen present in the pharmaceutical composition.

The amount of acetaminophen in an immediate release portion(s) of the pharmaceutical composition may range from about 20% (w/w) to about 95% (w/w) of the total weight of such immediate release portion of the composition. In various embodiments, an immediate release portion may comprise an amount of acetaminophen that is approximately about 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% (w/w) of the total weight of such immediate release portion. In one embodiment, the amount of acetaminophen in an immediate release portion may range from about 70% to about 80% (w/w) of the total weight of such immediate release portion of the pharmaceutical composition.

(iii) Excipients

The at least one immediate release portion(s) of pharmaceutical composition may further comprise at least one excipient. Suitable excipients include binders, fillers, disintegrants, lubricants, antioxidants, chelating agents, and color agents.

In one embodiment, the at least one immediate release portion(s) of the pharmaceutical composition may comprise at least one binder. Suitable binders include, without limit, starches (including corn starch and pregelatinized starch), gelatin, sugars (including sucrose, glucose, dextrose and lactose), polyethylene glycol, polyols, polyvinylalcohols, C12-C18 fatty acid alcohols, waxes, gums (e.g., guar gum, arabic gum, acacia gum, xantham gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, and polyvinyloxoazolidone. In one embodiment, the amount of binder or binders in an immediate release portion of the pharmaceutical composition may range from about 5% to about 10% (w/w) of the total weight of such immediate release portion. In various embodiments, an immediate release portion of the pharmaceutical composition may comprise at least one binder that is present in an amount that is about 5.0%, 5.25%, 5.5%, 5.75%, 6.0%, 6.25%, 6.5%, 6.75%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, or 8.7%, 8.8%, 8.9%, or 9.0% (w/w) of such immediate release portion of the composition.

In another embodiment, the at least one immediate release portion(s) of the pharmaceutical composition may comprise at least one filler. Suitable fillers include but are not limited to microcrystalline cellulose (MCC), dibasic calcium phosphate, tribasic calcium phosphate, magnesium carbonate, magnesium oxide, calcium silicate, magnesium aluminum silicate, silicon dioxide, titanium dioxide, alumina, talc, kaolin, polyvinylpyrrolidone, dibasic calcium sulfate, tribasic calcium sulfate, starch, calcium carbonate, magnesium carbonate, carbohydrates, modified starches, lactose, sucrose, dextrose, mannitol, sorbitol, and inorganic compounds. In one embodiment, the amount of filler or fillers in an immediate release portion may range from about 1.0% to about 10.0% (w/w) of the total weight of such immediate release portion. In various embodiments, an immediate release portion of the pharmaceutical composition may comprise at least one filler that is present in an amount that is about 1.0%, 1.5%, 2.0%, 2.5%, 3.0%, 3.5%, 4.0%, 4.5%, 5.0%, 5.5%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%, (w/w), of such immediate release portion of the pharmaceutical composition.

In still another embodiment, the at least one immediate release portion(s) of the pharmaceutical composition may further comprise at least one disintegrant. The disintegrant may be selected from the group consisting of croscarmellose sodium, crospovidone, alginic acid, carboxymethylcellulose calcium, carboxymethylcellulose sodium, low substituted hydroxypropylcellulose, microcrystalline cellulose, and sodium starch glycolate. In one embodiment, the amount of disintegrant in an immediate release portion may range from about 2.0% to about 15.0% (w/w) of the total weight of such immediate release portion. In some embodiments, the amount of disintegrant in an immediate release portion may be about 4.0%, 4.2%, 4.4%, 4.6%, 4.8%, 5.0%, 5.2%, 5.4%, 5.6%, 5.8%, 6.0%, 6.2%, 6.4%. 6.6%, 6.8%, or 7.0% (w/w) of such immediate release portion of the pharmaceutical composition.

In a further embodiment, the at least one immediate release portion(s) of the pharmaceutical composition may further comprise a lubricant. Useful lubricants include magnesium stearate, calcium stearate, stearic acid, and hydrogenated vegetable oil (preferably comprised of hydrogenated and refined triglycerides of stearic and palmitic acids). The lubricant may be present in an amount ranging from about 0.1% to about 3.0% (w/w) of the total weight of an immediate release portion. In certain embodiments, the amount of lubricant in at least one immediate release portion may be about 0.25%, 0.5%, 0.75%, 1.0%, 1.5%, 1.55%, 1.6%, 1.65%, 1.7%, 1.75%, 1.80%, 1.85%, 1.90%, 1.95%, or 2.0% (w/w) of the total weight of such immediate release portion.

In yet another embodiment, the at least one immediate release portion(s) of the pharmaceutical composition may comprise at least one antioxidant. Suitable antioxidants include, without limitation, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate. The amount of antioxidant present in an immediate release portion of the pharmaceutical composition may range from about 0.01% to about 4.0% (w/w), or from about 0.02% to about 0.10% (w/w) of the total weight of such immediate release portion. In various embodiments, the amount of antioxidant present in an immediate release portion of the pharmaceutical composition may be about 0.01%, 0.02%, 0.03%, 0.04%, 0.05%, 0.06%, 0.07%, 0.08%, 0.09%, 0.10%, 0.12%, 0.14%, 0.16%, 0.18%, 0.20%, 0.25%, 0.50%, 0.75%, 1.00%, 1.50%, or 2.00% (w/w) of the total weight of such immediate release portion.

In still another embodiment, the at least one immediate release portion(s) of the pharmaceutical composition may comprise at least one chelating agent. Suitable chelating agents include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA. The amount of chelating agent present in an immediate release portion of the pharmaceutical composition may range from about 0.001% to about 0.20% (w/w) of such immediate release portion. In some embodiments, the amount of chelating agent present in an immediate release portion of the pharmaceutical composition may be about 0.001%, 0.002%, 0.003%, 0.004%, 0.005%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of such immediate release portion.

In an alternate embodiment, the at least one immediate release portion of the pharmaceutical composition may comprise a color agent. Suitable color additives include, but are not limited to, food, drug and cosmetic colors (FD&C), drug and cosmetic colors (D&C), and external drug and cosmetic colors (Ext. D&C). In various embodiments, the amount of color agent present in an immediate release portion may range from about 2.0% to about 5.0% (w/w) of the total weight of such immediate release portion of the composition. In other embodiments, the amount of color agent present in an immediate release portion may be about 1.0%, 1.5%, 2.0%, 2.5%, 3.0%, 3.5%, 4.0%, 4.5%, or 5.0% (w/w) of the total weight of such immediate release portion.

### (c) Extended Release Portion

The pharmaceutical composition disclosed herein comprises at least one extended release portion. The at least one extended release portion may comprise at least one opioid, such as oxycodone, at least one other API, such as acetaminophen, or combinations thereof. The at least one extended release portion(s) further comprises at least one extended release component. The extended release component may comprise at least one extended release polymer.

The at least one extended release portion of the pharmaceutical composition is designed to release the active agents over an extended period of time. In general, the at least one extended release portion(s) provides release of the opioid(s), such as oxycodone, and/or the API(s), such as acetaminophen, for a period of time ranging from at least about 3 hours (hrs) to at least about 12 hrs. In one embodiment, the opioid(s) and/or the other API(s) may be released from the at least one extended release portion over a period of at least about 5 hours (hrs), or over a period of at least about 6 hours (hrs). In another embodiment, the at least one extended release portion may release the opioid(s) and/or the other API(s) over a period of at least about 7 hours (hrs), or over a period of at least about 8 hours (hrs). In still another embodiment, the opioid(s) and/or the other API(s) may be released from the at least one extended release portion over a period of at least about 9 hours (hrs), or over a period of at least about 10 hours (hrs). In a further embodiment, the at least one extended release portion may release the opioid(s) and/or the other API(s) over a period of at least about 11 hours (hrs), or over a period of at least about 12 hours (hrs).

(i) Opioids

At least one extended release portion of the pharmaceutical composition comprises at least one opioid. Suitable opioids are detailed above in Section (II)(a)(i). In one embodiment, the opioid may be codeine or a salt thereof. In another embodiment, the opioid may be hydrocodone or a salt thereof. In yet another embodiment, the opioid may be hydromorphone or a salt thereof. In still another embodiment, the opioid may be morphine or a salt thereof. In a further embodiment, the opioid may be oxymorphone or a salt thereof. In an alternate embodiment, the opioid may be tramadol or a salt thereof. In another embodiment, the opioid may be oxycodone or a salt thereof.

The amount of opioid present in the at least one extended release portion(s) can and will vary. In one embodiment, the amount of opioid in the at least one extended release portion may range from about 1 mg to about 300 mg. In another embodiment, the amount of opioid in the at least one extended release portion may range from about 3.75 mg to about 225 mg. In yet another embodiment, the amount of opioid in the at least one extended release portion may range from about 3.75 mg to about 120 mg. In a further embodiment, the at least one extended release portion of the pharmaceutical composition may comprise from about 1 mg to about 22.5 mg of opioid. In an additional embodiment, the at least one extended release portion of the pharmaceutical composition may comprise from about 1 mg to about 15 mg of opioid. In another embodiment, the amount of opioid in the at least one extended release portion may be from about 22.5 mg to about 45 mg. In yet another embodiment, the amount opioid in the at least one extended release portion may be from about 45 mg to about 90 mg. In still another embodiment, the amount of opioid in the at least one extended release portion may be from about 90 mg to about 225 mg. In yet another embodiment, the amount of opioid in the at least one extended release portion may be about 10 mg to about 30 mg. In yet another embodiment, the amount of opioid in the at least one extended release portion may be about 30 mg to about 60 mg.

In one embodiment, the amount of opioid in the at least one extended release portion may be from about 22 mg to about 23 mg, for example, about 22.5 mg. In another embodiment, the amount of opioid in the at least one extended release portion may be about 10 mg to about 12 mg, for example, about 11.25 mg.

In a further embodiment, the amount of opioid in the at least one extended release portion may be about 5.625 mg. In an additional embodiment, the amount of opioid in the at least one extended release portion may be about 10 mg to about 12.5 mg. In a further embodiment, the amount of opioid in the at least one extended release portion may be about 12 mg to about 18 mg. In another embodiment, the amount of opioid in the at least one extended release portion may be about 20 mg to about 25 mg. In a yet another embodiment, the amount of opioid in the at least one extended release portion may be about 2.5 mg to about 12.5 mg. In a further embodiment, the amount of opioid in the at least one extended release portion may be about 3 mg to about 8 mg. In another embodiment, the at least one extended release portion comprises about 5 mg to about 7 mg of opioid. In a further embodiment, the amount of opioid may be about 5.625 mg to about 11.25 mg. In a yet another embodiment, the amount of opioid in the at least one extended release portion may be about 3.75 mg. In a yet another embodiment, the amount of opioid in the at least one extended release portion may be about 5.625 mg. In still another embodiment, the amount of opioid in the at least one extended release portion may be about 7.5 mg. In still another embodiment, the amount of opioid in the at least one extended release portion may be about 11.25 mg. In an additional embodiment, the amount of opioid in the at least one extended release portion may be about 2.0 mg to about 7.0 mg. In a further embodiment, the amount of opioid in the at least one extended release portion may be about 3.0 mg to about 7.0 mg. In still a further embodiment, the amount of opioid in the at least one extended release portion may be about 4.0 mg to about 7.0 mg. In a another embodiment, the amount of opioid in the at least one extended release portion may be about 4.0 mg to about 6.5 mg. In yet another embodiment, the amount of opioid in the at least one extended release portion may be about 4.5 mg to about 6.5 mg.

In yet another embodiment, the amount of opioid in the at least one extended release portion may be about 1.0 mg, 1.5 mg, 2.0 mg, 2.5 mg, 3.0 mg, 3.5 mg, 3.75 mg, 4.0 mg, 4.5 mg, 5.0 mg, 5.5 mg, 5.625 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.25 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 22.5 mg, or 25 mg, 27.5 mg, 30 mg, 35 mg, 40 mg, 45 mg, or 50 mg.

The amount of opioid present in the at least one extended release portion(s) may be expressed as a percentage of the total amount of opioid in the pharmaceutical composition. In one embodiment, the at least one extended release portion of the pharmaceutical composition comprises from about 60% to about 80% (w/w) of the total amount of opioid present in the pharmaceutical composition. In certain embodiments, the percentage of opioid present in the at least one extended release portion of the pharmaceutical composition may be about 60%, 61%, 62%, 63%, 64%, 65%, 66%, 67%, 68%, 69% 70%, 71%, 72%, 73%, 74%, 75%, 76%, 77%, 78%, 79%, or 80% (w/w) of the total amount of opioid present in the composition. In one embodiment, the percentage of opioid present in the at least one extended release portion of the pharmaceutical composition may be about 75% of the total amount of opioid present in the pharmaceutical composition.

The amount of opioid in the extended release portion(s) also may be expressed as a percentage of the total weight of the extended release portion(s) of the pharmaceutical composition. In one embodiment, the amount of opioid in an extended release portion may range from about 0.3% to about 8.0% (w/w) of the total weight of the extended release portion of the pharmaceutical composition. In various embodiments, an extended release portion may comprise an amount of opioid that is approximately 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%, 4.5%, 5%, 5.5%, 6%, 6.5%, 7%, 7.5%, or 8% (w/w) of the total weight of such extended release portion of the pharmaceutical composition. In one embodiment, the amount of opioid in an extended release portion comprises about 0.5% to about 2% (w/w) of the total weight of such extended release portion of the pharmaceutical composition.

In some embodiments, the opioid of the at least one extended release portion of the composition(s) may be in the form of particles comprising the opioid and at least one excipient. Thus, the at least one extended release portion may comprise particles of opioid(s) which are admixed with the additional API(s), such as acetaminophen, and the extended release component, both of which are detailed below, as well as optional excipient(s). Suitable oxycodone particles are described in co-pending application U.S. application Ser. No. 13/166,770, filed Jun. 22, 2011, which is incorporated herein by reference in its entirety. The opioid particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

In embodiments in which the opioid is oxycodone, the amount of oxycodone in the at least one extended release portion(s) can and will vary. In one embodiment, the amount of oxycodone in the at least one extended release portion may range from about 1 mg to about 300 mg. In another embodiment, the amount of opioid in the at least one extended release portion may range from about 3.75 mg to about 225 mg. In another embodiment, the amount of opioid in the at least one extended release portion may range from about 3.75 mg to about 120 mg. In still another embodiment, the amount of opioid in the at least one extended release portion may range from about 45 mg to about 90 mg.

In a further embodiment, the at least one extended release portion of the pharmaceutical composition may comprise from about 1 mg to about 22.5 mg of oxycodone. In another embodiment, the amount of in the at least one extended release portion may be about 10 mg to about 30 mg. In yet another embodiment, the amount of oxycodone in the at least one extended release portion may be about 30 mg to about 60 mg. In still another embodiment, the amount of oxycodone in the at least one extended release portion may be about 22.5 mg to about 45 mg. In another embodiment, the at least one extended release portion comprises about 5 mg to about 7 mg of oxycodone. In a further embodiment, the amount of oxycodone may be about 5.625 mg to about 11.25 mg. In an additional embodiment, the amount of oxycodone may be about 10 mg to about 12.5 mg. In a further embodiment, the amount of oxycodone may be about 12 mg to about 18 mg. In another embodiment, the amount of oxycodone in the at least one extended release portion may be about 20 mg to about 25 mg. In an additional embodiment, the amount of oxycodone may be about 2.0 mg to about 7.0 mg. In a further embodiment, the amount of oxycodone may be about 3.0 mg to about 7.0 mg. In still a further embodiment, the amount of oxycodone may be about 4.0 mg to about 7.0 mg. In a another embodiment, the amount of oxycodone may be about 4.0 mg to about 6.5 mg. In yet another embodiment, the amount of oxycodone may be about 4.5 mg to about 6.5 mg.

In yet another embodiment, the amount of oxycodone may be about 1.0 mg, 1.5 mg, 2.0 mg, 2.5 mg, 3.0 mg, 3.5 mg, 4.0 mg, 4.5 mg, 5.0 mg, 5.5 mg, 5.625 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.25 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 22.5 mg, 25 mg, 27.5 mg, 30 mg, 35 mg, 40 mg, 45 mg, or 50 mg. In one embodiment, the amount of oxycodone in the at least one extended release portion may be from about 22 mg to about 23 mg, for example, about 22.5 mg. In another embodiment, the amount of oxycodone in the at least one extended release portion may be about 10 mg to about 12 mg, for example, about 11.25 mg. In still another embodiment, the amount of oxycodone in the at least one extended release portion may be from about 5 mg to about 6 mg, for example, about 5.625 mg.

The amount of oxycodone present in the at least one extended release portion(s) may be expressed as a percentage of the total amount of oxycodone in the pharmaceutical composition. In one embodiment, the at least one extended release portion of the pharmaceutical composition comprises from about 60% to about 80% (w/w) of the total amount of oxycodone present in the pharmaceutical composition. In certain embodiments, the percentage of oxycodone present in the at least one extended release portion of the pharmaceutical composition may be about 60%, 61%, 62%, 63%, 64%, 65%, 66%, 67%, 68%, 69% 70%, 71%, 72%, 73%, 74%, 75%, 76%, 77%, 78%, 79%, or 80% (w/w) of the total amount of oxycodone present in the composition. In one embodiment, the percentage of oxycodone present in the at least one extended release portion of the pharmaceutical composition may be about 75% of the total amount of oxycodone present in the pharmaceutical composition.

The amount of oxycodone in the extended release portion(s) also may be expressed as a percentage of the total weight of the extended release portion(s) of the pharmaceutical composition. In one embodiment, the amount of oxycodone in an extended release portion may range from about 0.3% to about 8.0% (w/w) of the total weight of the extended release portion of the pharmaceutical composition. In various embodiments, an extended release portion may comprise an amount of oxycodone that is approximately 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%, 4.5%, 5%, 5.5%, 6%, 6.5%, 7%, 7.5%, or 8% (w/w) of the total weight of such extended release portion of the pharmaceutical composition. In one embodiment, the amount of oxycodone in an extended release portion comprises about 0.5% to about 2% (w/w) of the total weight of such extended release portion of the pharmaceutical composition.

In some embodiments, the oxycodone of the at least one extended release portion of the composition(s) may be in the form of particles comprising oxycodone and at least one excipient. Thus, the at least one extended release portion may comprise particles of oxycodone which are admixed with the additional API(s), such as acetaminophen and the extended release component, both of which are detailed below, as well as optional excipients. Suitable oxycodone particles are described in co-pending application U.S. application Ser. No. 13/166,770, filed Jun. 22, 2011, which is incorporated herein by reference in its entirety. The oxycodone particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

(ii) Other API(s)

The at least one extended release portion of the pharmaceutical composition may comprise at least one other API. Examples of suitable APIs that may be included in the at least one extended release portion are presented above in Section (I)(a)(ii). In one embodiment, the other API may be acetylsalicylic acid or a salt thereof. In another embodiment, the API may be diclofenac or a salt thereof. In yet another embodiment, the API may be ibuprofen or a salt thereof. In still another embodiment, the API may be indomethacin or a salt thereof. In a further embodiment, the API may be ketoprofen or a salt thereof. In an alternate embodiment, the API may be naproxen or a salt thereof. In another embodiment, the API may be piroxicam or a salt thereof. In still another embodiment, the API may be prednisolone or a salt thereof. In one embodiment, the API may be acetaminophen or salt thereof.

The amount of the other API in the at least one extended release portion can and will vary. In one embodiment, the at least one extended release portion may comprise from about 0.5 mg to about 750 mg of the API. In another embodiment, the amount of API in the at least one extended release portion may range from about 50 mg to about 500 mg. In another embodiment, the amount of API in the at least one extended release portion may range from about 25 mg to about 250 mg. In another embodiment, the amount of API in the at least one extended release portion may range from about 150 mg to about 500 mg. In yet another embodiment, the amount of API in the at least one extended release portion may range from about 0.5 mg to about 5 mg. In one embodiment, the amount of API in the at least one extended release portion may range from about 125 mg to about 650 mg. In another embodiment, the amount of API in the at least one extended release portion may range from about 162.5 mg to about 325 mg. In still another embodiment, the amount of API in the at least one extended release portion may range from about 325 mg to about 650 mg. In yet another embodiment, the amount of API in the at least one extended release portion may range from about 100 mg to about 400 mg. In an additional embodiment, the amount of API in the at least one extended release portion may range from about 125 mg to about 325 mg.

The amount of other API(s) in the at least one extended release portion of the pharmaceutical composition can and will vary, depending upon the identity of the API(s). In general, the amount of other API present in the at least one extended release portion may range from about 30% to about 70% (w/w) of the total amount of other API in the composition. In one embodiment, the amount of other API present in the at least one extended release portion may range from about 40% to about 60% (w/w) of the total amount of other API in the composition. In various embodiments, the at least one extended release portion of the pharmaceutical composition may comprise about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% (w/w) of the total amount of other API in the composition.

The amount of other API in an extended release portion also may be expressed as a percentage of the total weight of such extended release portion of the pharmaceutical composition. In various embodiments, the amount of other API in an extended release portion may range from about 10% to about 70% (w/w) of the total weight of such extended release portion of the composition. In various embodiments, the amount of other API in an extended release portion may be about 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, 38%, 40%, 42%, 44%, 46%, 48%, 50%, 52%, 54%, 56%, 58%, 60%, 62%, 64%, 66%, 68%, or 70% (w/w) of the total weight of such extended release portion of the composition.

In embodiments in which the other API is acetaminophen, the amount of acetaminophen in the at least one extended release portion may range from about 40 mg to about 800 mg. In still another embodiment, the at least one extended release portion of the pharmaceutical composition may comprise from about 100 mg to about 600 mg of acetaminophen. In another embodiment, the at least one extended release portion may comprise from about 125 mg to about 400 mg of acetaminophen. In a further embodiment, the amount of acetaminophen in the at least one extended release portion may range from about 160 mg to about 325 mg. In yet another embodiment, the amount of acetaminophen in the at least one extended release portion may range from about 100 mg to about 400 mg. In an additional embodiment, the amount of acetaminophen in the at least one extended release portion may range from about 125 mg to about 325 mg.

In yet another embodiment, the amount of acetaminophen in the at least one extended release portion may be about 100 mg, 110 mg, 120 mg, 125 mg, 130 mg, 135 mg, 140 mg, 145 mg, 150 mg, 155 mg, 160 mg, 162.5 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 310 mg, 320 mg, 325 mg, 330 mg, 340 mg, 350 mg, 360 mg, 370 mg, 380 mg, 390 mg, 400 mg, 450 mg, 500 mg, 520 mg, 550 mg, 600 mg, 625 mg, 650 mg, 700 mg, 750 mg, 775 mg, 780 mg, or 800 mg. In one embodiment, the at least one extended release portion comprises about 325 mg of acetaminophen. In another embodiment, the amount of acetaminophen in the at least one extended release portion may be about 250 mg. In yet another embodiment, the amount of acetaminophen in the at least one extended release portion may be about 162.5 mg. In still another embodiment, the amount of acetaminophen in the at least one extended release portion may be about 125 mg.

The amount of acetaminophen in the at least one extended release portion(s) of the pharmaceutical composition may comprise from about 40% to about 60% of the total amount of acetaminophen present in the pharmaceutical composition. The amount of acetaminophen in the at least one extended release portion may be about 40%, 41%, 42%, 43%, 44%, 45%, 46%, 47%, 48%, 49%, 50%, 51%, 52%, 53%, 54%, 55%, 56%, 57%, 58%, 59%, or 60% (w/w) of the total amount of acetaminophen present in the pharmaceutical composition. In one embodiment, the percentage of acetaminophen present in the at least one extended release portion(s) of the pharmaceutical composition may be about 50% (w/w) of the total amount of acetaminophen present in the composition.

The amount of acetaminophen in an extended release portion of the pharmaceutical composition may range from about 15% to about 60% (w/w) of the total weight of such extended release portion of the pharmaceutical composition. In various embodiments, the amount of acetaminophen in an extended release portion may comprise an amount of acetaminophen that is approximately about 15%, 16%, 17%, 18%, 19%, 20%, 21%, 22%, 23%, 24%, 25%, 26%, 27%, 28%, 29%, 30%, 32%, 35%, 37%, 40%, 42%, 45%, 47%, 50%, 52%, 55%, 57%, or 60% (w/w) of the total weight of such extended release portion. In one embodiment, the amount of acetaminophen in an extended release portion may range from about 20% to about 40% (w/w) of the total weight of such extended release portion of the pharmaceutical composition.

(iii) Extended Release Component

The extended release portion(s) of the composition also comprise(s) an extended release component. Suitable extended release components include polymers, resins, hydrocolloids, hydrogels, and the like.

In one embodiment, the extended release component may comprise at least one extended release polymer. Suitable polymers for inclusion in the at least one extended release portion of the composition may be linear, branched, dendrimeric, or star polymers, and include synthetic hydrophilic polymers as well as semi-synthetic and naturally occurring hydrophilic polymers. The polymers may be homopolymers or copolymers, such as random copolymers, block copolymers, and graft copolymers. Suitable hydrophilic polymers include, but are not limited to: polyalkylene oxides, particularly poly(ethylene oxide), polyethylene glycol and poly(ethylene oxide)-poly(propylene oxide) copolymers; cellulosic polymers, such as methylcellulose, hydroxymethylcellulose, hydroxyethylcellulose, hydroxypropylcellulose, hydroxypropylmethylcellulose, and carboxymethylcellulose, microcrystalline cellulose, and polysaccharides and their derivatives; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); polyalkylene oxides; poly(olefinic alcohol)s such as poly(vinyl alcohol); poly(N-vinyl lactams) such as poly(vinyl pyrrolidone), poly(N-vinyl caprolactam), and copolymers thereof; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; xanthan gum; zein; and shellac, ammoniated shellac, shellac-acetyl alcohol, and shellac n-butyl stearate. The polymers may be used individually or in combination. Certain combinations will often provide a more controlled release of opioid(s), such as oxycodone, and API(s), such as acetaminophen, than their components when used individually. Suitable combinations include cellulose-based polymers combined with gums, such as hydroxyethyl cellulose or hydroxypropyl cellulose combined with xanthan gum, and poly(ethylene oxide) combined with xanthan gum.

In one embodiment, the extended release polymer(s) may be a cellulosic polymer, such as an alkyl substituted cellulose derivative as detailed above. In terms of their viscosities, one class of exemplary alkyl substituted celluloses includes those whose viscosity is within the range of about 100 to about 110,000 centipoise as a 2% aqueous solution at 20° C. Another class includes those whose viscosity is within the range of about 1,000 to about 4,000 centipoise as a 1% aqueous solution at 20° C.

In one embodiment, the extended release polymer(s) may be a polyalkylene oxide. In another aspect, the polyalkylene oxide may be poly(ethylene) oxide. In a further embodiment, the poly(ethylene) oxide may have an approximate molecular weight between 500,000 Daltons (Da) to about 10,000,000 Da or about 900,000 Da to about 7,000,000 Da. In yet a further embodiment, the poly(ethylene) oxide may have a molecular weight of approximately about 600,000 Da, about 700,000 Da, about 800,000 Da, about 900,000 Da, about 1,000,000 Da, about 2,000,000 Da, about 3,000,000 Da, about 4,000,000 Da, about 5,000,000 Da, about 6,000,000 Da, about 7,000,000 Da, about 8,000,000 Da, 9,000,000 Da, or 10,000,000 Da.

In another embodiment, the polyethylene oxide may be any desirable grade of POLYOX™ or any combination thereof. By way of example and without limitation, the POLYOX™ grade may be WSR N-10, WSR N-80, WSR N-750, WSR 205, WSR 1105, WSR N-12K, WSR N-60K, WSR-301, WSR Coagulant, WSR-303, WSR-308, WSR N-3000, UCARFLOC Polymer 300, UCARFLOC Polymer 302, UCARFLOC Polymer 304, and UCARFLOC Polymer 309. In one embodiment, the polyethylene oxide may have an average molecular weight of from about 100,000 Da to about 8,000,000 Da. In another embodiment, the polyethylene oxide may have an average molecular weight of about 100,000 Da, about 200,000 Da, about 300,000 Da, about 400,000 Da, about 500,000 Da, about 600,000 Da, about 700,000 Da, about 800,000 Da, about 900,000 Da, about 1,000,000 Da, about 2,000,000 Da, about 3,000,000 Da, about 4,000,000 Da, about 5,000,000 Da, about 6,000,000 Da, about 7,000,000 Da, or about 8,000,000 Da. In still another embodiment, the polyethylene oxide may have an average number of repeating ethylene oxide units (—CH2CH2O—) of about 2,000 to about 160,000. In yet another embodiment, the polyethylene oxide may have an average number of repeating ethylene oxide units of about 2,275, about 4,500, about 6,800, about 9,100, about 14,000, about 20,000, about 23,000, about 45,000, about 90,000, about 114,000, or about 159,000.

The release profile of the extended release compositions disclosed herein will depend partially upon the molecular weight of the extended release polymer(s). In certain embodiments, the polymers are of a moderate to high molecular weight (900,000 Da to 4,000,000 Da) to control release of the opioid, such as oxycodone and/or the API(s), such as acetaminophen from the composition via diffusion of the opioid(s) and/or other API out of the polymer and/or erosion of the polymer. An example of suitable polyethylene oxide polymers are those having molecular weights (viscosity average) on the order of about 900,000 Da to about 2,000,000 Da. Using a lower molecular weight (“MW”) polyethylene oxide, such as POLYOX® 1105 (900,000 MW), the release rates for drugs are higher. Using a higher molecular weight polyethylene oxide (such as POLYOX® N-60K (2,000,000 MW) or POLYOX® WSR-301 (4,000,000 MW) reduces the rate of release for drugs. In another embodiment of the invention, a hydroxypropylmethylcellulose polymer of such molecular weight is utilized so that the viscosity of a 2% aqueous solution is about 4000 cps to greater than about 100,000 cps.

The release profile of the extended release pharmaceutical composition disclosed herein may also depend upon the amount of the extended release polymer(s) in the pharmaceutical composition. In general, the release rates for all active agents may be decreased by increasing the amount of the extended release polymer(s) in the pharmaceutical composition. Stated another way, the release rates for the opioid, such as oxycodone, and/or the additional API, such as acetaminophen, may be slowed by increasing the amount of the extended release polymer(s) in the pharmaceutical composition. By way of example and without limitation, the release profile of all active agents (e.g., acetaminophen and oxycodone) may be decreased by increasing the amount of POLYOX® 1105 from about 25% by weight of the ER portion to about 35% by weight of the ER portion.

The amount of extended release polymer or polymers present in the extended release portion(s) of the pharmaceutical composition can and will vary. In one embodiment, the polymer present in an extended release portion of the composition may range from about 15% to about 70% (w/w), or about 20% to about 60% (w/w), or about 25% to about 55% (w/w) of the total weight of such extended release portion of the composition. In another embodiment, the amount of polymer present in an extended release portion of the pharmaceutical composition may range from about 30% to about 50% (w/w) of the total weight of such extended release portion. In still another embodiment, the amount of polymer present in an extended release portion of the pharmaceutical composition may range from about 35% to about 45% (w/w) of the total weight of such extended release portion. In yet another embodiment, the amount of polymer present in an extended release portion of the pharmaceutical composition may be about 30%, 35%, 40%, 45%, 50%, 55%, or 60% (w/w) of the total weight of such extended release portion. In one embodiment, the amount of polymer present in an extended release portion of the pharmaceutical composition may be about 35% (w/w) of the total weight of such extended release portion. In another embodiment, the amount of polymer present in an extended release portion of the pharmaceutical composition may be about 45% (w/w) of the total weight of such extended release portion. In one embodiment, the ER layer swells upon imbibition of fluid to a size which is about 15%, 20%, 25%, 30%, 35% 40%, 45%, 50%, 55%, 60%, 65%, 70%, 75%, 80%, 85%, 90%, 95% or 100% larger than the size of the ER layer prior to imbibition of fluid. In another embodiment, the ER layer swells upon imbibition of fluid to a size at least about 25% larger than the size of the ER layer prior to imbibition of fluid within about 15 minutes of the start of fluid imbibition. In still another embodiment, the ER layer swells upon imbibition of fluid to a size at least about 100% larger than the size of the ER layer prior to imbibition of fluid within about 45 min, 50 min, 60 min, 75 min, or 90 min of the start of fluid imbibitions.

(iv) Excipients

The extended release portion(s) of the pharmaceutical composition may further comprise at least one excipient. Suitable excipients include binders, fillers, lubricants, antioxidants, chelating agents, and color agents.

In one embodiment, the extended release portion(s) of the pharmaceutical composition may comprise at least one binder. Suitable binders include, without limit, starches (including corn starch and pregelatinized starch), gelatin, sugars (including sucrose, glucose, dextrose and lactose), polyethylene glycol, polyols, polyvinylalcohols, C12-C18 fatty acid alcohols, waxes, gums (e.g., guar gum, arabic gum, acacia gum, xanthan gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, and polyvinyloxoazolidone. In one embodiment, the amount of binder or binders in an extended release portion of the pharmaceutical composition may range from about 0.5% to about 8.0% (w/w) of such extended release portion. In various embodiments, an extended release portion of the pharmaceutical composition may comprise at least one binder that is present in an amount that is about 0.5%, 1.0%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, or 6.0%, 6.5%, 7.0%, 7.5%, or 8.0% (w/w) of such extended release portion of the composition.

In another embodiment, the at least one extended release portion(s) of the pharmaceutical composition may comprise at least one filler. Suitable fillers include but are not limited to microcrystalline cellulose (MCC), dibasic calcium phosphate, tribasic calcium phosphate, magnesium carbonate, magnesium oxide, calcium silicate, magnesium aluminum silicate, silicon dioxide, titanium dioxide, alumina, talc, kaolin, polyvinylpyrrolidone, dibasic calcium sulfate, tribasic calcium sulfate, starch, calcium carbonate, magnesium carbonate, carbohydrates, modified starches, lactose, sucrose, dextrose, mannitol, sorbitol, and inorganic compounds. In one embodiment, the amount of filler or fillers in an extended release portion may range from about 2% to about 50% (w/w) of the total weight of such extended release portion. In various embodiments, an extended release portion of the pharmaceutical composition may comprise at least one filler that is present in an amount that is about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% (w/w) of such extended release portion of the composition.

In a further embodiment, the extended release portion(s) of the pharmaceutical composition may further comprise a lubricant. Useful lubricants include magnesium stearate, calcium stearate, stearic acid, and hydrogenated vegetable oil (preferably comprised of hydrogenated and refined triglycerides of stearic and palmitic acids). The lubricant may be present in an amount ranging from about 0.1% to about 3.0% (w/w) of the total weight of the extended release portion. In certain embodiments, the amount of lubricant in an extended release portion may be about 0.25%, 0.5%, 0.75%, 1.0%, 1.5%, 1.75%, 1.80%, 1.85%, 1.90%, or 2.0% (w/w) of the total weight of such extended release portion of the composition.

In yet another embodiment, the extended release portion(s) of the pharmaceutical composition may comprise at least one antioxidant. Suitable antioxidants include, without limit, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate. The amount of antioxidant present in an extended release portion of the pharmaceutical composition may range from about 0.01% to about 4.0% (w/w), or from about 0.02% to about 0.10% (w/w). In various embodiments, the amount of antioxidant present in an extended release portion of the pharmaceutical composition may be about 0.01%, 0.02%, 0.03%, 0.04%, 0.05%, 0.06%, 0.07%, 0.08%, 0.09%, 0.10%, 0.12%, 0.14%, 0.16%, 0.18%. 0.20%, 0.25%, 0.50%, 0.75%, 1.00%, 1.50%, or 2.00% (w/w) of the total weight of such extended release portion.

In still another embodiment, the extended release portion(s) of the pharmaceutical composition may comprise at least one chelating agent. Suitable chelating agents include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA. The amount of chelating agent present in an extended release portion of the pharmaceutical composition may range from about 0.001% to about 0.20% (w/w) of such extended release portion. In some embodiments, the amount of chelating agent present in an extended release portion of the pharmaceutical composition may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of such extended release portion.

In an alternate embodiment, the extended release portion(s) of the pharmaceutical composition may comprise a color agent. Suitable color additives include, but are not limited to, food, drug and cosmetic colors (FD&C), drug and cosmetic colors (D&C), and external drug and cosmetic colors (Ext. D&C). In various embodiments, the amount of color agent present in an extended release portion may range from about 2.0% to about 5.0% (w/w) of such extended release portion of the composition. In other embodiments, the amount of color agent present in an extended release portion may be about 1.0%, 1.5%, 2.0%, 2.5%, 3.0%, 3.5%, 4.0%, 4.5%, or 5.0% (w/w) of such extended release portion.

### (d) Dosage Forms of the Pharmaceutical Composition

(i) Physical Properties

The physical form of the pharmaceutical composition disclosed herein can and will vary. In general, the pharmaceutical composition is a solid dosage form comprising at least one extended release portion and, optionally, at least one immediate release portion. Suitable solid dosage forms include tablets, caplets, capsules, encapsulated beads, and gelcaps. Non-limiting types of tablets include coated tablets, uncoated tablets, bilayer tablets, multiparticle tablets, monolithic tablets, matrix tablets, compressed tablets, and molded tablets. Non-limiting types of capsules include hard capsules and multi-layer capsules.

In one embodiment, the dosage form may be a capsule. Non-limiting examples of suitable hard capsules include hard starch capsules, hard gelatin capsules, hard cellulose capsules, and hydrogel capsules. In one example, the core of the capsule may comprise the at least one extended release portion and the shell of the capsule may comprise the at least one immediate release portion of the composition. In another example, the core of the capsule may comprises one extended release portion, comprising oxycodone, acetaminophen and an extended release component, and the shell of the capsule may comprise one immediate release portion of the composition comprising oxycodone and acetaminophen. In yet another example, the core of the capsule may comprise two extended release portions, each comprising an extended release component and one of oxycodone or acetaminophen, and the shell of the capsule may comprise two immediate release portions of the composition, each comprising one of the oxycodone and the acetaminophen. In still another embodiment, the dosage form may be a sustained release capsule comprising the oxycodone or the acetaminophen and exhibiting immediate release and/or extended release properties. In yet another embodiment, the dosage form may be a delayed release capsule comprising the oxycodone and/or acetaminophen and exhibiting immediate release and/or extended release properties. The capsule may comprise a coating. In one embodiment, the capsule may comprise an enteric coating.

In another embodiment, the dosage form may be a tablet comprising at least one extended release portion and at least one immediate release portion. The at least one immediate release portion may be adjacent to, abutting, or surrounding the at least one extended release portion. In one embodiment, the dosage form may be a bilayer tablet comprising one extended release layer comprising the oxycodone and the acetaminophen and one immediate release layer comprising the oxycodone and the acetaminophen. The bilayer tablet may comprise a coating. In another embodiment, the dosage form may be a multilayer tablet comprising two extended release portions, each comprising one of the oxycodone and the acetaminophen, and one immediate release portion comprising both the oxycodone and the acetaminophen. In yet another embodiment, the dosage form may be a multilayer tablet comprising two extended release portions, each comprising one of the oxycodone and the acetaminophen, and two immediate release portions, each comprising one of the oxycodone and the acetaminophen. In still another embodiment, the dosage form may be a sustained release tablet comprising the oxycodone and/or acetaminophen and exhibiting immediate release and/or extended release properties. In yet another embodiment, the dosage form may be a delayed release tablet comprising the oxycodone and/or acetaminophen and exhibiting immediate release and/or extended release properties. The bilayer tablet may comprise a coating. In one embodiment, the bilayer tablet may comprise an enteric coating.

In certain embodiments, the tablet may have a friability of no greater than about 0.1%, 0.2%, 0.3%, 0.4%, 0.5%, 0.7% or 1.0%. In another embodiment, the tablet may have a friability of greater than 0 but less that about 1.0%, greater than 0 but less than about 0.5%, greater than 0 but less than about 0.3%, or greater than 0 but less than about 0.2%. In still another embodiment, the tablet may have a friability of zero.

In another embodiment, the tablet may have a hardness of at least about 10 Kilopond (also known as kilopons) (kp). In some embodiments, the tablet may have a hardness of about 9 kp to about 25 kp, or about 12 kp to about 20 kp. In further embodiments, the tablet may have a hardness of about 11 kp, 12 kp, 13 kp, 14 kp, 15 kp, 16 kp, 17 kp, 18 kp, 19 kp, or 20 kp.

In additional embodiments, the tablet may have a content uniformity of from about 85 to about 115 percent by weight or from about 90 to about 110 percent by weight, or from about 95 to about 105 percent by weight. In other embodiments, the content uniformity may have a relative standard deviation (RSD) equal to or less than about 3.5%, 3.0%, 2.5%, 2.0%, 1.5%, 1.0%, or 0.5%.

The pharmaceutical composition disclosed herein includes one or more dosage forms that are designed to achieve the therapeutic concentrations of the active ingredients. In some embodiments, therefore, a therapeutically effective dose of the pharmaceutical composition may comprise one dosage form. In other embodiments, a therapeutically effective dose of the pharmaceutical composition may comprise two dosage forms. In additional embodiments, a therapeutically effective dose of the pharmaceutical composition may comprise three or more dosage forms.

In still other embodiments, prior to administration to a patient or immersion in fluid, the pharmaceutical composition may have (i) a length of approximately 18 mm, 18.01 mm, 18.02 mm, 18.03 mm, 18.04 mm, 18.05 mm, 18.06 mm, 18.07 mm, 18.08 mm, 18.09 mm, 18.1 mm, 18.11 mm, 18.12 mm, 18.13 mm, 18.14 mm, 18.15 mm, 18.16 mm, 18.17 mm, 18.18 mm, 18.19 mm, 18.2 mm, 18.21 mm, 18.22 mm, 18.23 mm, 18.24 mm, 18.25 mm, 18.26 mm, 18.27 mm, 18.28 mm, 18.29 mm, 18.3 mm, 18.31 mm, 18.32 mm, 18.33 mm, 18.34 mm, 18.35 mm, 18.36 mm, 18.37 mm, 18.38 mm, 18.39 mm, 18.4 mm, 18.41 mm, 18.42 mm, 18.43 mm, 18.44 mm, 18.45 mm, 18.46 mm, 18.47 mm, 18.48 mm, 18.49 mm, 18.5 mm, 18.51 mm, 18.52 mm, 18.53 mm, 18.54 mm, 18.55 mm, 18.56 mm, 18.57 mm, 18.58 mm, 18.59 mm, 18.6 mm, 18.61 mm, 18.62 mm, 18.63 mm, 18.64 mm, 18.65 mm, 18.66 mm, 18.67 mm, 18.68 mm, 18.69 mm, 18.7 mm, 18.71 mm, 18.72 mm, 18.73 mm, 18.74 mm, 18.75 mm, 18.76 mm, 18.77 mm, 18.78 mm, 18.79 mm, 18.8 mm, 18.81 mm, 18.82 mm, 18.83 mm, 18.84 mm, 18.85 mm, 18.86 mm, 18.87 mm, 18.88 mm, 18.89 mm, 18.9 mm, 18.91 mm, 18.92 mm, 18.93 mm, 18.94 mm, 18.95 mm, 18.96 mm, 18.97 mm, 18.98 mm, 18.99 mm, 19 mm, 19.01 mm, 19.02 mm, 19.03 mm, 19.04 mm, 19.05 mm, 19.06 mm, 19.07 mm, 19.08 mm, 19.09 mm, 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, 19.3 mm, 19.31 mm, 19.32 mm, 19.33 mm, 19.34 mm, 19.35 mm, 19.36 mm, 19.37 mm, 19.38 mm, 19.39 mm, 19.4 mm, 19.41 mm, 19.42 mm, 19.43 mm, 19.44 mm, 19.45 mm, 19.46 mm, 19.47 mm, 19.48 mm, 19.49 mm, 19.5 mm, 19.51 mm, 19.52 mm, 19.53 mm, 19.54 mm, 19.55 mm, 19.56 mm, 19.57 mm, 19.58 mm, 19.59 mm 19.6 mm, 19.61 mm, 19.62 mm, 19.63 mm, 19.64 mm, 19.65 mm, 19.66 mm, 19.67 mm, 19.68 mm, 19.69 mm, 19.7 mm, 19.71 mm, 19.72 mm, 19.73 mm, 19.74 mm, 19.75 mm, 19.76 mm, 19.77 mm, 19.78 mm, 19.79 mm, 19.8 mm, 19.81 mm, 19.82 mm, 19.83 mm, 19.84 mm, 19.85 mm, 19.86 mm, 19.87 mm, 19.88 mm, 19.89 mm, 19.9 mm, 19.91 mm, 19.92 mm, 19.93 mm, 19.94 mm, 19.95 mm, 19.96 mm, 19.97 mm, 19.98 mm, 19.99 mm, or 20 mm as measured on the major axis, (ii) a width of approximately 11 mm, 11.01 mm, 11.02 mm, 11.03 mm, 11.04 mm, 11.05 mm, 11.06 mm, 11.07 mm, 11.08 mm, 11.09 mm, 11.1 mm, 11.11 mm, 11.12 mm, 11.13 mm, 11.14 mm, 11.15 mm, 11.16 mm, 11.17 mm, 11.18 mm, 11.19 mm, 11.2 mm, 11.21 mm, 11.22 mm, 11.23 mm, 11.24 mm, 11.25 mm, 11.26 mm, 11.27 mm, 11.28 mm, 11.29 mm, 11.3 mm, 11.31 mm, 11.32 mm, 11.33 mm, 11.34 mm, 11.35 mm, 11.36 mm, 11.37 mm, 11.38 mm, 11.39 mm, 11.4 mm, 11.41 mm, 11.42 mm, 11.43 mm, 11.44 mm, 11.45 mm, 11.46 mm, 11.47 mm, 11.48 mm, 11.49 mm, 11.5 mm, 11.51 mm, 11.52 mm, 11.53 mm, 11.54 mm, 11.55 mm, 11.56 mm, 11.57 mm, 11.58 mm, 11.59 mm, 11.6 mm, 11.61 mm, 11.62 mm, 11.63 mm, 11.64 mm, 11.65 mm, 11.66 mm, 11.67 mm, 11.68 mm, 11.69 mm, 11.7 mm, 11.71 mm, 11.72 mm, 11.73 mm, 11.74 mm, 11.75 mm, 11.76 mm, 11.77 mm, 11.78 mm, 11.79 mm, 11.8 mm, 11.81 mm, 11.82 mm, 11.83 mm, 11.84 mm, 11.85 mm, 11.86 mm, 11.87 mm, 11.88 mm, 11.89 mm, 11.9 mm, 11.91 mm, 11.92 mm, 11.93 mm, 11.94 mm, 11.95 mm, 11.96 mm, 11.97 mm, 11.98 mm, 11.99 mm, 12 mm, 12.01 mm, 12.02 mm, 12.03 mm, 12.04 mm, 12.05 mm, 12.06 mm, 12.07 mm, 12.08 mm, 12.09 mm, 12.1 mm, 12.11 mm, 12.12 mm, 12.13 mm, 12.14 mm, 12.15 mm, 12.16 mm, 12.17 mm, 12.18 mm, 12.19 mm, 12.2 mm, 12.21 mm, 12.22 mm, 12.23 mm, 12.24 mm, 12.25 mm, 12.26 mm, 12.27 mm, 12.28 mm, 12.29 mm, 12.3 mm, 12.31 mm, 12.32 mm, 12.33 mm, 12.34 mm, 12.35 mm, 12.36 mm, 12.37 mm, 12.38 mm, 12.39 mm, 12.4 mm, 12.41 mm, 12.42 mm, 12.43 mm, 12.44 mm, 12.45 mm, 12.46 mm, 12.47 mm, 12.48 mm, 12.49 mm, 12.5 mm, 12.51 mm, 12.52 mm, 12.53 mm, 12.54 mm, 12.55 mm, 12.56 mm, 12.57 mm, 12.58 mm, 12.59 mm, 12.6 mm, 12.61 mm, 12.62 mm, 12.63 mm, 12.64 mm, 12.65 mm, 12.66 mm, 12.67 mm, 12.68 mm, 12.69 mm, 12.7 mm, 12.71 mm, 12.72 mm, 12.73 mm, 12.74 mm, 12.75 mm, 12.76 mm, 12.77 mm, 12.78 mm, 12.79 mm, 12.8 mm, 12.81 mm, 12.82 mm, 12.83 mm, 12.84 mm, 12.85 mm, 12.86 mm, 12.87 mm, 12.88 mm, 12.89 mm, 12.9 mm, 12.91 mm, 12.92 mm, 12.93 mm, 12.94 mm, 12.95 mm, 12.96 mm, 12.97 mm, 12.98 mm, 12.99 mm, or 13 mm, and (iii) a height or thickness of approximately 5 mm, 5.01 mm, 5.02 mm, 5.03 mm, 5.04 mm, 5.05 mm, 5.06 mm, 5.07 mm, 5.08 mm, 5.09 mm, 5.1 mm, 5.11 mm, 5.12 mm, 5.13 mm, 5.14 mm, 5.15 mm, 5.16 mm, 5.17 mm, 5.18 mm, 5.19 mm, 5.2 mm, 5.21 mm, 5.22 mm, 5.23 mm, 5.24 mm, 5.25 mm, 5.26 mm, 5.27 mm, 5.28 mm, 5.29 mm, 5.3 mm, 5.31 mm, 5.32 mm, 5.33 mm, 5.34 mm, 5.35 mm, 5.36 mm, 5.37 mm, 5.38 mm, 5.39 mm, 5.4 mm, 5.41 mm, 5.42 mm, 5.43 mm, 5.44 mm, 5.45 mm, 5.46 mm, 5.47 mm, 5.48 mm, 5.49 mm, 5.5 mm, 5.51 mm, 5.52 mm, 5.53 mm, 5.54 mm, 5.55 mm, 5.56 mm, 5.57 mm, 5.58 mm, 5.59 mm, 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, 5.8 mm, 5.81 mm, 5.82 mm, 5.83 mm, 5.84 mm, 5.85 mm, 5.86 mm, 5.87 mm, 5.88 mm, 5.89 mm, 5.9 mm, 5.91 mm, 5.92 mm, 5.93 mm, 5.94 mm, 5.95 mm, 5.96 mm, 5.97 mm, 5.98 mm, 5.99 mm, or 6 mm. In yet another embodiment, the pharmaceutical composition may have (i) a length of approximately 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, or 19.3 mm as measured on the major axis, (ii) a width of approximately 12.4 mm, 12.41 mm, 12.42 mm, 12.43 mm, 12.44 mm, 12.45 mm, 12.46 mm, 12.47 mm, 12.48 mm, 12.49 mm, or 12.5 mm, and (iii) a height or thickness of approximately 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, or 5.8 mm.

In additional embodiments, the pharmaceutical composition may expand upon immersion in fluid to have (i) a length of about 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, or 21 mm; and (ii) a width of about 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, or 14 mm within about 5 minutes of immersion in fluid. In other embodiments, the pharmaceutical composition may expand upon immersion in fluid to (i) a length of about 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, or 22 mm; and (ii) a width of about 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 10 minutes to about 15 minutes of immersion in fluid. In still other embodiments, the pharmaceutical composition may expand upon immersion in fluid to (i) a length of about 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, or 22.5 mm; and (ii) a width of about 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 20 minutes to about 25 minutes of immersion in fluid. In additional embodiments, the pharmaceutical composition may expand upon immersion in fluid to (i) a length of about 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, or 23 mm; and (ii) a width of about 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 30 minutes to about 35 minutes of immersion in fluid. In still other embodiments, the pharmaceutical composition may expand upon immersion in fluid to (i) a length of about 18 mm, 18.1 mm, 18.2 mm, 18.3 mm, 18.4 mm, 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, 23 mm, 23.1 mm, 23.2 mm, 23.3 mm, 23.4 mm, or 23.5; (ii) a width of about 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, or 16 mm; and (iii) a height or thickness of about 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, or 7 mm within about 50 minutes to about 55 minutes of immersion in fluid. In yet another embodiment, the pharmaceutical composition may expand upon immersion in fluid to (i) a length of about 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, 23 mm, 23.1 mm, 23.2 mm, 23.3 mm, 23.4 mm, or 23.5; (ii) a width of about 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, or 16 mm; and (iii) a height or thickness of about 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, or 7 mm within about 60 minutes of immersion in fluid.

In yet another embodiment, the length of the pharmaceutical composition increases by about 4%, 4.25%, 4.5% 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, or 13% within about 10 minutes of immersion in fluid. In still another embodiment, the length of the pharmaceutical composition increases by about 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 15 minutes of immersion in fluid. In yet another embodiment, the length of the pharmaceutical composition increases by about 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 20 minutes of immersion in fluid. In a further embodiment, the length of the pharmaceutical composition increases by about 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18% within about 30 minutes of immersion in fluid. In another embodiment, the length of the pharmaceutical composition increases by about 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, or 19% within about 45 minutes of immersion in fluid. In yet another embodiment, the length of the pharmaceutical composition increases by about 8%, 8.25%, 8.5%, 8.75%, 9%, 9.25% 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, or 19% within about 55 minutes of immersion in fluid. In still another embodiment, the length of the pharmaceutical composition increases by about 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, or 20% within about 60 minutes of immersion in fluid.

In a further embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 10 minutes of immersion in fluid. In still another embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18%, within about 15 minutes of immersion in fluid. In yet another embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18%, within about 20 minutes of immersion in fluid. In a further embodiment, the width of the pharmaceutical composition increases by about 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, or 24% within about 30 minutes of immersion in fluid. In another embodiment, the width of the pharmaceutical composition increases by about 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, or 25% within about 45 minutes of immersion in fluid. In yet another embodiment, the width of the pharmaceutical composition increases by about 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, or 25% within about 55 minutes of immersion in fluid. In still another embodiment, the width of the pharmaceutical composition increases by about 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, 25%, 25.25%, 25.5%, 25.75%, or 26% within about 60 minutes of immersion in fluid.

In some embodiments, the composition disclosed herein may have gastric retentive properties. These gastric retentive properties of the composition may be due to a combination of a physical property of the composition and/or the release of the opioid. In one embodiment, the gastric retentive properties of the opioid-containing extended release composition is provided by the use of a polymer. In one embodiment, the opioid-containing extended release composition comprises a gastric retentive polymer in an amount of about 1% to about 99%. In another embodiment, the opioid-containing extended release composition comprises a gastric retentive polymer in an amount of about 10% to about 80%. In yet another embodiment, the opioid-containing extended release composition comprises a gastric retentive polymer in an amount of about 20% to about 60%. In other embodiments, the opioid-containing extended release composition comprises a gastric retentive polymer in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In another embodiment, the composition may be expandable. That is, the composition has size that is small enough for oral intake, but the composition absorbs water from the gastric fluid and swells to a size that prevents its passage through the pylorus. Such a composition comprises at least one swellable, expandable material, such as a polymer, resin, hydrocolloid, hydrogel, or the like. In various embodiments, the composition may swell to a size that is about 110% to about 200% of the original volume within about 30 minutes of administration. For example, the composition may swell to approximately 115% of it original volume within 30 minutes of administration, and at a later time may swell to a volume that is 130% or more of the original volume. In other embodiments, the composition may exhibit a volume increase of two-fold or more. Additionally, the composition may become slippery upon absorption of water, which provides resistance to peristalsis and further promotes gastric retention. The swellable material degrades or erodes over a specified period of time (e.g., the dosing interval) such that the composition is no longer retained in the stomach. In one embodiment, the ER layer swells upon imbibition of fluid to a size which is about 15%, 20%, 25%, 30%, 35% 40%, 45%, 50%, 55%, 60%, 65%, 70%, 75%, 80%, 85%, 90%, 95% or 100% larger than the size of the ER layer prior to imbibition of fluid. In another embodiment, the ER layer swells upon imbibition of fluid to a size at least about 25% larger than the size of the ER layer prior to imbibition of fluid within about 15 minutes of the start of fluid imbibition. In still another embodiment, the ER layer swells upon imbibition of fluid to a size at least about 100% larger than the size of the ER layer prior to imbibition of fluid within about 45 min, 50 min, 60 min, 75 min, or 90 min of the start of fluid imbibitions.

In a further embodiment, the composition contains at least one swellable polymer. For example, the composition may include chitosan, methylcellulose, polyvinyl acetate, purified shellac, polyethylene oxide, polypropylene oxide, or an expansive polymeric film, such as one composed of polyvinyl acetate and shellac. In another embodiment, the composition may contain a combination of polymers in a matrix that is swellable. Exemplary swellable matrices are described in U.S. Pat. Nos. 6,723,340, 6,340,475, and 6,635,280, the disclosures of which are herein incorporated by reference in their entirety.

In still another embodiment, the physical property of the composition that imparts gastric retention may be the shape of the composition. For example, the composition may have a ring, tetrahedron, spiral, coil, planar disc, planar multilobe, continuous stick, sheet, oval, parallelogram, or string geometric configuration, wherein the composition is unable to pass through the pyloric sphincter. In some iterations, the composition may be folded into a pharmaceutical carrier (e.g., a gelatin capsule) or secured by readily dissolvable (e.g., gelatin) strips such that, upon dissolution of the carrier or strips, the composition unfolds in the stomach. In general, unfoldable compositions comprise biodegradable polymers such that the composition is degraded and/or reduced in size over a specified period of time (e.g., the dosing interval). In another embodiment, the composition has a diameter of greater than or equal to 7.5 mm. Exemplary shaped dosage forms are described in U.S. Pat. No. 6,488,962, the entirety of which is herein incorporated by reference.

In yet another embodiment, the physical property of the composition that imparts gastric retention may be the adhesivity of the composition. Bio-mucoadhesive compositions bind to the gastric epithelial cell surface, or mucin, and increase gastric retention time by increasing the intimacy and duration of contact between the composition and the biological membrane. Bio-mucoadhesive compositions generally comprise polycarbophil, carbopol, cholestyramine, chitosan, polymeric acids, or a natural or synthetic polymer that is capable of adhering to a biological membrane (e.g., a bioadhesive polymer) or the mucus lining of the stomach or intestinal tract (e.g., a mucoadhesive polymer). Exemplary adhesive polymers include anionic (e.g., carboxymethylcellulose, chondroitin sulfate, polyacrylic acid, pectin, carageenan, chitosan, and alginic acid), cationic (e.g., polylysine and polybrene), and neutral (e.g., polyethylene glycol, polyvinyl pyrrolidone, and dextran) polymers. Certain hydrophilic polymers tend to imbibe large amounts of water and become sticky, thereby acquiring bioadhesive properties. The adhesion of polymers to a mucus or epithelial cell surface may involve various bonding mechanisms, including physical-mechanical bonding and chemical bonding. Physical-mechanical bonding may result from the insertion of the adhesive material into the crevices or folds of the mucosa. Chemical bonds may be either covalent or non-covalent (e.g., ionic bonds, hydrogen bonds, van der Waals interactions, etc). Moreover, certain polymers may bind to specific receptor sites on the surface of cells, thereby enhancing the gastric retention. For example, certain plant lectins interact specifically with the sugar groups present in mucus or on the glycocalyx.

In still another embodiment, the physical property of the composition that imparts gastric retention may be the density of the composition. In one iteration, the composition may have a low density with sufficient buoyancy such that the composition floats over the gastric contents and remains in the stomach for a prolonged period. Floating compositions may be effervescent or noneffervescent. Effervescent compositions generally comprise matrices prepared with swellable polymers and an effervescent component. For example, the effervescent component can be either a carbonate or bicarbonate salt (e.g., sodium bicarbonate, calcium bicarbonate), an organic acid (e.g., citric acid, tartaric acid), or any combination thereof. The effervescent component can also be a floating chamber filled with vacuum, air, an inert gas, or a liquid that gasifies at body temperature. Floatability is generally achieved by generation of gas bubbles. Gas may be introduced into the floating chamber by the volatilization of an organic solvent, or by an effervescent reaction between a carbonate-bicarbonate salt and an organic acid. The matrices may be fabricated so that upon arrival in the stomach, carbon dioxide is liberated by the acidity of the gastric contents and is entrapped in the gellified matrix. This maintains the buoyancy of the composition, causing it to float. In another embodiment, the composition may also contain a polymer which exhibits floating characteristics, such as hydroxypropyl cellulose, hydroxypropyl methylcellulose, crospovidone, sodium carboxymethyl cellulose, or ethyl cellulose. In a further embodiment, the composition may comprise a device having a hollow deformable unit that converts from a collapsed to expanded form and vise versa. The unit is supported by a housing that is internally divided into two chambers separated by a pressure-sensitive movable bladder. The first chamber contains the therapeutic agent and the second contains a volatile liquid (e.g., cyclopentane, ether) that vaporizes at body temperature and imparts buoyancy to the system. The system also contains a bioerodible plug to aid in the exit from the body. Further embodiments of this two chamber system are disclosed in U.S. Pat. Nos. 3,901,232 and 3,786,813, which are hereby incorporated by reference. In still a further embodiment, the composition may contain hollow microspheres or microballoons, which cause the composition to float. The composition may also comprise floating microparticles such as polypropylene foam, Eudragit, ethyl cellulose, or polymethyl metha acylate (PMMA).

Noneffervescent compositions incorporate a high level of one or more gel-forming, highly swellable, cellulosic hydrocolloids. Upon contact with the gastric contents, these hydrocolloids hydrate and forms a colloidal gel barrier, wherein air trapped by the swollen hydrocolloid confers buoyancy to this composition. In another iteration, the composition may have a density that exceeds the density of normal gastric contents such the composition sinks to the bottom of the stomach (i.e., the antrum) where it is entrapped in the folds of the antrum and withstands the peristaltic waves of the gastric wall. In yet another iteration, the composition has a density that is greater than or equal to 1.3 g/mL.

In one embodiment, the composition is retained in the stomach due to the presence of an extended release polymer that absorbs water from the gastric contents and swells or expands to a size that cannot pass through the pyloric sphincter. As a consequence, the opioid and the other API are slowly released from the composition in the stomach and absorbed in the upper gastrointestinal tract.

In still another embodiment, the physical property of the composition that results in gastric retention may be the physical size of the composition. That is, the composition may have a size that is small enough to be orally ingested and enter the stomach, but large enough to prevent passage through the pyloric sphincter into the small intestine. In some embodiments in which the composition is designed for humans, the composition may have a length (or diameter) of more than about 7 mm, 8 mm, 9 mm, or 10 mm. In other embodiments in which the composition is designed for humans, the composition may have a length (or diameter) of more than about 11 mm, 12 mm, or 13 mm, 14 mm, 15 mm, 16 mm, 17 mm, 18 mm, 19 mm, 20 mm or longer. In still other embodiments, the composition may have (i) a length of approximately 19 mm, 19.1 mm, 19.2 mm, 19.3 mm, 19.4 mm, 19.5 mm, 19.6 mm, 19.7 mm, 19.8 mm, 19.9 mm, or 20 mm as measured on the major axis, (ii) a width of approximately 12 mm, 12.1 mm, 12.2 mm, 12.3 mm, 12.4 mm, 12.5 mm, 12.6 mm, 12.7 mm, 12.8 mm, 12.9 mm, or 13 mm as measured on the minor axis, and (iii) a height or thickness of approximately 5 mm, 5.1 mm, 5.2 mm, 5.3 mm, 5.4 mm, 5.5 mm, 5.6 mm, 5.7 mm, 5.8 mm, 5.9 mm, or 6 mm. In yet another embodiment, the composition may have (i) a length of approximately 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, or 19.3 mm as measured on the major axis, (ii) a width of approximately 12.4 mm, 12.41 mm, 12.42 mm, 12.43 mm, 12.44 mm, 12.45 mm, 12.46 mm, 12.47 mm, 12.48 mm, 12.49 mm, or 12.5 mm as measured on the minor axis, and (iii) a height or thickness of approximately 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, or 5.8 mm. In general, such compositions are designed to degrade, disintegrate, decrease in size, or collapse in a specified time interval (e.g., dosing interval) such that they may pass through the pyloric valve or be evacuated from the stomach by a housekeeper wave of gastric contractions.

In still another embodiment, the composition may contain an agent which delays the passage of the composition through the pyloric sphincter. For example, the composition may include triethanol amine myristate or propantheline.

(ii) Opioid Release

Because opioids, such as oxycodone, reduce gastric motility, the erosion time of the dosage form can be increased (thus, hindering drug release) if the opioid is not properly dosed. The gastric retentive extended release composition disclosed herein is engineered to release the opioid(s) at a rate that is sufficient to delay gastric emptying such that the composition is retained in the stomach for a longer period of time than a comparable composition that is not gastric retentive. For example, the composition may be designed to release the opioid(s) at a rate that delays gastric emptying by about 15 minutes, 30 minutes, 60 minutes, 90 minutes, 2.0 hours, 2.5 hours, 3.0 hours, 3.5 hours, 4.0 hours, 4.5 hours, or 5.0 hours. The rate of release of the opioid(s) may be manipulated by selecting a suitable extended release component for inclusion in an extended release portion of the composition. For example, in embodiments in which the extended release component is an extended release polymer, the extended release polymer generally is selected such that the composition releases the opioid(s) at a rate that delays gastric emptying by the desired amount. Additionally, the rate of release of the opioid(s) from the composition may be adjusted by selecting the proper ratio of opioid present in the at least one immediate release and the at least one extended release portions of the composition. For instance, the proportion of the opioid(s) in the at least one immediate release portion and the at least one extended release portion may be about 20:80, 21:79, 22:78, 23:77; 24:76, 25:75, 26:74, 27:73, 28;72, 29:71, 30:70, 31:69, 32:68, 33:67, 34:66, 35:65, 34:66, 35:65, 36:64, 37:63, 38:62, 39:61, or 40:60.

Additionally, the gastric retentive extended release composition is engineered to release the opioid(s) at a rate that is insufficient to cause any serious adverse gastrointestinal effects. Adverse gastrointestinal effects include, but are not limited to, intestinal hypomotility, intestinal blockage, intestinal pseudo-obstruction, abdominal distention, bloating, constipation, intestinal distress, severe intestinal contractions, colon spasms, hypoactive bowel, and increased anal sphincter tone.

(iii) Overall Composition

With the knowledge of the preferred dissolution and pharmacokinetic profiles for the opioid and the additional API, and the pharmacodynamics effects of the opioid and the additional API, as discovered by the applicants and first described herein, a composition exhibiting the same or similar dissolution and pharmacokinetic profiles and pharmacodynamics effects can be developed using any of the dosage forms discussed above. Moreover, a composition under the present invention can be developed using another dosage form that achieves the same or similar dissolution, pharmacokinetic, and pharmacodynamic profiles as the compositions disclosed herein. For example, in one embodiment, a controlled=release dosage form can be developed that exhibits pharmacokinetic and pharmacodynamic parameters (e.g., Cmax, AUC) which are within 80% to 125% at a confidence interval of 90% of those parameters for the compositions described herein. In another embodiment, a sustained release dosage form can be developed that exhibits pharmacokinetic and pharmacodynamic parameters which are within 80% to 125% at a confidence interval of 90% of those parameters for the compositions described herein. A composition could also be developed that lacks one of the specific gastric retentive dosage forms discussed above, yet, achieves the same dissolution and pharmacokinetic profiles, and exhibits the pharmacodynamic effects.

For example, a gastric retentive extended release composition as described herein may comprise an opioid, such as oxycodone, an additional API, such as acetaminophen, an immediate release portion, and a gastric retentive portion, wherein the immediate release and gastric retentive portions comprise a filler and a lubricant. In one embodiment, the immediate release and gastric retentive portions may each comprise a filler in an amount of about 5 mg to about 500 mg. In another embodiment, the immediate release and gastric retentive portions may each comprise a filler in an amount of about 20 mg to about 400 mg. In yet another embodiment, the immediate release and gastric retentive portions may each comprise a filler in an amount of about 40 mg to about 300 mg.

In one embodiment, the immediate release and gastric retentive portions may each comprise a lubricant in an amount of about 0.1 mg to about 25 mg. In another embodiment, the immediate release and gastric retentive portions may each comprise a lubricant in an amount of about 0.4 mg to about 15 mg. In still another embodiment, the immediate release and gastric retentive portions may each comprise a lubricant in an amount of about 0.7 mg to about 5 mg. In another aspect, the gastric retentive portion may further comprise between about 0 mg to about 50 mg of an effervescent agent, such as a bicarbonate salt.

As discussed above, an extended release composition without gastric retention is also described herein. In one embodiment, an extended release composition as described herein may comprise an opioid, such as oxycodone, an additional API, such as acetaminophen, an immediate release portion, and an extended release portion, wherein the immediate release and extended release portions comprise a filler in an amount of about 5 mg to about 500 mg and a lubricant in an amount of about 0.1 mg to about 25 mg. The extended release portion may comprise any suitable extended release polymer. In one embodiment, the extended release polymer is present in an amount of about 5 mg to about 500 mg. In another embodiment, the extended release polymer is present in an amount of about 20 mg to about 400 mg. In a further embodiment, the extended release polymer is present in an amount of about 40 mg to about 300 mg

For example, some exemplary formulations of the gastric retentive (Examples A-I) or sustained release (Examples J-R) dosage forms described above are as follows:

CHART A
Exemplary Gastric Retentive and Sustained Release Dosage Forms.

| | A | B | C |
|---|---|---|---|
| Immediate Release | Oxycodone HCl | Oxycodone HCl | Oxycodone HCl |
| Portion | (0-15 mg) | (0-7.5 mg) | (0-15 mg) |
| | APAP (0-325 mg) | APAP (0-175 mg) | APAP (200-325 mg) |
| | Filler (5-100 mg) | Filler (5-250 mg) | Filler (50-75 mg) |
| | Lubricant (0.1-5 mg) | Stearate salt (0.1-10 | Lubricant (2-3 mg) |
| | | mg) | |
| Gastric Retentive | Oxycodone HCl | Oxycodone HCL | Oxycodone HCl |
| Portion | (0-15 mg) | (0-7.5 mg) | (0-15 mg) |
| | APAP (0-325 mg) | APAP (0-175 mg) | APAP (100-325 mg) |
| | Polymer (5-500 mg) | Polymer (50-750 | Polymer (100-250 |
| | Filler (5-100 mg) | mg) | mg) |
| | Lubricant (0.1-5 mg) | Filler (5-250 mg) | Filler (25-50 mg) |
| | | Stearate Salt | Lubricant (1-3 mg) |
| | | (0.1-10 mg) | |
| Immediate Release | Oxycodone HCl | Oxycodone HCl | Oxycodone HCl (2-5 |
| Portion | (5-10 mg) | (5-10 mg) | mg) |
| | APAP (100-400 mg) | APAP (100-400 mg) | APAP (300-450 mg) |
| | Filler (25-50 mg) | Filler (25-50 mg) | Filler (25-75 mg) |
| | Lubricant (3-5 mg) | Lubricant (3-5 mg) | Lubricant (2-5 mg) |
| Gastric Retentive | Oxycodone HCl | Oxycodone HCl | Oxycodone HCl |
| Portion | (5-10 mg) | (5-10 mg) | (3-10 mg) |
| | APAP (50-250 mg) | APAP (50-250 mg) | APAP (50-300 mg) |
| | Filler (50-75 mg) | Filler (50-75 mg) | Filler (50-75 mg) |
| | Polycarbophil | Polymer (5-500 mg) | Polymer (100-450 mg) |
| | (5-500 mg) | Bicarbonate salt | Bicarbonate salt |
| | Lubricant (3-5 mg) | (0-10 mg) | (0-10 mg) |
| | | Lubricant (3-5 mg) | Lubricant (3-5 mg) |
| Immediate Release | Oxycodone HCl | Oxycodone HCl | Oxycodone HCl |
| Portion | (1-10 mg) | (0-15 mg) | (0-15 mg) |
| | APAP (100-325 mg) | APAP (0-325 mg) | APAP (0-325 mg) |
| | Filler (25-75 mg) | Filler (5-100 mg) | Filler (5-100 mg) |
| | Lubricant (2-5 mg) | Lubricant (0.1-5 mg) | Lubricant (0.1-5 mg) |
| Gastric Retentive | Oxycodone HCl | Oxycodone HCl | Oxycodone HCl |
| Portion | (3-10 mg) | (0-15 mg) | (0-15 mg) |
| | APAP (100-450 mg) | APAP (0-325 mg) | APAP (0-325 mg) |
| | Filler (5-100 mg) | Polyacrylate | Cholestyramine |
| | Carbopol (5-500 | (100-300 mg) | (100-300 mg) |
| | mg) | Filler (5-100 mg) | Filler (5-100 mg) |
| | Lubricant (3-5 mg) | | |
| Immediate Release | Oxycodone HCl | Oxycodone HCl | Oxycodone HCl |
| Portion | (0-15 mg) | (0-7.5 mg) | (0-15 mg) |
| | APAP (0-325 mg) | APAP (0-175 mg) | APAP (200-325 mg) |
| | Filler (5-100 mg) | Filler (5-250 mg) | Filler (50-75 mg) |
| | Lubricant (0.1-5 mg) | Stearate salt (0.1-10 mg) | Lubricant (2-3 mg) |
| Extended Release | Oxycodone HCl | Oxycodone HCL | Oxycodone HCl |
| Portion | (0-15 mg) | (0-7.5 mg) | (0-15 mg) |
| | APAP (0-325 mg) | APAP (0-175 mg) | APAP (100-325 mg) |
| | Polymer (5-500 mg) | Polymer (50-750 | Polymer (100-250 |
| | Filler (5-100 mg) | mg) | mg) |
| | Lubricant (0.1-5 mg) | Filler (5-250 mg) | Filler (25-50 mg) |
| | | Stearate Salt | Lubricant (1-3 mg) |
| | | (0.1-10 mg) | |
| Immediate Release | Oxycodone HCl | Oxycodone HCl | Oxycodone HCl (2-5 |
| Portion | (5-10 mg) | (5-10 mg) | mg) |
| | APAP (100-400 mg) | APAP (100-400 mg) | APAP (300-450 mg) |
| | Filler (25-50 mg) | Filler (25-50 mg) | Filler (25-75 mg) |
| | Lubricant (3-5 mg) | Lubricant (3-5 mg) | Lubricant (2-5 mg) |
| Extended Release | Oxycodone HCl | Oxycodone HCl | Oxycodone HCl |
| Portion | (5-10 mg) | (5-10 mg) | (3-10 mg) |
| | APAP (50-250 mg) | APAP (50-250 mg) | APAP (50-300 mg) |
| | Filler (50-75 mg) | Filler (50-75 mg) | Filler (50-75 mg) |
| | Methacrylate | Hydroxy | Propylmethyl |
| | copolymer (5-500 | propylmethyl | cellulose (100-450 |
| | mg) | cellulose (5-500 mg) | mg) |
| | Lubricant (0.1-10 | Lubricant (0.1-10 | Lubricant (0.1-10 |
| | mg) | mg) | mg) |
| Immediate Release | Oxycodone HCl | Oxycodone HCl | Oxycodone HCl |
| Portion | (1-10 mg) | (0-15 mg) | (0-15 mg) |
| | APAP (100-325 mg) | APAP (0-325 mg) | APAP (0-325 mg) |
| | Filler (25-75 mg) | Filler (5-100 mg) | Filler (5-100 mg) |
| | Lubricant (2-5 mg) | Lubricant (0.1-5 mg) | Lubricant (0.1-5 mg) |
| Extended Release | Oxycodone HCl | Oxycodone HCl | Oxycodone HCl |
| Portion | (3-10 mg) | (0-15 mg) | (0-15 mg) |
| | APAP (100-450 mg) | APAP (0-325 mg) | APAP (0-325 mg) |
| | Filler (5-100 mg) | Ethylcellulose | Polyacrylate (5-500 |
| | Alginate (5-500 mg) | (5-500 mg) | mg) |
| | Lubricant (3-5 mg) | Filler (5-100 mg) | Filler (5-100 mg) |
| Sustained Release | Oxycodone HCl | Oxycodone HCl | Oxycodone HCl |
| Formulation | (1-15 mg) | (1-15 mg) | (1-15 mg) |
| | APAP (50-650 mg) | APAP (50-650 mg) | APAP (50-650 mg) |
| | Filler (0-100 mg) | Filler (0-100 mg) | Filler (0-100 mg) |
| | Methacrylate | Hydroxy | Propylmethyl |
| | copolymer (5-500 | propylmethyl | cellulose (5-550 mg) |
| | mg) | cellulose (5-500 mg) | Lubricant (0.1-10 |
| | Lubricant (0.1-10 | Lubricant (0.1-10 | mg) |
| | mg) | mg) | |
| Sustained Release | Oxycodone HCl | Oxycodone HCl | Oxycodone HCl |
| Formulation | (1-15 mg) | (1-15 mg) | (1-15 mg) |
| | APAP (50-650 mg) | APAP (50-650 mg) | APAP (50-650 mg) |
| | Filler (0-100 mg) | Filler (0-100 mg) | Filler (0-100 mg) |
| | Alginate (5-500 mg) | Polysorbate (5-500 | Polyacrylate (5-550 |
| | Lubricant (0.1-10 | mg) | mg) |
| | mg) | Lubricant (0.1-10 | Lubricant (0.1-10 |
| | | mg) | mg) |

### (e) Abuse and Tamper Resistant Properties of the Composition

Extended release pain medications have provided many benefits to patients in the management of their chronic pain by providing a sustained release over time of a larger quantity of drug than is typically contained in an immediate release formulation. Consequently, these dosage forms (especially if they contain opioids) are attractive targets for drug abusers looking to defeat the extended release formulation to allow immediate bolus administration or “dose-dumping” of the entire drug contents of the dosage form.

Dosage forms of the pharmaceutical composition disclosed herein may be more resistant to crushing, grinding, pulverizing, or other common means used to produce a powder than an immediate release product. Accordingly, some embodiment forms are tamper resistant and less prone to abuse or misuse. For example, certain embodiments may not be crushed into a powder and snorted. Additionally, some embodiments comprising an extended release polymer may not be crushed, mixed with an aqueous solution, and injected (i.e., the resultant mixture becomes extremely viscous and cannot be effectively drawn into a syringe.)

For example, dosage forms of the pharmaceutical composition disclosed herein form a pasty semi-solid mixture when dissolved. Thus, the pharmaceutical composition is difficult to draw into a syringe and inject intravenously. The yield of active pharmaceutical ingredient(s) obtained from the pharmaceutical composition is also low (less than 20%).

Further, dosage forms of the pharmaceutical composition disclosed herein cannot easily be snorted. In order for a drug abuser to successfully snort a drug obtained from a dosage form, he must prepare a crushed, finely divided powder form of the dosage form for insufflating the powder into the nasal cavity. However, the pharmaceutical compositions disclosed herein form a clumpy, solid mass upon insufflation and do not allow acceptable absorption through the nasal tissue.

Dosage forms of the pharmaceutical composition disclosed herein also do not allow “dose dumping” caused by the deliberate introduction of alcohol into a drug abuser's stomach which accelerates the release of active ingredient(s) from the time-release formulation. The pharmaceutical compositions disclosed herein are resistant to the accelerated release of active ingredient(s).

In addition, dosage forms of the pharmaceutical composition disclosed herein do not allow for “free basing.” Successful free basing by a drug abuser requires the generation of a salt free form of the active pharmaceutical ingredient(s). This requires physical and chemical manipulation to release the active pharmaceutical ingredient(s) from its salt(s) and selective extraction from other matrix excipients. The pharmaceutical composition disclosed herein cannot be easily manipulated to generate a free base preparation.

Moreover, the tamper resistance properties of the pharmaceutical compositions disclosed herein may be increased by increasing the average molecular weight of the extended release polymer used in the pharmaceutical composition. In another embodiment, the tamper resistance properties of the pharmaceutical compositions disclosed herein may be increased by increasing the amount of the extended release polymer used in the pharmaceutical composition.

In further embodiments, the solid oral dosage forms of the pharmaceutical compositions disclosed herein exhibit substantial differences in the release profiles of oxycodone and acetaminophen when the dosage forms are crushed or ground. Indeed, the intact solid oral dosage forms surprisingly exhibit a higher release rate of both active ingredients than one that is crushed or ground. This suggests that upon grinding or crushing the solid oral dosage forms disclosed herein, the immediate release portion and extended release portion of the dosage form combine, and the hydration and swelling of the polymer(s) in the extended release portion of the dosage form retards the release of the oxycodone and acetaminophen in the immediate release portion, and may also retard the release of the oxycodone and acetaminophen in the extended release portion. Hence the incorporation of the ground or crushed components from the immediate release portion into a mixture with the ground or crushed components of the extended release portion causes the pharmaceutical composition to lose its immediate release characteristics. This feature may effectively negate a drug abuser's purpose for crushing the solid oral dosage form in the first place—to obtain an early onset of analgesia. Indeed, when the dosage forms disclosed herein are crushed or ground, the absorption of oxycodone and acetaminophen is delayed, thereby delaying the onset of euphoria as compared to when the dosage forms are ingested in an intact state. Thus, this is an unexpected tamper resistant property of the pharmaceutical compositions disclosed herein.

It was also surprisingly discovered that when an extended release dosage form disclosed herein (such as a bilayer tablet comprising an immediate release layer and an extended release layer), containing oxycodone and acetaminophen was administered to a subject, all the AUC measurements for oxycodone and acetaminophen were higher when the tablet was administered in an intact state versus when the tablet was administered in a crushed or ground state. For example, in one embodiment, the AUC measurements for either oxycodone and/or acetaminophen were about 5% to about 60% higher when a subject ingested the tablet in an intact state versus a crushed or ground state. In another embodiment, the AUC measurements for either oxycodone and/or acetaminophen were about 10% to about 50% higher when a subject ingested the tablet in an intact state versus a crushed or ground. In yet another embodiment, the AUC measurements for either oxycodone and/or acetaminophen were about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% higher when a subject ingested the tablet in an intact state versus in a crushed or ground state.

In a further embodiment, the AUC₍₀₋₁ₕᵣ₎ for either oxycodone and/or acetaminophen was about 50%, 100%, 150%, 200%, 250%, 300%, 350%, 400%, 450%, 500%, 550%, 600%, 650% 700%, 750%, 800%, 850%, 900%, 950%, or 1000% higher when a subject ingested the tablet in an intact state versus in a crushed or ground state. In another embodiment, the AUC₍₀₋₁ₕᵣ₎ for either oxycodone and/or acetaminophen will be about 50% to about 1000% higher when a subject ingested the tablet in an intact state versus in a crushed or ground state. In a further embodiment, the AUC₍₀₋₁ₕᵣ₎ for either oxycocodone and/or acetaminophen will be about 100% to about 900% higher when a subject ingested the tablet in an intact state versus in a crushed or ground state. In still a further embodiment, the AUC₍₀₋₁ₕᵣ₎ for either oxycodone and/or acetaminophen will be about 200% to about 800% higher when a subject ingested the tablet in an intact state versus in a crushed or ground state. In yet another embodiment, the AUC₍₀₋₁ₕᵣ₎ for either oxycodone and/or acetaminophen was about 300% to about 700% higher when a subject ingested the tablet in an intact state versus in a crushed or ground state.

In another embodiment, the AUC₍₀₋₂ₕᵣ₎ for either oxycodone and/or acetaminophen was about 50%, 100%, 150%, 200%, 250%, 300%, 350%, 400%, 450%, or 500% higher when a subject ingested the tablet in an intact state versus in a crushed or ground state. In another embodiment, the AUC₍₀₋₂ₕᵣ₎ for either oxycodone and/or acetaminophen will be about 50% to about 500% higher when a subject ingested the tablet in an intact state versus in a crushed or ground state. In a further embodiment, the AUC₍₀₋₂ₕᵣ₎ for either oxycodone and/or acetaminophen will be about 100% to about 400% higher when a subject ingested the tablet in an intact state versus in a crushed or ground state. In still a further embodiment, the AUC₍₀₋₂ₕᵣ₎ for either oxycodone and/or acetaminophen will be about 150% to about 300% higher when a subject ingested the tablet in an intact state versus in a crushed or ground state. In an additional embodiment, the AUC₍₀₋₂ₕᵣ₎ for either oxycodone and/or acetaminophen was about 50% to about 250% higher when a subject ingested the tablet in an intact state versus in a crushed or ground state.

In another embodiment, the AUC₍₀₋₄ₕᵣ₎ for either oxycodone and/or acetaminophen will be about 5%, 10%, 15%, 20%, 25%, 30%, 35%, 40%, 45%, 50%, 55%, 60%, 65%, 70%, 75%, 80%, 85%, 90%, 95%, or 100% higher when a subject ingested the tablet in an intact state versus in a crushed or ground state. In a further embodiment, the AUC₍₀₋₄ₕᵣ₎ for either oxycodone and/or acetaminophen will be about 25% to about 75% higher when a subject ingested the tablet in an intact state versus in a crushed or ground state. In still another embodiment, the AUC₍₀₋₈ₕᵣ₎ for either oxycodone and/or acetaminophen will be about 10%, 20%, 30%, 40%, 50%, 60%, 70% or 80% higher when a subject ingested the tablet in an intact state versus in a crushed or ground state. In an additional embodiment, the AUC₍₀₋₈ₕᵣ₎ for either oxycodone and/or acetaminophen will be about 10% to about 45% higher when a subject ingested the tablet in an intact state versus in a crushed or ground state.

In still another embodiment, the AUC₍₀₋₈ₕᵣ₎ for either oxycodone and/or acetaminophen was about 10%, 20%, 30%, 40%, 50%, 60%, 70% or 80% higher when a subject ingested the tablet in an intact state versus in a crushed or ground state. In an additional embodiment, the AUC₍₀₋₈ₕᵣ₎ for either oxycodone and/or acetaminophen was about 10% to about 45% higher when a subject ingested the tablet in an intact state versus in a crushed or ground state.

In another embodiment, the AUC_{(0-inf)} for either oxycodone and/or acetaminophen will be about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher when a subject ingested the tablet in an intact state versus in a crushed or ground state. In still another embodiment, the AUC_{(0-inf)} for either oxycodone and/or acetaminophen will be from about 5% to about 40% higher when a subject ingested the tablet in an intact state versus in a crushed or ground state. In still another embodiment, the AUC_{(0-inf)} for either oxycodone and/or acetaminophen will be from about 7% to about 30% higher when a subject ingested the tablet in an intact state versus in a crushed or ground state. In a further embodiment, the AUC_{(0-inf)} for either oxycodone and/or acetaminophen will be from about 10% to about 30% higher when a subject ingested the tablet in an intact state versus in a crushed or ground state.

In another embodiment, the AUC₍₀₋ₜ₎ for either oxycodone and/or acetaminophen will be about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher when a subject ingested the tablet in an intact state versus in a crushed or ground state. In another embodiment, the AUC₍₀₋ₜ₎ for either oxycodone and/or acetaminophen will be from about 2% to about 40% higher when a subject ingested the tablet in an intact state versus in a crushed or ground state. In still another embodiment, the AUC₍₀₋ₜ₎ for either oxycodone and/or acetaminophen will be from about 3% to about 30% higher when a subject ingested the tablet in an intact state versus in a crushed or ground state. In a further embodiment, the AUC₍₀₋ₜ₎ for either oxycodone and/or acetaminophen will be from about 4% to about 30% higher when a subject ingested the tablet in an intact state versus in a crushed or ground state. In another embodiment, the AUC₍₀₋ₜ₎ for either oxycodone and/or acetaminophen will be from about 5% to about 20% higher when a subject ingested the tablet in an intact state versus in a crushed or ground state.

Unexpectedly, the Tₘₐₓ for both oxycodone and/or acetaminophen was lower when the tablet was administered in an intact state versus when the tablet was administered in a crushed or ground state. For instance, in one embodiment, the Tₘₐₓ for either oxycodone and/or acetaminophen was lower by about 5% to about 70% when the tablet was administered in an intact state versus when the tablet was administered in a crushed or ground state. In an additional embodiment, the Tₘₐₓ for either oxycodone and/or acetaminophen was lower by about 10% to about 40% when the tablet was administered in an intact state versus when the tablet was administered in a crushed or ground state. In another embodiment, the Tₘₐₓ for either hydrocodone and/or acetaminophen will be about 20% to about 60%. In still another embodiment, the Tₘₐₓ for either oxycodone and/or acetaminophen was about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 46%, 48%, 49% or 50% higher when a subject ingested the tablet in a crushed or ground state versus in an intact state. In yet another embodiment, the Tₘₐₓ for either oxycodone and/or acetaminophen was about 25%, 50%, 75%, 100%, 125%, 150%, 175%, 200%, 225%, 250%, 300% or 325% higher when a subject ingested the tablet in a crushed or ground state versus in an intact state. In an additional embodiment, administration of the tablet to a subject produces a mean Tₘₐₓ for either oxycodone or acetaminophen that is at least about 30 minutes greater when the tablet is administered in a crushed or ground state as compared to an intact state. In a further embodiment, administration of the tablet to a subject produces a mean Tₘₐₓ for either oxycodone or acetaminophen that is at least about 30 minutes, 45 minutes, 60 minutes, 75 minutes, 90 minutes, 105 minutes, 120 minutes, 135 minutes, or 150 minutes greater when the tablet is administered in a crushed or ground state as compared to an intact state.

And the Cₘₐₓ for acetaminophen was higher when the tablet was administered in an intact state versus when the tablet was administered in a crushed or ground state. For example, in one embodiment, the Cmax for acetaminophen was about 5% to about 50% higher when the tablet was administered in an intact state versus when the tablet was administered in a crushed or ground state. In yet another embodiment, the Cₘₐₓ for acetaminophen was about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 46%, 48%, 49% or 50% higher when the tablet was administered in an intact state versus when the tablet was administered in a crushed or ground state.

In one embodiment, the abuse quotient of the tablet will be higher when the tablet is administered in an intact state versus when the tablet is administered in a crushed or ground state. For example, in another embodiment, the abuse quotient may decrease in an amount of from about 5% to about 90% when the tablet is administered in a crushed or ground state versus in an intact state. In a further embodiment, the abuse quotient may decrease in an amount from about 10% to about 80% when the tablet is administered in a crushed or ground state versus in an intact state. In yet another embodiment, the abuse quotient may decrease in an amount from about 15% to about 80% when the tablet is administered in a crushed or ground state versus in an intact state. In still another embodiment, the abuse quotient may decrease in an amount of from about 20% to about 70% when the tablet is administered in a crushed or ground state versus in an intact state. In another embodiment, the abuse quotient may decrease in an amount of about 5%, 10%, 15%, 20%, 25%, 30%, 35%, 40%, 45%, 50%, 55%, 60%, 65%, 70%, 75%, 80%, 85%, or 90% when the tablet is administered in a crushed or ground state versus in an intact state.

As a result of these pharmacokinetic parameters, a drug abuser is more likely to take the extended release dosage forms disclosed herein that comprise an immediate release portion and an extended release portion in an intact form rather than in a crushed form. Moreover, drug abusers “like” the dosage forms disclosed herein better when the dosage forms are taken in an intact state rather than in a crushed or ground state. See FDA's Guidance for Industry Document titled, “Assessment of Abuse Potential of Drug,” dated January 2010. Both overall and “at the moment” drug liking may be assessed on a bipolar visual analog scale (VAS) anchored by “strong disliking” (0), “neutral” (50), and “strong liking” (100).

In another embodiment, as the amount of oxycodone in the pharmaceutical composition increases, so does the duration of gastric retention after administration to a subject. Consequently, if a subject either intentionally or accidentally ingests a larger dose of the pharmaceutical composition than prescribed, the pharmaceutical composition will be retained in the stomach for a longer time period than an IR or traditional ER pharmaceutical composition, thereby giving a medical provider additional time to perform gastric lavage, induce vomiting, or administer activated charcoal to prevent the body from absorbing the oxycodone. In a further embodiment, the pharmaceutical composition provides a medical provider with about an additional 15 minutes, 30 minutes, 45 minutes, 60 minutes, 75 minutes, 90 minutes, 105 minutes, 2.0 hours, 2.25 hours, 2.5 hours, 2.75 hours, 3.0 hours, 3.25 hours, 3.5 hours, 3.75 hours, or 4 hours in which to prevent the absorption of oxycodone in the subject. In another embodiment, the pharmaceutical composition provides a medical provider with sufficient time to treat a subject who has overdosed on oxycodone so that death, difficulty breathing, cardiac arrest, and limp muscles do not occur in the subject.

In yet another embodiment, if vomiting is induced or naturally occurs as a result of an increased dose of oxycodone, the entire pharmaceutical composition is expelled from the subject. Thus, toxic concentrations of the oxycodone due to absorption into the subject's blood are prevented by removing the further release of oxycodone. In still another embodiment, if vomiting is induced or naturally occurs as a result of the increased dose of oxycodone about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the pharmaceutical composition is expelled from the subject. In yet another embodiment, if vomiting is induced or naturally occurs within about 30 minutes to about 60 minutes after ingestion of the increased dose of oxycodone about 50% to about 65% of the oxycodone dose is expelled from the subject.

### (f) In Vitro Release Properties of the Composition

The in vitro release rates of oxycodone and acetaminophen from the pharmaceutical compositions disclosed herein may be measured in 900 mL of 0.1 N HCl using a USP type II paddle apparatus and at a paddle speed of either about 100 rpm or 150 rpm and a constant temperature of 37° C.

In one embodiment, the at least one immediate release portion of the composition may have in vitro release rates of oxycodone and acetaminophen as follows: more than about 90% of the oxycodone and/or the acetaminophen present in the at least one immediate release portion may be released within about 15 minutes, or essentially 100% of the oxycodone and/or the acetaminophen present in the at least one immediate release portion may be released within about 15 minutes. In another embodiment, more than about 90% of the oxycodone and/or the acetaminophen present in the at least one immediate release portion may be released within about 5 minutes. In yet another embodiment, essentially 100% of the oxycodone and/or acetaminophen present in the at least one immediate release portion may be released within about 5 minutes.

In one embodiment, the at least one extended release portion of the composition may have in vitro release rates of oxycodone as follows: from about 1% to about 20% of the oxycodone present in the at least one extended release portion may be released within about 15 minutes, from about 35% to about 55% of the oxycodone present in the at least one extended release portion may be released within about 2 hours, from about 65% to about 85% of the oxycodone present in the at least one extended release portion may be released within about 4 hours, and at least about 90% of the oxycodone present in the at least one extended release portion may be released within about 8 hours.

In yet another embodiment, the at least one extended release portion may have in vitro release rates of oxycodone as follows: from about 1% to about 10% of the oxycodone present in the at least one extended release portion may be released within about 15 minutes, from about 40% to about 50% of the oxycodone present in the at least one extended release portion may be released within about 2 hours, from about 70% to about 80% of the oxycodone present in the at least one extended release portion may be released within about 4 hours, and from about 90% to about 100% of the oxycodone present in the at least one extended release portion may be released within about 8 hours.

In one embodiment, the at least one extended release portion may have in vitro release rates of acetaminophen as follows: from about 1′)/0 to about 15% of the acetaminophen present in the at least one extended release portion may be released within about 15 minutes, from about 25% to about 40% of the acetaminophen present in the at least one extended release portion may be released within about 2 hours, from about 50% to about 65% of the acetaminophen present in the at least one extended release portion may be released within about 4 hours, and from about 80% to about 95% of the acetaminophen present in the at least one extended release portion may be released within about 8 hours.

In another embodiment, the at least one extended release portion of the composition may have in vitro release rates of acetaminophen as follows: from about 1′)/0 to about 10% of the acetaminophen present in the at least one extended release portion may be released within about 15 minutes, from about 25% to about 35% of the acetaminophen present in the at least one extended release portion may be released within about 2 hours, from about 55% to about 65% of the acetaminophen present in the at least one extended release portion may be released within about 4 hours, and from about 80% to about 90% of the acetaminophen present in the at least one extended release portion may be released within about 8 hours.

In another embodiment, the at least one extended release portion of the composition may have in vitro release rates of acetaminophen as follows: from about 1% to about 10% of the acetaminophen present in the at least one extended release portion may be released within about 15 minutes, from about 20% to about 50% of the acetaminophen present in the at least one extended release portion may be released within about 2 hours, from about 35% to about 75% of the acetaminophen present in the at least one extended release portion may be released within about 4 hours, and from about 65% to about 100% of the acetaminophen present in the at least one extended release portion may be released within about 8 hours.

In one embodiment, the in vitro release rates of oxycodone from the composition may be as follows: about 20% to about 45% of oxycodone may be released from the composition within about 15 minutes, from about 50% to about 75% of oxycodone may be released from the composition in about 2 hours, from about 70% to about 95% of oxycodone may be released from the composition within about 4 hours, and from about 90% to about 100% of oxycodone may be released from the composition within about 8 hours.

In one embodiment, the in vitro release rates of oxycodone from the composition may be as follows: about 25% to about 35% of oxycodone may be released from the composition within about 15 minutes, from about 40% to about 80% of oxycodone may be released from the composition in about 2 hours, from about 70% to about 85% of oxycodone may be released from the composition within about 4 hours, and from about 90% to about 100% of oxycodone may be released from the composition within about 8 hours.

In another embodiment, the pharmaceutical composition disclosed herein may have in vitro release rates of oxycodone as follows: about 25% to about 30% of oxycodone may be released from the pharmaceutical composition within about 15 minutes, from about 50% to about 60% of oxycodone may be released from the pharmaceutical composition within about 2 hours, from about 70% to about 80% of oxycodone may be released from the pharmaceutical composition within about 4 hours, and from about 90% to about 95% of oxycodone may be released from the pharmaceutical composition within about 8 hours.

In one embodiment, the in vitro release rates of acetaminophen from the composition may be as follows: from about 40% to about 65% of acetaminophen may be released from the composition in about 15 minutes, from about 55% to about 80% of acetaminophen may be released from the composition in about 2 hours, from about 65% to about 95% of acetaminophen may be released from the composition in about 4 hours, and from about 80% to about 100% of acetaminophen may be released from the composition in about 8 hours.

In one embodiment, the in vitro release rates of acetaminophen from the composition may be as follows: from about 30% to about 70% of acetaminophen may be released from the composition in about 15 minutes, from about 50% to about 90% of acetaminophen may be released from the composition in about 2 hours, from about 60% to about 95% of acetaminophen may be released from the composition in about 4 hours, and from about 90% to about 100% of acetaminophen may be released from the composition in about 8 hours.

In another embodiment, the in vitro release rates of acetaminophen from the pharmaceutical composition disclosed herein may be as follows: from about 50% to about 55% of acetaminophen may be released from the pharmaceutical composition within about 15 minutes, from about 60% to about 70% of acetaminophen may be released from the pharmaceutical composition within about 2 hours, from about 75% to about 85% of acetaminophen may be released from the pharmaceutical composition within about 4 hours, and from about 90% to about 100% of acetaminophen may be released from the pharmaceutical composition within about 8 hours.

In another embodiment, about 90% to about 100% of the IR dose of acetaminophen is released within about 15 minutes, 30 minutes, 45 minutes or 60 minutes after oral administration. In one embodiment, the pharmaceutical composition provides a dissolution profile wherein about 20% to about 65%, about 35% to about 55% or about 40% to about 50% of the ER dose of acetaminophen remains in the ER layer between about 1 and 2 hours after administration.

In yet another embodiment, the pharmaceutical composition provides a dissolution profile wherein about 50% to about 95% of the ER dose of acetaminophen remains in the ER layer between about 1 and 2 hours after administration. In another embodiment, the dosage form provides a dissolution profile wherein about 15% to about 40% of the ER dose of acetaminophen is released from the ER layer between about 1 and 2 hours after administration. In one embodiment, not more than 50% of the ER dose of acetaminophen is released within about the first hour. In a further embodiment, not more than 45% or not more than 40% of the ER dose of acetaminophen is released within about the first hour.

In another embodiment, not more than 85% of the ER dose of acetaminophen is released within about 4 hours. In yet another embodiment, not less than 50% is released after about 6 hours. In yet another embodiment, not less than 55% is released after about 6 hours. In one embodiment, the ER dose of acetaminophen is released over a time period of about 6 to 12, about 8 to 10, or about 9 to 10 hours in vitro. In another embodiment, the ER dose of acetaminophen is released over a time period of about 7 hours, 8 hours, 9 hours, 10 hours, 11 hours or 12 hours in vitro. In one embodiment, at least 80% or 85% of the ER dose of acetaminophen is released over a time period of about 7 hours, 8 hours, 9 hours, 10 hours, 11 hours or 12 hours in vitro. In another embodiment, at least 90% or 95% of the ER dose of acetaminophen is released over a time period of about 7 hours, 8 hours, 9 hours, 10 hours, 11 hours or 12 hours in vitro.

Additionally, the in vitro release rates of oxycodone and acetaminophen from the pharmaceutical composition generally are not affected by low concentrations of ethanol (i.e., from about 5% v/v to about 20% v/v) when measured in 900 mL of 0.1 N HCl containing the desired percentage of ethanol using a USP type II paddle apparatus and at a paddle speed of about 150 rpm and a constant temperature of 37° C. For example, from about 25% to about 35% of oxycodone and about 50% to about 55% of acetaminophen may be released from the pharmaceutical composition within about 15 minutes when measured in the presence of 5% to 20% ethanol, and from about 50% to about 65% of oxycodone and from about 60% to about 70% of acetaminophen may be released from the pharmaceutical composition within about 2 hour when measured in the presence of 5% to 20% ethanol.

The in vitro release rates of oxycodone and acetaminophen from the pharmaceutical compositions disclosed herein generally are reduced, however, in the presence of 40% ethanol. For example, from about 5% to about 15% of the oxycodone and from about 15% to about 25% of the acetaminophen may be released from the pharmaceutical composition within about 15 minutes when measured in the presence of 40% ethanol, and from about 35% to about 45% of oxycodone and from about 45% to about 55% of acetaminophen may be released from the pharmaceutical composition within about 2 hours when measured in the presence of 40% ethanol.

Stated another way, less oxycodone is extracted from the pharmaceutical composition by a solution of 0.1 N HCl and 40% ethanol than is extracted by a solution of 0.1 N HCl. In some embodiments, less than about 75% of the oxycodone that is released in the presence of 0.1 N HCl may be released in the presence of 0.1 N HCl containing 40% ethanol. In additional embodiments, less than about 70%, 65%, 60%, 55%, 50%, 45%, or 40% of the oxycodone that may be released in the presence of 0.1 N HCl may be released in the presence of 0.1 N HCl and 40% ethanol. For example, less than about 40% of the oxycodone that may be released in the presence of 0.1 N HCl in about 15 minutes may be released in the presence of 0.1N HCl and 40% ethanol within about 15 minutes. In other embodiments, less than about 60% of the oxycodone that may be released in the presence of 0.1 N HCl in about 30 minutes may be released in the presence of 0.1 N HCl and 40% ethanol within about 30 minutes. In additional embodiments, less than about 75% of the oxycodone that may be released in the presence of 0.1 N HCl in about 2 hours may be released in the presence of 0.1 N HCl and 40% ethanol within about 2 hours.

### (g) Stability Data for the Pharmaceutical Composition

In one embodiment, p-aminophenol may be present in the pharmaceutical composition as a degradation product of acetaminophen in any amount up to and including, but no more than, about 100 ppm. In other embodiments, p-aminophenol may be present in the pharmaceutical composition as a degradation product of acetaminophen in an amount of about 0.2 ppm to about 6.0 ppm after storage for about 1, 2, or 3 months at a temperature of about 25° C. to about 40° C. and at about 60% to about 75% relative humidity. In yet another embodiment, p-aminophenol may be present in the pharmaceutical composition as a degradation product of acetaminophen in an amount of about 0.6 ppm to about 6.0 ppm after storage for about 1, 2, or 3 months at a temperature of about 25° C. to about 40° C. and at about 60% to about 75% relative humidity. In still another embodiment, p-aminophenol may be present in the pharmaceutical composition as a degradation product of acetaminophen in an amount of about 0.2 ppm, 0.3 ppm, 0.4 ppm, 0.5 ppm, 0.6 ppm, 0.7 ppm, 0.8 ppm, 0.9 ppm, 1.0 ppm, 1.1 ppm, 1.2 ppm, 1.3 ppm, 1.4 ppm, 1.5 ppm, 1.6 ppm, 1.7 ppm, 1.8 ppm, 1.9 ppm, 2.0 ppm, 2.1 ppm, 2.2 ppm, 2.3 ppm, 2.4 ppm, 2.5 ppm, 2.6 ppm, 2.7 ppm, 2.8 ppm, 2.9 ppm, 3.0 ppm, 3.1 ppm, 3.2 ppm, 3.3 ppm, 3.4 ppm, 3.5 ppm, 3.6 ppm, 3.7 ppm, 3.8 ppm, 3.9 ppm, 4.0 ppm, 4.1 ppm, 4.2 ppm, 4.3 ppm, 4.4 ppm, 4.5 ppm, 4.6 ppm, 4.7 ppm, 4.8 ppm, 4.9 ppm, 5.0 ppm, 5.1 ppm, 5.2 ppm, 5.3 ppm, 5.4 ppm, 5.5 ppm, 5.6 ppm, 5.7 ppm, 5.8 ppm, 5.9 ppm, and 6.0 ppm after storage for about 1, 2, or 3 months at a temperature of 25° C. to about 40° C. and at about 60% to about 75% relative humidity

In one embodiment, oxycodone N-oxide may be present in the pharmaceutical composition as a degradation product of oxycodone in any amount up to and including about 0.5% by weight of the oxycodone. In other embodiments, oxycodone N-oxide may be present in the pharmaceutical composition as a degradation product of oxycodone in an amount of about 0.01% to about 0.5% by weight of the oxycodone after storage for about 1, 2, or 3 months at a constant temperature of about 25° C. to 40° C. and at about 60% to 75% relative humidity. In yet another embodiment, oxycodone N-oxide may be present in the pharmaceutical composition as a degradation product of oxycodone in an amount of about 0.05% to about 0.5% by weight of the oxycodone after storage for about 1, 2, or 3 months at a constant temperature of about 25° C. to 40° C. and at about 60% to 75% relative humidity. In additional embodiments, oxycodone N-oxide may be present in the pharmaceutical composition as a degradation product of oxycodone in an amount of about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, and 0.5% by weight of the oxycodone after storage for about 1, 2, or 3 months at a constant temperature of about 25° C. to about 40° C. and at about 60% to about 75% relative humidity.

In one embodiment, Related Substance A (i.e., C-Normorphinan-6-carboxylic acid, 4,5-epoxy-6,14-dihydroxy-3-methoxy-17-methyl-, (5α,6α)-) may be present in the pharmaceutical composition as a degradation product of oxycodone in a maximum amount of about 0.5% by weight of the oxycodone. In other embodiments, Related Substance A may be present in the pharmaceutical composition as a degradation product of oxycodone in an amount of about 0.01% to about 0.5% by weight of the oxycodone after storage for about 1, 2, or 3 months at a temperature of about 25° C. to about 40° C. and at about 60% to about 75% relative humidity. In yet another embodiment, Related Substance A may be present in the pharmaceutical composition as a degradation product of oxycodone in an amount of about 0.05% to about 0.5% by weight of the oxycodone after storage for about 1, 2, or 3 months at a temperature of about 25° C. to about 40° C. and at about 60% to about 75% relative humidity. In other embodiments, Related Substance A may be present in the pharmaceutical composition as a degradation product of oxycodone in an amount of about 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, and 0.5% by weight of the oxycodone after storage for about 1, 2, or 3 months at a temperature of about 25° C. to about 40° C. and at about 60% to about 75% relative humidity.

In one embodiment, each unspecified acetaminophen degradation product may be present in the pharmaceutical composition in any amount up to about 0.15% by weight of the acetaminophen. In another embodiment, each unspecified acetaminophen degradation product may be present in the pharmaceutical composition as a degradation product of acetaminophen in an amount of about 0.01% and about 0.15% by weight of the acetaminophen after storage for about 1, 2, or 3 months at a temperature of about 25° C. to about 40° C. and at about 60% to about 75% relative humidity. In still another embodiment, each unspecified acetaminophen degradation product may be present in the pharmaceutical composition as a degradation product of acetaminophen in an amount of about 0.05% and about 0.15% by weight of the acetaminophen after storage for about 1, 2, or 3 months at a temperature of about 25° C. to about 40° C. and at about 60% to about 75% relative humidity. In other embodiments, each unspecified acetaminophen degradation product may be present in the pharmaceutical composition as a degradation product of acetaminophen in an amount of about 0.01%, 0.02%, 0.03%, 0.04%, 0.05%, 0.06%, 0.07%, 0.08%, 0.09%, 0.1%, 0.11%, 0.12%, 0.13%, 0.14%, and 0.15% by weight of the acetaminophen after storage for about 1, 2, or 3 months at a temperature of about 25° C. to about 40° C. and at about 60% to about 75% relative humidity.

In one embodiment, each unspecified oxycodone HCl degradation product may be present in the pharmaceutical composition in a maximum amount of about 0.2% by weight of the oxycodone. In other embodiments, each unspecified oxycodone HCl degradation product may be present in the pharmaceutical composition in an amount of about 0.01% to about 0.2% by weight of the oxycodone after storage for about 1, 2, or 3 months at a temperature of about 25° C. to about 40° C. and at about 60% to about 75% relative humidity. In yet another embodiment, each unspecified oxycodone HCl degradation product may be present in the pharmaceutical composition in an amount of about 0.05% to about 0.2% by weight of the oxycodone after storage for about 1, 2, or 3 months at a temperature of about 25° C. to about 40° C. and at about 60% to about 75% relative humidity. In further embodiments, each unspecified oxycodone HCl degradation product may be present in the pharmaceutical composition in an amount of about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, and 0.2% by weight of the oxycodone after storage for about 1, 2, or 3 months at a temperature of about 25° C. to about 40° C. and at about 60% to about 75% relative humidity.

In one embodiment, the total acetaminophen degradation products may be present in the pharmaceutical composition in a maximum amount of about 1.0% by weight of the acetaminophen. In other embodiments, the total acetaminophen degradation products may be present in the pharmaceutical composition in an amount of about 0.05% to about 1.0% by weight of the acetaminophen after storage for about 1, 2, or 3 months at a temperature of about 25° C. to about 40° C. and at about 60% to about 75% relative humidity. In further embodiments, the total acetaminophen degradation products may be present in the pharmaceutical composition in an amount of about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, and 1.0% by weight of the acetaminophen after storage for about 1, 2, or 3 months at a temperature of about 25° C. to about 40° C. and at about 60% to about 75% relative humidity.

In one embodiment, the total oxycodone degradation products may be present in the pharmaceutical composition in a maximum amount of about 1.0% by weight of the oxycodone. In further embodiments, the total oxycodone degradation products may be present in the pharmaceutical composition in an amount of about 0.05% to about 1.0% by weight of the oxycodone after storage for about 1, 2, or 3 months at a temperature of about 25° C. to about 40° C. and at about 60% to about 75% relative humidity. In yet other embodiments, the total oxycodone degradation products may be present in the pharmaceutical composition in an amount of about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, and 1.0% by weight of the oxycodone after storage for about 1, 2, or 3 months at a temperature of about 25° C. to about 40° C. and at about 60% to about 75% relative humidity.

### (h) In Vivo and Pharmacokinetic Properties of the Pharmaceutical Composition

The pharmaceutical composition disclosed herein comprises at least one immediate release portion for immediate release of oxycodone and acetaminophen such that therapeutic plasma concentrations are quickly attained (e.g., within one hour) and the initial onset of action is achieved within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition upon oral administration to a subject. The pharmaceutical composition disclosed herein also comprises at least one extended release portion for sustained release of oxycodone and acetaminophen over an extended period of time, e.g., about 3 to about 12 hours, or about 4 to about 9 hours, or at least about 6 hours, or at least about 8 hours, to the upper gastrointestinal tract where acetaminophen, and potentially oxycodone, is best absorbed.

The pharmaceutical composition may be orally administered to a subject once in a 24 hour period (q.d. or once-daily), two times in a 24 hour period (b.i.d. or twice-daily), or three times in a 24 hour period (t.i.d. or three times daily). In one embodiment, the pharmaceutical composition may be orally administered to the subject twice a day (i.e., every 12 hours). In another embodiment, the pharmaceutical composition may be orally administered to the subject at time zero (t=0), and then the subject may be administered a subsequent dose of the pharmaceutical composition either after eight hours (t=8) or twelve hours (t=12). The subject may be a mammal, and in certain embodiments, the subject may be a human.

In another embodiment, the subject may be administered a first or loading dose of the pharmaceutical composition. This first or loading dose may assist the subject in more quickly attaining steady state blood levels of the active drugs. In a further embodiment, the subject may be administered a first or loading dose of the pharmaceutical composition comprising about 22.5 mg of oxycodone and about 975 mg of acetaminophen. In yet another embodiment, the subject may be administered a first or loading dose of the pharmaceutical composition comprising 2 tablets, each tablet comprising about 11.25 mg of oxycodone and about 462.5 mg of acetaminophen. In yet another embodiment, the subject may be administered a first or loading dose of the pharmaceutical composition comprising 3 tablets, each tablet comprising about 7.5 mg of oxycodone and about 325 mg of acetaminophen. In still another embodiment, the subject may be administered a first or loading dose of the pharmaceutical composition comprising 4 tablets, each tablet comprising about 5.625 mg of oxycodone and about 231.25 mg of acetaminophen. In yet another embodiment, the subject may be administered a first or loading dose of the pharmaceutical composition comprising 2 capsules, each capsule comprising about 11.25 mg of oxycodone and about 462.5 mg of acetaminophen. In yet another embodiment, the subject may be administered a first or loading dose of the pharmaceutical composition comprising 3 capsules, each capsules comprising about 7.5 mg of oxycodone and about 325 mg of acetaminophen. In still another embodiment, the subject may be administered a first or loading dose of the pharmaceutical composition comprising 4 capsules, each capsules comprising about 5.625 mg of oxycodone and about 231.25 mg of acetaminophen.

Upon oral administration to a subject, the pharmaceutical composition disclosed herein may maintain a therapeutic blood plasma concentration of oxycodone of at least about 5 ng/mL from about 0.75 hours to about 12 hours after administration of the composition. In another embodiment, the plasma concentration of oxycodone may be maintained at a concentration of at least about 7.5 ng/mL from about 1 hour to about 12 hours after administration of the composition. In a further embodiment, the plasma concentration of oxycodone may be maintained at a concentration of at least about 7.5 ng/mL from about 0.75 hour to about 10 hours after administration of the composition. In a further embodiment, the plasma concentration of oxycodone may be maintained at a concentration of at least about 10 ng/mL from about 2 hour to about 10 hours after administration of the composition. In yet another embodiment, the plasma concentration of oxycodone may be maintained at a concentration of at least about 10 ng/mL from about 1 hour to about 10 hours after administration of the composition. In still another embodiment, the plasma concentration of oxycodone may be maintained at a concentration of at least about 10 ng/mL from about 0.75 hour to about 10 hours after administration of the composition.

In another embodiment, the pharmaceutical composition, when orally administered to a subject, may produce a plasma profile characterized by a mean Cₘₐₓ (peak plasma concentration) for oxycodone from about 0.9 ng/mL/mg to about 1.6 ng/mL/mg. In another embodiment, the mean Cₘₐₓ for oxycodone may range from about 1.0 ng/mL/mg to about 1.5 ng/mL/mg. In an additional embodiment, the mean Cₘₐₓ for oxycodone may be about 0.9, 1.0, 1.1, 1.2, 1.3, 1.4, 1.5, or 1.6 ng/mL/mg. Moreover, the mean Cₘₐₓ for oxycodone at steady state may range from about 1.5 ng/mL/mg to about 2.0 ng/mL/mg, from about 1.6 ng/mL/mg to about 1.95 ng/mL/mg, or from about 1.7 ng/mL/mg to about 1.85 ng/mL/mg.

In a further embodiment, the pharmaceutical composition, when orally administered to a subject, surprisingly may produce a blood plasma concentration profile characterized by a biphasic increase in blood plasma concentrations of oxycodone. Deconvolution of the pharmaceutical composition and the target plasma profiles can be done in WinNonLin (version 5.2, Pharsight Corp., Mountain View, Calif.). The results of such a deconvolution analysis for oxycodone is depicted in FIG. 23. The biphasic absorption of oxycodone may be characterized by an initial rapid absorption resulting in a first peak in plasma concentration between about 1 hour and 2 hours, which contributes to the early onset of action, and a second peak in plasma concentrations between about 3 hours and 7 hours as a result of slower absorption taking place from the at least one extended release portion after administration of the composition, which contributes to the duration or maintenance of analgesia. In some instances, the second peak may correspond to the overall Cₘₐₓ of the composition. The biphasic increase in blood plasma concentrations of oxycodone may be characterized by a plasma concentration-time profile for oxycodone in which the slope of a line drawn between 0 hour and about 2 hours is greater than the slope of a line drawn between about 2 hours and about 5 hours. See FIG. 23.

This biphasic increase in oxycodone levels resulting from the composition has several benefits. For example, providing rapid but not too high concentrations of oxycodone for quick onset of analgesia followed by maintenance of oxycodone levels over an extended time period could prevent a human subject from developing liking or dependence (abuse) for oxycodone. Further fluctuations in the oxycodone plasma levels could also prevent development of tolerance at the active site. Thus, the biphasic increase in oxycodone levels helps to prevent this acute tolerance.

In an additional embodiment, the pharmaceutical composition, when orally administered to a subject, may produce a plasma profile characterized by a mean AUC for oxycodone from about 9.0 ng·hr/mL/mg to about 18.5 ng·hr/mL/mg. In a further embodiment, the mean AUC for oxycodone may be from about 12.0 ng·hr/mL/mg to about 16.0 ng·hr/mL/mg. In another embodiment, the mean AUC for oxycodone may be about 9.0, 9.5, 10.0, 10.5, 11.0, 11.5, 12.0, 12.5, 13.0, 13.5, 14.0, 14.5, 15.0, 15.5, or 16.0 ng·hr/mL/mg. Additionally, the mean AUC for oxycodone at steady state may range from about 11.0 ng·hr/mL/mg to about 17.0 ng·hr/mL/mg, from about 12.0 ng·hr/mL/mg to about 16.0 ng·hr/mL/mg, or from about 13.0 ng·hr/mL/mg to about 15.0 ng·hr/mL/mg.

In a further embodiment, the pharmaceutical composition, when orally administered to a subject, may produce a plasma profile characterized by a median Tₘₐₓ (time to peak plasma concentration) for oxycodone from about 2.0 hours to about 7.0 hours. In an alternate embodiment, the median Tₘₐₓ for oxycodone may be from about 3.0 hours to about 6.0 hours. In another embodiment, the median Tₘₐₓ for oxycodone may be about 2.0, 2.5, 3.0, 3.5, 4.0, 4.5, 5.0, 5.5 or 6.0 hours. Moreover, the median Tₘₐₓ for oxycodone at steady state may range from about 1.5 hours to about 3.5 hours, or from about 2 hours to about 3 hours.

In still another embodiment, the pharmaceutical composition, when orally administered to a subject, may produce a plasma profile characterized by a median tlag for oxycodone from about 0 hours to about 0.5 hours. In an alternate embodiment, the median tlag for oxycodone may be from about 0 hours to about 0.25 hours.

Rates of absorption are often assessed by comparing standard pharmacokinetic parameters such as Tₘₐₓ and Cₘₐₓ. The extent of absorption is assessed by the AUC. A short Tmax has been used to indicate rapid absorption. The U.S. FDA, Guidance for Industry: Bioavailability and Bioequivalence Studies for Orally Administered Drug Products—General Considerations (March 2003) and related publications (Chen et al, Clin. Pharmacokinet. 40(8):565-72, 2001) also recommends the use of partial AUC for some modified-release drugs (“MR drugs”), such as the pharmaceutical compositions disclosed herein. A partial AUC calculation may be used to measure early exposure to a drug, which may signify an initial onset of pain relief and/or to measure prolonged exposure of a drug in achieving sustained relief. Partial AUC calculations can also demonstrate whether two MR drugs are truly bioequivalent by comparing, for example, an early partial AUC, which will be associated with a drug's response onset, and a late partial AUC, which will be associated with a drug's sustained response. The parameters for compositions vary greatly between subjects. The parameters also vary depending on aspects of the study protocol such as the sampling scheduling, subject posture and general subject health. Values quoted in this specification are given as mean±standard deviation unless otherwise noted.

For partial AUC calculations, the standard linear trapezoidal summation over each time interval is used. The partial AUCs are calculated from the mean pharmacokinetic profile. For time 0 to 1 hour the partial AUC is AUC(0-1 hr); for time 0 to 2 hours the partial AUC is AUC₍₀₋₂ₕᵣ₎; for time 0-4 hours the partial AUC is AUC₍₀₋₄ₕᵣ₎; for time 0 to 6 hour the partial AUC is AUC₍₀₋₆ₕᵣ₎; for f time 0 to 8 hours the partial AUC is AUC₍₀₋₈ₕᵣ₎; and for time 0 to the last measurable time point (“x”) the partial AUC is AUC(0-(x)hr) where each partial AUC is calculated according to standard pharmaceutical industry pharmacokinetic calculation methodologies as given by:

AUC₍₀₋₁ₕᵣ₎—Area under the drug concentration-time curve calculated using linear trapezoidal summation from time zero to time 1 hour.

AUC₍₀₋₂ₕᵣ₎—Area under the drug concentration-time curve calculated using linear trapezoidal summation from time zero to time 2 hours.

AUC₍₀₋₄ₕᵣ₎—Area under the drug concentration-time curve calculated using linear trapezoidal summation from time zero to time 4 hours.

AUC₍₀₋₆ₕᵣ₎—Area under the drug concentration-time curve calculated using linear trapezoidal summation from time zero to time 6 hours.

AUC₍₀₋₈ₕᵣ)—Area under the drug concentration-time curve calculated using linear trapezoidal summation from time zero to time 8 hours.

AUC₀₋₍ₜ₎ₕᵣ₎—Area under the drug concentration-time curve calculated using linear trapezoidal summation from time zero to the last measurable time point.

AUC_{(0-(Tmax of IR product+2SD))}—Area under the drug concentration-time curve calculated using linear trapezoidal summation from time zero to the time of the mean peak (Tmax) for the immediate release version of the drug plus two standard deviations (“2SD”) for the immediate release drug. The FDA has identified this calculation in association with an early onset of response for certain modified-release dosage forms, which show complex pharmacokinetic characteristics. (See supra March 2003 Guidance; Draft Guidance on Dexmethylphenidate Hydrochloride (March 2012); Draft Guidance on Methylphenidate Hydrocholoride (November 2011)).

AUC_{((Tmax of IR product+2SD)-t)}—Area under the drug concentration-time curve calculated using linear trapezoidal summation from the time of the mean peak (Tₘₐₓ) for the immediate release version of the drug plus two standard deviations (“2SD”) for the immediate release drug to the last measurable time point. The FDA has identified this parameter in association with sustaining the response for modified-release dosage forms, which shows complex pharmacokinetic characteristics. (See March 2003 Guidance supra; Draft Guidance on Dexmethylphenidate Hydrochloride (March 2012); Draft Guidance on Methylphenidate Hydrocholoride (November 2011)).

AUC_{(x-(y)hr}—Area under the drug concentration-time curve calculated using linear trapezoidal summation from time “x” (e.g., any measurable time point, such as 8 hours) to time “y” (e.g., any other measurable time point later than “x”, such as 12 hours).

AUC_{(0-∞)}—Area under the drug concentration-time curve calculated using linear trapezoidal summation from time 0 to infinity.

Further, partial AUC may be calculated using trapezoidal summation from time Tmax to time t (the last measured time point of plasma concentration profile).

In one embodiment, the pharmaceutical composition, when orally administered to a subject, may produce a plasma profile characterized by an AUC₀₋₁ₕᵣ for oxycodone from about 0.10 ng·hr/mL/mg to about 0.45 ng·hr/mL/mg, from about 0.15 ng·hr/mL/mg to about 0.25 ng·hr/mL/mg, or from about 0.25 ng·hr/mL/mg to about 0.35 ng·hr/mL/mg. In another embodiment, the AUC₀₋₁ₕᵣ for oxycodone may be about 0.10, 0.15, 0.20, 0.25, 0.30, 0.35, 0.40, or 0.45 ng·hr/mL/mg.

In an additional embodiment, the pharmaceutical composition, when orally administered to a subject, may produce a plasma profile characterized by an AUC₀₋₂ₕᵣ for oxycodone from about 0.65 ng·hr/mL/mg to about 1.50 ng·hr/mL/mg, from about 0.80 ng·hr/mL/mg to about 1.0 ng·hr/mL/mg, or from about 1.0 ng·hr/mL/mg to about 1.2 ng·hr/mL/mg. In another embodiment, the AUC₀₋₂ₕᵣ for oxycodone may be about 0.65, 0.70, 0.75, 0.80, 0.85, 0.90, 0.95, 1.0, 1.05, 1.10, 1.15, 1.20, 1.25, 1.30, 1.35, 1.40, 1.45, or 1.50 ng·hr/mL/mg.

In an additional embodiment, the pharmaceutical composition, when orally administered to a subject under fasted conditions, may produce a plasma profile characterized by an AUC₀₋₂ₕᵣ for oxycodone from about 0.8 ng·hr/mL/mg to about 1.50 ng·hr/mL/mg, from about 0.80 ng·hr/mL/mg to about 1.0 ng·hr/mL/mg, or from about 1.0 ng·hr/mL/mg to about 1.2 ng·hr/mL/mg. In another embodiment, the AUC₀₋₂ₕᵣ for oxycodone may be about 0.80, 0.85, 0.90, 0.95, 1.0, 1.05, 1.10, 1.15, 1.20, 1.25, 1.30, 1.35, 1.40, 1.45, or 1.50 ng·hr/mL/mg.

In a further embodiment, the pharmaceutical composition, when orally administered to a subject under fed conditions (high fat meal), may produce a plasma profile characterized by an AUC₀₋₂ₕᵣ for oxycodone from about 0.65 ng·hr/mL/mg to about 1.30 ng·hr/mL/mg, from about 0.80 ng·hr/mL/mg to about 1.0 ng·hr/mL/mg, or from about 1.0 ng·hr/mL/mg to about 1.2 ng·hr/mL/mg. In another embodiment, the AUC₀₋₂ₕᵣ for oxycodone may be about 0.65, 0.70, 0.75, 0.80, 0.85, 0.90, 0.95, 1.0, 1.05, 1.10, 1.15, 1.20, 1.25, or 1.30 ng·hr/mL/mg.

In a further embodiment, the pharmaceutical composition, when orally administered to a subject under fed conditions (low fat meal), may produce a plasma profile characterized by an AUC₀₋₂ₕᵣ for oxycodone from about 0.65 ng·hr/mL/mg to about 1.30 ng·hr/mL/mg, from about 0.80 ng·hr/mL/mg to about 1.0 ng·hr/mL/mg, or from about 1.0 ng·hr/mL/mg to about 1.2 ng·hr/mL/mg. In another embodiment, the AUC₀₋₂ₕᵣ for oxycodone may be about 0.65, 0.70, 0.75, 0.80, 0.85, 0.90, 0.95, 1.0, 1.05, 1.10, 1.15, 1.20, 1.25, or 1.30 ng·hr/mL/mg.

In an additional embodiment, the pharmaceutical composition, when orally administered to a subject, may produce a plasma profile characterized by an AUC₂₋₄₈ₕᵣ for oxycodone from about 8.0 ng·hr/mL/mg to about 17.8 ng·hr/mL/mg, from about 10.0 ng·hr/mL/mg to about 11.0 ng·hr/mL/mg, or from about 11.0 ng·hr/mL/mg to about 12.0 ng·hr/mL/mg, or from about 12.0 ng·hr/mL/mg to about 13.0 ng·hr/mL/mg, or from about 13.0 ng·hr/mL/mg to about 14.0 ng·hr/mL/mg, or from about 14.0 ng·hr/mL/mg to about 15.0 ng·hr/mL/mg. In another embodiment, the AUC₂₋₄₈ₕᵣ for oxycodone may be about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, or 17.8 ng·hr/mL/mg.

In an additional embodiment, the pharmaceutical composition, when orally administered to a subject under fasted conditions, may produce a plasma profile characterized by an AUC₂₋₄₈ₕᵣ for oxycodone from about 8.0 ng·hr/mL/mg to about 15.1 ng·hr/mL/mg, from about 10.0 ng·hr/mL/mg to about 11.0 ng·hr/mL/mg, or from about 11.0 ng·hr/mL/mg to about 12.0 ng·hr/mL/mg, or from about 12.0 ng·hr/mL/mg to about 13.0 ng·hr/mL/mg, or from about 13.0 ng·hr/mL/mg to about 14.0 ng·hr/mL/mg, or from about 14.0 ng·hr/mL/mg to about 15.0 ng·hr/mL/mg. In another embodiment, the AUC₂₋₄₈ₕᵣ for oxycodone may be about 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, or 15.1 ng·hr/mL/mg.

In an additional embodiment, the pharmaceutical composition, when orally administered to a subject under fed conditions (high fat meal), may produce a plasma profile characterized by an AUC₂₋₄₈ₕᵣ for oxycodone from about 9.5 ng·hr/mL/mg to about 17.8 ng·hr/mL/mg, from about 10.0 ng·hr/mL/mg to about 11.0 ng·hr/mL/mg, or from about 11.0 ng·hr/mL/mg to about 12.0 ng·hr/mL/mg, or from about 12.0 ng·hr/mL/mg to about 13.0 ng·hr/mL/mg, or from about 13.0 ng·hr/mL/mg to about 14.0 ng·hr/mL/mg, or from about 14.0 ng·hr/mL/mg to about 15.0 ng·hr/mL/mg, or from about 14.0 ng·hr/mL/mg to about 15.0 ng·hr/mL/mg. In another embodiment, the AUC₂₋₄₈ₕᵣ for oxycodone may be about 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, or 17.8 ng·hr/mL/mg.

In an additional embodiment, the pharmaceutical composition, when orally administered to a subject under fed conditions (low fat meal), may produce a plasma profile characterized by an AUC₂₋₄₈ₕᵣ for oxycodone from about 9.5 ng·hr/mL/mg to about 17.8 ng·hr/mL/mg, from about 10.0 ng·hr/mL/mg to about 11.0 ng·hr/mL/mg, or from about 11.0 ng·hr/mL/mg to about 12.0 ng·hr/mL/mg, or from about 12.0 ng·hr/mL/mg to about 13.0 ng·hr/mL/mg, or from about 13.0 ng·hr/mL/mg to about 14.0 ng·hr/mL/mg, or from about 14.0 ng·hr/mL/mg to about 15.0 ng·hr/mL/mg, or from about 14.0 ng·hr/mL/mg to about 15.0 ng·hr/mL/mg. In another embodiment, the AUC₂₋₄₈ₕᵣ for oxycodone may be about 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, or 17.8 ng·hr/mL/mg.

In an additional embodiment, the pharmaceutical composition, when orally administered to a subject, may produce a plasma profile characterized by an AUC₈₋₁₀ₕᵣ for oxycodone from about 0.90 ng·hr/mL/mg to about 2.30 ng·hr/mL/mg, from about 0.80 ng·hr/mL/mg to about 1.0 ng·hr/mL/mg, or from about 1.0 ng·hr/mL/mg to about 1.2 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg, or from about 1.6 ng·hr/mL/mg to about 1.8 ng·hr/mL/mg, or from about 1.8 ng·hr/mL/mg to about 2.0 ng·hr/mL/mg. In another embodiment, the AUC₈₋₁₀ₕᵣ for oxycodone may be about 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, or 2.30 ng·hr/mL/mg.

In an additional embodiment, the pharmaceutical composition, when orally administered to a subject under fasted conditions, may produce a plasma profile characterized by an AUC₈₋₁₀ₕᵣ for oxycodone from about 0.90 ng·hr/mL/mg to about 1.70 ng·hr/mL/mg, from about 0.90 ng·hr/mL/mg to about 1.0 ng·hr/mL/mg, or from about 1.0 ng·hr/mL/mg to about 1.2 ng·hr/mL/mg, or from about 1.2 ng·hr/mL/mg to about 1.4 ng·hr/mL/mg, or from about 1.4 ng·hr/mL/mg to about 1.6 ng·hr/mL/mg. In another embodiment, the AUC₈₋₁₀ₕᵣ for oxycodone may be about 0.90, 0.95, 1.0, 1.05, 1.10, 1.15, 1.20, 1.25, 1.30, 1.35, 1.40, 1.45, 1.50, 1.55, 1.60, 1.65, or 1.70 ng·hr/mL/mg.

In an additional embodiment, the pharmaceutical composition, when orally administered to a subject under fed conditions (high fat meal), may produce a plasma profile characterized by an AUC₈₋₁₀ₕᵣ for oxycodone from about 1.15 ng·hr/mL/mg to about 2.30 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg, or from about 1.8 ng·hr/mL/mg to about 2.0 ng·hr/mL/mg. In another embodiment, the AUC₈₋₁₀ₕᵣ for oxycodone may be about 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, or 2.30 ng·hr/mL/mg.

In an additional embodiment, the pharmaceutical composition, when orally administered to a subject under fed conditions (low fat meal), may produce a plasma profile characterized by an AUC₈₋₁₀ₕᵣ for oxycodone from about 1.20 ng·hr/mL/mg to about 2.30 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg, or from about 1.8 ng·hr/mL/mg to about 2.0 ng·hr/mL/mg. In another embodiment, the AUC₈₋₁₀ₕᵣ for oxycodone may be about 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, or 2.30 ng·hr/mL/mg.

In an additional embodiment, the pharmaceutical composition, when orally administered to a subject, may produce a plasma profile characterized by an AUC₁₀₋₁₂ₕᵣ for oxycodone from about 0.70 ng·hr/mL/mg to about 2.0 ng·hr/mL/mg, from about 0.80 ng·hr/mL/mg to about 1.0 ng·hr/mL/mg, or from about 1.0 ng·hr/mL/mg to about 1.2 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg, or from about 1.6 ng·hr/mL/mg to about 1.8 ng·hr/mL/mg. In another embodiment, the AUC₁₀₋₁₂ₕᵣ for oxycodone may be about 0.70, 0.75, 0.80, 0.85, 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or 2.0 ng·hr/mL/mg.

In an additional embodiment, the pharmaceutical composition, when orally administered to a subject under fasted conditions, may produce a plasma profile characterized by an AUC₁₀₋₁₂ₕᵣ for oxycodone from about 0.70 ng·hr/mL/mg to about 1.4 ng·hr/mL/mg, from about 0.80 ng·hr/mL/mg to about 1.0 ng·hr/mL/mg, or from about 1.0 ng·hr/mL/mg to about 1.2 ng·hr/mL/mg, or from about 1.2 ng·hr/mL/mg to about 1.4 ng·hr/mL/mg. In another embodiment, the AUC₁₀₋₁₂ₕᵣ for oxycodone may be about 0.70, 0.75, 0.80, 0.85, 0.90, 0.95, 1.0, 1.05, 1.10, 1.15, 1.20, 1.25, 1.30, 1.35, or 1.40 ng·hr/mL/mg.

In an additional embodiment, the pharmaceutical composition, when orally administered to a subject under fed conditions (high fat meal), may produce a plasma profile characterized by an AUC₁₀₋₁₂ₕᵣ for oxycodone from about 1.0 ng·hr/mL/mg to about 1.95 ng·hr/mL/mg, or from about 1.0 ng·hr/mL/mg to about 1.2 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg. In another embodiment, the AUC₁₀₋₁₂ₕᵣ for oxycodone may be about 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, or 1.95 ng·hr/mL/mg.

In an additional embodiment, the pharmaceutical composition, when orally administered to a subject under fed conditions (low fat meal), may produce a plasma profile characterized by an AUC₁₀₋₁₂ₕᵣ for oxycodone from about 0.95 ng·hr/mL/mg to about 1.85 ng·hr/mL/mg, or from about 1.0 ng·hr/mL/mg to about 1.2 ng·hr/mL/mg, or from about 1.2 ng·hr/mL/mg to about 1.4 ng·hr/mL/mg, or from about 1.4 ng·hr/mL/mg to about 1.6 ng·hr/mL/mg, or from about 1.6 ng·hr/mL/mg to about 1.8 ng·hr/mL/mg. In another embodiment, the AUC₁₀₋₁₂ₕᵣ for oxycodone may be about 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, or 1.85 ng·hr/mL/mg.

In a further embodiment, the pharmaceutical composition, when orally administered to a subject, may produce a plasma profile characterized by an AUC₀₋₄ₕᵣ for oxycodone from about 2.0 ng·hr/mL/mg to about 4.0 ng·hr/mL/mg, from about 2.5 ng·hr/mL/mg to about 3.0 ng·hr/mL/mg, or from about 3.0 ng·hr/mL/mg to about 3.5 ng·hr/mL/mg. In another embodiment, the AUC₀₋₄ₕᵣ for oxycodone may be about 2.0, 2.5, 3.0, 3.5, or 4.0 ng·hr/mL/mg.

In yet another embodiment, the pharmaceutical composition, when orally administered to a subject, may produce a plasma profile characterized by an AUC_{Tmax-t} for oxycodone from about 5.0 ng·hr/mL/mg to about 16.0 ng·hr/mL/mg, from about 8.0 ng·hr/mL/mg to about 10.5 ng·hr/mL/mg, or from about 10.5 ng·hr/mL/mg to about 14.0 ng·hr/mL/mg. In another embodiment, the AUC_{Tmax-t} for oxycodone may be about 5.0, 6.0, 7.0, 8.0, 9.0, 10.0, 11.0, 12.0, 12.5, 13.0, 13.5, 14.0, 14.5, 15.0 or 16.0 ng·hr/mL/mg.

In still another embodiment, the pharmaceutical composition, when orally administered to a subject, may produce a plasma profile characterized by an AUC_{(0-(Tmax of IR product+2SD))} for oxycodone after a single dose from about 1.0 ng·hr/mL/mg to about 3.0 ng·hr/mL/mg, from about 1.50 ng·hr/mL/mg to about 2.5 ng·hr/mL/mg, or from about 1.75 ng·hr/mL/mg to about 2.25 ng·hr/mL/mg. In another embodiment, the AUC_{(0-(Tmax of IR product+2SD))} for oxycodone may be about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, or 2.75 ng·hr/mL/mg.

In one embodiment, the immediate release product referenced for the Partial AUC calculations is Percocet in the fasted state and the following calculation was used to determine AUC_{(0-(Tmax of IR product+2SD))}:

oxycodone mean±SD=1.0 h±0.89 h; Tmax+2SD=2.8 hours

In such embodiment, the pharmaceutical composition, when orally administered to a subject, may produce a plasma profile characterized by an AUC_{(0-2.8)} for oxycodone after a single dose from about 1.0 ng·hr/mL/mg to about 3.0 ng·hr/mL/mg, from about 1.50 ng·hr/mL/mg to about 2.5 ng·hr/mL/mg, or from about 1.75 ng·hr/mL/mg to about 2.25 ng·hr/mL/mg. In another embodiment, the AUC_{(0-2.8)} for oxycodone may be about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, or 2.75 ng·hr/mL/mg.

In yet another embodiment, the pharmaceutical composition, when orally administered to a subject, may produce a plasma profile characterized by an AUC₍₂₈₋₄₈₎ for oxycodone after a single dose from about 7.5 ng·hr/mL/mg to about 15.0 ng·hr/mL/mg, from about 8.45 ng·hr/mL/mg to about 13.7 ng·hr/mL/mg, or from about 9.5 ng·hr/mL/mg to about 11.5 ng·hr/mL/mg. In another embodiment, the AUC₍₂₈₋₄₈₎ for oxycodone may be about 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5 ng·hr/mL/mg.

In one embodiment, the immediate release product referenced for the Partial AUC calculations is Percocet in the fed state and the following calculation was used to determine AUC_{(0-(Tmax of IR product+2SD))}:

oxycodone mean±SD=1.9 h±1.2 h; Tmax+2SD=4.3 hours

In such embodiment, the pharmaceutical composition, when orally administered to a subject, may produce a plasma profile characterized by an AUC_{(0-4.3)} for oxycodone after a single dose from about 1.5 ng·hr/mL/mg to about 5.5 ng·hr/mL/mg, from about 2.0 ng·hr/mL/mg to about 5.0 ng·hr/mL/mg, from about 2.5 ng·hr/mL/mg to about 4.5 ng·hr/mL/mg, or from about 3.0 ng·hr/mL/mg to about 4.0 ng·hr/mL/mg. In another embodiment, the AUC_{(0-4.3)} for oxycodone may be about 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, or 5.5 ng·hr/mL/mg.

In yet another embodiment, the pharmaceutical composition, when orally administered to a subject, may produce a plasma profile characterized by an AUC_{(4.3-48)} for oxycodone after a single dose from about 5.0 ng·hr/mL/mg to about 15.0 ng·hr/mL/mg, from about 7.5 ng·hr/mL/mg to about 13.5 ng·hr/mL/mg, from about 9.0 ng·hr/mL/mg to about 12.0 ng·hr/mL/mg, or from about 9.5 ng·hr/mL/mg to about 11.5 ng·hr/mL/mg. In another embodiment, the AUC_{(4.3-48)} for oxycodone may be about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0 ng·hr/mL/mg.

In one embodiment, the pharmaceutical composition, when orally administered to a subject in a fasted state, may produce a plasma profile characterized by an AUC8-12 hr for oxycodone from about 3% to about 33% of the AUC0-t, from about 10% to about 27% of the AUC0-t, or from about 15% to about 22% of the AUC0-t. In another embodiment, the pharmaceutical composition, when orally administered to a subject in a fed state, may produce a plasma profile characterized by an AUC8-12 hr for oxycodone from about 5% to about 35% of the AUC0-t, from about 12% to about 30% of the AUC0-t, or from about 15% to about 25% of the AUC0-t.

In an alternate embodiment, the pharmaceutical composition, when orally administered to a subject, may provide a mean half-life of oxycodone that ranges from about 3.5 hours to about 5.5 hours, or from about 4 hours to about 5 hours. In various embodiments, the mean half-life of oxycodone may be about 3.8, 4.0, 4.2, 4.4, 4.6, 4.8, 5.0, or 5.2 hours.

In yet another embodiment, the pharmaceutical composition, when orally administered to a subject, produces a plasma profile characterized by an abuse quotient for oxycodone from about 3 to about 5. In other embodiments, the abuse quotient for oxycodone may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

Moreover, upon oral administration, the pharmaceutical composition disclosed herein may maintain a therapeutic plasma concentration of acetaminophen of at least about 2 mg/mL from about 1 hour to about 6 hours after administration. In another embodiment, the pharmaceutical composition may maintain a therapeutic plasma concentration of acetaminophen of at least about 2 mg/mL from about 0.75 hour to about 6.5 hours after administration. In yet another embodiment, the composition may maintain a plasma concentration of acetaminophen of at least about 1 mg/mL from about 0.5 hour to about 12 hours after administration.

In another embodiment, the pharmaceutical composition, when orally administered to a subject, may produce a plasma profile characterized by a mean Cₘₐₓ for acetaminophen from about 4.0 ng/mL/mg to about 11.0 ng/mL/mg. In other embodiments, the mean Cₘₐₓ for acetaminophen may be from about 4.0, 4.5, 5.0, 5.5, 6.0, 6.5, 7.0, 7.5, 8.0, 8.5, 9.0, 9.5, 10.0, 10.5, or 11.0 ng/mL/mg. Moreover, the mean Cₘₐₓ for acetaminophen at steady state may range from about 6.0 ng/mL/mg to about 9.0 ng/mL/mg, from about 6.5 ng/mL/mg to about 8.5 ng/mL/mg, or from about 7.0 ng/mL/mg to about 8.0 ng/mL/mg.

In a further embodiment, the pharmaceutical composition, when orally administered to a subject, surprisingly may produce a blood plasma concentration profile characterized by a biphasic increase in blood plasma concentrations of acetaminophen. The biphasic absorption of acetaminophen may characterized by an initial rapid absorption resulting in first peak in plasma concentrations between about 0.5 hour and 2 hours, which contributes to the early onset on action, and a second peak in plasma concentrations between about 3 hours and 7 hours after administration of the composition, which contributes to the duration or maintenance of analgesia. In some instances, the second peak may correspond to the overall Cmax of the composition. The biphasic increase in blood plasma concentrations of acetaminophen is characterized by a plasma concentration-time profile for acetaminophen in which the slope of a line drawn between 0 hour and 2 hour is greater than the slope of a line drawn between about 2 hours and 5 hours. See FIG. 24.

This biphasic increase in acetaminophen levels resulting from the composition has several benefits. For example, the initial rapid rise in plasma levels produce quick onset of analgesia and the slower absorption provides maintenance of analgesia for an extended period of time.

In a further embodiment, the pharmaceutical composition, when orally administered to a subject, may produce a plasma profile characterized by a mean AUC for acetaminophen from about 35.0 ng·hr/mL/mg to about 80.0 ng·hr/mL/mg. In a further embodiment, the mean AUC for acetaminophen may range from about 35.0 ng·hr/mL/mg to about 60.0 ng·hr/mL/mg. In other embodiments, the mean AUC for acetaminophen may be about 35.0, 40.0, 45.0, 50.0, 55.0, 60.0, 65.0, 70.0, 75.0, or 80.0 ng·hr/mL/mg. Additionally, the mean AUC for acetaminophen at steady state may range from about 40.0 ng·hr/mL/mg to about 50.0 ng·hr/mL/mg, from about 35.0 ng·hr/mL/mg to about 45.0 ng·hr/mL/mg, or from about 37.0 ng·hr/mL/mg to about 42.0 ng·hr/mL/mg.

In yet another embodiment, the pharmaceutical composition when orally administered to a subject, may produce a plasma profile characterized by a median Tmax for acetaminophen from about 0.5 hours to about 6.0 hours. In another embodiment, the median Tmax for acetaminophen may be from about 1.0 hour to about 5.0 hours. In a further embodiment, the median Tmax for acetaminophen may range from about 0.5 hour to about 4.0 hours. In still another embodiment, the median Tmax for acetaminophen may range from about 0.75 to about 1.5 hours. In other embodiments, the median Tmax may be about 0.5, 0.6, 0.7, 0.8, 0.9, 1.0, 1.1, 1.2, 1.3, 1.4, 1.5, 1.6, 1.7 1.8, 1.9, 2.0, 2.2, 2.4, 2.6, 2.8, 3.0, 3.2, 3.4, 3.6, 3.8, 4.0, 4.2, 4.4, 4.6, 4.8, or 5.0 hours. Moreover, the median Tmax for acetaminophen at steady state may range from about 0.5 hour to about 1.0 hour, or from about 0.5 hour to about 0.75 hour.

In a further embodiment, the pharmaceutical composition, when orally administered to a subject, may produce a plasma profile characterized by a median tlag for acetaminophen from about 0 hour to about 0.5 hour. In an alternate embodiment, the median tlag for acetaminophen may be from about 0 hour to about 0.25 hour. In one embodiment, the median tlag for acetaminophen may be 0 hour. In another embodiment, the median tlag for acetaminophen may be 0.25 hour.

In one embodiment, the pharmaceutical composition, when orally administered to a subject, may produce a plasma profile characterized by various partial AUCs for acetaminophen. The partial AUCs for acetaminophen are calculated as described above for oxycodone. The pharmaceutical composition, when orally administered to a subject, may produce a plasma profile characterized by an AUC0-1 hr for acetaminophen from about 1.25 ng·hr/mL/mg to about 3.25 ng·hr/mL/mg, from about 1.60 ng·hr/mL/mg to about 2.0 ng·hr/mL/mg, or from about 2.0 ng·hr/mL/mg to about 2.75 ng·hr/mL/mg. In another embodiment, the AUC0-1 hr for acetaminophen may be about 1.25, 1.30, 1.40, 1.50, 1.55, 1.60, 1.65, 1.70, 1.75, 1.80, 1.85, 1.90, 1.95, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, or 2.90 or ng·hr/mL/mg.

In an additional embodiment, the pharmaceutical composition, when orally administered to a subject, may produce a plasma profile characterized by an AUC₀₋₂ₕᵣ for acetaminophen from about 4.25 ng·hr/mL/mg to about 14.0 ng·hr/mL/mg, or from about 5.50 ng·hr/mL/mg to about 6.0 ng·hr/mL/mg, or from about 6.0 ng·hr/mL/mg to about 7.25 ng·hr/mL/mg, or from about 7.25 ng·hr/mL/mg to about 8.5 ng·hr/mL/mg, or from about 8.5 ng·hr/mL/mg to about 9.75 ng·hr/mL/mg, or from about 9.75 ng·hr/mL/mg to about 11.0 ng·hr/mL/mg, or from about 11.0 ng·hr/mL/mg to about 12.25 ng·hr/mL/mg. In another embodiment, the AUC₀₋₂ₕᵣ for acetaminophen may be about 4.25, 4.5, 4.75, 5.0, 5.25, 5.5, 5.75, 6.0, 6.25, 6.5, 6.75, 7.0, 7.25, 7.50, 7.75 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, or 14.0 ng·hr/mL/mg.

In an additional embodiment, the pharmaceutical composition, when orally administered to a subject under fasted conditions, may produce a plasma profile characterized by an AUC₀₋₂ₕᵣ for acetaminophen from about 7.25 ng·hr/mL/mg to about 14.0 ng·hr/mL/mg, or from about 7.25 ng·hr/mL/mg to about 8.5 ng·hr/mL/mg, or from about 8.5 ng·hr/mL/mg to about 9.75 ng·hr/mL/mg, or from about 9.75 ng·hr/mL/mg to about 11.0 ng·hr/mL/mg, or from about 11.0 ng·hr/mL/mg to about 12.25 ng·hr/mL/mg. In another embodiment, the AUC₀₋₂ₕᵣ for acetaminophen may be about 7.25, 7.50, 7.75 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, or 14.0 ng·hr/mL/mg.

In an additional embodiment, the pharmaceutical composition, when orally administered to a subject under fed conditions (high fat meal), may produce a plasma profile characterized by an AUC₀₋₂ₕᵣ for acetaminophen from about 4.5 ng·hr/mL/mg to about 8.75 ng·hr/mL/mg, or from about 5.0 ng·hr/mL/mg to about 6.0 ng·hr/mL/mg, or from about 6.0 ng·hr/mL/mg to about 7.0 ng·hr/mL/mg, or from about 7.0 ng·hr/mL/mg to about 8.0 ng·hr/mL/mg. In another embodiment, the AUC₀₋₂ₕᵣ for acetaminophen may be about 4.5, 4.75, 5.0, 5.25, 5.5, 5.75, 6.0, 6.25, 6.5, 6.75, 7.0, 7.25, 7.50, 7.75 8.0, 8.25, 8.5, or 8.75 ng·hr/mL/mg.

In an additional embodiment, the pharmaceutical composition, when orally administered to a subject under fed conditions (low fat meal), may produce a plasma profile characterized by an AUC₀₋₂ₕᵣ for acetaminophen from about 4.5 ng·hr/mL/mg to about 8.75 ng·hr/mL/mg, or from about 5.0 ng·hr/mL/mg to about 6.0 ng·hr/mL/mg, or from about 6.0 ng·hr/mL/mg to about 7.0 ng·hr/mL/mg, or from about 7.0 ng·hr/mL/mg to about 8.0 ng·hr/mL/mg. In another embodiment, the AUC₀₋₂ₕᵣ for acetaminophen may be about 4.5, 4.75, 5.0, 5.25, 5.5, 5.75, 6.0, 6.25, 6.5, 6.75, 7.0, 7.25, 7.50, 7.75 8.0, 8.25, 8.5, or 8.75 ng·hr/mL/mg.

In an additional embodiment, the pharmaceutical composition, when orally administered to a subject, may produce a plasma profile characterized by an AUC₂₋₄₈ₕᵣ for acetaminophen from about 26.0 ng·hr/mL/mg to about 53.5 ng·hr/mL/mg, or from about 32.0 ng·hr/mL/mg to about 35.0 ng·hr/mL/mg, or from about 35.0 ng·hr/mL/mg to about 38.0 ng·hr/mL/mg, or from about 38.0 ng·hr/mL/mg to about 41.0 ng·hr/mL/mg, or from about 41.0 ng·hr/mL/mg to about 44.0 ng·hr/mL/mg, or from about 44.0 ng·hr/mL/mg to about 47.0 ng·hr/mL/mg, or from about 47.0 ng·hr/mL/mg to about 50.0 ng·hr/mL/mg. In another embodiment, the AUC₂₋₄₈ₕᵣ for acetaminophen may be about 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, or 53.5 ng·hr/mL/mg.

In an additional embodiment, the pharmaceutical composition, when orally administered to a subject under fasted conditions, may produce a plasma profile characterized by an AUC₂₋₄₈ₕᵣ for acetaminophen from about 26.0 ng·hr/mL/mg to about 49.0 ng·hr/mL/mg, or from about 32.0 ng·hr/mL/mg to about 35.0 ng·hr/mL/mg, or from about 35.0 ng·hr/mL/mg to about 38.0 ng·hr/mL/mg, or from about 38.0 ng·hr/mL/mg to about 41.0 ng·hr/mL/mg, or from about 41.0 ng·hr/mL/mg to about 44.0 ng·hr/mL/mg, or from about 44.0 ng·hr/mL/mg to about 47.0 ng·hr/mL/mg. In another embodiment, the AUC₂₋₄₈ₕᵣ for acetaminophen may be about 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, or 49.0.

In an additional embodiment, the pharmaceutical composition, when orally administered to a subject under fed conditions (high fat meal), may produce a plasma profile characterized by an AUC₂₋₄₈ₕᵣ for acetaminophen from about 28.5 ng·hr/mL/mg to about 53.5 ng·hr/mL/mg, or from about 32.0 ng·hr/mL/mg to about 35.0 ng·hr/mL/mg, or from about 35.0 ng·hr/mL/mg to about 38.0 ng·hr/mL/mg, or from about 38.0 ng·hr/mL/mg to about 41.0 ng·hr/mL/mg, or from about 41.0 ng·hr/mL/mg to about 44.0 ng·hr/mL/mg, or from about 44.0 ng·hr/mL/mg to about 47.0 ng·hr/mL/mg, or from about 47.0 ng·hr/mL/mg to about 50.0 ng·hr/mL/mg. In another embodiment, the AUC₂₋₄₈ₕᵣ for acetaminophen may be about 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, or 53.5 ng·hr/mL/mg.

In an additional embodiment, the pharmaceutical composition, when orally administered to a subject under fed conditions (low fat meal), may produce a plasma profile characterized by an AUC₂₋₄₈ₕᵣ for acetaminophen from about 28.0 ng·hr/mL/mg to about 52.5 ng·hr/mL/mg, or from about 32.0 ng·hr/mL/mg to about 35.0 ng·hr/mL/mg, or from about 35.0 ng·hr/mL/mg to about 38.0 ng·hr/mL/mg, or from about 38.0 ng·hr/mL/mg to about 41.0 ng·hr/mL/mg, or from about 41.0 ng·hr/mL/mg to about 44.0 ng·hr/mL/mg, or from about 44.0 ng·hr/mL/mg to about 47.0 ng·hr/mL/mg, or from about 47.0 ng·hr/mL/mg to about 50.0 ng·hr/mL/mg. In another embodiment, the AUC₂₋₄₈ₕᵣ for acetaminophen may be about 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, or 52.5 ng·hr/mL/mg.

In an additional embodiment, the pharmaceutical composition, when orally administered to a subject, may produce a plasma profile characterized by an AUC₈₋₁₀ₕᵣ for acetaminophen from about 2.3 ng·hr/mL/mg to about 6.0 ng·hr/mL/mg, or from about 2.50 ng·hr/mL/mg to about 3.0 ng·hr/mL/mg, or from about 3.0 ng·hr/mL/mg to about 3.5 ng·hr/mL/mg, or from about 3.5 ng·hr/mL/mg to about 4.0 ng·hr/mL/mg, or from about 4.0 ng·hr/mL/mg to about 4.5 ng·hr/mL/mg, or from about 4.5 ng·hr/mL/mg to about 5.0 ng·hr/mL/mg, or from about 5.0 ng·hr/mL/mg to about 5.5 ng·hr/mL/mg. In another embodiment, the AUC₈₋₁₀ₕᵣ for acetaminophen may be about 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 ng·hr/mL/mg.

In an additional embodiment, the pharmaceutical composition, when orally administered to a subject under fasted conditions, may produce a plasma profile characterized by an AUC₈₋₁₀ₕᵣ for acetaminophen from about 2.3 ng·hr/mL/mg to about 4.5 ng·hr/mL/mg, or from about 2.50 ng·hr/mL/mg to about 3.0 ng·hr/mL/mg, or from about 3.0 ng·hr/mL/mg to about 3.5 ng·hr/mL/mg, or from about 3.5 ng·hr/mL/mg to about 4.0 ng·hr/mL/mg. In another embodiment, the AUC₈₋₁₀ₕᵣ for acetaminophen may be about 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 ng·hr/mL/mg.

In an additional embodiment, the pharmaceutical composition, when orally administered to a subject under fed conditions (high fat meal), may produce a plasma profile characterized by an AUC₈₋₁₀ₕᵣ for acetaminophen from about 3.0 ng·hr/mL/mg to about 5.8 ng·hr/mL/mg, or from about 3.5 ng·hr/mL/mg to about 4.0 ng·hr/mL/mg, or from about 4.0 ng·hr/mL/mg to about 4.5 ng·hr/mL/mg, or from about 4.5 ng·hr/mL/mg to about 5.0 ng·hr/mL/mg, or from about 5.0 ng·hr/mL/mg to about 5.5 ng·hr/mL/mg. In another embodiment, the AUC₈₋₁₀ₕᵣ for acetaminophen may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, or 5.8 ng·hr/mL/mg.

In an additional embodiment, the pharmaceutical composition, when orally administered to a subject under fed conditions (low fat meal), may produce a plasma profile characterized by an AUC₈₋₁₀ₕᵣ for acetaminophen from about 3.0 ng·hr/mL/mg to about 6.0 ng·hr/mL/mg, or from about 3.5 ng·hr/mL/mg to about 4.0 ng·hr/mL/mg, or from about 4.0 ng·hr/mL/mg to about 4.5 ng·hr/mL/mg, or from about 4.5 ng·hr/mL/mg to about 5.0 ng·hr/mL/mg, or from about 5.0 ng·hr/mL/mg to about 5.5 ng·hr/mL/mg. In another embodiment, the AUC₈₋₁₀ₕᵣ for acetaminophen may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 ng·hr/mL/mg.

In an additional embodiment, the pharmaceutical composition, when orally administered to a subject, may produce a plasma profile characterized by an AUC₁₀₋₁₂ₕᵣ for acetaminophen from about 1.8 ng·hr/mL/mg to about 5.0 ng·hr/mL/mg, or from about 2.0 ng·hr/mL/mg to about 2.5 ng·hr/mL/mg, or from about 2.5 ng·hr/mL/mg to about 3.0 ng·hr/mL/mg, or from about 3.0 ng·hr/mL/mg to about 3.5 ng·hr/mL/mg, or from about 3.5 ng·hr/mL/mg to about 4.0 ng·hr/mL/mg, or from about 4.0 ng·hr/mL/mg to about 4.5 ng·hr/mL/mg. In another embodiment, the AUC₁₀₋₁₂ₕᵣ for acetaminophen may be about 1.8. 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 ng·hr/mL/mg.

In an additional embodiment, the pharmaceutical composition, when orally administered to a subject under fasted conditions, may produce a plasma profile characterized by an AUC₁₀₋₁₂ₕᵣ for acetaminophen from about 1.8 ng·hr/mL/mg to about 3.5 ng·hr/mL/mg, or from about 2.0 ng·hr/mL/mg to about 2.5 ng·hr/mL/mg, or from about 2.5 ng·hr/mL/mg to about 3.0 ng·hr/mL/mg, or from about 3.0 ng·hr/mL/mg to about 3.5 ng·hr/mL/mg. In another embodiment, the AUC₁₀₋₁₂ₕᵣ for acetaminophen may be about 1.8. 1.9, 2.0, 2.1, 2.2, 2.3, 2.4, 2.5, 2.6, 2.7, 2.8, 2.9, 3.0, 3.1, 3.2, 3.3, 3.4, or 3.5 ng·hr/mL/mg.

In an additional embodiment, the pharmaceutical composition, when orally administered to a subject under fed conditions (high fat meal), may produce a plasma profile characterized by an AUC₁₀₋₁₂ₕᵣ for acetaminophen from about 2.7 ng·hr/mL/mg to about 5.0 ng·hr/mL/mg, or from about 3.0 ng·hr/mL/mg to about 3.5 ng·hr/mL/mg, or from about 3.5 ng·hr/mL/mg to about 4.0 ng·hr/mL/mg, or from about 4.0 ng·hr/mL/mg to about 4.5 ng·hr/mL/mg. In another embodiment, the AUC₁₀₋₁₂ₕᵣ for acetaminophen may be about 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 ng·hr/mL/mg.

In an additional embodiment, the pharmaceutical composition, when orally administered to a subject under fed conditions (low fat meal), may produce a plasma profile characterized by an AUC₁₀₋₁₂ₕᵣ for acetaminophen from about 2.4 ng·hr/mL/mg to about 4.5 ng·hr/mL/mg, or from about 2.5 ng·hr/mL/mg to about 3.0, or from about 3.0 ng·hr/mL/mg to about 3.5 ng·hr/mL/mg, or from about 3.5 ng·hr/mL/mg to about 4.0 ng·hr/mL/mg, or from about 4.0 ng·hr/mL/mg to about 4.5 ng·hr/mL/mg. In another embodiment, the AUC₁₀₋₁₂ₕᵣ for acetaminophen may be about 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 ng·hr/mL/mg.

In a further embodiment, the pharmaceutical composition, when orally administered to a subject, may produce a plasma profile characterized by an AUC₀₋₄ₕᵣ for acetaminophen from about 10.0 ng·hr/mL/mg to about 20.0 ng·hr/mL/mg, from about 13.0 ng·hr/mL/mg to about 14.5 ng·hr/mL/mg, or from about 14.5 ng·hr/mL/mg to about 16.5 ng·hr/mL/mg. In another embodiment, the AUC_{0-4 hr} for acetaminophen may be about 10.0, 11.0, 12.0, 13.0, 13.5, 14.0, 14.5, 15.0, 15.5, 16.0, 16.5, or 17.0 ng·hr/mL/mg.

In yet another embodiment, the pharmaceutical composition, when orally administered to a subject, may produce a plasma profile characterized by an AUC_{Tmax-t} for acetaminophen from about 20.0 ng·hr/mL/mg to about 40.0 ng·hr/mL/mg, from about 23.5 ng·hr/mL/mg to about 36.0 ng·hr/mL/mg, or from about 29.0 ng·hr/mL/mg to about 31.0 ng·hr/mL/mg. In another embodiment, the AUC_{Tmax-t} for acetaminophen may be about 20.0, 21.0, 22.0, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5 or 36.0 ng·hr/mL/mg.

In yet another embodiment, the pharmaceutical composition, when orally administered to a subject, may produce a plasma profile characterized by an AUC(0-(Tmax of IR product+2SD)) for acetaminophen after a single dose from about 5.0 ng·hr/mL/mg to about 13.0 ng·hr/mL/mg, from about 7.2 ng·hr/mL/mg to about 11.6 ng·hr/mL/mg, or from about 8.5 ng·hr/mL/mg to about 10.0 ng·hr/mL/mg. In another embodiment, the AUC(0-(Tmax of IR product+2SD)) for acetaminophen may be about 5.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In one embodiment, the immediate release product referenced for the Partial AUC calculations is Percocet in the fasted state and the following calculation was used to determine AUC(0-(Tmax of IR product+2SD)):

acetaminophen mean±SD=0.596 h±0.529 h; Tmax+2SD=1.65 hour

In such embodiment, the pharmaceutical composition, when orally administered to a subject, may produce a plasma profile characterized by an AUC(0-1.7) for acetaminophen after a single dose from about 5.0 ng·hr/mL/mg to about 13.0 ng·hr/mL/mg, from about 7.2 ng·hr/mL/mg to about 11.6 ng·hr/mL/mg, or from about 8.5 ng·hr/mL/mg to about 10.0 ng·hr/mL/mg. In another embodiment, the AUC(0-1.7) for acetaminophen may be about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In still a further embodiment, the pharmaceutical composition, when orally administered to a subject, may produce a plasma profile characterized by an AUC(1.7-48) for acetaminophen after a single dose from about 25.0 ng·hr/mL/mg to about 75.0 ng·hr/mL/mg, from about 31.5 ng·hr/mL/mg to about 55.0 ng·hr/mL/mg, or from about 35.0 ng·hr/mL/mg to about 50.0 ng·hr/mL/mg. In another embodiment, the AUC(1.7-48) for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg.

In one embodiment, the immediate release product referenced for the Partial AUC calculations is Percocet in the fed state and the following calculation was used to determine AUC(0-(Tmax of IR product+2SD)):

acetaminophen mean±SD=1.48 h±0.875 h; Tmax+2SD=3.2 hour

In such embodiment, the pharmaceutical composition, when orally administered to a subject, may produce a plasma profile characterized by an AUC(0-3.2) for acetaminophen after a single dose from about 7.0 ng·hr/mL/mg to about 21.0 ng·hr/mL/mg, from about 9.0 ng·hr/mL/mg to about 18.0 ng·hr/mL/mg, from about 10.0 ng·hr/mL/mg to about 16.0 ng·hr/mL/mg, or from about 12.0 ng·hr/mL/mg to about 15.0 ng·hr/mL/mg. In another embodiment, the AUC(0-3.2) for acetaminophen may be about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, or 21.0 ng·hr/mL/mg.

In still a further embodiment, the pharmaceutical composition, when orally administered to a subject, may produce a plasma profile characterized by an AUC(3.2-48) for acetaminophen after a single dose from about 15.0 ng·hr/mL/mg to about 75.0 ng·hr/mL/mg, from about 25.0 ng·hr/mL/mg to about 55.0 ng·hr/mL/mg, from about 27.5 ng·hr/mL/mg to about 45.0 ng·hr/mL/mg, or from about 30.0 ng·hr/mL/mg to about 40.0 ng·hr/mL/mg. In another embodiment, the AUC(3.2-48) for acetaminophen may be about 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, or 75.0 ng·hr/mL/mg.

In one embodiment, the pharmaceutical composition, when orally administered to a subject, may produce a plasma profile characterized by an AUC0-12 hr for acetaminophen from about 20.0 ng·hr/mL/mg to about 60.0 ng·hr/mL/mg, from about 30 ng·hr/mL/mg to about 50 ng·hr/mL/mg, from about 35 to about 45 ng·hr/mL/mg, or from about 37.5 ng·hr/mL/mg to about 42.5 ng·hr/mL/mg. In another embodiment, the pharmaceutical composition, when orally administered to a subject, may produce a plasma profile characterized by an AUC0-12 hr for acetaminophen from about 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0. In a further embodiment, at AUC0-12 hr between about 70%-95%, about 75%-92%, or about 77%-90% of the acetaminophen has been cleared. In still another embodiment, about 80% of the acetaminophen has been cleared.

In another embodiment, the pharmaceutical composition, when orally administered to a subject, may produce a plasma profile characterized by an AUC1-12 hr for acetaminophen from about 15.0 ng·hr/mL/mg to about 55.0 ng·hr/mL/mg, from about 25.0 ng·hr/mL/mg to about 45.0 ng·hr/mL/mg, or from about 30.0 to about 40.0 ng·hr/mL/mg. In another embodiment, the pharmaceutical composition, when orally administered to a subject, may produce a plasma profile characterized by an AUC1-12 hr for acetaminophen from about 15, 16, 17, 18, 19, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, or 50.0 ng·hr/mL/mg.

In yet another embodiment, the pharmaceutical composition, when orally administered to a subject, may produce a plasma profile characterized by an AUC12-36 hr for acetaminophen from about 5.0 ng·hr/mL/mg to about 25.0 ng·hr/mL/mg, from about 7.5 ng·hr/mL/mg to about 20.0 ng·hr/mL/mg, or from about 10.0 ng·hr/mL/mg to about 15.0. In other embodiments, the pharmaceutical composition, when orally administered to a subject, may produce a plasma profile characterized by an AUC12-36 hr for acetaminophen from about 5.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0 ng·hr/mL/mg.

In another embodiment, the pharmaceutical composition, when orally administered to a subject, may produce a plasma profile characterized by an AUC8-12 hr for acetaminophen from about 1.5 ng·hr/mL/mg to about 15.5 ng·hr/mL/mg, from about 2 ng·hr/mL/mg to about 12.25 ng·hr/mL/mg, from about 3.5 ng·hr/mL/mg to about 10 ng·hr/mL/mg, or from about 4.5 ng·hr/mL/mg to about 6.5 ng·hr/mL/mg. In other embodiments, the pharmaceutical composition, when orally administered to a subject, may produce a plasma profile characterized by an AUC8-12 hr for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In one embodiment, the pharmaceutical composition, when orally administered to a subject, may produce a plasma profile characterized by an AUC(0-3 hr) for acetaminophen from about 5 ng·hr/mL/mg to about 30 ng·hr/mL/mg, from about 10 ng·hr/mL/mg to about 20 ng·hr/mL/mg, or from about 13 ng·hr/mL/mg to about 17 ng·hr/mL/mg. In other embodiments, the pharmaceutical composition, when orally administered to a subject, may produce a plasma profile characterized by an AUC(0-3 hr) for acetaminophen from about 5.0, 6.0, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 ng·hr/mL/mg.

In another embodiment, the pharmaceutical composition, when orally administered to a subject, may produce a plasma profile characterized by an AUC(3-36 hr) for acetaminophen from about 20 ng·hr/mL/mg to about 50 ng·hr/mL/mg, from about 20 ng·hr/mL/mg to about 40 ng·hr/mL/mg, or from about 25 ng·hr/mL/mg to about 35 ng·hr/mL/mg. In other embodiments, the pharmaceutical composition, when orally administered to a subject, may produce a plasma profile characterized by an AUC(3-36 hr) for acetaminophen from about 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng·hr/mL/mg.

In one embodiment, the pharmaceutical composition, when orally administered to a subject, may produce a plasma profile characterized by an AUC0-12 hr for acetaminophen from about 50% to about 90% of the AUC0-t, from about 55% to about 85% of the AUC0-t, or from about 75% to about 85% of the AUC0-t. In other embodiments, the pharmaceutical composition, when orally administered to a subject, may produce a plasma profile characterized by an AUC0-12 hr for acetaminophen that is about 50%, 55%, 60%, 65%, 70%, 75%, 76%, 77%, 78%, 79%, 80%, 81%, 82%, 83%, 84% or 85% of the AUC0-t.

In one embodiment, the pharmaceutical composition, when orally administered to a subject, may produce a plasma profile characterized by an AUC1-12 hr for acetaminophen from about 40% to about 90% of the AUC0-t, from about 55% to about 85% of the AUC0-t, or from about 60% to about 75% of the AUC0-t. In other embodiments, the pharmaceutical composition, when orally administered to a subject, may produce a plasma profile characterized by an AUC1-12 hr for acetaminophen of about 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, or 80% of the AUC0-t.

In one embodiment, the pharmaceutical composition, when orally administered to a subject, may produce a plasma profile characterized by an AUC12-36 hr for acetaminophen from about 10% to about 40% of the AUC0-t, from about 15% to about 35% of the AUC0-t, or from about 20% to about 30% of the AUC0-t. In other embodiments, the pharmaceutical composition, when orally administered to a subject, may produce a plasma profile characterized by an AUC12-36 hr for acetaminophen of about 10%, 12%, 14%, 16%, 18%, 20%, 21%, 22%, 23%, 24%, 25%, 26%, 27%, 28%, 29%, or 30% of the AUC0-t.

In one embodiment, the pharmaceutical composition, when orally administered to a subject, may produce a plasma profile characterized by an AUC8-12 hr for acetaminophen from about 5% to about 30% of the AUC0-t, from about 7% to about 25% of the AUC0-t, or from about 10% to about 20% of the AUC0-t. In other embodiments, the pharmaceutical composition, when orally administered to a subject, may produce a plasma profile characterized by an AUC8-12 hr for acetaminophen of about 5%, 6%, 7%, 8%, 9%, 10%, 11%, 12%, 13%, 14%, 15%, 16%, 17%, 18%, 19%, 20%, 21%, 22%, 23%, 24%, or 25% of the AUC0-t.

In an alternate embodiment, the pharmaceutical composition, when orally administered to a subject, may have a mean half-life of acetaminophen that ranges from about 2 hours to about 10 hours, or from about 3 hours to about 6 hours. In another embodiment, the pharmaceutical composition, when orally administered to a subject, may have a mean half-life of acetaminophen that ranges from about 3 hours to about 5 hours. In still another embodiment, the pharmaceutical composition, when orally administered to a subject, may have a mean half-life of acetaminophen that ranges from about 4 hours to about 5 hours. In various embodiments, the mean half-life of acetaminophen may be about 2.5, 3.0, 3.5, 4.0, 4.5, 5.0, 5.5, 6.0, 6.5, 6.0, 7.0, 7.5, or 8 hours. In additional embodiments, the pharmaceutical composition, when orally administered to a subject, has a mean observed half-life of acetaminophen that is more than the mean half-life of commercially available immediate release acetaminophen products.

In another embodiment, upon administration of the pharmaceutical composition to a subject, the composition may provide at least about 4 hours to about 12 hours of drug delivery to the upper gastrointestinal tract, which includes the duodenum, jejunum, and ileum of the small intestine. In another embodiment, the composition may provide at least about 6 hours of drug delivery to the upper gastrointestinal tract. In yet a further embodiment, the composition may provide at least about 8 hours of drug delivery to the upper gastrointestinal tract. In yet a further embodiment, the composition may provide at least about 9 hours, or at least about 10 hours of drug delivery to the upper gastrointestinal tract.

In yet another embodiment, upon administration of the pharmaceutical composition to a subject, APAP undergoes presystemic metabolism in the gut and/or liver allowing only a fraction of the drug to reach the systemic circulation. The fraction of drug that is originally absorbed prior to pre-systemic metabolism is referred to as the fraction absorbed and denoted “Fab.” This is different from the fraction bioavailable “F,” which is the fraction that reaches the systemic circulation after the metabolism in the gut and liver.

In another embodiment, 60-90% of the acetaminophen in the pharmaceutical composition, which is available for absorption into the systemic circulation, is absorbed in the upper gastrointestinal tract. In still another embodiment, 60-85% of acetaminophen in the pharmaceutical composition, which is available for absorption into the systemic circulation, is absorbed in the duodenum and jejunum. See FIG. 27. Greater than 50% absorption of acetaminophen in the upper gastrointestinal tract is beneficial to a human subject because acetaminophen is poorly absorbed in the stomach and well absorbed in the small intestine and particularly, the upper segment of the gastrointestinal tract. It is therefore critical that acetaminophen is available in upper small intestine for its absorption. In one embodiment acetaminophen is released in stomach and reaches quickly into upper part of the small intestine for the absorption to take place.

In another embodiment, when about 60% to about 75% of the acetaminophen is released from the dosage form in the stomach within 2 hours following oral administration, about 10% to about 25% of the total amount of the acetaminophen in the dosage form, which is available for absorption into the systemic circulation, is absorbed in the duodenum, about 25% to about 40% is absorbed in the proximal jejunum (noted as “jejunum 1” in FIG. 27), about 15% to about 20% is absorbed in the distal jejunum (noted as “jejunum 2” in FIG. 27), and about 5% to about 15% is absorbed in the ileum.

In another embodiment, when about 70% to about 90% of the acetaminophen is released from the dosage form in the stomach within 4 hours following oral administration, about 10% to about 25% of the total amount of the acetaminophen in the dosage form, which is available for absorption into the systemic circulation, is absorbed in the duodenum, about 25% to about 40% is absorbed in the proximal jejunum (noted as “jejunum 1” in FIG. 27), about 15% to about 20% is absorbed in the distal jejunum (noted as “jejunum 2” in FIG. 27), and about 5% to about 15% is absorbed in the ileum.

In yet another embodiment, when at least about 55% of the total amount of the acetaminophen is released from the dosage form in the stomach within 1 hour after oral administration and when at least about 60% of the acetaminophen is released in the stomach after 2 hours, about 15% to about 20% of the total amount of the acetaminophen in the dosage form, which is available for absorption into the systemic circulation, is absorbed in the duodenum, about 30% to about 37% is absorbed in the proximal jejunum, about 15% to about 18% is absorbed in the distal jejunum, and about 8% to about 10% is absorbed in the ileum.

In still another embodiment, upon administration of the pharmaceutical composition to a subject, the opioid undergoes presystemic metabolism in the gut and/or liver allowing only a fraction of the drug to reach the systemic circulation. The fraction of drug that is originally absorbed prior to pre-systemic metabolism is referred to as the fraction absorbed and denoted “Fab.” In one embodiment, the opioid is oxycodone. This is different from the fraction bioavailable “F,” which is the fraction that reaches the systemic circulation after metabolism in the gut and liver.

In a further embodiment, 70-95% of the oxycodone in the pharmaceutical composition, which is available for absorption into the systemic circulation, is absorbed in the upper gastrointestinal tract. In still another embodiment, 80-95% of oxycodone in the pharmaceutical composition, which is available for absorption into the systemic circulation, is absorbed in the duodenum and jejunum. See FIG. 28.

In one embodiment, the composition releases the opioid and other API in the stomach to optimize drug absorption in the duodenum and jejunum. For example, when about 25% to about 50% of oxycodone is released from the dosage form in the stomach within 1 hour following oral administration, about 10% to about 45% of the total amount of the oxycodone in the dosage form, which is available for absorption into the systemic circulation, is absorbed in the duodenum, about 25% to about 50% is absorbed in the proximal jejunum (noted as “jejunum 1” in FIG. 28), about 7% to about 20% is absorbed in the distal jejunum (noted as “jejunum 2” in FIG. 28), and about 2% to about 15% is absorbed in the ileum.

In another embodiment, when about 45% to about 65% of oxycodone is released from the dosage form in the stomach within 2 hours following oral administration, about 10% to about 50% of the total amount of the oxycodone in the dosage form, which is available for absorption into the systemic circulation, is absorbed in the duodenum, about 25% to about 55% is absorbed in the proximal jejunum (noted as “jejunum 1” in FIG. 28), about 5% to about 25% is absorbed in the distal jejunum (noted as “jejunum 2” in FIG. 28), and about 2% to about 15% is absorbed in the ileum.

In another embodiment, when about 60% to about 85% of oxycodone is released from the dosage form in the stomach within 4 hours following oral administration, about 10% to about 55% of the total amount of the oxycodone in the dosage form, which is available for absorption into the systemic circulation, is absorbed in the duodenum, about 30% to about 60% is absorbed in the proximal jejunum (noted as “jejunum 1” in FIG. 28), about 10% to about 30% is absorbed in the distal jejunum (noted as “jejunum 2” in FIG. 28), and about 2% to about 20% is absorbed in the ileum.

In yet another embodiment, when at least 25% of the total amount of the oxycodone is released from the dosage form in the stomach within 1 hour after oral administration and when at least 45% of the oxycodone is released in the stomach after 2 hours, about 30% to about 45% of the total amount of oxycodone in the dosage form, which is available for absorption into the systemic circulation, is absorbed in the duodenum, about 37% to about 43% is absorbed in the proximal jejunum (noted as “jejunum 1” in FIG. 28), about 10% to about 15% is absorbed in the distal jejunum (noted as “jejunum 2” in FIG. 28), and about 2% to about 8% is absorbed in the ileum.

In another embodiment, about 90% to about 100% of the IR dose of acetaminophen is released within about 15 minutes, 30 minutes, 45 minutes or 60 minutes after oral administration. In one embodiment, the dosage form provides a dissolution profile wherein about 20% to about 65%, about 35% to about 55% or about 40% to about 50% of the ER dose of acetaminophen remains in the ER layer between about 1 and 2 hours after administration. In one embodiment, not more than 50% of the ER dose of acetaminophen is released within about the first hour. In a further embodiment, not more than 45% or not more than 40% of the ER dose of acetaminophen is released within about the first hour. In another embodiment, not more than 85% of the ER dose of acetaminophen is released within about 4 hours. In yet another embodiment, not less than 50% is released after about 6 hours. In yet another embodiment, not less than 60% is released after about 6 hours. In one embodiment, the ER dose of acetaminophen is released over a time period of about 6 to 12, about 8 to 10, or about 9 to 10 hours in vitro. In another embodiment, the ER dose of acetaminophen is released over a time period of about 7 hours, 8 hours, 9 hours, 10 hours, 11 hours or 12 hours in vitro. In another embodiment, at least 90% or 95% of the ER dose of acetaminophen is released over a time period of about 7 hours, 8 hours, 9 hours, 10 hours, 11 hours or 12 hours in vitro.

In one embodiment, the pharmaceutical compositions disclosed herein rapidly achieve therapeutic plasma drug levels of oxycodone and acetaminophen similar to an immediate release product, which provides an early onset of action within about the first 5 minutes, 10 minutes, 15 minutes, 20 minutes, 25 minutes, 30 minutes, 35 minutes, 40 minutes, 45 minutes, 50 minutes, 55 minutes or 60 minutes after administration of the composition, but unlike an immediate release product, the pharmaceutical composition is able to maintain those therapeutic plasma drug levels of oxycodone and acetaminophen over an extended period of time (e.g., up to 12 hours). Currently, there is no pharmaceutical composition available comprising oxycodone and acetaminophen which is able to provide a patient with quick onset of analgesia and maintenance of analgesia for an extended period of time.

In yet another embodiment, upon average, within one hour of administration to a subject, the pharmaceutical composition achieves a Cmax for acetaminophen. The Cmax achieved by the pharmaceutical composition disclosed herein is comparable to the Cmax obtained from a commercially-available immediate release product containing acetaminophen formulated at half the strength of the commercially-available immediate release product. The acetaminophen continues to be released from the pharmaceutical composition at a rate less than the clearance rate for the acetaminophen, so that the acetaminophen levels fall smoothly until all of the acetaminophen is absorbed. Stated another way, the acetaminophen released by the pharmaceutical composition is eliminated by the body faster than it is being absorbed. The absorption of the acetaminophen released from the pharmaceutical composition is complete in about 8 to about 10 hours so that for one half life of acetaminophen the blood supply reaching the subject's liver via the portal vein contains no additional amounts of acetaminophen beyond the amounts present in the subject's general circulation.

These additional amounts of acetaminophen delivered to the liver from the subject's portal vein are frequently caused by the absorption of acetaminophen in the subject's gastrointestinal tract. Indeed, blood from the subject's intestines passes through the liver and then on to the general circulation. When acetaminophen is undergoing absorption, blood containing acetaminophen from the absorption process passes through the subject's liver prior to entering the general circulation where the acetaminophen is diluted by the distribution and clearance processes. The metabolism of these higher acetaminophen concentrations in blood coming into the subject's liver is termed the “first pass effect.” Hence, the absorption process for acetaminophen taxes a subject's metabolic systems in the liver due to these higher “first pass” concentrations. Once the absorption process is complete, the concentration of acetaminophen in the blood reaching the subject's liver through the portal vein will be the same concentration of acetaminophen as found in blood throughout the rest of the subject's body. Thus, the pharmaceutical compositions disclosed herein provide a Cmax comparable to a commercially-available immediate-release acetaminophen product (dosed at half strength) while providing a less taxing burden on the subject's metabolic systems in the liver because the acetaminophen released by the pharmaceutical composition is eliminated by the subject's body faster than it is being absorbed. This results in decreased levels of acetaminophen in a subject's liver as compared to an immediate release dosage form of acetaminophen dosed every 6 hours.

(i) the Pharmacokinetic Profiles of the Pharmaceutical Compositions of the Invention are not Affected by the Fed or Fasted State of the Subject

Food can play a significant role in both the rate and extent of absorption of a drug. As is known, the primary function of the small intestine is to absorb food. During and after a meal, the intestine normally shows very irregular or unsynchronized contractions that move the food content back and forth and mix it with the digestive enzymes that are secreted into the intestine. However, these contractions are not entirely unsynchronized; they move the contents of the intestine slowly towards the large intestine. It normally takes about 90-120 minutes for the first part of a meal to reach the large intestine, and the last portion of the meal may not reach the large intestine for five (5) hours. Between meals, the intestine shows cycles of activity that repeat about every 90-120 minutes. The cycle consists of a short period of very few contractions (Phase I), followed by a long period of unsynchronized contractions that appear similar to the fed pattern (pre-burst, Phase II), and then a burst of strong, regular contractions that move down the intestine in a peristaltic fashion (Phase III). Phase III represents a continuation of the “housekeeper waves” that start in the stomach; its function is to sweep undigested food particles and bacteria out of the small intestine and ultimately into the large intestine.

Because non-opioid GR dosage forms of the prior art, as well as prior art extended release opioid formulations, demonstrate food effects, Applicants expected to likewise see a food effect with the pharmaceutical compositions of the present invention. Here, however, Applicants have surprisingly discovered that the pharmacokinetic profiles of a pharmaceutical composition that comprises oxycodone and acetaminophen are not substantially affected by the fed or fasted state of a human subject ingesting the composition.

In general, a fed state is defined as having consumed food within about 30 min prior to administration of the composition. The food may be a high fat meal, a low fat meal, a high calorie meal, or a low calorie meal. A fasted state may be defined as not having ingested food for at least 10 hours prior to administration of the composition. In some embodiments, the subject may have fasted for at least 10 hours prior to the first dose and refrains from ingesting food for at least one hour prior to administration of subsequent doses. In other embodiments, the fasted subject may not have ingested food for at least 1 hour, 2 hours, 3 hours, 4 hours, 5 hours, 6 hours, 7 hours, 8 hours, 9 hours, or 10 hours prior to administration of each dose of the composition.

As the pharmacokinetic profiles of a pharmaceutical composition that comprises oxycodone and acetaminophen are not substantially affected by the fed or fasted state of a human subject, there is no substantial difference in the quantity of drug absorbed or the rate of drug absorption when the oxycodone/acetaminophen compositions are administered in the fed versus the fasted state. Without being bound to theory, Applicants believe that in a fasted state the opioid acts to reduce gastric motility in an amount sufficient to retain the dosage form in the stomach thereby mitigating the “housekeeper waves” described above.

As shown in Examples 6 and 9, the pharmacokinetic parameters of the compositions of the invention are similar when the composition is administered in the fed and fasted states. Benefits of a dosage form, which substantially eliminates the effect of food, include an increase in convenience, thereby increasing patient compliance, as the patient does not need to ensure that they are taking a dose either with or without food. This is significant because poor patient compliance can lead to adverse therapeutic outcomes.

The invention also encompasses an oxycodone/APAP pharmaceutical composition in which administration of the composition to a human subject in a fasted state is bioequivalent to administration of the composition to a human subject in a fed state wherein bioequivalence is established by: (1) a 90% Confidence Interval (CI) for AUC which is between 80% and 125%, and (2) a 90% CI for Cmax, which is 80% and 125%. In a further embodiment, the compositions disclosed herein may by administered to a subject in need thereof without regard to food.

In other embodiments, the difference in absorption of either the opioids and/or the APIs of the invention, when administered in the fed versus the fasted state, is less than about 35%, less than about 30%, less than about 25%, less than about 20%, less than about 15%, less than about 10%, less than about 5%, or less than about 3%. The pharmacokinetic parameter of the other API(s) that is independent of food may be, but is not limited to, Cₘₐₓ, C₁ₕᵣ, C₂ₕᵣ, AUC, partial AUC, Tₘₐₓ, and T_{lag}. Additionally, the opioid(s) in the composition produce a plasma profile characterized by at least one pharmacokinetic parameter that differs by less than about 30% under fed and fasted conditions. In various embodiments, the pharmacokinetic parameter may vary by less than about 25%, 20%, 15%, 10%, or 5% under fed and fasted conditions. In one embodiment, the pharmacokinetic parameter of the opioid that is independent of food may be, but is not limited to, Cmax, C1 hr, C2 hr, AUC, partial AUC, Tmax, and Tlag.

### (i) Exemplary Compositions

In one embodiment, the pharmaceutical composition for extended release of oxycodone and acetaminophen comprises at least one extended release portion comprising acetaminophen, oxycodone or a combination thereof, and the at least one extended release portion of the composition comprises an extended release component and oxycodone, acetaminophen, or a combination thereof. In yet another embodiment, the composition comprises an immediate release portion comprising oxycodone and acetaminophen and an extended release portion comprising oxycodone, acetaminophen and an extended release component. In still yet another embodiment, the compositions comprises two extended release portions, each comprising an extended release component and one of oxycodone or acetaminophen, and an immediate release portion comprising oxycodone and acetaminophen. In another embodiment, the composition comprises two extended release portions, each comprising an extended release component and one of oxycodone or acetaminophen, and two immediate release portions, each comprising one of oxycodone or acetaminophen. In one embodiment, the extended release component comprise at least one extended release polymer. In one exemplary embodiment, the at least one extended release polymer comprises a polyethylene oxide. The molecular weight of the polyethylene oxide may be from about 500,000 Daltons to about 10,000,000 Daltons.

In another embodiment, the pharmaceutical composition may comprise from about 5 mg to about 30 mg of oxycodone and from about 250 mg to about 1300 mg of acetaminophen. In one embodiment, the composition may comprise about 15 mg of oxycodone and about 650 mg of acetaminophen. In another exemplary embodiment, the composition may comprise about 15 mg of oxycodone and about 500 mg of acetaminophen. In still another embodiment, the composition may comprise about 15 mg of oxycodone and about 325 mg of acetaminophen. In yet another exemplary embodiment, the composition may comprise about 7.5 mg of oxycodone and about 325 mg of acetaminophen. In yet another exemplary embodiment, the composition may comprise about 5 mg of oxycodone and about 325 mg of acetaminophen. In still another exemplary embodiment, the pharmaceutical composition may comprise about 10 mg of oxycodone and about 325 mg of acetaminophen. In a further exemplary embodiment, the pharmaceutical composition may comprise about 20 mg of oxycodone and about 650 mg of acetaminophen. In another exemplary embodiment, the composition may comprise about 30 mg of oxycodone and about 650 mg of acetaminophen.

In another embodiment, the composition may comprise from about 5 mg to about 30 mg of opioid and from about 250 mg to about 1300 mg of at least one other API. In one embodiment, the composition may comprise about 15 mg of opioid and about 650 mg of at least one other API. In another embodiment, the composition may comprise about 15 mg of opioid and about 500 mg of at least one other API. In a further embodiment, the composition may comprise about 30 mg of opioid and about 500 mg of at least one other API. In still another embodiment, the composition may comprise about 15 mg of opioid and about 325 mg of at least one other API. In yet another exemplary embodiment, the composition may comprise about 7.5 mg of opioid and about 325 mg of at least one other API. In yet another exemplary embodiment, the composition may comprise about 5 mg of opioid and about 325 mg of at least one other API. In still another exemplary embodiment, the pharmaceutical composition may comprise about 10 mg of opioid and about 325 mg of at least one other API. In a further exemplary embodiment, the pharmaceutical composition may comprise about 20 mg of opioid and about 650 mg of at least one other API. In another exemplary embodiment, the composition may comprise about 30 mg of opioid and about 650 mg of at least one other API. In yet another exemplary embodiment, the composition may comprise about 22.5 mg of opioid and about 925 mg of at least one other API.

In a further embodiment, a single dosage form of the pharmaceutical composition disclosed herein (e.g., one tablet) will provide a subject with approximately the same therapeutic benefit and pharmacokinetic profile as either two dosage forms (e.g., two tablets) of the composition formulated at half the strength, or three dosage forms (e.g., three tablets) of the composition formulated at a third of the strength. In yet another exemplary embodiment, the pharmaceutical composition comprising 15 mg of oxycodone and 650 mg of acetaminophen in a single dosage form (e.g., one tablet) will provide a subject with approximately the same therapeutic benefit and pharmacokinetic profile as two dosage forms of the pharmaceutical composition formulated at half the strength (e.g., each tablet comprising 7.5 mg of oxycodone and 325 mg of acetaminophen). In still another exemplary embodiment, the pharmaceutical composition comprising 15 mg of oxycodone and 650 mg of acetaminophen in a single dosage form (e.g., one tablet) will provide a subject with approximately the same therapeutic benefit and pharmacokinetic profile as three dosage forms of the pharmaceutical composition formulated at a third of the strength (e.g., each tablet comprising 5 mg of oxycodone and about 216.7 mg of acetaminophen). In yet another embodiment, the pharmaceutical composition comprising 15 mg of oxycodone and 325 mg of acetaminophen in a single dosage form (e.g., one tablet) taken together with another tablet comprising 7.5 mg of oxycodone and 325 mg of acetaminophen in a single dosage form will provide a subject with approximately the same therapeutic benefit and pharmacokinetic profile as a single tablet comprising 22.5 mg of oxycodone and 650 mg of acetaminophen. In still another exemplary embodiment, the pharmaceutical composition comprising 15 mg of oxycodone and 325 mg of acetaminophen in a single dosage form (e.g., one tablet) taken together with another tablet comprising 15 mg of oxycodone and 325 mg of acetaminophen in a single dosage form will provide a subject with approximately the same therapeutic benefit and pharmacokinetic profile as a single tablet configuration totaling 30 mg of oxycodone and 650 mg of acetaminophen. In yet a further exemplary embodiment, a pharmaceutical composition comprising 21 mg of oxycodone and 650 mg of acetaminophen in a single dosage form (e.g., one tablet) will provide a subject with approximately the same therapeutic benefit and pharmacokinetic profile as two dosage forms of the pharmaceutical composition formulated at half the strength (e.g., each tablet comprising 10.5 mg of oxycodone and 325 mg of acetaminophen). In yet another exemplary embodiment, a pharmaceutical composition comprising 22.5 mg of oxycodone and 925 mg of acetaminophen in a single dosage form (e.g., one tablet) will provide a subject with approximately the same therapeutic benefit and pharmacokinetic profile as three dosage forms of the pharmaceutical composition formulated at a third of the strength (e.g., each tablet comprising 7.5 mg of oxycodone and 325 mg of acetaminophen).

In yet another embodiment, the at least one extended release portion of the composition may comprise from about 40% to about 60% (w/w) of the total amount of acetaminophen in the composition and from about 70% to about 80% (w/w) of the total amount of oxycodone the composition, whereas the at least one immediate release portion may comprise from about 40% to about 60% (w/w) of the total amount of acetaminophen in the composition and from about 20% to about 30% (w/w) of the total amount of oxycodone in the composition. In still another embodiment, the at least one extended release portion may comprise about 50% (w/w) of the total amount of acetaminophen in the composition and about 75% (w/w) of the total amount of oxycodone.

In yet another embodiment, an immediate release portion of the composition may comprise, by weight of such immediate release portion, from about 70% to about 80% acetaminophen and from about 0.5% to about 1% of oxycodone, and an extended release portion of the composition may comprise, by weight of such extended release portion, from about 30% to about 50% of the extended release polymer, from about 20% to about 40% of acetaminophen, and from about 0.5% to about 2% of oxycodone. In another embodiment, the at least one immediate release portion may comprise about 50% (w/w) of total amount of acetaminophen in the composition and about 25% (w/w) of the total amount of oxycodone in the composition.

In another embodiment, an extended release portion of the composition may comprise, by weight of such extended release portion, from about 30% to about 50% of the extended release polymer, from about 20% to about 40% of acetaminophen, and from about 0.5% to about 2% of oxycodone; and an immediate release portion may comprise, by weight of such immediate release portion, from about 70% to about 80% acetaminophen and from about 0.5% to about 1% of oxycodone.

In yet another embodiment, the pharmaceutical composition may comprise from about 7.5 mg to about 30 mg of oxycodone and from about 325 mg to about 650 mg of acetaminophen, wherein the at least one immediate release portion may comprise about 25% (w/w) of the total amount of oxycodone in the composition and about 50% (w/w) of the total amount of acetaminophen in the composition, and the at least one extended release portion may comprise about 75% (w/w) of the total amount of oxycodone in the composition, about 50% (w/w) of the total amount of acetaminophen in the composition, and about 35% to about 45%, by weight of the at least one extended release portion, of an extended release polymer comprising a polyethylene oxide.

In yet another embodiment, the pharmaceutical composition may comprise about 5 mg of oxycodone and about 325 mg of acetaminophen, wherein the at least one immediate release portion may comprise about 25% (w/w) of the total amount of oxycodone in the composition and about 50% (w/w) of the total amount of acetaminophen in the composition, and the at least one extended release portion may comprise about 75% (w/w) of the total amount of oxycodone in the composition, about 50% (w/w) of the total amount of acetaminophen in the composition.

In a further embodiment, the pharmaceutical composition may comprise about 5 mg of oxycodone and about 325 mg of acetaminophen, wherein the at least one immediate release portion may comprise about 20% (w/w) to about 30% (w/w) of the total amount of oxycodone in the composition, and about 40% (w/w) to about 60% (w/w) of the total amount of acetaminophen in the composition; and the at least one extended release portion may comprise about 70% (w/w) to about 80% (w/w) of the total amount of oxycodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 5 mg of oxycodone and about 325 mg of acetaminophen, wherein the at least one immediate release portion may comprise about 1.25 mg of oxycodone and about 162.5 mg of acetaminophen, and the at least one extended release portion may comprise about 3.75 mg of oxycodone and about 162.5 mg of acetaminophen.

In still another embodiment, the pharmaceutical composition may comprise about 5 mg of oxycodone and about 325 mg of acetaminophen, wherein the at least one immediate release portion may comprise about 0.75 mg to about 2 mg of oxycodone and about 125 mg to about 325 mg of acetaminophen; and the at least one extended release portion may comprise about 3 mg to about 4.5 mg of oxycodone and about 125 mg to about 325 mg of acetaminophen.

In yet another embodiment, the pharmaceutical composition may comprise about 7.5 mg of oxycodone and about 325 mg of acetaminophen, wherein the at least one immediate release portion may comprise about 25% (w/w) of the total amount of oxycodone in the composition and about 50% (w/w) of the total amount of acetaminophen in the composition, and the at least one extended release portion may comprise about 75% (w/w) of the total amount of oxycodone in the composition, about 50% (w/w) of the total amount of acetaminophen in the composition.

In a further embodiment, the pharmaceutical composition may comprise about 7.5 mg of oxycodone and about 325 mg of acetaminophen, wherein the at least one immediate release portion may comprise about 20% (w/w) to about 30% (w/w) of the total amount of oxycodone in the composition, and about 40% (w/w) to about 60% (w/w) of the total amount of acetaminophen in the composition; and the at least one extended release portion may comprise about 70% (w/w) to about 80% (w/w) of the total amount of oxycodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 7.5 mg of oxycodone and about 325 mg of acetaminophen, wherein the at least one immediate release portion may comprise about 1.875 mg of oxycodone and about 162.5 mg of acetaminophen, and the at least one extended release portion may comprise about 5.625 mg of oxycodone and about 162.5 mg of acetaminophen.

In still another embodiment, the pharmaceutical composition may comprise about 7.5 mg of oxycodone and about 325 mg of acetaminophen, wherein the at least one immediate release portion may comprise about 1 mg to about 3 mg of oxycodone and about 125 mg to about 325 mg of acetaminophen; and the at least one extended release portion may comprise about 4.75 mg to about 6.5 mg of oxycodone and about 125 mg to about 325 mg of acetaminophen.

In yet another embodiment, the pharmaceutical composition may comprise about 10 mg of oxycodone and about 325 mg of acetaminophen, wherein the at least one immediate release portion may comprise about 25% (w/w) of the total amount of oxycodone in the composition and about 50% (w/w) of the total amount of acetaminophen in the composition, and the at least one extended release portion may comprise about 75% (w/w) of the total amount of oxycodone in the composition, about 50% (w/w) of the total amount of acetaminophen in the composition.

In a further embodiment, the pharmaceutical composition may comprise about 10 mg of oxycodone and about 325 mg of acetaminophen, wherein the at least one immediate release portion may comprise about 20% (w/w) to about 30% (w/w) of the total amount of oxycodone in the composition, and about 40% (w/w) to about 60% (w/w) of the total amount of acetaminophen in the composition; and the at least one extended release portion may comprise about 70% (w/w) to about 80% (w/w) of the total amount of oxycodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 10 mg of oxycodone and about 325 mg of acetaminophen, wherein the at least one immediate release portion may comprise about 2.5 mg of oxycodone and about 162.5 mg of acetaminophen, and the at least one extended release portion may comprise about 7.5 mg of oxycodone and about 162.5 mg of acetaminophen.

In still another embodiment, the pharmaceutical composition may comprise about 10 mg of oxycodone and about 325 mg of acetaminophen, wherein the at least one immediate release portion may comprise about 1.5 mg to about 3.5 mg of oxycodone and about 125 mg to about 325 mg of acetaminophen; and the at least one extended release portion may comprise about 6.25 mg to about 8.75 mg of oxycodone and about 125 mg to about 325 mg of acetaminophen.

In yet another embodiment, the pharmaceutical composition may comprise about 15 mg of oxycodone and about 325 mg of acetaminophen, wherein the at least one immediate release portion may comprise about 25% (w/w) of the total amount of oxycodone in the composition and about 50% (w/w) of the total amount of acetaminophen in the composition, and the at least one extended release portion may comprise about 75% (w/w) of the total amount of oxycodone in the composition, about 50% (w/w) of the total amount of acetaminophen in the composition.

In a further embodiment, the pharmaceutical composition may comprise about 15 mg of oxycodone and about 325 mg of acetaminophen, wherein the at least one immediate release portion may comprise about 20% (w/w) to about 30% (w/w) of the total amount of oxycodone in the composition, and about 40% (w/w) to about 60% (w/w) of the total amount of acetaminophen in the composition; and the at least one extended release portion may comprise about 70% (w/w) to about 80% (w/w) of the total amount of oxycodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 15 mg of oxycodone and about 325 mg of acetaminophen, wherein the at least one immediate release portion may comprise about 3.75 mg of oxycodone and about 162.5 mg of acetaminophen, and the at least one extended release portion may comprise about 11.25 mg of oxycodone and about 162.5 mg of acetaminophen.

In still another embodiment, the pharmaceutical composition may comprise about 15 mg of oxycodone and about 325 mg of acetaminophen, wherein the at least one immediate release portion may comprise about 2.5 mg to about 5 mg of oxycodone and about 125 mg to about 325 mg of acetaminophen; and the at least one extended release portion may comprise about 10 mg to about 12.5 mg of oxycodone and about 125 mg to about 325 mg of acetaminophen.

Other exemplary formulations are set forth in Charts 1-2 below:

CHART 1
Representative Oxycodone/Acetaminophen Formulations.

| | | Formulation No. | | | | | | | | | |
|---|---|---|---|---|---|---|---|---|---|---|---|
| | | 1 | 2 | 3 | 4 | 5 | 6 | 7 | 8 | 9 | 10 |
| Immediate Release Layer | APAP | 185.3 | 175.0 | 180.0 | 160.4 | 200.0 | 193.4 | 118.8 | 162.5 | 139.0 | 150.0 |
| | Oxycodone hydrochloride | 1.100 | 1.75 | 2.00 | 2.50 | 1.25 | 1.00 | 2.75 | 1.875 | 1.75 | 1.875 |
| | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended | APAP | 185.3 | 150.0 | 145.0 | 155.2 | 125.0 | 100.5 | 146.9 | 162.5 | 207.4 | 150.0 |
| | Oxycodone hydrochloride | 6.900 | 5.75 | 5.50 | 5.00 | 6.25 | 6.50 | 7.25 | 5.625 | 4.75 | 6.625 |
| | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox N12K | 292.8 | — | — | — | 287.7 | — | — | — | 155.5 | — |
| | Polyox 1105 | — | — | 244.2 | — | — | — | 275.5 | 321.8 | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 11 | 12 | 13 | 14 | 15 | 16 | 17 | 18 | 19 | 20 |
| Immediate Release Layer | APAP | 300.0 | 150.0 | 200.0 | 150.0 | 100.0 | 160.0 | 190.0 | 75.0 | 90.0 | 125.0 |
| | Oxycodone hydrochloride | 2.00 | 1.00 | 1.50 | 3.50 | 2.75 | 1.25 | 1.25 | 2.50 | 1.75 | 3.00 |
| | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended Release | APAP | 150.0 | 150.0 | 125.0 | 75.0 | 100.0 | 165.0 | 135.0 | 225.0 | 210.0 | 150.0 |
| | Oxycodone hydrochloride | 8.00 | 6.50 | 6.00 | 6.50 | 3.25 | 6.25 | 6.25 | 5.00 | 6.25 | 5.50 |
| | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox N12K | — | — | 252.4 | — | 290.3 | — | 248.2 | 279.2 | 175.2 | — |
| | Polyox 1105 | 275.8 | — | — | — | — | — | — | — | — | 224.5 |
| | Hydroxypropyl methyl | — | 101.1 | — | 110.5 | — | 192.1 | — | — | 140.9 | 185.6 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |
| | | Formulation No. | | | | | | | | | |
| | | 21 | 22 | 23 | 24 | 25 | 26 | 27 | 28 | 29 | 30 |
| Immediate Release Layer | APAP | 185.3 | 175.0 | 180.0 | 160.4 | 200.0 | 193.4 | 118.8 | 162.5 | 139.0 | 150.0 |
| | Oxycodone hydrochloride | 1.100 | 1.75 | 2.00 | 2.50 | 1.25 | 1.00 | 2.75 | 1.875 | 1.75 | 1.875 |
| | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended Release | APAP | 185.3 | 150.0 | 145.0 | 155.2 | 125.0 | 100.5 | 146.9 | 162.5 | 207.4 | 150.0 |
| | Oxycodone hydrochloride | 6.900 | 5.75 | 5.50 | 5.00 | 6.25 | 6.50 | 7.25 | 5.625 | 4.75 | 6.625 |
| | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox N60K | 292.8 | — | — | — | 287.7 | — | — | — | 155.5 | — |
| | Polyox 205 | — | — | 244.2 | — | — | — | 275.5 | 321.8 | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 31 | 32 | 33 | 34 | 35 | 36 | 37 | 38 | 39 | 40 |
| Immediate Release Layer | APAP | 300.0 | 150.0 | 200.0 | 150.0 | 100.0 | 160.0 | 190.0 | 75.0 | 90.0 | 125.0 |
| | Oxycodone hydrochloride | 2.00 | 1.00 | 1.50 | 3.50 | 2.75 | 1.25 | 1.25 | 2.50 | 1.75 | 3.00 |
| | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended Release | APAP | 150.0 | 150.0 | 125.0 | 75.0 | 100.0 | 165.0 | 135.0 | 225.0 | 210.0 | 150.0 |
| | Oxycodone hydrochloride | 8.00 | 6.50 | 6.00 | 6.50 | 3.25 | 6.25 | 6.25 | 5.00 | 6.25 | 5.50 |
| | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox N60K | — | 45.5 | 249.9 | 24.3 | 282.0 | 49.8 | 200.1 | 240.1 | 186.8 | — |
| | Polyox 205 | 268.4 | — | 53.6 | 70.2 | — | — | 36.3 | 10.4 | — | 259.3 |
| | Hydroxypropyl methyl | — | 90.5 | — | 65.4 | — | 192.1 | — | — | 127.3 | 142.0 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |
| | | Formulation No. | | | | | | | | | |
| | | 41 | 42 | 43 | 44 | 45 | 46 | 47 | 48 | 49 | 50 |
| Immediate Release Layer | APAP | 185.3 | 175.0 | 180.0 | 160.4 | 200.0 | 193.4 | 118.8 | 162.5 | 139.0 | 150.0 |
| | Oxycodone hydrochloride | 1.100 | 1.75 | 2.00 | 2.50 | 1.25 | 1.00 | 2.75 | 1.875 | 1.75 | 1.875 |
| | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended Release | APAP | 185.3 | 150.0 | 145.0 | 155.2 | 125.0 | 100.5 | 146.9 | 162.5 | 207.4 | 150.0 |
| | Oxycodone hydrochloride | 6.900 | 5.75 | 5.50 | 5.00 | 6.25 | 6.50 | 7.25 | 5.625 | 4.75 | 6.625 |
| | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox N-750 | 292.8 | — | — | — | 287.7 | — | — | — | 155.5 | — |
| | Polyox 301 | — | — | 244.2 | — | 13 | — | 275.5 | 321.8 | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 51 | 52 | 53 | 54 | 55 | 56 | 57 | 58 | 59 | 60 |
| Immediate Release Layer | APAP | 300.0 | 150.0 | 200.0 | 150.0 | 100.0 | 160.0 | 190.0 | 75.0 | 90.0 | 125.0 |
| | Oxycodone hydrochloride | 2.00 | 1.00 | 1.50 | 3.50 | 2.75 | 1.25 | 1.25 | 2.50 | 1.75 | 3.00 |
| | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended Release | APAP | 150.0 | 150.0 | 125.0 | 75.0 | 100.0 | 165.0 | 135.0 | 225.0 | 210.0 | 150.0 |
| | Oxycodone hydrochloride | 8.00 | 6.50 | 6.00 | 6.50 | 3.25 | 6.25 | 6.25 | 5.00 | 6.25 | 5.50 |
| | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox N-750 | 63.4 | 30.1 | 125.9 | 100.3 | 149.2 | 63.2 | 150.5 | 140.3 | 94.3 | — |
| | Polyox 301 | 210.4 | — | 175.8 | 60.7 | 175.8 | — | 160.5 | 149.7 | 100.8 | 194.6 |
| | Hydroxypropyl methyl | — | 128.3 | — | 65.4 | — | 227.7 | — | — | 127.3 | 142.0 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |

*All weights in mg.

CHART 2
Additional Oxycodone/Acetaminophen Formulations.

| | Formulation No. | | | | | | | | | |
|---|---|---|---|---|---|---|---|---|---|---|
| | 61 | 62 | 63 | 64 | 65 | 66 | 67 | 68 | 69 | 70 |
| Immediate Release Layer | | | | | | | | | | |
| APAP | 250.0 | 250.0 | 250.0 | 250.0 | 250.0 | 250.0 | 325.0 | 325.0 | 162.5 | 162.5 |
| Oxycodone hydrochloride | 3.75 | 3.75 | 3.75 | 7.5 | 7.5 | 7.5 | 3.75 | 3.75 | 2.5 | 3.75 |
| Microcrystalline cellulose | 23.72 | 23.72 | 23.72 | 32.42 | 32.42 | 32.42 | 28.10 | 28.10 | 15.50 | 18.40 |
| Pregelatinized starch | 0.50 | 0.50 | 0.50 | 1.00 | 1.00 | 1.00 | 0.50 | 0.50 | 0.33 | 0.50 |
| Citric Acid Anhydrous | 0.25 | 0.25 | 0.25 | 0.50 | 0.50 | 0.50 | 0.25 | 0.25 | 0.17 | 0.25 |
| EDTA disodium salt, | 0.05 | 0.05 | 0.05 | 0.10 | 0.10 | 0.10 | 0.05 | 0.05 | 0.033 | 0.05 |
| dihydrate | | | | | | | | | | |
| Hydroxypropyl cellulose | 25.23 | 25.23 | 25.23 | 26.43 | 26.43 | 26.43 | 32.24 | 32.23 | 16.32 | 16.72 |
| Croscarmellose sodium | 19.21 | 19.21 | 19.21 | 20.13 | 20.13 | 20.13 | 12.09 | 25.087 | 12.70 | 13.01 |
| Silicon dioxide | 1.63 | 1.63 | 1.63 | 1.70 | 1.70 | 1.70 | 2.09 | 2.09 | 1.06 | 1.08 |
| Magnesium stearate | 0.81 | 0.81 | 0.81 | 0.85 | 0.85 | 0.85 | 1.045 | 1.045 | 0.53 | 0.54 |
| Extended Release Layer | | | | | | | | | | |
| APAP | 250.0 | 250.0 | 250.0 | 250.0 | 250.0 | 250.0 | 325.0 | 325.0 | 162.5 | 162.5 |
| Oxycodone hydrochloride | 11.25 | 11.25 | 11.25 | 22.5 | 22.5 | 22.5 | 11.25 | 11.25 | 7.5 | 11.25 |
| Microcrystalline cellulose | 175.24 | 103.74 | 103.74 | 159.62 | 88.12 | 88.12 | 23.85 | 23.85 | 201.02 | 195.80 |
| Pregelatinized starch | 1.50 | 1.50 | 1.50 | 3.00 | 3.00 | 3.00 | 1.50 | 1.50 | 1.00 | 1.50 |
| Citric Acid Anhydrous | 0.75 | 0.75 | 0.75 | 1.50 | 1.50 | 1.50 | 0.75 | 0.75 | 0.50 | 0.75 |
| EDTA disodium salt, | 0.15 | 0.15 | 0.15 | 0.30 | 0.30 | 0.30 | 0.15 | 0.15 | 0.10 | 0.15 |
| dihydrate | | | | | | | | | | |
| Hydroxypropyl cellulose | 15.13 | 15.13 | 15.13 | 17.11 | 17.11 | 17.11 | — | 19.16 | 9.91 | 10.57 |
| Polyox 1105 | 250.25 | 321.75 | — | 250.25 | 321.75 | — | 321.02 | 321.02 | 321.75 | 321.75 |
| Polyox N60K | — | — | 321.75 | — | — | 321.75 | — | — | — | — |
| Silicon Dioxide | 3.58 | 3.58 | 3.58 | 3.58 | 3.58 | 3.58 | 3.57 | 3.57 | 3.58 | 3.58 |
| Magnesium Stearate | 7.15 | 7.15 | 7.15 | 7.15 | 7.15 | 7.15 | 7.13 | 7.13 | 7.15 | 7.15 |

*All weights in mg.

### III. METHODS FOR PREPARING SOLID DOSAGE FORMS OF THE PHARMACEUTICAL COMPOSITION

Another aspect of the disclosure provides methods for preparing solid dosage forms of the pharmaceutical composition that provide extended release of oxycodone and acetaminophen. Solid dosage compositions in the form of tablets may be produced using any suitable method known in the art including but not limited to wet granulation, dry granulation, direct compression, and combinations thereof.

Granulation is a manufacturing process which increases the size and homogeneity of active pharmaceutical ingredients and excipients that comprise a solid dose composition. The granulation process, which is often referred to as agglomeration, changes important physical characteristics of the dry composition, with the aim of improving manufacturability and, thereby, product quality, as well as providing desired release kinetics. Wet granulation is by far the more prevalent agglomeration process utilized within the pharmaceutical industry. Most wet granulation procedures follow some basic steps; the active agent(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. The wet granulation process may be a high shear granulation process or a fluid bed granulation process. Several methods of granulation are described in co-pending application U.S. application Ser. No. 13/166,770, filed Jun. 22, 2011, which is incorporated herein by reference in its entirety.

After granulation and drying of the resultant particles, batches are characterized with respect to properties such as final Loss on Drying (LOD), bulk density, tap density, and particle size. Loss on Drying (LOD) typically is determined after each granulation using the Moisture Analyzer. Several 1 g samples may be taken and loaded into the moisture analyzer. The samples may be run for 5 minutes at a temperature of 105° C. In another embodiment, the samples may be run at 105° C. until there is no weight fluctuation in order to determine the LOD.

Bulk and tap densities may be determined as follows. A graduated cylinder is filled with a certain amount of material (e.g., 30-40 g or 82-88 g), and the volume recorded to determine the material bulk density. Tap density can be determined with a help of a Tap Density Tester by exposing the material to 100 taps per test and recording the new volume.

Particle size determination generally is performed immediately after granulation, after sieving through 20 mesh screen to remove agglomerates. Particle diameter may be determined with a sieve-type particle diameter distribution gauge using sieves with openings of 30, 40, 60, 80, 120, and 325 mesh. Fractions may be weighed on a Mettler balance to estimate size distribution. This provides determination of the quantitative ratio by particle diameter of composition comprising extended release particles. Sieve analysis according to standard United States Pharmacopoeia methods (e.g., USP-23 NF 18), may be done such as by using a Meinzer II Sieve Shaker.

In one embodiment, the method for preparing dosage forms of the pharmaceutical composition may comprise wet granulating a first mixture comprising the opioid, such as oxycodone, the API, such as acetaminophen, and a binder to produce a first granulation mixture. The wet granulation process may be a fluid bed granulation process. In additional embodiments, the first mixture may further comprise at least one additional excipient selected from the group consisting of fillers, lubricants, antioxidants, chelating agents, and color agents. The first granulation mixture may be blended with an extended release polymer and one or more excipients, as listed above, to form at least one extended release portion of a dosage form. In certain embodiments, the extended release polymer may be a polyethylene oxide.

In another embodiment, the method further comprises wet granulating a second mixture comprising the opioid, such as oxycodone, the API, such as acetaminophen, and a binder to form a second granulation mixture. The wet granulation process may be a fluid bed granulation process. In some embodiments, the second mixture may further comprise at least one additional excipient selected from the group consisting of fillers, lubricants, disintegrants, antioxidants, chelating agents, and color agents. The second granulation mixture may be blended with one or more excipients, as listed above, to form an immediate release portion of a dosage form.

In an additional embodiment, the method may further comprise compressing the at least one extended release portion and the at least one immediate release portion into a tablet. The tablet may be a bilayer tablet. The tablet may be coated with a tablet coating.

In another embodiment, the method may comprise granulating via a high shear wet granulation process a mixture comprising the opioid (e.g., oxycodone) and at least one excipient to form opioid (e.g., oxycodone) particles. The opioid particles may be dried at a suitable temperature. The opioid particles may be granulated via a fluid bed granulation process with the API (e.g., acetaminophen), a binder, and an optional excipient to form the granulation mixture. The granulation mixture may be blended with an extended release polymer and at least one excipient to form an extended release portion of a solid dosage form.

In a further embodiment, the method may further comprise granulating via a fluid bed granulation process opioid particles with the API, a binder, and an optional excipient to form another granulation mixture. This granulation mixture may be blended with one or more excipients to form an immediate release portion of a solid dosage form.

In an additional embodiment, the method may further comprise compressing the at least one extended release portion comprising opioid particles and the at least one immediate release portion comprising opioid particles into a tablet. In one embodiment, the method comprises compressing one extended release portion comprising opioid particle and one immediate release portion comprising opioid particles into a bilayer tablet. The tablet may be coated with a tablet coating.

In another embodiment, wet granulation of either mixture may produce particles with a bulk density ranging from about 0.30 to 0.40 grams/milliliter (g/mL). In other aspects, the wet granulation may produce particles with a tap density ranging from about 0.35 g/mL to about 0.45 g/mL. In other embodiments, the wet granulation may produce particles, wherein at least about 50% of the particles have a size greater than 125 microns. In still other embodiments, the wet granulation may produce particles wherein about 20% to about 65% of the particles have a size greater than about 125 microns and less than about 250 microns.

Tablets generally are characterized with respect to disintegration and dissolution release profiles as well as tablet hardness, friability, and content uniformity.

In vitro dissolution profiles for the tablets may be determined using a USP Type II apparatus, with a paddle speed of either about 100 rpm or 150 rpm, in 0.1 N HCl, at 37° C. Samples of 5 mL at each time-point, may be taken without media replacement at 0.08, 0.25, 0.5, 1, 2, 4, 6, 8 and 12 hours, for example. In some embodiments, the dissolution profiles may be determined at varying pH values, such as at a pH of about 3.0, 3.5, 4.0, 4.5, 5.0, 5.5, 6.0 or 6.5. The fluid used may be, for example, HCl, phosphate buffer, or simulated gastric fluid. The resulting cumulative dissolution profiles for the tablets are based upon a theoretical percent active added to the pharmaceutical compositions.

A tablet preferably disintegrates before it dissolves. A disintegration tester measures the time it takes a tablet to break apart in solution. The tester suspends tablets in a solution bath for visual monitoring of the disintegration rate. Both the time to disintegration and the disintegration consistency of all tablets may be measured. The disintegration profile may be determined in a USP Disintegration Tester in pH 5.8 phosphate buffer or 0.1 N HCl of pH 1.2. The fluid used may be, for example, HCl, phosphate buffer, or simulated gastric fluid. Samples, 1-5 mL at each time-point, may be taken, for example, without media replacement at 0.5, 1, 2, 3, 4, 5, 6, 7 and 8 hours. The resulting cumulative disintegration profiles are based upon a theoretical percent active added to the pharmaceutical compositions.

After tablets are formed by compression, it is desired that the tablets have a strength of at least 9-25 Kiloponds (kp), or at least about 12-20 (kp). A hardness tester generally is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially important during any transport of the dosage form as any fracturing of the final dosage form may result in a subject receiving less than the prescribed medication. Friability may be determined using a Roche Friability Drum according to standard USP guidelines which specifies the number of samples, the total number of drum revolutions, and the drum rpm to be used. Friability values of from 0.8 to 1.0% generally are regarded as constituting the upper limit of acceptability.

The prepared tablets generally are tested for content uniformity to determine if they meet the pharmaceutical requirement of an acceptance value of 15 or less. Each tablet may be placed in a solution of 60% methanol/40% isopropanol and stirred at room temperature until the tablet disintegrates. The solution containing the dissolved tablet may be further diluted in 90% water/10% isopropanol/0.1% heptafluorobutyric acid and generally is analyzed by HPLC.

### IV. METHOD FOR REDUCING THE RISK OF ACETAMINOPHEN-INDUCED HEPATIC DAMAGE

The present disclosure also provides methods for reducing the risk of acetaminophen-induced hepatic damage in a subject being treated for pain with a dosage regimen that comprises administering to the subject at least two consecutive doses of a pharmaceutical composition comprising oxycodone and acetaminophen. The method comprises administering a first dose of a pharmaceutical composition comprising at least one extended release portion comprising the acetaminophen, the oxycodone or a combination thereof, and an extended release component to the subject, wherein the composition maintains a therapeutic blood plasma concentration of oxycodone of at least 5 ng/mL from about 0.75 hours to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration. The method further comprises administering a second dose of the pharmaceutical composition to the subject at about 12 hours after administration of the first dose.

Avoiding toxic intermediate formation is an important strategy in addressing product safety. Indeed, acetaminophen is absorbed from the stomach and small intestine and primarily metabolized by conjugation in the liver to nontoxic, water-soluble compounds that are eliminated in the urine. When the maximum daily dose (“MDD”) is exceeded over a prolonged period, metabolism by conjugation becomes saturated, and excess acetaminophen is oxidatively metabolized by the CYP enzymes (CYP2E1, 1A2, 2A6, 3A4) to a reactive metabolite, N-acetyl-p-benzoquinone-imine (NAPQI). NAPQI has an extremely short half-life, and rapidly conjugates with available glutathione, which acts as a sulfhydryl donor. The reduced NAPQI is then renally excreted. The liver plays a central role in the turnover of glutathione in the body. Given that toxicity due to NAPQI formation occurs via necrosis of the liver following the formation of toxic adducts, minimizing glutathione depletion and enhancing glutathione regeneration in the liver is an important concern.

Human erythrocyte data resulting from hepatic turnover demonstrate a time-delayed response to redox and free radical insults via glutathione depletion and regeneration. The hepatic dynamics of glutathione formation and depletion in animal data using hepatic models can also be reviewed. In Swiss mice, the dynamics of glutathione depletion was investigated in detail for acetaminophen doses ranging from (100 mg/kg to 600 mg/kg) in work done by Brzeznicka and Piotrowski (1989). Under one embodiment of the present invention, the intended dosage for patients with acute pain is 1.3 g/day of acetaminophen. Assuming a subject's weight of 70 kg, this is 1.229×10-4 moles/kg/day in human subjects. In Swiss mice, 400 mg/kg and 600 mg/kg are 2.65×10-3 moles/kg/day and 3.97×10-3 moles/kg/day, respectively, resulting in a 22-fold and a 32-fold safety exposure ratio, as compared with human levels. The bioequivalence level is 95%. Brzeiznicka and Piotrowski report that circulating hepatic GSH changes in mice began within 15 min after acetaminophen administration, and depletion followed a pattern that was strictly dose dependent, reaching a minimum GSH level 2 hrs after injection for the all dose groups, rebounding to initial levels between hours 8 and 12. Taken together, these results support the hypothesis that exposing subjects to the lower end of the therapeutic window of acetaminophen may provide benefit in terms of the patient's ability to regenerate physiologically protective levels of glutathione. Thus, the pharmaceutical formulations disclosed herein, which are designed to allow for a two hour break in acetaminophen exposure in each twelve hour exposure window allows for restorative hepatic regeneration of the subject's glutathione levels during that period when the acetaminophen concentrations are at their lowest or absent, while still preserving the considerable benefits of the potentiating effects of combination analgesia.

As mentioned above, acetaminophen is primarily metabolized via conjugation reactions, e.g., glucuronidation and sulfation, in the liver to nontoxic, water-soluble compounds that are rapidly eliminated from the body. A small proportion of acetaminophen is metabolized by the cytochrome P450 system to the reactive metabolite, NAPQI. Generally, this toxic metabolite is rapidly detoxified by conjugation to glutathione to form a non-toxic metabolite that is renally excreted. However, if the conjugation pathways become saturated and more acetaminophen is metabolized via the cytochrome P450 pathway, the pool of available glutathione may become depleted. With insufficient glutathione to bind to and inactivate NAPQI, this toxic metabolite is able to react with the sulfhydryl groups of cellular proteins initiating a cascade of cellular damage, which may lead to liver necrosis, and, ultimately, liver failure.

The method disclosed herein addresses the problem of depleted stores of glutathione by providing a period of time during the later part of the dosing interval during which the release of acetaminophen is low because most of the acetaminophen has already been released from the composition. The period of time during which the release of acetaminophen is low is called the acetaminophen “time-off” period. As a consequence of this acetaminophen time-off period, the plasma levels of acetaminophen fall to sufficiently low levels such that the metabolic burden on the liver is reduced, thereby allowing the depleted stores of glutathione to be replenished via the continuous glutathione manufacturing pathway comprising the glutathione synthase pathway. Because the levels of glutathione are able to be restored before the next dose, the risk of acetaminophen-induced hepatic damage is significantly reduced.

Additionally, the acetaminophen time-off period provided by the compositions disclosed herein may provide an added and beneficial precaution for any subject undergoing acetaminophen therapy to avoid an inadvertent reduction in glutathione stores and any potential acetaminophen-induced hepatic damage. In particular, the acetaminophen time-off period provided by the compositions disclosed herein may be especially useful during chronic administration of analgesic compositions comprising acetaminophen. The subject may be at increased risk for developing acetaminophen-induced hepatic damage because of frequent and regular user of alcohol (i.e., ethanol), concurrent administration of acetaminophen from another source (e.g., an over-the-counter medication), poor diet, and/or compromised liver function.

In general, the compositions disclosed herein are formulated such that the rate of release of acetaminophen is high during the first several hours of the dosing interval and the rate of release of acetaminophen is low during the last several hours of the dosing interval. More specifically, the compositions are formulated to release from about 40% to about 65% of the acetaminophen in about 30 minutes, from about 55% to about 80% of the acetaminophen in about 2 hours, from about 65% to about 92% of the acetaminophen in about 4 hours, and from about 67% to about 95% of the acetaminophen in about 8 hours, wherein the dosing interval is about 12 hours. In another, the compositions are formulated to release from about 45% to about 60% of the acetaminophen in about 30 minutes, from about 57% to about 75% of the acetaminophen in about 2 hours, from about 67% to about 90% of the acetaminophen in about 4 hours, and from about 70% to about 95% of the acetaminophen in about 8 hours, wherein the dosing interval is about 12 hours. In yet another embodiment, during the final 4 hours of a 12 hour dosing interval, only about 5% of the acetaminophen remains to be released from the composition.

The subject may be a mammal, and in certain embodiments, the subject may be a human. In various embodiments, the at least two consecutive doses of the analgesic composition may be administered to the subject at 8 hour intervals, 10 hour intervals, 12 hour intervals, 18 hour intervals, or 24 hour intervals.

The method for reducing the risk of acetaminophen-induced hepatic damage disclosed herein may further comprise administering additional doses of the pharmaceutical composition at regular dosing intervals, such as e.g., at 12 hour intervals. During the latter part of each dosing interval, therefore, the acetaminophen time-off period allows depleted stores of glutathione to be replenished, thereby reducing the risk of acetaminophen-induced hepatic damage in subjects being treated for pain with a composition comprising acetaminophen.

### V. METHOD FOR TREATING PAIN

Also provided is a method for treating pain in a subject in need of such treatment with a pharmaceutical composition that comprises an opioid, such as oxycodone, and an additional API, such as acetaminophen, wherein the method comprises administering an effective amount of any of the pharmaceutical compositions disclosed herein. For example, the method comprises orally administering to the subject an effective amount of a pharmaceutical composition comprising at least one extended release portion comprising oxycodone, acetaminophen and combination thereof, and an extended release component, wherein the composition maintains a therapeutic plasma concentration of oxycodone of at least about 5 ng/mL from about 0.75 hour to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration.

In some embodiments, the subject may be suffering from or diagnosed with chronic pain. In yet another embodiment, the subject may be suffering from or diagnosed with acute pain. In still another embodiment, the subject may be suffering from or diagnosed with moderate to severe acute pain. In yet other embodiments, the subject may be suffering from or diagnosed with both chronic and acute pain. The subject may be a mammal, and in certain embodiments, the subject may be a human.

In additional embodiments, the method comprises orally administering to the subject an effective amount of a gastric retentive pharmaceutical composition to the subject, wherein the subject is in a fasted state. Moreover, upon administration of the pharmaceutical composition, the opioid in the composition produces a plasma profile characterized by at least one pharmacokinetic parameter that differs by less than about 30% when the subject is in a fasted state as compared to a fed state.

The pharmacokinetic parameter of the active agent(s) of the pharmaceutical composition that differs by less that about 30% under fed and fasted conditions may be, but is not limited to, Cₘₐₓ, C₁ₕᵣ, C₂ₕᵣ, AUC, partial AUC, Tₘₐₓ, and T_{lag}. In various embodiments, the pharmacokinetic parameter may vary by less than about 25%, 20%, 15%, 10%, or 5% under fed and fasted conditions.

In embodiments in which the pharmaceutical composition comprises oxycodone and acetaminophen, the Cₘₐₓ or AUC of oxycodone and the Cₘₐₓ or AUC of acetaminophen may each individually vary by less than about 30%, 29%, 28%, 27%, 26%, 25%, 24%, 23%, 22%, 21%, 20%, 19%, 18%, 17%, 16%, 15%, 14%. 13%, 12%, 11%, 10%, 9%, 8%, 7%, 6%, 5%, 4%, 3%, 2%, or 1% under fed and fasted conditions.

In some embodiments, an effective amount of the pharmaceutical composition may be administered to a subject in a fed state. In general, a fed state is defined as having consumed food within about 30 min prior to administration of the pharmaceutical composition. The food may be a high fat meal, a low fat meal, a high calorie meal, or a low calorie meal. In other embodiments, an effective amount of the pharmaceutical composition may be administered to a subject in a fasted state. In general, a fasted state is defined as not having ingested food for at least 10 hours prior to administration of the pharmaceutical composition. In some embodiments, the pharmaceutical composition may be administered to a subject who has fasted for at least 10 hours prior to the first dose and who fasts for at least one hour prior to administration of subsequent doses. In other embodiments, the pharmaceutical composition may be administered to a subject who has fasted for at least 1 hour, 2 hours, 3 hours, 4 hours, 5 hours, 6 hours, 7 hours, 8 hours, 9 hours, or 10 hours prior to administration of each dose.

An effective amount of the pharmaceutical composition may comprise from about 5 mg to about 300 mg of the opioid and from about 100 mg to about 1300 mg of the other API. In embodiments in which the opioid is oxycodone and the API is acetaminophen, the pharmaceutical composition may comprise from about 7.5 mg to about 30 mg of oxycodone and from about 250 mg to about 1300 mg of acetaminophen.

In one embodiment, an effective amount of the pharmaceutical composition may be 15 mg of oxycodone and 650 mg of acetaminophen. For example, one solid dosage form comprising 15 mg of oxycodone and 650 mg of acetaminophen may be administered. Alternatively, two solid dosage forms with each comprising 7.5 mg of oxycodone and 325 mg of acetaminophen may be administered. In another embodiment, an effective amount of the pharmaceutical composition may be 7.5 mg of oxycodone and 325 mg of acetaminophen, wherein one solid dosage form comprising 7.5 mg of oxycodone and 325 mg of acetaminophen may be administered. In yet another embodiment, the effective amount of a pharmaceutical composition may be 20 mg of oxycodone and 650 mg of acetaminophen. For example, one solid dosage form comprising 20 mg of oxycodone and 650 mg of acetaminophen may be administered. Alternatively, two solid dosage forms with each comprising 10 mg of oxycodone and 325 mg of acetaminophen may be administered. In another embodiment, the effective amount of a pharmaceutical composition may be 10 mg of oxycodone and 325 mg of acetaminophen, wherein one solid dosage form comprising 10 mg of oxycodone and 325 mg of acetaminophen may be administered. In still yet another embodiment, the effective amount of a pharmaceutical composition may be 30 mg of oxycodone and 650 mg of acetaminophen. For example, one solid dosage form comprising 30 mg of oxycodone and 650 mg of acetaminophen may be administered. Alternatively, two solid dosage forms with each comprising 15 mg of oxycodone and 325 mg of acetaminophen may be administered. In another embodiment, the effective amount of a pharmaceutical composition may be 15 mg of oxycodone and 325 mg of acetaminophen, wherein one solid dosage form comprising 15 mg of oxycodone and 325 mg of acetaminophen may be administered.

The dosing intervals of the effective amount of the pharmaceutical composition can and will vary. For example, an effective amount of the pharmaceutical composition may be administered once a day, twice a day, or three times a day. In another embodiment, an effective amount of the pharmaceutical composition may be administered twice a day.

In general, therapeutic plasma concentrations of the opioid (e.g., oxycodone) and the additional API (e.g., acetaminophen) are attained within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the first dose of the pharmaceutical composition. Accordingly, depending upon the severity of the pain, onset on analgesia may be attained within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition. Onset of analgesia may be measured by the double stopwatch method or other pain assessments as described in Example 12 below. Generally, analgesia or pain relief will be maintained throughout the duration of the dosing interval. For example, in one embodiment, analgesia or pain relief will be maintained for 12 hours. Upon administration of the next dose of the pharmaceutical composition, therefore, analgesia or pain relief may be maintained. Accordingly, analgesia or pain relief will be maintained as long as therapeutic amounts of the pharmaceutical composition are administered at regular dosing intervals. Moreover, pain relief may be managed such that no break-through episodes of pain occur.

The extended-release formulations of the present invention are useful for treating numerous pain states that are currently being treated with conventional immediate release compositions comprising acetaminophen and oxycodone. These and additional pain states include, by way of illustration and not limitation, headache pain, pain associated with migraine, neuropathic pain selected from the group consisting of diabetic neuropathy, HIV sensory neuropathy, post-herpetic neuralgia, post-thoracotomy pain, trigeminal neuralgia, radiculopathy, neuropathic pain associated with chemotherapy, reflex sympathetic dystrophy, back pain, peripheral neuropathy, entrapment neuropathy, phantom limb pain, and complex regional pain syndrome, dental pain, pain associated with a surgical procedure and or other medical intervention, bone cancer pain, joint pain associated with psoriatic arthritis, osteoarthritic pain, rheumatoid arthritic pain, juvenile chronic arthritis associated pain, juvenile idiopathic arthritis associated pain, Spondyloarthropathies (such as ankylosing spondylitis (Mb Bechterew) and reactive arthritis (Reiters syndrome) associated pain), pain associated with psoriatic arthritis, gout pain, pain associated with pseudogout (pyrophosphate arthritis), pain associated with systemic lupus erythematosus (SLE), pain associated with systemic sclerosis (scleroderma), pain associated with Behcet's disease, pain associated with relapsing polychondritis, pain associated with adult Still's disease, pain associated with transient regional osteoporosis, pain associated with neuropathic arthropathy, pain associated with sarcoidosis, arthritic pain, rheumatic pain, joint pain, osteoarthritic joint pain, rheumatoid arthritic joint pain, juvenile chronic arthritis associated joint pain, juvenile idiopathic arthritis associated joint pain, Spondyloarthropathies (such as ankylosing spondylitis (Mb Bechterew) and reactive arthritis (Reiter's syndrome) associated joint pain), gout joint pain, joint pain associated with pseudogout (pyrophosphate arthritis), joint pain associated with systemic lupus erythematosus (SLE), joint pain associated with systemic sclerosis (scleroderma), joint pain associated with Behcet's disease, joint pain associated with relapsing polychondritis, joint pain associated with adult Still's disease, joint pain associated with transient regional osteoporosis, joint pain associated with neuropathic arthropathy, joint pain associated with sarcoidosis, arthritic joint pain, rheumatic joint pain, acute pain, acute joint pain, chronic pain, chronic joint pain, inflammatory pain, inflammatory joint pain, mechanical pain, mechanical joint pain, pain associated with the fibromyalgia syndrome (FMS), pain associated with polymyalgia rheumatica, monarticular joint pain, polyarticular joint pain, nociceptive pain, psychogenous pain, pain of unknown etiology, pain mediated by IL-6, IL-6 soluble receptor, or IL-6 receptor, pain associated with a surgical procedure in a patient with a clinical diagnosis of OA, pain like static allodynia, pain like dynamic allodynia, and/or pain associated with Crohn's disease.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

### VI. ILLUSTRATIVE BENEFITS FOR THE PRESENT INVENTION

A non-exhaustive description of certain advantages of the present invention over the prior art is described below. For example, one goal of the present invention was to develop an opioid/API formulation, such as an oxycodone/acetaminophen formulation, that has, among other things, the following characteristics:
Rapid onset of analgesia (e.g., within approximately 30 minutes);
Extended duration of analgesia for 12 hours;
Use in the treatment of acute pain;
Administration of the dosage form without regard to food;
Acetaminophen absorption primarily in a patient's upper gastrointestinal tract (upper part of small intestine, e.g., duodenum, jejunum), where acetaminophen is best absorbed;
Prolonged retention of the dosage form in the stomach;
Obtain the optimal amount and release of oxycodone in the dosage form in order to prevent inhibition of gastric emptying;
Minimize oxycodone's effect on gastric emptying, which can blunt acetaminophen's absorption, by finding the desirable dosing splits of each agent;
Achieve concentrations of acetaminophen in the latter part of the dosing cycle that are comparable to pre-dose concentrations of acetaminophen from immediate-release tablets (in a multiple-dose setting), allowing a patient's glutathione synthase enzyme cycle to replenish its levels of glutathione to avoid the formation of toxic intermediates with subsequent or concomitant doses of acetaminophen; and
Formulate an acetaminophen/oxycodone product that achieves acute and prolonged analgesia with low amounts of acetaminophen and oxycodone.

While these characteristics provided a general road map for the development work, several of these characteristics appeared to be irreconcilable. For example, administration of the dosage form to patients without regard to food was a very important characteristic, as patients suffering from acute pain often are unable to eat and retain food. Yet, in order to achieve prolonged retention of the dosage form in the stomach, one of skill in the art would administer the dosage form with food because the presence of food in the stomach decreases the stomach's migrating motor complex or “housekeeping wave.”

The “housekeeping wave” is a distinct pattern of cyclic activity observed in gastrointestinal smooth muscle during the periods between meals. The cycle recurs every 1.5 to 2 hours and consists of 4 phases: (1) a period of smooth muscle quiescence lasting 45 to 60 minutes, during which time there are very minimal stomach contractions, if any; (2) a period of roughly 30 minutes in which peristaltic contractions occur and progressively increase in frequency, (3) a period lasting 5 to 15 minutes in which rapid, evenly spaced peristaltic contractions occur, and the pylorus remains open, allowing any indigestible particles to pass into the small intestine; and (4) a short period of transition between the barrage of contractions in phase 3 and the inactivity of phase 1.

So, in the absence of food, strong peristaltic waves would force the dosage form out of the stomach and into the small intestine within approximately 1.5 to 2 hours. Therefore, the dosage form would be unable to gradually release acetaminophen from the stomach into a patient's upper gastrointestinal tract, where acetaminophen is best absorbed, over a prolonged period of time (e.g., greater than 2 hours).

On the other hand, in the presence of food, a rapid and distinct change in the motor pattern of the upper gastrointestinal tract occurs. The change is observed almost simultaneously upon the ingestion of food at all sites along the gastrointestinal tract. For example, a peristaltic contraction originates in the mid-stomach as a shallow similar to those of a housekeeping wave, but with about half the amplitude. At the start of the contraction, the pylorus is open, allowing liquids and small particles to leave the stomach and enter the small intestine. However, the pylorus constricts as the contraction continues, producing a mass contraction (termed a terminal antral contraction). This mass contraction hits the closed pylorus, and this contact forcefully sends the solid particles back through the constricted antral ring producing a shearing effect, and fragmenting the solid particles. This pattern of activity goes on for several hours, causing larger particles to be retained in the stomach for about 4 to 6 hours. Thus, gastric emptying is very different in a fasted state versus a fed state, which can significantly impact the absorption site of a dosage form.

As illustrated above, in the development of the improved opioid/API formulations, the inventors had to balance several, often conflicting variables. Whether the formulation could be administered in a fed state versus a fasted state was just one of those variables. As shown in Table A below, there were several characteristics that were at odds, or in tension with, with each other in the development process.

TABLE A
Targeted Characteristics

| One Desired | | Another Desired |
|---|---|---|
| Characteristic | | Characteristic |
| Rapid onset of analgesia | In tension with . . . | Extended duration of |
| (e.g., within | | analgesia for 12 hours |
| approximately | | |
| 30 minutes) | | |
| Oxycodone/ | In tension with . . . | Oxycodone known to affect |
| acetaminophen | | gastric emptying altering the |
| combination product | | absorption of acetaminophen |
| Acute pain indication | In tension with . . . | Extended-release formulation |
| Administration of the | In tension with . . . | Prolonged retention of the |
| dosage form without | | dosage form in the stomach, |
| regard to food | | which requires food |
| Achieve low plasma | In tension with . . . | Need sufficient pain relief |
| concentrations of | | throughout the entire dosing |
| acetaminophen | | cycle |
| in the latter part | | |
| of the dosing cycle | | |
| Need therapeutically | In tension with . . . | Need to lessen the effects |
| effective doses of | | caused by oxycodone on |
| oxycodone | | gastric emptying |
| Lower amounts of | In tension with . . . | Maintenance of adequate |
| oxycodone and | | pain relief over the entire |
| acetaminophen | | dosing cycle (e.g., avoid |
| | | breakthrough pain in the |
| | | 8- to 12-hour timeframe) |

In light of the inherent tension between these targeted characteristics, several road blocks had to be overcome in the development process. Further, the inventors recognized that it would likely not be able to formulate a dosage form that possessed all of the target characteristics (e.g., the dosage form would have to be administered with food).

Yet, as a result of the amounts of oxycodone and acetaminophen in the IR and ER portions, the inventors were able to develop a formulation with all of the targeted characteristics. However, this result was unexpected by one skilled in the art. Indeed, during the development work, the inventors were informed by several FDA consultants and physicians specializing in the treatment of pain, that formulations like the ones disclosed herein, would not be effective in the treatment of acute pain. Specifically, these consultants were concerned that the lower amounts of oxycodone in the IR and ER portions would not provide a patient with adequate pain relief (i.e., be subtherapeutic).

Further, one of the goals of the development work was to develop a formulation that would provide a patient suffering from acute pain with 12 hours of adequate pain relief. One skilled in the art would realize that decreasing the amounts of oxycodone in the IR and ER layers from the amounts disclosed in the art would not provide a longer period of pain relief, and would likely result in break-through pain. One skilled in the art would also realize that lower doses of oxycodone in the IR portion would increase the time it takes for a patient to achieve the maximum plasma concentration for oxycodone, which would be undesirable in the treatment of acute pain. Thus, in developing an effective 12-hour, extended-release formulation for the treatment of acute pain, the skilled person would conclude that he or she must increase the amount of oxycodone in the formulations disclosed in the art, not decrease it. Accordingly, one skilled in the art would not take the teaching of the art and lower the amounts of oxycodone in the IR and ER layers based on their teachings.

In fact, during the development of the formulations disclosed herein, the inventors actually performed pharmacokinetic studies on dogs, in which it varied the amounts of oxycodone in the IR and ER layers. They then took the dog pharmacokinetic data and used it to stimulate the corresponding human pharmacokinetic data. They performed these tests to assist them in determining the amounts of oxycodone and acetaminophen that would be required to achieve the target pharmacokinetic parameters.

As illustrated in FIG. 106, those studies showed that formulations comprising (1) 3 mg, or 20% of the oxycodone in the IR layer, and 12 mg, or 80% of the oxycodone in the ER layer, and (2) 0 mg, or 0% of the oxycodone in the IR layer, and 15 mg, or 100% of the oxycodone in the ER layer, did not provide sufficient blood levels over the dosing interval to anticipate that a patient would receive adequate pain relief.

The results shown in FIG. 106 indicate that these formulations would be subtherapeutic. As a result, this data confirms that one of skill in the art would not decrease the dose of oxycodone, but rather, would believe that the dose must be increased in order to obtain adequate pain relief. Consequently, one skilled in the art would not have lowered the dose of oxycodone based on the teachings of the art, as FIG. 106 shows that lower doses of oxycodone would not provide patients with adequate pain relief.

Yet, the inventors surprisingly found that it was able to formulate an extended-release oxycodone/acetaminophen formulation with all of the desired characteristics set forth above and with lower amounts of oxycodone. Indeed, they developed improved extended-release oxycodone/acetaminophen formulations that possess the following unexpected characteristics: (1) the formulations may be administered without regard to food; (2) the formulations achieve the desired pharmacokinetic parameters, such as, a rapid onset of analgesia, an extended duration of pain relief, and low plasma concentrations of acetaminophen in the latter part of the dosing cycle; and (3) the formulations provide sufficient acute pain relief.

These unexpected characteristics are a result of the unique formulations disclosed herein. For example, the improved oxycodone/acetaminophen formulations have optimal amounts of oxycodone and acetaminophen in the IR layer(s) and the ER layer(s). Indeed, in one representative example, the IR layer(s) comprises about 1.5 mg to about 4.0 mg of oxycodone and about 125 mg to about 325 mg of acetaminophen, and the ER layer(s) comprises about 4.5 mg to about 6.5 mg of oxycodone and about 125 mg to about 325 mg of acetaminophen. While the specific mechanism of action is not known, these “splits” or amounts of oxycodone and acetaminophen in the IR/ER layers have several positive and unpredictable benefits.

First, the “split” of oxycodone between the IR and ER layers allows the formulations disclosed herein to be administered without regard to food. Indeed, these formulations allow an optimal amount of oxycodone to be released early on in the dosing cycle. Once absorbed, the oxycodone interacts with receptors in the upper gastrointestinal tract, delaying gastric emptying. The delay in gastric emptying caused by the amount and release of oxycodone from the dosage form allows the dosage form to be retained in the stomach—even in a fasted state. This results in prolonged retention of the dosage form in the stomach, allowing the acetaminophen to be slowly released from the dosage form and optimally absorbed in a patient's upper gastrointestinal tract, where I believe it is best absorbed. Consequently, it was observed that the amount and release of oxycodone is unexpectedly just enough to slow down the stomach (like food) without causing severe inhibition of gastric emptying, which can often be seen when higher doses of oxycodone are administered to a patient. Observing these effects with the first dose is also remarkable and desirable for treating acute pain.

The observation that the formulations disclosed herein can be administered without regard to food was surprising, but also remarkable as this effect occurs at the very first dose of administration. By way of example, many formulations have effects that can only be seen after the patient has been administered three to five doses of the formulation and has reached “steady state” plasma concentrations of the drug. However, for the formulations disclosed herein, a patient does not have to achieve a steady state plasma concentration for oxycodone to exhibit a lack of food effect. This is highly beneficial to the patient, and also critical to the formulations disclosed herein, which treat acute pain and require an early onset of action to be effective.

In essence, at the amounts disclosed herein, the effect of oxycodone on the stomach resembles the effect that food has on the stomach from the initial dose. Thus, there is no need for a patient to take the improved oxycodone/acetaminophen formulations with food in order to achieve the ideal absorption of acetaminophen in the upper gastrointestinal tract. This freedom provides a significant clinical benefit to patients in need of pain relief as they often are unable to eat and/or retain food.

This result, however, was surprising in view of the teaching of the art. For instance, the art specifically teaches that the dosage forms disclosed herein must be taken with food. See, e.g., U.S. Patent Publication No. 2010/0015222 (“Han”) at ¶¶56, 89, 90, 118, 126, 181-82, 238-40, and 244. For example, Han provides that “[i]t has been determined that once the fed mode [i.e., ‘the presence of food in the stomach’] has been induced, larger particles are retained in the stomach for a longer period of time than smaller particles. Thus, the fed mode is typically induced in a patient by the presence of food in the stomach.” Id. at 89. Han also provides that “dosage forms provide controlled delivery of acetaminophen, and an opioid analgesic to the upper GI tract by a polymer matrix that swells unrestrained dimensionally, and is retained in the stomach when taken with food, i.e., in the fed mode.” Id. at 118. Nevertheless, as explained above, the inventors surprisingly discovered that the claimed amounts of oxycodone in the IR/ER layers and their release into gastric fluids are all that is needed to retain the dosage form in the stomach.

Second, the “splits” of oxycodone and acetaminophen between the IR and ER layers allows the formulations disclosed herein to achieve the desired pharmacokinetic parameters. For instance, the splits produce (1) a rapid onset of analgesia (e.g., within approximately 30 minutes), which includes a Tₘₐₓ and a Cₘₐₓ for acetaminophen that is comparable to an immediate-release acetaminophen product; (2) low plasma concentrations of acetaminophen in the latter part of the dosing cycle; and (3) an extended duration of analgesia for 12 hours.

As explained above, the formulations disclosed herein achieve a rapid onset of analgesia (e.g., within approximately 30 minutes). Further, these improved extended-release formulations also achieve a Tₘₐₓ and a Cₘₐₓ of acetaminophen that is comparable to an immediate-release acetaminophen product. These results were unexpected for a few reasons.

In general, extended-release formulations often have a longer Tₘₐₓ (i.e., the time after administration of a drug when the maximum plasma concentration, or Cₘₐₓ, is reached in a patient) in order to provide sufficient pain relief over the entire dosing interval (e.g., 12 hours). It is also well established that opioids, like oxycodone, blunt the Cₘₐₓ of acetaminophen when the two active ingredients are administered together. Yet, the formulations disclosed herein surprisingly provide a patient with an early onset of analgesia and a Cₘₐₓ of acetaminophen that is not affected by the amount of oxycodone that is present in the formulation.

Moreover, the absorption of the acetaminophen is complete in about 8 to about 10 hours. So, for at least one half life of acetaminophen, the blood supply reaching the patient's liver via the portal vein contains no additional amounts of acetaminophen beyond the amounts present in the patient's circulation. As a result, the concentrations of acetaminophen in the latter part of the dosing interval are surprisingly comparable to pre-dose concentrations of acetaminophen seen with immediate release tablets in a multiple dose setting. This allows the patient's glutathione synthase enzyme cycle (i.e., the cycle that metabolizes acetaminophen) to replenish his/her levels of glutathione to avoid the formation of toxic intermediates with subsequent or concomitant doses of acetaminophen.

Further, even though the plasma concentration of acetaminophen falls at the latter end of the dosing cycle, the formulations disclosed herein provide a patient with 12 hours of analgesia. This extended duration of pain relief is a direct result of the amounts of oxycodone and acetaminophen between the IR and ER layers, and the differing patterns of release of these two drugs. Particularly, the formulations were designed to have acetaminophen and oxycodone work together to provide pain relief in a complimentary way by taking advantage of their differing solubilities, mechanisms of action, and pharmacodynamic responses. The formulations were also designed to try and take advantage of the known synergy that can occur with the “combination of an optimum dose of acetaminophen and oxycodone.” See Gammaitoni et al., Effectiveness and Safety of New Oxycodone/Acetaminophen Formulations With Reduced Acetaminophen for the Treatment of Low Back Pain, Pain Medicine, Vol. 4, No. 1, at 28 (2003). Thus, the pharmacokinetic profiles of the oxycodone and the acetaminophen were not designed to be identical. See FIG. 107. Rather, as a result of the splits, the pharmacokinetic profiles of oxycodone and acetaminophen were designed to be offset—capitalizing on the different mechanisms of action and solubilities of the two active ingredients.

Accordingly, as illustrated by the pharmacokinetic curves set forth in FIG. 107 above, a patient who is administered an improved oxycodone/acetaminophen formulation disclosed herein initially experiences a rapid onset of analgesia with the acetaminophen contributing a larger share of relief. However, at the end of the dosing cycle, the patient experiences prolonged analgesia with the oxycodone contributing a larger share of relief. Consequently, the optimal amounts of oxycodone and acetaminophen between the IR and ER layers, and the differing patterns of release of these two drugs, provide a patient with immediate relief and effective 12-hour pain relief.

In contrast, and as explained above, one skilled in the art would not lower the amounts of oxycodone based on the teachings of the art, in an attempt to provide 12 hours of pain relief. Rather, the skilled person would increase the amount of oxycodone an attempt to achieve pain relief over a 12-hour period.

The offset pharmacokinetic curves for oxycodone and acetaminophen resulting from the “splits” of those drugs in the IR and ER layers allows the extended-release formulations disclosed herein to be used in the treatment of acute pain. This is surprising as extended-release formulations typically cannot provide a patient with adequate pain relief early on and throughout the first dosing cycle. In fact, in inventors are not aware of any other extended-release opioid/acetaminophen formulation that is approved by the U.S. Food and Drug Administration for the treatment of acute pain.

However, as explained above and illustrated in the pharmacokinetic data presented in FIG. 107, the extended-release formulations disclosed herein surprisingly provide a patient in need thereof with a rapid onset of analgesia and an extended duration of analgesia of 12 hours.

Accordingly, the formulations disclosed herein yield several unexpected results that are not taught or disclosed by the teachings of the art.

As presented in Table B below, the extended-release formulations of the present invention also exhibit a lower incidence of treatment-emergent adverse events (TEAE) as compared to the commercially-available immediate release OC/APAP.

TABLE B
Comparison of TEAE between the Present Invention and a
Commercially-Available Immediate Release
Oxycodone/Acetaminophen Product

| | ER OC/APAP | | Commercially- | |
|---|---|---|---|---|
| | Disclosed Herein | | Available | |
| | (See Chart No. 1) | | IR OC/APAP | |
| | 30/1,300 | 15/650 | 15/650 | 30/1,300 |
| System Organ Class | (n = 202) | (n = 94) | (n = 185) | (n = 67) |
| Preferred Term | (%) | (%) | (%) | (%) |

Gastrointestinal Disorders

| | | | | |
|---|---|---|---|---|
| Constipation | 0 | 1.1 | 0.5 | 0 |
| Nausea | 28.7 | 26.6 | 30.3 | 49.3 |
| Vomiting | 17.3 | 4.3 | 11.4 | 28.4 |

Nervous System Disorders

| | | | | |
|---|---|---|---|---|
| Dizziness | 13.4 | 14.9 | 11.9 | 22.4 |
| Somnolence | 9.9 | 10.6 | 6.5 | 19.4 |

Having described the invention in detail, it will be apparent that modifications and variations are possible without departing from the scope of the invention defined in the appended claims.

### EXAMPLES

The following examples are included to demonstrate certain embodiments of the invention. Those of skill in the art should, however, in light of the present disclosure, appreciate that modifications can be made in the specific embodiments that are disclosed and still obtain a like or similar result without departing from the spirit and scope of the invention, therefore all matter set forth is to be interpreted as illustrative and not in a limiting sense.

### Example 1

### In Vitro Dissolution of Controlled-Release Bilayer Tablets

Controlled-release bilayer tablets were prepared containing 15 mg of oxycodone and 500 mg of acetaminophen (APAP), or 30 mg of oxycodone and 500 mg APAP. (See selected examples from Chart No. 2.) The ER layer contained 75% of the total amount of oxycodone in the tablet, 50% of the total amount of APAP in the tablet, and either 35% w/w POLYOX® 1105 (for fast release), 45% w/w POLYOX® 1105 (for medium release), or 45% w/w POLYOX® N60K (for slow release). The IR layer contained 25% of the total amount of oxycodone in the tablet and 50% of the total amount of APAP in the tablet.

Dissolution profiles for the three above-described compositions were determined in USP Type II apparatus. Six tablets of each composition were weighed, placed in a sinker, and dropped into an equilibrated dissolution bath vessel that contained 900 mL of (helium sparged) 0.1 N HCl that was heated to 37° C.±0.5° C. The mixture was stirred at 150±6 rpm and the temperature was maintained at 37° C.±0.5° C. for 12 hr. The bath vessel was covered with a low evaporation vessel cover. Samples (5 mL) were removed at 0.25, 0.5, 1, 2, 4, 6, 8, and 12 hours. Each sample was filtered through a 0.45 μm filter and analyzed by HPLC using standard procedures.

The cumulative release of oxycodone and APAP from 15 mg oxycodone/500 mg APAP tablets is presented in Table 1. Table 2 presents the cumulative release of oxycodone and APAP from 30 mg oxycodone/500 mg APAP (30/500) tablets. FIG. 1 presents the release profile of oxycodone from the 15/500 and 30/500 tablets. The dissolution profile of APAP from the 15/500 and 30/500 tablets is shown in FIG. 2. The release of oxycodone and APAP from the fast release and medium release tablets was essentially linear during the first half of the 12 hour time period but then plateaued during the last half of the 12 hour time period. The release of oxycodone and APAP from the slow release tablets was essentially linear during the entire 12 hour time period.

TABLE 1
Cumulative Release - 15 mg oxycodone/500 mg APAP Tablets

| | Oxycodone (%) | | | APAP (%) | | |
|---|---|---|---|---|---|---|
| Time (hr) | Fast | Medium | Slow | Fast | Medium | Slow |
| 0.25 | 27.56 | 25.70 | 25.68 | 54.78 | 53.06 | 53.01 |
| 0.5 | 34.33 | 31.31 | 30.39 | 57.55 | 55.73 | 54.89 |
| 1.0 | — | 40.85 | 37.81 | — | 60.03 | 58.03 |
| 2.0 | 59.88 | 55.67 | 49.50 | 71.42 | 68.16 | 63.27 |
| 4.0 | 83.46 | 77.94 | 67.43 | 86.17 | 81.55 | 72.31 |
| 6.0 | 97.48 | 92.12 | 80.53 | 96.19 | 91.62 | 79.97 |
| 8.0 | 101.26 | 99.26 | 90.20 | 100.16 | 96.96 | 86.06 |
| 12.0 | 101.57 | 101.23 | 99.36 | 100.10 | 99.16 | 94.41 |

TABLE 2
Cumulative Release - 30 mg oxycodone/500 mg APAP Tablets

| | Oxycodone (%) | | | APAP (%) | | |
|---|---|---|---|---|---|---|
| Time (hr) | Fast | Medium | Slow | Fast | Medium | Slow |
| 0.25 | 31.65 | 30.27 | 29.78 | 54.17 | 52.97 | 52.97 |
| 0.5 | 37.55 | 35.91 | 34.42 | 56.96 | 55.64 | 54.97 |
| 1.0 | 47.18 | 45.21 | 41.12 | 61.81 | 60.19 | 58.15 |
| 2.0 | 62.51 | 59.63 | 52.40 | 70.60 | 68.04 | 63.61 |
| 4.0 | 84.72 | 80.44 | 70.01 | 85.28 | 81.56 | 73.04 |
| 6.0 | 96.97 | 93.98 | 82.49 | 94.57 | 91.42 | 80.94 |
| 8.0 | 100.23 | 99.63 | 91.78 | 97.91 | 96.48 | 87.26 |
| 12.0 | 100.57 | 101.13 | 99.60 | 98.09 | 98.14 | 95.25 |

The cumulative in vitro release of oxycodone and APAP from 7.5 mg oxycodone/325 mg APAP medium release tablets is presented in Table 3. The ER layer of these tablets contained 5.625 mg of oxycodone, 162.5 mg of APAP, and 45% (w/w) POLYOX® 1105, and the IR layer contained 1.875 mg of oxycodone and 162.5 mg of APAP. (See selected example from Chart 1.) The dissolution profile was determined essentially as described above, except that samples were collected at 0.08 hour (˜5 min) in addition to the later time points.

TABLE 3
Cumulative Release 7.5 mg oxycodone/
325 mg APAP Tablets

| | Oxycodone (%) | | APAP (%) | |
|---|---|---|---|---|
| Time | Mean | % RSD | Mean | % RSD |
| (hr) | (%) | (6) | (%) | (%) |
| 0.08 | 26.6 | 4.3 | 49.0 | 3.4 |
| 0.25 | 31.5 | 4.2 | 51.3 | 3.1 |
| 0.5 | 37.5 | 2.7 | 53.8 | 2.9 |
| 1.0 | 45.9 | 1.6 | 58.2 | 2.5 |
| 2.0 | 60.1 | 1.7 | 66.0 | 2.3 |
| 4.0 | 81.4 | 1.1 | 78.7 | 1.7 |
| 6.0 | 95.4 | 1.4 | 88.4 | 1.9 |
| 8.0 | 101.8 | 0.9 | 93.9 | 1.4 |
| 12.0 | 103.2 | 1.2 | 94.9 | 1.1 |

FIG. 3 and FIG. 4 present the percentage of oxycodone and APAP, respectively, released from two different lots of 7.5/325 tablets as compared to 15/650 tablets (see Example 27 for the dissolution data of the 15 mg oxycodone/650 acetaminophen tablets). The dissolution profiles were similar among all the tablets.

The release of oxycodone and APAP from each layer was analyzed by determining the calculated release from the ER layer and actual release from the total composition. For this, the tablets contained 7.5 mg of oxycodone HCl and 325 mg of APAP (i.e., the ER layer contained 5.625 mg of oxycodone HCl, 162.5 mg of APAP, and 45% (w/w) POLYOX® 1105; and the IR layer contained 1.875 mg of oxycodone HCl and 162.5 mg of APAP). The dissolution profile was determined essentially as described above. The calculated cumulative release of oxycodone HCl from the ER layer and the total tablet is presented in Table 4, and the calculated cumulative release of APAP from the ER layer and the total tablet is presented in Table 5. These data show that essentially all of the 1.875 mg of oxycodone HCl in the IR layer was released within about 5 minutes and essentially all of the 162.5 mg of APAP in the IR layer was released within about 15 minutes.

TABLE 4
Split Release of Oxycodone 7.5 mg
oxycodone/325 mg APAP Tablets

| Time | Total | Total | ER | ER |
|---|---|---|---|---|
| (hr) | (%) | (mg) | (%) | (mg) |
| 0.08 | 26.6 | 2.00 | 2.1 | 0.12 |
| 0.25 | 31.5 | 2.36 | 8.7 | 0.49 |
| 0.5 | 37.5 | 2.81 | 16.7 | 0.94 |
| 1.0 | 45.9 | 3.44 | 27.9 | 1.57 |
| 2.0 | 60.1 | 4.51 | 46.8 | 2.63 |
| 4.0 | 81.4 | 6.11 | 75.2 | 4.23 |
| 6.0 | 95.4 | 7.16 | 93.9 | 5.28 |
| 8.0 | 101.8 | 7.64 | 102.4 | 5.76 |
| 12.0 | 103.2 | 7.74 | 104.3 | 5.87 |

TABLE 5
Split Release of APAP 7.5 mg
oxycodone/325 mg APAP Tablets

| Time | Total | Total | ER | ER |
|---|---|---|---|---|
| (hr) | (%) | (mg) | (%) | (mg) |
| 0.08 | 49.0 | 159.25 | 0.0 | 0.00 |
| 0.25 | 51.3 | 166.73 | 2.6 | 4.22 |
| 0.5 | 53.8 | 174.85 | 7.6 | 12.35 |
| 1.0 | 58.2 | 189.15 | 16.4 | 26.65 |
| 2.0 | 66.0 | 214.50 | 32.0 | 52.00 |
| 4.0 | 78.7 | 255.78 | 57.4 | 93.28 |
| 6.0 | 88.4 | 287.30 | 76.8 | 124.80 |
| 8.0 | 93.9 | 305.18 | 87.8 | 142.68 |
| 12.0 | 94.9 | 308.43 | 89.8 | 145.93 |

### Example 2

### Clinical Pharmacokinetic Analysis of Controlled-Release 15 mg Oxycodone/500 mg Acetaminophen Bilayer Tablets—Single Dose

An open-label, single dose, four-period crossover study was conducted to evaluate the pharmacokinetics (PK) and bioavailability of three controlled-release bilayer tablets comprising 15 mg oxycodone (OC) and 500 mg APAP as compared to a commercially available immediate-release tablet containing 7.5 mg oxycodone/325 mg acetaminophen. The three controlled release formulations—fast, medium, and slow—are described above. (See selected examples from Chart No. 2.) One tablet of each of the controlled-release bilayer formulations was administered to the test subjects under fed conditions. One tablet of the commercially available immediate-release tablet containing 7.5 mg oxycodone/325 mg acetaminophen was administered every 6 hours (Q6 h) for two doses under fed conditions. The test subjects were about 40 normal, healthy male subjects between 21-45 years of age.

Subjects were randomly assigned to Treatments A, B, C, and D using a four-period, eight-sequence, crossover design as follows:
Treatment A: One (1) tablet of 15 mg OC/500 mg APAP, Fast Release administered orally under fed conditions.
Treatment B: One (1) tablet of 15 mg OC/500 mg APAP, Medium Release administered orally under fed conditions.
Treatment C: One (1) tablet of 15 mg OC/500 mg APAP, Slow Release administered orally under fed conditions.
Treatment D: One (1) tablet of a commercially available immediate-release tablet containing 7.5 mg oxycodone/325 mg administered orally Q6 h for two (2) doses under fed conditions.

The crossover design allowed for within-subject comparisons among the test formulations with differing release profiles. Subjects received each of the study drug treatments (A-D) separated by at least a 7-day interval between the start of each period at Hour 0. During each period, subjects remained in the clinical facility from the time of check-in (on the day prior to dosing) until discharge on Day 3 (after the 48 hour blood draw).

Physical examinations, electrocardiograms and clinical laboratory tests were performed at screening and at the conclusion of the study (or early termination). Vital sign measurements (including pulse oximetry) and adverse events were monitored during the study. Subjects were administered a 50 mg naltrexone tablet 12 hours prior to Hour 0 dosing, at Hour 0, and 12 hours post-dose to block the effects and potential risks of oxycodone. After a 10 hour overnight fast, subjects were served a standardized FDA high-fat breakfast to be consumed in 30 minutes or less prior to Hour 0 dosing for the first oral dosage. All subjects in each period were served a standardized meal to be consumed in 30 minutes or less prior to Hour 6. Only subjects randomized to Treatment D were administered the second oral dosage of the commercially available immediate-release tablet containing 7.5 mg oxycodone/325 mg acetaminophen at Hour 6 in each period.

Blood was drawn at designated times for PK analysis. Samples (6 mL in pre-chilled vacuum blood collection tubes, containing K2EDTA as the anticoagulant) were taken pre-dose (up to 60 minutes prior to dose), 10 min, 20 min, 30 min, 40 min and 1, 2, 3, 4, 5, 6, 6.5, 7, 8, 9, 10, 12, 16, 18, 20, 24, 36 and 48 hours post-dose. The collected plasma samples were analyzed for the active pharmaceutical ingredients (APIs), i.e., oxycodone and acetaminophen, using validated liquid chromatography/tandem mass spectrometry (LC-MS/MS) assays.

The following PK parameters were calculated for oxycodone and acetaminophen using standard non-compartmental methods:
area under the plasma concentration curve to last quantifiable concentration AUC₍₀₋ₜ₎
area under the plasma concentration curve to infinite time AUC_{(0-inf)}
maximum observed plasma concentration (Cₘₐₓ)
time observed maximum plasma concentration (tₘₐₓ)
lag time (t_{lag})
apparent first-order terminal elimination rate constant (kₑₗ)
apparent plasma terminal elimination half-life (t_{1/2})

Parametric general linear model (GLM) methodology was used in the analysis of all pharmacokinetic parameters. The SAS GLM procedure was used to perform analysis of variance (ANOVA) on each pharmacokinetic parameter with sequence, treatment, period, and subjects nested within sequences, as sources of variation. For each formulation, least squares means and the associated standard errors were obtained using the LSMEANS option. All treatment pairwise comparisons were performed, without adjustment for multiplicity. AUC and Cₘₐₓ were dose-adjusted for comparative purposes for acetaminophen and the commercially available immediate-release tablet containing 7.5 mg oxycodone/325 mg acetaminophen.

The pharmacokinetic data for oxycodone and APAP are presented in Tables 6-8 and 9-11, respectively.

TABLE 6
Oxycodone Pharmacokinetics (15/500)

| | | | | | Commercially |
|---|---|---|---|---|---|
| | | | | | available |
| | Fast Release Formulation | | | | immediate- |
| | Mean | LSM | 90% CI | | release tablet |
| Parameter | (% CV) | Ratio | Lower | Upper | Mean (% CV) |
| Cₘₐₓ (ng/mL) | 18.803 | 82.92 | 78.02 | 88.12 | 22.428 |
| | (21) | | | | (20) |
| C_{1 hr} (ng/mL) | 6.891 | 72.79 | 49.02 | 108.1 | 10.226 |
| | (77) | | | | (65) |
| C_{2 hr}^{a} (ng/mL) | 12.355 | 80.74 | 71.2 | 91.56 | 14.94 |
| | (32) | | | | (26) |
| AUC₀₋ₜ | 209.949 | 89.73 | 86.52 | 93.06 | 229.788 |
| (ng · hr/mL) | (26) | | | | (22) |
| AUC_{0-inf} | 211.8 | 89.95 | 86.77 | 93..24 | 231.421 |
| (ng · hr/mL) | (25) | | | | (22) |
| AUC_{0-1 hr} | 2.565 | 61.32 | 37.64 | 99.92 | 4.334 |
| (ng · hr/mL) | (104) | | | | (80) |
| AUC_{0-2 hr}^{b} | 12.189 | 70.16 | 55.97 | 87.95 | 16.917 |
| (ng · hr/mL) | (53) | | | | (46) |
| AUC_{0-4 hr}^{c} | 41.3 | 88.76 | 80.61 | 97.73 | 45.699 |
| (ng · hr/mL) | (29) | | | | (24) |
| Tₘₐₓ (hr) | 4.954 | na | na | na | 7.954 |
| | (34) | | | | (22) |
| T_{lag} (hr) | 0.31 | na | na | na | 0.219 |
| | (68) | | | | (77) |
| T_{1/2} (hr) | 4.584 | na | na | na | 4.495 |
| | (17) | | | | (14) |
| Kₑₗ (1/hr) | 0.155 | na | na | na | 0.157 |
| | (16) | | | | (13) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2 SD for commercially-available immediate release tablet

TABLE 7
Oxycodone Pharmacokinetics (15/500)

| | | | | | Commercially- |
|---|---|---|---|---|---|
| | | | | | available |
| | | | | | immediate |
| | | | | | release |
| | Medium Release Formulation | | | | tablet |
| | Mean | LSM | 90% CI | | Mean |
| Parameter | (%CV) | Ratio | Lower | Upper | (%CV) |
| Cₘₐₓ (ng/mL) | 18.266 | 80.87 | 76.09 | 85.95 | 22.428 |
| | (25) | | | | (20) |
| C_{1 hr} (ng/mL) | 7.364 | 67.62 | 45.75 | 99.95 | 10.226 |
| | (81) | | | | (65) |
| C_{2 hr}^{a} (ng/mL) | 12.388 | 79.04 | 69.69 | 89.64 | 14.94 |
| | (45) | | | | (26) |
| AUC₀₋ₜ | 217.188 | 94.19 | 90.82 | 97.68 | 229.788 |
| (ng · hr/mL) | (23) | | | | (22) |
| AUC_{0-inf} | 218.545 | 94.09 | 90.77 | 97.54 | 231.421 |
| (ng · hr/mL) | (23) | | | | (22) |
| AUC_{0-1 hr} | 3.248 | 64.69 | 39.93 | 104.8 | 4.334 |
| (ng · hr/mL) | (118) | | | | (80) |
| AUC_{0-2 hr}^{b} | 13.124 | 71.74 | 57.22 | 89.96 | 16.917 |
| (ng · hr/mL) | (70) | | | | (46) |
| AUC_{0-4 hr}^{c} | 42.101 | 88.61 | 80.47 | 97.58 | 45.699 |
| (ng · hr/mL) | (43) | | | | (24) |
| Tₘₐₓ (hr) | 5.31 | na | na | na | 7.954 |
| | (38) | | | | (22) |
| T_{lag} (hr) | 0.264 | na | na | na | 0.219 |
| | (64) | | | | (77) |
| T_{1/2} (hr) | 4.557 | na | na | na | 4.495 |
| | (16) | | | | (14) |
| Kₑₗ (1/hr) | 0.156 | na | na | na | 0.157 |
| | (16) | | | | (13) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2 SD for commercially-available immediate release tablet

TABLE 8
Oxycodone Pharmacokinetics (15/500)

| | | | | | Commercially- |
|---|---|---|---|---|---|
| | | | | | available |
| | | | | | immediate |
| | | | | | release |
| | Slow Release Formulation | | | | tablet |
| | Mean | LSM | 90% CI | | Mean |
| Parameter | (% CV) | Ratio | Lower | Upper | (% CV) |
| Cₘₐₓ (ng/mL) | 17.403 | 76.75 | 72.21 | 81.58 | 22.428 |
| | (25) | | | | (20) |
| C_{1 hr} (ng/mL) | 7.601 | 69.63 | 47.08 | 102.97 | 10.226 |
| | (79) | | | | (65) |
| C_{2 hr}^{a} (ng/mL) | 11.237 | 73.55 | 64.84 | 83.43 | 14.94 |
| | (39) | | | | (26) |
| AUC₀₋ₜ | 222.096 | 95.62 | 92.2 | 99.18 | 229.788 |
| (ng · hr/mL) | (25) | | | | (22) |
| AUC_{0-inf} | 223.553 | 95.61 | 92.22 | 99.11 | 231.421 |
| (ng · hr/mL) | (25) | | | | (22) |
| AUC_{0-1 hr} | 2.893 | 57.34 | 35.37 | 92.95 | 4.334 |
| (ng · hr/mL) | (112) | | | | (80) |
| AUC_{0-2 hr}^{b} | 12.312 | 68.63 | 54.72 | 86.08 | 16.917 |
| (ng · hr/mL) | (66) | | | | (46) |
| AUC_{0-4 hr}^{c} | 38.842 | 83.46 | 75.78 | 91.92 | 45.699 |
| (ng · hr/mL) | (35) | | | | (24) |
| Tₘₐₓ (hr) | 5.655 | na | na | na | 7.954 |
| | (27) | | | | (22) |
| T_{lag} (hr) | 0.299 | na | na | na | 0.219 |
| | (74) | | | | (77) |
| T_{1/2} (hr) | 4.647 | na | na | na | 4.495 |
| | (19) | | | | (14) |
| Kₑₗ (1/hr) | 0.154 | na | na | na | 0.157 |
| | (18) | | | | (13) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2 SD for commercially-available immediate release tablet

TABLE 9
Acetaminophen Pharmacokinetics (15/500)

| | | | | | Commercially- |
|---|---|---|---|---|---|
| | | | | | available |
| | | | | | immediate |
| | | | | | release |
| | Fast Release Formulation | | | | tablet* |
| | Mean | LSM | 90% CI | | Mean |
| Parameter | (% CV) | Ratio | Lower | Upper | (% CV) |
| Cₘₐₓ (ng/mL) | 2612 | 94.46 | 87.25 | 102.26 | 2721 |
| | (26) | | | | (22) |
| C_{1 hr} (ng/mL) | 1627 | 113.22 | 84.91 | 150.98 | 1516 |
| | (66) | | | | (58) |
| C_{2 hr}^{a} (ng/mL) | 2248 | 118.49 | 107.61 | 130.48 | 1841 |
| | (30) | | | | (20) |
| AUC₀₋ₜ | 21944 | 98.78 | 95.91 | 101.75 | 21962 |
| (ng · hr/mL) | (27) | | | | (22) |
| AUC_{0-inf} | 23090 | 98.73 | 95.85 | 101.7 | 23104 |
| (ng · hr/mL) | (27) | | | | (21) |
| AUC_{0-1 hr} | 823 | 105.42 | 68.75 | 161.64 | 814 |
| (ng · hr/mL) | (96) | | | | (82) |
| AUC_{0-2 hr}^{b} | 2761 | 106.73 | 86.55 | 131.62 | 2492 |
| (ng · hr/mL) | (52) | | | | (47) |
| AUC_{0-4 hr}^{c} | 7006 | 119.91 | 110.42 | 130.2 | 5726 |
| (ng · hr/mL) | (28) | | | | (22) |
| Tₘₐₓ (hr) | 2.328 | na | na | na | 6.971 |
| | (58) | | | | (34) |
| T_{lag} (hr) | 0.276 | na | na | na | 0.219 |
| | (81) | | | | (98) |
| T_{1/2} (hr) | 5.235 | na | na | na | 6.461 |
| | (35) | | | | (66) |
| Kₑₗ (1/hr) | 0.145 | na | na | na | 0.137 |
| | (28) | | | | (39) |

*Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2 SD for commercially-available immediate release tablet

TABLE 10
Acetaminophen Pharmacokinetics (15/500)

| | | | | | Commercially- |
|---|---|---|---|---|---|
| | | | | | available |
| | | | | | immediate |
| | Medium Release Formulation | | | | release tablet* |
| | Mean | LSM | 90% CI | | Mean |
| Parameter | (% CV) | Ratio | Lower | Upper | (% CV) |
| Cₘₐₓ | 2720 | 99.19 | 91.61 | 107.39 | 2721 |
| (ng/mL) | (22) | | | | (22) |
| C_{1 hr} | 1831 | 121.62 | 91.51 | 161.65 | 1516 |
| (ng/mL) | (54) | | | | (58) |
| C_{2 hr}^{a} | 2170 | 116.69 | 105.96 | 128.51 | 1841 |
| (ng/mL) | (23) | | | | (20) |
| AUC₀₋ₜ | 22184 | 100.68 | 97.74 | 103.7 | 21962 |
| (ng · hr/mL) | (22) | | | | (22) |
| AUC_{0-inf} | 23554 | 101.39 | 98.43 | 104.44 | 23104 |
| (ng · hr/mL) | (22) | | | | (21) |
| AUC_{0-1 hr} | 974 | 124.39 | 81.52 | 189.79 | 814 |
| (ng · hr/mL) | (85) | | | | (82) |
| AUC_{0-2 hr}^{b} | 2974 | 117.9 | 95.58 | 145.43 | 2492 |
| (ng · hr/mL) | (47) | | | | (47) |
| AUC_{0-4 hr}^{c} | 7122 | 123.98 | 114.17 | 134.64 | 5726 |
| (ng · hr/mL) | (23) | | | | (22) |
| Tₘₐₓ | 2.069 | na | na | na | 6.971 |
| (hr) | (66) | | | | (34) |
| T_{lag} | 0.218 | na | na | na | 0.219 |
| (hr) | (77) | | | | (98) |
| T_{1/2} | 5.696 | na | na | na | 6.461 |
| (hr) | (33) | | | | (66) |
| Kₑₗ | 0.133 | na | na | na | 0.137 |
| (1/hr) | (29) | | | | (39) |

*Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 11
Acetaminophen Pharmacokinetics (15/500)

| | | | | | Commercially- |
|---|---|---|---|---|---|
| | | | | | available |
| | | | | | immediate |
| | Slow Release Formulation | | | | release tablet* |
| | Mean | LSM | 90% CI | | Mean |
| Parameter | (% CV) | Ratio | Lower | Upper | (% CV) |
| Cₘₐₓ | 2521 | 93.6 | 86.44 | 101.35 | 2721 |
| (ng/mL) | (18) | | | | (22) |
| C_{1 hr} | 1766 | 126.26 | 94.96 | 167.87 | 1516 |
| (ng/mL) | (51) | | | | (58) |
| C_{2 hr}^{a} | 2113 | 116.18 | 105.48 | 127.96 | 1841 |
| (ng/mL) | (18) | | | | (20) |
| AUC₀₋ₜ | 21947 | 99.61 | 96.7 | 102.61 | 21962 |
| (ng · hr/mL) | (25) | | | | (22) |
| AUC_{0-inf} | 23279 | 100.47 | 97.53 | 103.49 | 23104 |
| (ng · hr/mL) | (25) | | | | (21) |
| AUC_{0-1 hr} | 872 | 115.25 | 75.49 | 175.95 | 814 |
| (ng · hr/mL) | (83) | | | | (82) |
| AUC_{0-2 hr}^{b} | 2811 | 116.49 | 94.42 | 143.73 | 2492 |
| (ng · hr/mL) | (43) | | | | (47) |
| AUC_{0-4 hr}^{c} | 6828 | 120.68 | 111.11 | 131.07 | 5726 |
| (ng · hr/mL) | (19) | | | | (22) |
| Tₘₐₓ | 2.184 | na | na | na | 6.971 |
| (hr) | (59) | | | | (34) |
| T_{lag} | 0.253 | na | na | na | 0.219 |
| (hr) | (86) | | | | (98) |
| T_{1/2} | 5.366 | na | na | na | 6.461 |
| (hr) | (32) | | | | (66) |
| Kₑₗ | 0.141 | na | na | na | 0.137 |
| (1/hr) | (28) | | | | (39) |

*Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

The pharmacokinetic parameters for the medium release 15/500 formulation and the commercially-available immediate release tablet are shown in Table 12.

TABLE 12
Pharmacokinetic Profile (Mean ± SD) of Oxycodone/APAP versus commercially-
available immediate release tablet (N = 29)

| | Cₘₐₓ | AUC₀₋ₜ | AUC_{0-inf} | Tₘₐₓ | Kₑₗ | t_{1/2} |
|---|---|---|---|---|---|---|
| Dosage | (ng/mL) | ng · hr/mL) | (ng · hr/mL) | (hr) | (1/hr) | (hr) |
| Oxycodone | | | | | | |
| 15 mg OC/500 mg APAP | 18.3 ± 4.6 | 217 ± 49.2 | 219 ± 49.5 | 5.3 ± | 0.156 ± | 4.6 ± |
| Commercially - | | | | 2.0 | 0.024 | 0.7 |
| available immediate | 22.4 ± 4.5* | 230 ± 49.8 | 231 ± 50.0 | 8.0 ± | 0.157 ± | 4.5 ± |
| release tablet (7.5 mg | | | | 1.7* | 0.020 | 0.6 |
| OC/325 mg APAP) | | | | | | |
| Acetaminophen | | | | | | |
| 15 mg OC/500 mg APAP | 2720 ± 608 | 221184 ± | 23554 ± | 2.1 ± | 0.137 ± | 5.7 ± |
| Commercially - | | 4804 | 5234 | 1.4 | 0.039 | 1.9 |
| available immediate | 2721 ± 584* | 21962 ± | 23104 ± | 7.0 ± | 0.137 ± | 6.5 ± |
| release tablet^{a} (7.5 mg | | 4772 | 4882 | 2.4* | 0.054 | 4.3 |
| OC/325 mg APAP) | | | | | | |

*Most values occurred after the second dose.
^{a}AUC and Cₘₐₓ dose-normalized to 500 mg for APAP.

The oxycodone mean plasma concentration as a function of time after administration of 15/500 tablets is shown in Table 13 and FIG. 5. The APAP mean plasma concentration over time after administration of 15/500 tablets is shown in Table 14 and FIG. 6.

TABLE 13
Time Course of Oxycodone Plasma Concentration (ng/mL)

| | | | | | | | Mean | |
|---|---|---|---|---|---|---|---|---|
| | | | | | | | commercially- | |
| | | | | | | | available | |
| Time | Mean | | Mean | | Mean | | immediate | |
| (hr) | Fast | SEM | Medium | SEM | Slow | SEM | release tablet | SEM |
| 0 | 0 | 0 | 0 | 0 | 0 | 0 | 0 | 0 |
| 0.17 | 0 | 0 | 0.13 | 0.11 | 0.06 | 0.02 | 0.03 | 0.03 |
| 0.33 | 0.65 | 0.29 | 1.08 | 0.44 | 0.93 | 0.41 | 1.16 | 0.36 |
| 0.5 | 2.09 | 0.55 | 2.98 | 0.95 | 2.55 | 0.96 | 4.03 | 0.9 |
| 0.67 | 3.74 | 0.91 | 5.29 | 1.25 | 4.15 | 1.1 | 7.04 | 0.93 |
| 1 | 6.89 | 0.98 | 7.36 | 1.11 | 7.6 | 1.24 | 10.23 | 1.11 |
| 2 | 12.36 | 0.74 | 12.39 | 1.04 | 11.24 | 0.73 | 14.94 | 0.81 |
| 3 | 14.77 | 0.82 | 14.73 | 0.91 | 13.35 | 0.53 | 14.84 | 0.62 |
| 4 | 16.33 | 0.8 | 16.1 | 0.82 | 15.12 | 0.44 | 12.95 | 0.58 |
| 5 | 16.28 | 0.67 | 15.89 | 0.81 | 15.83 | 0.41 | 10.58 | 0.8 |
| 6 | 17.4 | 0.72 | 16.43 | 0.81 | 15.76 | 0.41 | 9.1 | 0.67 |
| 6.5 | 16.59 | 0.64 | 15.89 | 0.72 | 15.22 | 0.96 | 10.76 | 0.7 |
| 7 | 15.28 | 0.58 | 14.83 | 0.69 | 14.49 | 1.43 | 16.84 | 0.69 |
| 8 | 14.02 | 0.6 | 14.29 | 0.64 | 13.77 | 0.85 | 19.7 | 0.7 |
| 9 | 13.13 | 0.57 | 13.39 | 0.55 | 13 | 0.78 | 19.08 | 0.65 |
| 10 | 11.9 | 0.64 | 12.52 | 0.53 | 11.92 | 0.68 | 16.63 | 0.57 |
| 12 | 8.86 | 0.6 | 9.59 | 0.49 | 10.04 | 0.59 | 10.88 | 0.53 |

TABLE 14
Time Course of Acetaminophen Plasma Concentration (ng/mL)

| | | | | | | | Mean | |
|---|---|---|---|---|---|---|---|---|
| | | | | | | | commer- | |
| | | | | | | | cially | |
| | | | | | | | available | |
| | | | | | | | imme- | |
| | | | | | | | diately | |
| Time | Mean | | Mean | | Mean | | release | |
| (hr) | Fast | SEM | Medium | SEM | Slow | SEM | tablet | SEM |
| 0 | 0 | 0 | 0 | 0 | 0 | 0 | 0 | 0 |
| 0.17 | 31 | 18 | 284 | 151 | 220 | 88 | 107 | 47 |
| 0.33 | 673 | 210 | 751 | 221 | 678 | 197 | 607 | 173 |
| 0.5 | 1216 | 266 | 1299 | 275 | 1133 | 248 | 1181 | 229 |
| 0.67 | 1624 | 301 | 1922 | 301 | 1647 | 252 | 1653 | 255 |
| 1 | 2116 | 258 | 2380 | 239 | 2296 | 217 | 1971 | 210 |
| 2 | 2922 | 160 | 2821 | 123 | 2747 | 93 | 2393 | 90 |
| 3 | 2736 | 129 | 2719 | 90 | 2636 | 94 | 2150 | 65 |
| 4 | 2643 | 120 | 2524 | 103 | 2424 | 110 | 1717 | 71 |
| 5 | 2376 | 112 | 2246 | 121 | 2130 | 118 | 1290 | 59 |
| 6 | 2263 | 100 | 2080 | 143 | 1965 | 107 | 1006 | 58 |
| 6.5 | 2068 | 93 | 1903 | 126 | 1774 | 102 | 1742 | 212 |
| 7 | 1830 | 80 | 1744 | 116 | 1644 | 98 | 2749 | 232 |
| 8 | 1577 | 81 | 1573 | 103 | 1495 | 93 | 2790 | 114 |
| 9 | 1416 | 79 | 1407 | 88 | 1330 | 80 | 2482 | 111 |
| 10 | 1286 | 82 | 1314 | 84 | 1198 | 71 | 1968 | 105 |
| 12 | 1069 | 89 | 1131 | 86 | 1089 | 66 | 1188 | 82 |

### Example 3

### Clinical Pharmacokinetic Analysis of Controlled-Release 30 mg Oxycodone/500 mg Acetaminophen Bilayer Tablets—Single Dose

A single dose, four-period crossover study was conducted essentially as described in Example 2, except the controlled-release bilayer tablets contained 30 mg oxycodone and 500 mg APAP. (See selected examples from Chart No. 2.) Tables 15-17 and 18-20 present the PK data for oxycodone and APAP, respectively. The plasma concentrations of oxycodone and APAP are presented in FIG. 7 and FIG. 8, respectively.

TABLE 15
Oxycodone Pharmacokinetics (30/500)

| | | | | | Commercially- |
|---|---|---|---|---|---|
| | | | | | available |
| | | | | | immediate |
| | Fast Release Formulation | | | | release tablet |
| | Mean | LSM | 90% CI | | Mean |
| Parameter | (% CV) | Ratio | Lower | Upper | (% CV) |
| Cₘₐₓ | 39.159 | 82.17 | 75.96 | 88.9 | 47.597 |
| (ng/mL) | (28) | | | | (26) |
| C_{1 hr} | 20.462 | 77.25 | 54.37 | 109.76 | 25.911 |
| (ng/mL) | (74) | | | | (67) |
| C_{2 hr}^{a} | 28.221 | 95.18 | 83.82 | 108.08 | 29.579 |
| (ng/mL) | (39) | | | | (32) |
| AUC₀₋ₜ | 393.952 | 92.84 | 89.3 | 96.53 | 425.978 |
| (ng · hr/mL) | (30) | | | | (29) |
| AUC_{0-inf} | 396.135 | 92.4 | 88.94 | 95.99 | 430.196 |
| (ng · hr/mL) | (29) | | | | (29) |
| AUC_{0-1 hr} | 9.106 | 71.09 | 46.05 | 109.76 | 11.55 |
| (ng · hr/mL) | (100) | | | | (93) |
| AUC_{0-2 hr}^{b} | 33.448 | 82.59 | 67.9 | 100.46 | 39.295 |
| (ng · hr/mL) | (61) | | | | (53) |
| AUC_{0-4 hr}^{c} | 96.47 | 101.27 | 91.51 | 112.06 | 93.706 |
| (ng · hr/mL) | (38) | | | | (29) |
| AUC_{4 hr-t}^{d} | 395.522 | 92.4 | 88.95 | 95.99 | 429.507 |
| | (29) | | | | (29) |
| Tₘₐₓ | 4.057 | na | na | na | 6.948 |
| (hr) | (51) | | | | (33) |
| T_{lag} | 0.213 | na | na | na | 0.184 |
| (hr) | (107) | | | | (66) |
| T_{1/2} | 4.398 | na | na | na | 4.32 |
| (hr) | (15) | | | | (15) |
| Kₑₗ | 0.161 | na | na | na | 0.164 |
| (1/hr) | (15) | | | | (16) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 16
Oxycodone Pharmacokinetics (30/500)

| | | | | | Commercially- |
|---|---|---|---|---|---|
| | | | | | available |
| | | | | | immediate |
| | Medium Release Formulation | | | | release tablet |
| | Mean | LSM | 90% CI | | Mean |
| Parameter | (% CV) | Ratio | Lower | Upper | (% CV) |
| Cₘₐₓ | 36.731 | 77.14 | 71.27 | 83.48 | 47.597 |
| (ng/mL) | (30) | | | | (26) |
| C_{1 hr} | 19.758 | 86.12 | 60.48 | 122.62 | 25.911 |
| (ng/mL) | (70) | | | | (67) |
| C_{2 hr}^{a} | 27.655 | 93.53 | 82.31 | 106.28 | 29.579 |
| (ng/mL) | (39) | | | | (32) |
| AUC₀₋ₜ | 396.026 | 94.17 | 90.55 | 97.92 | 425.978 |
| (ng · hr/mL) | (29) | | | | (29) |
| AUC_{0-inf} | 398.084 | 93.68 | 90.16 | 97.34 | 430.196 |
| (ng · hr/mL) | (29) | | | | (29) |
| AUC_{0-1 hr} | 8.988 | 93.06 | 60.12 | 144.04 | 11.55 |
| (ng · hr/mL) | (85) | | | | (93) |
| AUC_{0-2 hr}^{b} | 32.695 | 86.02 | 70.64 | 104.74 | 39.295 |
| (ng · hr/mL) | (56) | | | | (53) |
| AUC_{0-4 hr}^{c} | 91.998 | 98.13 | 88.63 | 108.65 | 93.706 |
| (ng · hr/mL) | (36) | | | | (29) |
| AUC_{4 hr-t}^{d} | 397.436 | 93.68 | 90.16 | 97.34 | 429.507 |
| | (29) | | | | (29) |
| Tₘₐₓ | 4.523 | na | na | na | 6.948 |
| (hr) | (51) | | | | (33) |
| T_{lag} | 0.207 | na | na | na | 0.184 |
| (hr) | (95) | | | | (66) |
| T_{1/2} | 4.369 | na | na | na | 4.32 |
| (hr) | (14) | | | | (15) |
| Kₑₗ | 0.162 | na | na | na | 0.164 |
| (1/hr) | (14) | | | | (16) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 17
Oxycodone Pharmacokinetics (30/500)

| | | | | | Commercially- |
|---|---|---|---|---|---|
| | | | | | available |
| | | | | | immediate |
| | Slow Release Formulation | | | | release tablet |
| | Mean | LSM | 90% CI | | Mean |
| Parameter | (% CV) | Ratio | Lower | Upper | (% CV) |
| Cₘₐₓ | 32.976 | 68.96 | 63.74 | 74.6 | 47.597 |
| (ng/mL) | (29) | | | | (26) |
| C_{1 hr} | 17.897 | 73.61 | 52.01 | 104.18 | 25.911 |
| (ng/mL) | (74) | | | | (67) |
| C_{2 hr}^{a} | 23.183 | 78.42 | 69.06 | 89.05 | 29.579 |
| (ng/mL) | (33) | | | | (32) |
| AUC₀₋ₜ | 399.623 | 94.5 | 90.9 | 98.25 | 425.978 |
| (ng · hr/mL) | (26) | | | | (29) |
| AUC_{0-inf} | 401.362 | 93.88 | 90.36 | 97.52 | 430.196 |
| (ng · hr/mL) | (26) | | | | (29) |
| AUC_{0-1 hr} | 7.643 | 69.93 | 45.52 | 107.44 | 11.55 |
| (ng · hr/mL) | (96) | | | | (93) |
| AUC_{0-2 hr}^{b} | 28.183 | 71.58 | 58.85 | 87.06 | 39.295 |
| (ng · hr/mL) | (59) | | | | (53) |
| AUC_{0-4 hr}^{c} | 82.171 | 86.17 | 77.87 | 95.35 | 93.706 |
| (ng · hr/mL) | (36) | | | | (29) |
| AUC_{4 hr-t}^{d} | 400.56 | 93.85 | 90.34 | 97.49 | 429.507 |
| | (26) | | | | (29) |
| Tₘₐₓ | 3.96 | na | na | na | 6.948 |
| (hr) | (48) | | | | (33) |
| T_{lag} | 0.201 | na | na | na | 0.184 |
| (hr) | (78) | | | | (66) |
| T_{1/2} | 4.418 | na | na | na | 4.32 |
| (hr) | (17) | | | | (15) |
| Kₑₗ | 0.161 | na | na | na | 0.164 |
| (1/hr) | (17) | | | | (16) |

^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 18
Acetaminophen Pharmacokinetics (30/500)

| | | | | | Commercially- |
|---|---|---|---|---|---|
| | | | | | available |
| | | | | | immediate |
| | Fast Release Formulation | | | | release tablet |
| | Mean | LSM | 90% CI | | Mean |
| Parameter | (% CV) | Ratio | Lower | Upper | (% CV) |
| Cₘₐₓ | 3138 | 101.52 | 91.58 | 122.53 | 3085 |
| (ng/mL) | (32) | | | | (29) |
| C_{1 hr} | 2163 | 130.98 | 101.04 | 169.78 | 1777 |
| (ng/mL) | (59) | | | | (59) |
| C_{2 hr}^{a} | 2386 | 125.37 | 113.22 | 138.82 | 1892 |
| (ng/mL) | (32) | | | | (28) |
| AUC₀₋ₜ | 21742 | 98.53 | 95.07 | 102.13 | 21897 |
| (ng · hr/mL) | (26) | | | | (23) |
| AUC_{0-inf} | 22798 | 99.02 | 95.5 | 102.66 | 22881 |
| (ng · hr/mL) | (26) | | | | (23) |
| AUC_{0-1 hr} | 1260 | 122.71 | 85.05 | 177.03 | 1005 |
| (ng · hr/mL) | (85) | | | | (80) |
| AUC_{0-2 hr}^{b} | 3534 | 120.52 | 100.69 | 144.26 | 2839 |
| (ng · hr/mL) | (53) | | | | (48) |
| AUC_{0-4 hr}^{c} | 8038 | 130.54 | 119.98 | 142.02 | 6041 |
| (ng · hr/mL) | (33) | | | | (27) |
| AUC_{4 hr-t}^{d} | 14707 | 86.22 | 82.35 | 90.27 | 16720 |
| | (32) | | | | (26) |
| Tₘₐₓ | 1.908 | na | na | na | 5.615 |
| (hr) | (69) | | | | (54) |
| T_{lag} | 0.236 | na | na | na | 0.178 |
| (hr) | (106) | | | | (90) |
| T_{1/2} | 4.798 | na | na | na | 5.3 |
| (hr) | (26) | | | | (43) |
| Kₑₗ | 0.153 | na | na | na | 0.152 |
| (1/hr) | (25) | | | | (36) |

* Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 19
Acetaminophen Pharmacokinetics (30/500)

| | | | | | Commercially- |
|---|---|---|---|---|---|
| | | | | | available |
| | | | | | immediate |
| | Medium Release Formulation | | | | release tablet |
| | Mean | LSM | 90% CI | | Mean |
| Parameter | (% CV) | Ratio | Lower | Upper | (% CV) |
| Cₘₐₓ | 2940 | 93.8 | 84.57 | 104.03 | 3085 |
| (ng/mL) | (38) | | | | (29) |
| C_{1 hr} | 2161 | 139.29 | 107.29 | 180.84 | 1777 |
| (ng/mL) | (56) | | | | (59) |
| C_{2 hr}^{a} | 2349 | 125.86 | 113.61 | 139.44 | 1892 |
| (ng/mL) | (27) | | | | (28) |
| AUC₀₋ₜ (ng · hr/mL) | 21822 | 99.42 | 95.9 | 103.06 | 21897 |
| | (26) | | | | (23) |
| AUC_{0-inf} (ng · hr/mL) | 23107 | 100.76 | 97.16 | 104.49 | 22881 |
| | (26) | | | | (23) |
| AUC_{0-1 hr} (ng · hr/mL) | 1342 | 155.89 | 107.81 | 225.4 | 1005 |
| | (81) | | | | (80) |
| AUC_{0-2 hr}^{b} (ng · hr/mL) | 3596 | 129.14 | 107.79 | 154.73 | 2839 |
| | (52) | | | | (48) |
| AUC_{0-4 hr}^{c} (ng · hr/mL) | 7880 | 130.08 | 119.51 | 141.59 | 6041 |
| | (32) | | | | (27) |
| AUC_{4 hr-t}^{d} | 15040 | 88.93 | 84.92 | 93.13 | 16720 |
| | (29) | | | | (26) |
| Tₘₐₓ | 1.724 | na | na | na | 5.615 |
| (hr) | (62) | | | | (54) |
| T_{lag} | 0.19 | na | na | na | 0.178 |
| (hr) | (114) | | | | (90) |
| T_{1/2} | 6.116 | na | na | na | 5.3 |
| (hr) | (63) | | | | (43) |
| Kₑₗ | .0139 | na | na | na | 0.152 |
| (1/hr) | (37) | | | | (36) |

* Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

TABLE 20
Acetaminophen Pharmacokinetics (30/500)

| | | | | | Commercially- |
|---|---|---|---|---|---|
| | | | | | available |
| | | | | | immediate |
| | Slow Release Formulation | | | | release tablet |
| | Mean | LSM | 90% CI | | Mean |
| Parameter | (% CV) | Ratio | Lower | Upper | (% CV) |
| Cₘₐₓ | 2734 | 88.33 | 79.68 | 97.91 | 3085 |
| (ng/mL) | (33) | | | | (29) |
| C_{1 hr} | 1989 | 120.26 | 93.05 | 155.44 | 1777 |
| (ng/mL) | (53) | | | | (59) |
| C_{2 hr}^{a} | 2131 | 112.77 | 101.84 | 124.86 | 1892 |
| (ng/mL) | (25) | | | | (28) |
| AUC₀₋ₜ | 21272 | 97.1 | 93.68 | 100.64 | 21897 |
| (ng · hr/mL) | (23) | | | | (23) |
| AUC_{0-inf} | 22504 | 98.45 | 94.95 | 102.07 | 22881 |
| (ng · hr/mL) | (22) | | | | (23) |
| AUC_{0-1 hr} | 1092 | 120.91 | 84.15 | 173.72 | 1005 |
| (ng · hr/mL) | (76) | | | | (80) |
| AUC_{0-2 hr}^{b} | 3152 | 112.74 | 94.19 | 134.94 | 2839 |
| (ng · hr/mL) | (45) | | | | (48) |
| AUC_{0-4 hr}^{c} | 7217 | 119.31 | 109.5 | 129.61 | 6041 |
| (ng · hr/mL) | (26) | | | | (27) |
| AUC_{4 hr-t}^{d} | 15227 | 90.59 | 86.52 | 94.85 | 16720 |
| | (26) | | | | (26) |
| Tₘₐₓ | 1.897 | na | na | na | 5.615 |
| (hr) | (56) | | | | (54) |
| T_{lag} | 0.196 | na | na | na | 0.178 |
| (hr) | (79) | | | | (90) |
| T_{1/2} | 4.843 | na | na | na | 5.3 |
| (hr) | (27) | | | | (43) |
| Kₑₗ | 0.152 | na | na | na | 0.152 |
| (1/hr) | (24) | | | | (36) |

* Dose Normalized to 500 mg
^{a}Concentration at the median Tₘₐₓ for commercially-available immediate release tablet
^{b}AUC from zero the median Tₘₐₓ for commercially-available immediate release tablet
^{c}AUC from the zero to the median Tₘₐₓ + 2SD for commercially-available immediate release tablet

The pharmacokinetic parameters for the medium release 30/500 formulation and the commercially-available immediate release tablet are shown in Table 21.

TABLE 21
Pharmacokinetic Profile (Mean ± SD) of Oxycodone/APAP versus
Commercially-available immediate release tablet (N = 29)

| | Cₘₐₓ | AUC₀₋ₜ | AUC_{0-inf} | Tₘₐₓ | Kₑₗ | t_{1/2} |
|---|---|---|---|---|---|---|
| Dosage | (ng/mL) | (ng · hr/mL) | (ng · hr/mL) | (hr) | (1/hr) | (hr) |
| Oxycodone | | | | | | |
| 30 mg OC/500 mg | 36.7 ± | 396 ± 116 | 398 ± 115 | 4.5 ± | 0.162 ± | 4.4 ± |
| APAP | 10.9 | | | 2.3 | 0.023 | 0.6 |
| Commercially- | 47.6 ± | 426 ± 125 | 430 ± 124 | 6.9 ± | 0.164 ± | 4.3 ± |
| available | 12.3* | | | 2.3* | 0.026 | 0.6 |
| immediate release | | | | | | |
| tablet^{a} (7.5 mg OC/325 | | | | | | |
| mg APAP) | | | | | | |
| Acetaminophen | | | | | | |
| 30 mg OC/500 mg | 2940 ± | 21822 ± | 23107 ± | 1.7 ± | 0.139 ± | 6.1 ± |
| APAP | 1105 | 5630 | 5927 | 1.1 | 0.052 | 3.9 |
| Commercially | 3085 ± | 21897 ± | 22881 ± | 5.6 ± | 0.152 ± | 5.3 ± |
| available- | 899* | 5125 | 5362 | 3.0* | 0.055 | 2.3 |
| immediate release | | | | | | |
| tablet^{a} (7.5 mg OC/325 | | | | | | |
| mg APAP) | | | | | | |

*Most values occurred after the second dose.
^{a}AUC and Cmax dose-normalized to 30 mg for OC and 500 mg for APAP.

### Example 4

### Clinical Pharmacokinetic Analysis of Controlled-Release 15 mg Oxycodone/650 mg Acetaminophen Bilayer Tablets—Single Dose

The following study evaluated the bioavailability, pharmacokinetics, dose-proportionality, and safety of 1 or 2 tablets of a formulation comprising 15 mg OC/650 mg APAP (1 dose) (see selected example from Chart No. 1) compared to 1 tablet of the commercially-available immediate release tablet under fed conditions. The ER layer contained 75% of the total amount of the oxycodone in the tablet, 50% of the total amount of APAP in the tablet, and 45% (w/w) POLYOX® 1105. The IR layer contained 25% of the total amount of oxycodone in the tablet and 50% of the total amount of APAP. This study was conducted in 42 male and female healthy subjects.

PK parameters for oxycodone are presented in Table 22. Plasma concentrations of OC for the 1 tablet dosing configuration of 15/650 showed a median t_{lag} of 0.25 hours, while there was no lag time for plasma concentrations of OC for the 2 tablet dosing configuration of 15/650 and the commercially-available immediate release tablet under fed conditions. As illustrated in FIG. 9 demonstrating the plasma concentrations of oxycodone versus time of treatment (i.e., Treatment A was one tablet of 15 mg oxycodone/650 mg acetaminophen administered orally under fed conditions; Treatment B was two tablets of 15 mg oxycodone/650 mg acetaminophen administered orally one at a time under fed conditions; and Treatment C was one tablet of the commercially-available immediate release tablet (7.5 mg oxycodone/325 mg acetaminophen) administered orally every 6 hours for 2 doses under fed conditions). Plasma concentrations of OC rose rapidly after administration of 15/650 formulation in a similar fashion to commercially-available immediate release tablet. Peak plasma levels of OC for the 15/650 tablets, however, were biphasic. Peak levels were observed at about 2-3 hours and about 6 hours for the 1 or 2 tablet dosing configuration of the 15/650 formulation. In contrast, the peak plasma level of OC for the commercially-available immediate release tablet was about 7-8 hours after the initial dose of the commercially-available immediate release tablet (˜1-2 hr after the second dose). Mean plasma concentrations of OC from 15/650 formulations were detectable through 48 hours following all treatments and t₁₁₂ was about 4 hours across all treatments.

TABLE 22
Pharmacokinetic Parameter Estimates (Mean ± SD) of Oxycodone Following
Administration of 15 mg Oxycodone/650 mg APAP versus Commercially-available
immediate release tablet

| | Cₘₐₓ | AUC₀₋ₜ | AUC_{0-inf} | Tₘₐₓ^{a} | T_{lag}^{a} | t_{1/2} |
|---|---|---|---|---|---|---|
| Dosage | (ng/mL) | (nghr/mL) | (nghr/mL) | (hr) | (hr) | (hr) |
| One tablet | 17.68 | 199.60 | 201.6 | 3.00 | 0.25 | 4.18 |
| (N = 25) | (4.42) | (59.52) | (59.27) | (1.00 − | (0.00 − | (0.77) |
| Treatment A | | | | 12.45) | 0.75) | |
| Two tablets | 29.18 | 414.73 | 417.41^{b} | 5.00 | 0.00 | 4.11b |
| (N = 25) | (6.53) | (109.87) | (112.17) | (1.00 - | (0.00 − | (0.67) |
| Treatment B | | | | 12.00) | 0.50) | |
| Commercially- | 20.34 | 199.63 | 201.76 | 7.00 | 0.00 | 4.08 |
| available | (4.81) | (60.53) | (60.24) | (0.50 − | (0.00 − | (0.64) |
| immediate | | | | 9.00) | 1.00) | |
| release tablet | | | | | | |
| (7.5 mg | | | | | | |
| OC/325 mg | | | | | | |
| APAP | | | | | | |
| (N = 25) | | | | | | |
| Treatment C | | | | | | |

^{a}Tₘₐₓ and t_{lag} median (minimum − maximum)
^{b}N = 24

PK parameters for APAP are presented in Table 23. Plasma concentrations of APAP for the 1 tablet dosing configuration of 15/650 showed a median t_{lag} of 0.25 hour, while there was no lag in the appearance of APAP in plasma for the 2 tablet dosing configuration of 15/650 and the commercially-available immediate release tablet. Plasma concentrations of APAP rose rapidly after administration of the 15/650 formulations, similar to that observed with RDL. (See FIG. 10). Peak plasma levels of APAP following administration of the 1 tablet and 2 tablet dosing configurations of 15/650 were observed at approximately 2 hours (with a shoulder peak at 5-6 hours) after dosing compared with 1 hour after the second dose of the commercially-available immediate release tablet. Mean plasma concentrations of APAP were detectable through 36 hours following all treatments and the mean t_{1/2} was approximately 6 to 8 hours across treatment groups.

TABLE 23
Pharmacokinetic Parameter Estimates (Mean ± SD) of APAP Following
Administration of 15 mg Oxycodone/650 mg APAP versus Commercially-available
immediate release tablet

| | Cₘₐₓ | AUC₀₋ₜ | AUC_{0-inf} | Tₘₐₓ^{a} | T_{lag}^{a} | t_{1/2} |
|---|---|---|---|---|---|---|
| Dosage | (ng/mL) | (ng · hr/mL) | (ng · hr/mL) | (hr) | (hr) | (hr) |
| One tablet | 3822 | 30239 | 32194^{c} | 2.00 | 0.25 | 6.17^{c} |
| (N = 25) | (874) | (5673) | (6437) | (0.50 − | (0.00 − 1.00) | (2.22) |
| | | | | 4.00) | | |
| Two tablets | 6941 | 64783 | 67600^{d} | 2.00 | 0.00 | 7.67^{d} |
| (N = 25) | (1989) | (15017) | (14655) | (0.50 − | (0.00 − 0.50) | (4.06) |
| | | | | 5.00) | | |
| Commercially- | 3629 | 30137 | 30802^{c} | 6.50 | 0.00 | 5.89^{c} |
| available immediate | (841) | (6426) | (6697) | (0.50 − | (0.00 − 1.00) | (2.63) |
| release tablet | | | | 9.00) | | |
| (7.5 mg OC/325 mg | | | | | | |
| APAP (N = 25) | | | | | | |

^{a}Tₘₐₓ and t_{lag} median (minimum − maximum)
^{c}N = 21
^{d}N = 23

### Example 5

### Clinical Pharmacokinetic Analysis of Controlled-Release 15 mg Oxycodone/650 mg Acetaminophen Bilayer Tablets—Multiple Doses

The following study evaluated the steady state bioavailability, pharmacokinetics, and safety of a 15 mg OC/650 mg APAP composition administered (see selected example from Chart No. 2) orally as 1 tablet (Treatment A) or 2 tablets (Treatment B) every 12 hours (9 doses) compared to 2 tablets of the commercially-available immediate release tablet (2×7.5 mg OC/325 mg APAP) (Treatment C) dosed every 6 hours for 4.5 days (18 doses) under fed conditions with 48 male and female subjects in equal distribution.

The pharmacokinetic (PK) parameters of OC are presented in Table 24. The PK behavior of OC on Study Day 1 was similar to that observed in the single dose study (see Table 22). There was a slight lag (median tlag 0.25 hr) in the appearance of OC following the 1 tablet dose of 15 mg OC/650 mg APAP. No lag was observed following dosing with 2 tablets of 15 mg OC/650 mg APAP or the commercially-available immediate release tablet. Peak plasma levels were observed at 4 and 6 hours after administration of 1 and 2 tablets of the 15/650 formulation, respectively, and at 1.5 hours after the second dose of the commercially-available immediate release tablet. (See FIG. 11). Minimum (trough) plasma concentrations (Cₘᵢₙ) of OC achieved steady-state levels by Day 2 for 15/650 formulations and by Day 3 for the commercially-available immediate release tablet.

TABLE 24
Oxycodone Pharmacokinetic Parameters

| | Cₘₐₓ | AUC₀₋ₜ | Tₘₐₓ^{a} | T_{lag}^{a} | t_{1/2} |
|---|---|---|---|---|---|
| Dosage | (ng/mL) | (ng · hr/mL) | (hr) | (hr) | (hr) |
| A: One tablet | 18.79 | 149.68^{c} | 4.00 | 0.25 | Day 1 |
| Day 1 | (5.00) | (37.92) | (2.00-8.00) | (0.00-0.50) | |
| (N = 20) | | | | | |
| B: Two tablets | 33.57 | 280.45^{c} | 5.93 | 0.00 | Day 1 |
| Day 1 | (8.41) | (62.61) | (1.00-11.92) | (0.00-0.25) | |
| (N = 20) | | | | | |
| C: | 36.02 | 278.60^{c} | 7.50 | 0.00 | Day 1 |
| Commercially-available | (10.52) | (67.17) | (0.75-11.92) | (0.00-0.33) | |
| immediate release tablet | | | | | |
| (7.5 mg OC/325 mg | | | | | |
| APAP | | | | | |
| Day 1 | | | | | |
| (N = 20) | | | | | |
| A: One tablet | 27.26 | 223.10^{c} | 3.00 | Day 5 | 6.06^{d} |
| Day 5 | (6.33) | (59.45) | (1.00-5.92) | | (1.91) |
| (N = 20) | | | | | |
| B: Two tablets | 50.70 | 433.37^{c} | 3.00 | Day 5 | 6.35 |
| Day 5 | (10.95) | (93.21) | (2.00-7.00) | | (1.89) |
| (N = 20) | | | | | |
| C: | 52.41 | 435.70^{c} | 2.00 | Day 5 | 5.93^{d} |
| Commercially-available | (12.40) | (98.68) | (0.50-8.02) | | (1.68) |
| immediate release tablet | | | | | |
| (7.5 mg OC/325 mg | | | | | |
| APAP | | | | | |
| Day 5 | | | | | |
| (N = 20) | | | | | |

^{a}Tₘₐₓ and t_{lag} median (minimum-maximum)
^{c}Day 1 - AUC_{0-12 h}; Day 5 - AUC_{0-12 h} ss
^{d}N = 19

On Day 5 of the study, the maximum plasma OC concentration at steady-state (Cₘₐₓ^{ss}) was 27.3 ng/mL following 4.5 days of dosing with 1 tablet of 15 mg OC/650 mg APAP administered every 12 hours. Cₘₐₓ^{ss} following 2 tablets of 15 mg OC/650 mg APAP administered every 12 hours or the commercially-available immediate release tablet administered Q6 hours for 4.5 days were 50.7 ng/mL and 52.4 ng/mL, respectively. Median Tₘₐₓ^{ss} was observed at 3 hours following 1 tablet or 2 tablets of 15/650 and at 2 hours following the first daily dose of the commercially-available immediate release tablet.

PK parameters for APAP are presented in Table 25. Acetaminophen was rapidly absorbed following a single dose of 1 or 2 tablets of 15/650 and in a similar fashion to the commercially-available immediate release tablet (see FIG. 12). There was no lag in plasma concentrations following any of the three dosing regimens. Peak APAP plasma concentrations were observed at 1 hour after administration of 1 or 2 tablets of 15/650 and at 0.9 hours after the first dose of the commercially-available immediate release tablet on Day 1. After a single administration of 15/650, Cₘₐₓ for APAP was proportional with respect to the amount of APAP in 1 or 2 tablets of 15/650 (i.e., 1 tablet—3942 ng/mL; 2 tablets—7536 ng/mL). Minimum (trough) concentrations (Cₘᵢₙ) of APAP achieved steady-state levels by Day 2 for 1 tablet of 15/650, by Day 4 for 2 tablets of 15/650 and by the second dose on Day 1 for the commercially-available immediate release tablet.

TABLE 25
Acetaminophen Pharmacokinetic Parameters

| | Cₘₐₓ | AUC₀₋ₜ | Tₘₐₓ^{a} | T_{lag}^{a} | t_{1/2} |
|---|---|---|---|---|---|
| Dosage | (ng/mL) | (ng · hr/mL) | (hr) | (hr) | (hr) |
| A: One tablet | 3942 | 22928^{g} | 1.00 | 0.00 | Day 1 |
| Day 1 | (1168) | (7331) | (0.50-5.93) | (0.00-0.28) | |
| (N = 20) | | | | | |
| B: Two tablets | 7536 | 44254^{g} | 1.00 | 0.00 | Day 1 |
| Day 1 | (2205) | (13885) | (0.28-4.00) | (0.00-0.25) | |
| (N = 20) | | | | | |
| C: | 6757 | 43634^{g} | 0.90 | 0.00 | Day 1 |
| Commercially-available | (1949) | (12357) | (0.32-11.92) | (0.00-0.25) | |
| immediate release tablet | | | | | |
| (7.5 mg OC/325 mg | | | | | |
| APAP | | | | | |
| Day 1 | | | | | |
| (N = 20) | | | | | |
| A: One tablet | 4635 | 26968^{g} | 1.00 | Day 5 | 7.06 |
| Day 5 | (1330) | (9134) | (0.50-3.00) | | (2.24) |
| (N = 20) | | | | | |
| B: Two tablets | 8206 | 50221^{g} | 1.00 | Day 5 | 7.46 |
| Day 5 | (2666) | (18415) | (0.30-4.00) | | (1.85) |
| (N = 20) | | | | | |
| C: | 7433 | 50678^{g} | 1.50 | Day 5 | 6.79^{h} |
| Commercially-available | (1979) | (15565) | (0.25-8.02) | | (2.47) |
| immediate release tablet | | | | | |
| (7.5 mg OC/325 mg | | | | | |
| APAP | | | | | |
| Day 5 | | | | | |
| (N = 20) | | | | | |

^{a}Tₘₐₓ and t_{lag} median (minimum-maximum)
^{g}Day 1 - AUC_{0-12 h}; Day 5 - AUC_{0-12 h}^{ss}
^{h}N = 17

On Day 5 of the study, median Tₘₐₓ^{ss} for APAP was observed at 1 hour following 1 or 2 tablets of 15/650 and at 1.5 hours following the first daily dose of the commercially-available immediate release tablet on Day 5. Maximum plasma APAP concentration at steady-state (Cₘₐₓ^{ss}) was 4635 ng/mL following 4.5 days of dosing with 1 tablet of 15/650 every 12 hours (Table 25). Cₘₐₓ^{ss} following 2 tablets of 15/650 administered every 12 hours and for the commercially-available immediate release tablet administered Q6 hours for 4.5 days were 8206 and 7433 ng/mL, respectively.

### Example 6

### Clinical Pharmacokinetic Analysis of Controlled-Release 15 mg Oxycodone/650 mg Acetaminophen Bilayer Tablets Under Fed and Fasted Conditions

Two open-label, randomized, two-period crossover studies were conducted to evaluate the effect of food on the pharmacokinetics, bioavailability and safety of the 15 mg oxycodone/650 mg APAP composition (see selected example from Chart No. 2) using a 1 tablet or 2 tablet dosing configuration in normal, healthy subjects. Studies were conducted in 48 subjects under fed (FDA high fat breakfast) or fasted conditions.

Tables 26 and 27 present the pharmacokinetic data for oxycodone (OC) and APAP, respectively. FIGS. 13 and 14 present the plasma concentration of OC following administration of one tablet and two tablets, respectively, under fed (Treatment A) or fasted (Treatment B) conditions. FIGS. 15 and 16 present the plasma concentration of APAP following administration of one tablet and two tablets, respectively, under fed (Treatment A) or fasted (Treatment B) conditions.

TABLE 26
Oxycodone Pharmacokinetics (15/650)

| | | | AUC₀₋ₜ | AUC_{0-inf} | | | |
|---|---|---|---|---|---|---|---|
| | State | Cₘₐₓ | (ng · hr/ | (ng · hr/ | Tₘₐₓ^{a} | t_{lag}^{a} | t_{1/2} |
| Dose | (N) | (ng/mL) | mL) | mL) | (hr) | (hr) | (hr) |
| One | fed | 19.03 (4.20) | 219.23 | 221.06 | 5.00 | 0.25 | 3.94 |
| tablet | (28) | | (55.99) | (55.88) | (1.00 − | (0.00 − | (0.69) |
| | | | | | 12.00) | 0.50) | |
| Two | fed | 30.58 (6.57) | 414.01 | 415.88 | 5.00 | 0.25 | 4.42 |
| tablets | (17) | | (104.76) | (104.86) | (0.75 − | (0.00 − | (0.97) |
| | | | | | 12.00) | 0.27) | |
| One | fasted | 18.31 (4.67) | 196.51 | 198.33 | 3.50 | 0.00 | 4.25 |
| tablet | (28) | | (53.04) | (52.82) | (0.50 − | (0.00 − | (0.59) |
| | | | | | 10.00) | 0.25) | |
| Two | fasted | 33.69 (7.45) | 390.33 | 392.15 | 5.00 | 0.00 | 4.80 |
| tablets | (17) | | (145.27) | (145.81) | (2.00 − | (0.00 − | (1.07) |
| | | | | | 5.20) | 0.25) | |

^{a}Tₘₐₓ and t_{lag} median (minimum − maximum)

Plasma concentrations (Table 26; FIGS. 13 and 14) of OC rose rapidly with the median Tₘₐₓ observed at about 4 to 5 hr under both fed and fasted conditions for both the 1- and 2-tablet dose configurations. OC plasma levels were biphasic—with a first peak at about 3 hours and a second peak at about 5 hours. The Cₘₐₓ values (at 5 hours) for OC under fed (1 and 2 tablets, 19.0 and 30.6 ng/mL) conditions were equivalent to those observed under fasted (1 and 2 tablets, 18.3 and 33.7 ng/mL) conditions for both the 1 tablet and 2 tablet dosing configurations.

TABLE 27
Acetaminophen Pharmacokinetics (15/650)

| | State | Cₘₐₓ | AUC₀₋ₜ | AUC_{0-inf} | Tₘₐₓ^{a} | t_{lag}^{a} | t_{1/2} |
|---|---|---|---|---|---|---|---|
| Dose | (N) | (ng/mL) | (nghr/mL) | (nghr/mL) | (hr) | (hr) | (hr) |
| One tablet | fed | 4374 | 31480 | 32552 | 1.00 | 0.00 | 4.65 |
| | (28) | (1286) | (9316) | (9489) | (0.50, | (0.00 − 0.50) | (1.26) |
| | | | | | 5.00) | | |
| Two tablets | fed | 6341 | 62904 | 68839^{b} | 2.00 | 0.00 | 7.02^{b} |
| | (17) | (1698) | (19294) | (19826) | (0.75 − | (0.00 − 0.25) | (1.77) |
| | | | | | 6.00) | | |
| One tablet | fasted | 5511 | 31876 | 33860 | 0.75 | 0.00 | 5.19^{e} |
| | (28) | (2095) | (103339) | (10731) | (0.25, | (0.00 − 0.25) | (1.50) |
| | | | | | 5.00) | | |
| Two tablets | fasted | 10428 | 61164 | 65281 | 0.75 | 0.00 | 5.6 |
| | (17) | (3529) | (16652) | (15711) | (0.25 − | (0.00 − 0.00) | (1.49) |
| | | | | | (5.00) | | |

^{a}Tₘₐₓ and t_{lag} median (minimum − maximum)
^{b} N = 12
^{e} N = 27
^{f} N = 13

Plasma concentrations (Table 27; FIGS. 15 and 16) of APAP rose rapidly following 1 tablet dosed under fed and fasted conditions with similar Tₘₐₓ values (1.0 hour and 0.8 hour). Tₘₐₓ was observed sooner following 2 tablets given under fasted conditions (0.8 hour) than under fed conditions (2 hours). Plasma concentrations of APAP were lower under fed conditions than under fasted conditions with fed Cₘₐₓ values of 4374 ng/mL (1 tablet) and 6341 ng/mL (2 tablets) and fasted Cₘₐₓ values of 5511 ng/mL (1 tablet) and 10,428 ng/mL (2 tablets). Nevertheless, the peak concentrations demonstrate that there was only a slight, minimal food effect on the absorption of APAP, which is consistent with that observed for other oxycodone and acetaminophen products. Thus, there is no meaningful food effect seen with this composition, and as such, the composition can to be administered without regard to food.

### Example 7

### Abuse Potential of Controlled-Release Formulations

It has long been theorized that the desirability of a drug of abuse is related to the speed with which it reaches maximum concentration in the plasma of the user. Basic science and clinical observation suggest that a shortened time to maximum plasma concentration (tₘₐₓ) and a heightened maximum plasma concentration (Cₘₐₓ) would increase the euphoric effects conferred by a drug. The abuse quotient (AQ) is a relatively new concept that attempts to predict the abuse potential of drugs. The AQ refers to the two PK parameters expressed as a ratio: AQ=Cₘₐₓ/tₘₐₓ. The abuse potential of a drug increases as the value of the AQ increases, either by heightening Cₘₐₓ or shortening tₘₐₓ.

Table 28 presents the AQs for various extended release formulations disclosed herein (see, e.g., selected examples from Chart Nos. 1 and 2) and several commercially available formulations.

TABLE 28
Abuse Quotient

| Formulation | Cₘₐₓ (ng/mL) | tₘₐₓ (hr) | AQ |
|---|---|---|---|
| 15/500 - Fast | 18.8 | 4.95 | 3.80 |
| 15/500 - Medium | 18.27 | 5.31 | 3.44 |
| 15/500 - Slow | 17.4 | 5.66 | 3.07 |
| 15/650 - 1 tablet | 17.68 | 3.90 | 4.53 |
| 15/650 - 2 tablets | 14.59* | 5.03 | 2.90 |
| 7.5/325 - 1 tablet | 16.82 | 3.71 | 4.53 |
| 7.5/325 - 2 tablets | 16.39 | 3.17 | 5.17 |
| Percocet | 22.43 | 2.16 | 10.38 |
| Oxycontin | 17.35 | 3.54 | 4.90 |
| OxyER | 19.61 | 4.11 | 4.77 |

*dose normalized to 15 mg

### Example 8

### Ethanol Release Testing at a 150 rpm Paddle Speed

To assess the potential for dose dumping, the in vitro dissolution of oxycodone and APAP from 7.5 mg OC/325 mg APAP tablets was tested in 0.1 N HCl containing 0%, 5%, 20%, or 40% v/v ethanol. The ER layer of the 7.5/325 tablets contained 5.625 mg of OC, 162.5 mg of APAP, and 45% (w/w) POLYOX® 1105, and the IR layer contained 1.875 mg of OC and 162.5 mg of APAP. (See selected example from Chart No. 1.) For each profile, twelve tablets were weighed, placed in a sinker, and dropped into an equilibrated USP Type II apparatus (paddles) that contained 900 mL of (helium sparged) 0.1 N HCl (containing either 0%, 5%, 20%, or 40% ethanol) heated to ˜37° C. The mixture was stirred at ˜150 rpm and the temperature was maintained at ˜37° C. for 120 minutes. The bath vessel was covered with a low evaporation vessel cover. Samples were removed at 15, 30, 45, 60, 75, 90, 105, and 120 minutes. Each sample was filtered through a 0.45 μm filter and analyzed by HPLC using standard procedures.

Tables 29, 30, 31, and 32 present the percent release of OC and APAP in the presence of 0%, 5%, 20%, and 40% ethanol, respectively. FIG. 17 presents dissolution profiles for OC and FIG. 18 presents dissolution profiles for APAP in the presence of 0%, 5%, 20%, and 40% ethanol. These data reveal that, for both OC and APAP, the dissolution in 5%, 20%, or 40% ethanol was either comparable or slower than the dissolution in 0% ethanol, indicating no dose dumping for this formulation.

TABLE 29

| | Percent Release in 0% Ethanol | | | | | | | |
|---|---|---|---|---|---|---|---|---|
| | OC | | | | APAP | | | |
| Time (Min) | Mean | RSD | Minimum | Maximum | Mean | RSD | Minimum | Maximum |
| 15 | 32.0 | 2.7 | 31.1 | 33.4 | 52.9 | 2.7 | 50.6 | 56.0 |
| 30 | 37.6 | 2.4 | 36.5 | 39.2 | 55.6 | 2.5 | 53.5 | 58.6 |
| 45 | 42.3 | 2.6 | 40.9 | 44.4 | 58.1 | 2.5 | 56.0 | 61.1 |
| 60 | 46.5 | 2.5 | 45.0 | 48.7 | 60.5 | 2.4 | 58.4 | 63.5 |
| 75 | 50.4 | 2.5 | 48.7 | 52.5 | 62.9 | 2.4 | 60.8 | 65.9 |
| 90 | 54.1 | 2.4 | 52.1 | 56.2 | 65.0 | 2.3 | 62.9 | 68.0 |
| 105 | 57.7 | 2.1 | 55.6 | 59.8 | 67.1 | 2.3 | 65.0 | 70.1 |
| 120 | 61.1 | 2.2 | 58.9 | 63.5 | 69.1 | 2.2 | 66.9 | 72.1 |

TABLE 30

| | Percent Release in 5% Ethanol | | | | | | | |
|---|---|---|---|---|---|---|---|---|
| | OC | | | | APAP | | | |
| Time (Min) | Mean | RSD | Minimum | Maximum | Mean | RSD | Minimum | Maximum |
| 15 | 31.2 | 2.4 | 30.2 | 32.4 | 52.1 | 1.5 | 50.5 | 53.5 |
| 30 | 36.9 | 3.2 | 35.1 | 39.0 | 54.9 | 1.6 | 53.4 | 56.4 |
| 45 | 41.5 | 3.3 | 39.1 | 44.0 | 57.2 | 1.5 | 55.7 | 58.7 |
| 60 | 45.5 | 3.5 | 43.4 | 48.2 | 59.4 | 1.5 | 57.9 | 60.9 |
| 75 | 49.4 | 2.6 | 47.9 | 52.5 | 61.5 | 1.5 | 60.0 | 63.0 |
| 90 | 52.9 | 3.5 | 50.7 | 56.1 | 63.4 | 1.5 | 61.9 | 65.0 |
| 105 | 56.2 | 1.8 | 54.0 | 57.8 | 65.4 | 1.5 | 63.8 | 66.9 |
| 120 | 59.3 | 2.8 | 56.7 | 61.7 | 67.2 | 1.5 | 65.6 | 68.7 |

TABLE 31
Percent Release in 20% Ethanol

| | OC | | | | APAP | | | |
|---|---|---|---|---|---|---|---|---|
| Time (Min) | Mean | RSD | Minimum | Maximum | Mean | RSD | Minimum | Maximum |
| 15 | 28.5 | 4.1 | 26.5 | 30.3 | 51.3 | 2.9 | 48.2 | 53.1 |
| 30 | 33.6 | 3.3 | 32.3 | 35.7 | 54.1 | 2.3 | 51.3 | 55.7 |
| 45 | 38.3 | 2.8 | 35.7 | 39.9 | 56.3 | 2.2 | 53.7 | 58.0 |
| 60 | 41.8 | 3.6 | 38.1 | 44.1 | 58.3 | 2.1 | 55.6 | 59.9 |
| 75 | 45.6 | 3.0 | 43.4 | 48.8 | 60.2 | 2.0 | 57.7 | 61.8 |
| 90 | 48.7 | 3.3 | 46.1 | 52.0 | 62.0 | 2.0 | 59.4 | 63.6 |
| 105 | 51.4 | 3.0 | 49.1 | 53.7 | 63.7 | 1.9 | 61.1 | 65.2 |
| 120 | 54.3 | 2.7 | 51.3 | 56.7 | 65.4 | 1.9 | 62.9 | 66.8 |

TABLE 32
Percent Release in 40% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 10.3 | 16.3 | 7.8 | 13.7 | 20.7 | 16.3 | 15.8 | 25.9 |
| 30 | 20.7 | 8.6 | 16.5 | 23.0 | 37.1 | 7.7 | 31.4 | 41.4 |
| 45 | 28.6 | 10.4 | 24.4 | 33.4 | 44.4 | 2.6 | 42.2 | 45.8 |
| 60 | 31.3 | 5.9 | 29.2 | 35.0 | 47.0 | 1.4 | 45.9 | 48.0 |
| 75 | 34.5 | 6.5 | 30.3 | 38.1 | 49.0 | 1.4 | 47.7 | 49.8 |
| 90 | 36.8 | 7.0 | 33.9 | 41.2 | 50.5 | 1.5 | 49.2 | 51.6 |
| 105 | 38.5 | 6.8 | 35.3 | 44.0 | 51.9 | 1.7 | 50.4 | 53.1 |
| 120 | 40.7 | 4.5 | 38.0 | 43.5 | 53.2 | 1.4 | 51.5 | 54.1 |

### Example 9

### Clinical Pharmacokinetic Analysis of an Extended Release Formulation of Oxycodone/Acetaminophen Administered Under Fed and Fasted Conditions

An open-label, randomized, three-period crossover study was conducted to evaluate the pharmacokinetics (PK), bioavailability, and safety of two tablets of a multi-layer extended-release formulation (each tablet comprising 7.5 mg oxycodone hydrochloride/325 mg acetaminophen), administered as a single dose in normal, healthy subjects under fed (high-fat or low-fat meal) and fasted conditions (i.e., 10 hr fast).

This single center, open-label, randomized, 3-period, 6-sequence crossover study in normal, healthy subjects was designed to evaluate the effect of a high-fat and low-fat meal on the PK, bioavailability, and safety of a multilayer ER tablet formulation of 7.5 mg OC/325 mg APAP (see selected example from Chart No. 1). The formulation was orally administered as 2 tablets (15 mg OC/650 mg APAP total dose) under 2 types of fed (high-fat and low-fat) and fasted conditions.

The study population included 48 normal, healthy male and female subjects aged 18 to 55 years. Eligibility criteria included: body mass index (BMI) between ≧19 and ≦30 kg/m², with a minimum weight of 130 lb; if female, non-pregnant and non-lactating; commitment to the use of 2 methods of birth control in subjects of child-bearing potential; ability to consume the entire standardized FDA high-fat meal or a low-fat meal in 30 minutes prior to dosing. Exclusion criteria included: history of drug abuse or treatment for abuse; positive urine test results for drugs of abuse, alcohol, and/or nicotine; history of smoking or use of nicotine-containing products within 6 months prior to study onset; history of drug allergy, hypersensitivity, or intolerance, including to oxycodone, APAP, or any opioid analgesic; history of any condition that may interfere with the absorption, distribution, metabolism, or excretion of study medication; or previous gastric bypass or gastric band surgery. Each crossover period required confinement of approximately 60 hours, and each period was separated by a minimum 7-day washout period. For subjects with ongoing adverse events, a follow-up visit occurred ≧7 days after the conclusion of the study; if a serious adverse event was ongoing at follow-up, the investigator continued to follow the subject for up to 28 days.

Following a 10 hour overnight fast, subjects randomized to Treatment A consumed an entire standardized FDA high-fat breakfast (approximately 1,000±100 calories and approximately 50% from fat); those receiving Treatment B consumed an entire low-fat breakfast (approximately 800±80 calories and approximately 25% to 30% from fat). Breakfasts were consumed within 30 minutes prior to Hour 0 study drug administration. Subjects who could not consume the entire breakfast in the allotted time were dropped from the study. Subjects randomized to Treatment C were administered study drug under fasted conditions following an overnight fast of at least 10 hours. No food was allowed for the first 4 hours postdose. Blood samples were collected pre-dose (up to 60 minutes prior to dose), and at 15 min, 30 min, 45 min and 1, 2, 3, 4, 5, 6, 7, 8, 9, 10, 12, 16, 18, 20, 24, 36 and 48 hours post-dose, and the resulting plasma samples were analyzed for OC and APAP using a validated liquid chromatography-tandem mass spectrometry assay with a linear range of 0.100 to 100 ng/mL for OC and 100 to 50,000 ng/mL for APAP. Pharmacokinetic parameters, as detailed above in Example 2, were determined.

PK parameters of oxycodone and APAP were estimated using plasma concentration versus time data by standard non-compartmental methods. The Wilcoxon signed-rank test was utilized to compare untransformed time to maximum observed plasma concentration (Tₘₐₓ) and time prior to the first measurable concentration (t_{lag}); P≦0.05 was considered a significant difference between treatments. Analysis of variance was performed to compare data from Treatments A, B, and C using the natural log transformed PK parameters area under the plasma drug concentration versus time curve from time zero to the last quantifiable data point (AUC₀₋ₜ), AUC from time zero to infinity (AUC_{0-inf}), and maximum observed plasma concentration (Cₘₐₓ). A 90% confidence interval (CI) of the geometric least-squares (LS) means ratio contained within 80% to 125% concluded no difference between treatments.

A total of 48 subjects enrolled and received study medication (safety population). Of these subjects, 31 (65%) completed all 3 periods of the study (completers, PK population). Of the 17 subjects (35%) who discontinued early from the study, 14 withdrew due to vomiting (withdrawn per protocol) and 3 met other withdrawal criteria. Baseline demographics were comparable between the enrolled subjects and completers, except that there was a higher percentage of male subjects among the completers (68% vs 52%).

Tables 33 and 34 presents PK parameters for OC under the three treatment conditions, and FIG. 19 presents plasma OC concentration-time profiles for the treatments. Mean plasma concentration profiles of OC revealed that OC was rapidly absorbed under both fed (high and low fat meal) and fasted conditions. There was a slight lag (median 0.25 hours) when the formulation was administered after a meal (high and low fat). The median of the time of observed maximum plasma concentrations (Tₘₐₓ) were 4 hours and 3 hours after administration under low fat and fasted conditions, respectively. Median Tₘₐₓ for OC under high fat conditions was statistically significantly delayed, as compared to fasted conditions (5 hr vs. 3 hr; P<0.05). Average maximum plasma OC concentrations (Cₘₐₓ) were 19.94 ng/mL after a low fat breakfast, 17.90 ng/mL after a high fat breakfast, and 15.91 ng/mL under fasted conditions.

TABLE 33
Pharmacokinetic Parameters for Oxycodone Following Administration of
ER OC/APAP (2 Tablets of 7.5/325) Under Fed and Fasted Conditions (n = 31)

| | Treatment A | | Treatment B | | Treatment C | |
|---|---|---|---|---|---|---|
| | High Fat | | Low Fat | | Fasted | |
| | Mean (SD) | | Mean (SD) | | Mean (SD) | |
| Parameter | (N = 31) | | (N = 31) | | (N = 31) | |
| AUC₀₋ₜ (ng · h/mL) | 219.41 | (54.07) | 219.49 | (57.29) | 190.70 | (50.03) |
| AUC_{0-inf} (ng · h/mL) | 221.00 | (54.14) | 221.38 | (56.95) | 192.63 | (49.69) |
| Cₘₐₓ (ng/mL) | 17.90 | (4.25) | 19.94 | (4.66) | 15.91 | (3.43) |
| Tₘₐₓ (h)^{a} | 5.00 | (1.00-12.00) | 4.00 | (1.00-5.00) | 3.00 | (0.75-8.00) |
| Kₑₗ (1/h) | 0.1682 | (0.0298) | 0.1693 | (0.0321) | 0.1502 | (0.0269) |
| t_{lag} (h)^{a} | 0.25 | (0.00-1.00) | 0.25 | (0.00-0.75) | 0.00 | (0.00-0.25) |
| t_{1/2} (h) | 4.26 | (0.83) | 4.26 | (0.91) | 4.76 | (0.87) |

^{a}Median (minimum-maximum).

A comparison of Cₘₐₓ showed that OC concentrations were 12% and 25% higher when the formulation was given under high fat (Treatment A) and low fat (Treatment B) conditions, compared to fasted conditions (Treatment C; see Table 33). The Cₘₐₓ for Treatment A was bioequivalent to both Treatments B (84%-96%) and C (105%-120%) as the 90% CIs for the geometric ratios were contained within 80% to 125% (see Table 34). The Cₘₐₓ observed for Treatment B was not bioequivalent to Treatment C (117%-134%). AUCs were approximately 15% higher when the formulation was administered under fed conditions (high and low fat), as compared to fasted conditions (Table 33). AUC for both Treatments A and B (high fat and low fat) were bioequivalent to Treatment C (fasted; 111%-121% and 111%-120% for AUC0-t and 111%-120% and 110%-120% for AUC0-inf) (Table 34). The apparent plasma terminal elimination half-life (t½) for OC was similar when the formulation was administered under fed (4 hours) and fasted conditions (5 hours).

TABLE 34
Oxycodone Geometric LS Mean Ratios (AUC and
Cₘₐₓ) and 90% Cls for the Comparison of
ER OC/APAP Under Fed and Fasted Conditions (n = 31)

| | Treatment A/C | Treatment B/C | Treatment A/B |
|---|---|---|---|
| | Fed (High | Fed (Low | Fed (High Fat)/ |
| Parameter | Fat)/Fasted | Fat)/Fasted | Fed (Low Fat) |
| AUC_{0-inf} | 115.41 | 115.09 | 100.28 |
| (ng · h/mL)^{a} | (110.63, 120.41) | (110.38, 120.01) | (96.18, 104.55) |
| AUC₀₋ₜ | 115.85 | 115.30 | 100.47 |
| (ng · h/mL)^{a} | (111.00, 120.90) | (110.54, 120.27) | (96.34, 104.79) |
| Cₘₐₓ (ng/mL)^{a} | 112.11 | 125.16 | 89.57 |
| | (104.61, 120.16) | (116.88, 134.03) | (83.67, 95.90) |

^{a}N = 31.

PK parameters for APAP are presented in Tables 35 and 36 and the plasma APAP concentration-time profiles are presented in FIG. 20. APAP was rapidly absorbed following administration under fed (high and low fat meals) and fasted conditions. There was a slight lag when the formulation was administered after a low fat breakfast (median lag time [t_{lag}] 0.25 hours). There was no lag in the absorption of APAP when administered following a high fat breakfast or after fasting. The time to Cₘₐₓ was significantly (P<0.05) longer when administered after a meal (high and low fat; median Tₘₐₓ=2 hours) than when administered under fasted conditions (median Tₘₐₓ=0.5 hour). Average Cₘₐₓ values for APAP were lower after a high (3,775 ng/mL) and low fat (3,863 ng/mL) meal than when administered under fasted conditions (5,175 ng/mL). Geometric mean ratios for Cₘₐₓ following Treatments A and B were 24% to 23% lower than for Treatment C (Table 36). The 90% CIs for Cₘₐₓ following Treatment A (70%-82%) and Treatment B (72%-83%) with reference to fasted state were outside the bioequivalent range of 80%-125%. The AUCs for APAP were almost identical when the formulation was administered under high fat, low fat, or fasting conditions. (Comparison of geometric mean ratios of AUC₀₋ₜ and AUC_{0 inf} for Treatments A (90% CI 97%-103% and 96%-102%) and B (90% CI 96%-101% and 94% to 100%) with those for Treatment C showed that treatments were bioequivalent. The t_{1/2} for APAP after the formulation was administered after a high or low fat meal (5 hours) was slightly shorter than when administered under fasted conditions (7 hours).

TABLE 35
Pharmacokinetic Parameters for APAP Following Administration of
ER OC/APAP (2 Tablets of 7.5/325) Under Fed and Fasted Conditions (n = 31)

| | Treatment A | | Treatment B | | Treatment C | |
|---|---|---|---|---|---|---|
| | Fed (High Fat) | | Fed (Low Fat) | | Fasted | |
| | Mean (SD) | | Mean (SD) | | Mean (SD) | |
| Parameter | (N = 31) | | (N = 31) | | (N = 31) | |
| AUC₀₋ₜ (ng · h/mL) | 29617.96 | (7765.99) | 29346.82 | (7869.75) | 29763.19 | (7592.89) |
| AUC_{0-inf} (ng · h/mL) | 31457.06 | (7973.16)^{a} | 30550.48 | (8051.47) | 31807.70 | (7923.30)^{a} |
| Cₘₐₓ (ng/mL) | 3774.52 | (949.84) | 3862.90 | (978.08) | 5175.48 | (1731.31) |
| Tₘₐₓ (h)^{b} | 2.00 | (0.50-5.00) | 2.00 | (0.50-5.00) | 0.53 | (0.23-5.00) |
| Kₑₗ (1/h) | 0.1564 | (0.0363)^{a} | 0.1593 | (0.0408) | 0.1146 | (0.0360)^{a} |
| t_{lag} (h)^{b} | 0.00 | (0.00-1.00) | 0.25 | (0.00-0.50) | 0.00 | (0.00-0.25) |
| t_{1/2} (h) | 4.66 | (1.08)^{a} | 4.71 | (1.60) | 6.63 | (1.99)^{a} |

^{a}N = 29
^{b}Median (minimum-maximum).

TABLE 36
Acetaminophen Geometric LS Mean Ratios (AUC and
Cₘₐₓ) and 90% Cls for the Comparison of
ER OC/APAP Under Fed and Fasted Conditions

| | | | |
|---|---|---|---|
| | Treatment A/C | Treatment B/C | Treatment A/B |
| | Fed (High | Fed (Low | Fed (High Fat)/ |
| Parameter | Fat)/Fasted | Fat)/Fasted | Fed (Low Fat) |

| | | | |
|---|---|---|---|
| AUC_{0-inf} | 98.60 | 96.56 | 102.12 |
| (ng · h/mL)^{a} | (95.75, 101.54) | (93.80, 99.39) | (99.20, 105.11) |
| AUC₀₋ₜ | 99.88 | 98.79 | 101.10 |
| (ng · h/mL)^{b} | (97.31, 102.52) | (96.27, 101.37) | (98.54, 103.74) |
| Cₘₐₓ (ng/mL)^{b} | 76.00 | 77.18 | 98.48 |
| | (70.49, 81.94) | (71.65, 83.13) | (91.45, 106.05) |

^{a}N = 27
^{b}N = 31

Reports of adverse events were also collected and monitored throughout the study. Table 36A presents a summary of the most frequently occurring (>10% overall) treatment-emergent adverse events. 33 participants (68.8%) reported ≧1 TEAE. The most frequently reported TEAEs were nausea, vomiting, and dizziness, and there were no notable differences between treatment groups. All TEAEs were determined by the investigator to be either mild or moderate in severity; all resolved after discontinuation of treatment.

TABLE 36A
Summary of Most Frequently Occurring (>10% Overall)
Treatment Emergent Adverse Events

| | High-Fat Meal | Low-Fat Meal | Fasted | Overall |
|---|---|---|---|---|
| TEAE, n (%) | (n = 37) | (n = 38) | (n = 39) | (N = 48) |
| Any TEAE | 16 (43.2) | 17 (44.7) | 15 (38.5) | 33 (68.8) |
| Nausea | 10 (27.0) | 8 (21.1) | 8 (20.5) | 21 (43.8) |
| Vomiting | 5 (13.5) | 5 (13.2) | 5 (12.8) | 15 (31.3) |
| Dizziness | 5 (13.5) | 6 (15.8) | 2 (5.1) | 10 (20.8) |
| Somnolence | 1 (2.7) | 2 (5.3) | 3 (7.7) | 6 (12.5) |
| Euphoric mood | 2 (5.4) | 1 (2.6) | 4 (10.3) | 6 (12.5) |
| Pruritus | 3 (8.1) | 4 (10.5) | 0 | 5 (10.4) |

The results of this study demonstrate that total oxycodone and APAP exposures (AUC) were not significantly affected by food. Minimal changes in Cₘₐₓ for oxycodone and APAP were noted in the presence of food, but these changes were comparable to food effects on peak exposure for other oxycodone and APAP products. Food marginally delayed the Tₘₐₓ of oxycodone and APAP. ER OC/APAP was generally well tolerated. There was no indication that safety or tolerability were affected by food. These findings demonstrate that ER OC/APAP can be administered with or without food.

### Example 10

### Clinical Pharmacokinetic Analysis of an Extended Release Formulation of 7.5 mg Oxycodone/325 mg Acetaminophen—Single Dose

A single-center, open-label, randomized, phase 1, 3-period crossover study was performed to evaluate the single dose pharmacokinetic (PK) parameters, bioavailability, and safety of an extended-release formulation containing 7.5 mg OC/325 mg APAP (see selected example from Chart No. 1) in healthy subjects under fasted conditions. The PK and bioavailability of the extended-release formulation administered as 1 or 2 tablets were compared to the commercially-available immediate release tablet (immediate release 7.5 mg OC/325 mg APAP) administered as 1 or 2 tablets every 6 hours for 2 doses.

This study was conducted in 48 male and female subjects, with equal gender distribution. Normal healthy male or non-lactating, non-pregnant female subjects aged 18 to 55 years with body mass index of ≧9 to ≦30 kg/m² and a minimum weight of 130 lb were eligible for participation. Exclusion criteria included smoking or use of nicotine-containing products in the previous 6 months; history of drug or alcohol use or positive urine test for drugs of abuse; use of prescription or over-the-counter drugs within 14 days of study check-in; history of drug allergy, hypersensitivity, or intolerance of opioid drug products (including oxycodone) or APAP; history of any condition that may interfere with the absorption, distribution, metabolism, or excretion of study drug; or previous gastric bypass or gastric band surgery. A total of 48 adults were enrolled; 33 completed 3 treatment periods (primary completers), and 27 completed all 4 treatment periods (secondary completers).

Completers of the first 3 periods of this study entered a fourth treatment period that served as a second phase of the study. The 4 treatment periods (A, B, C, and D) each utilized a unique dosing scheme. Subjects were randomized to receive the following treatments in a 3-way crossover design under fasted conditions:
Treatment A: 1-tablet dosing of ER OC/APAP (7.5 mg OC/325 mg APAP) (see selected example from Chart No. 1) taken once
Treatment B: 2-tablet dosing of ER OC/APAP (total, 15 mg OC/650 mg APAP) (see selected example from Chart No. 1) taken once
Treatment C: 1-tablet dosing of commercially-available IR OC/APAP (7.5 mg OC/325 mg APAP) taken every 6 hours for 2 doses (total, 15 mg OC/650 mg APAP)

Subjects who completed periods 1, 2, and 3 returned for period 4 and received Treatment D (2-tablet dosing of IR OC/APAP every 6 hours for 2 doses [total, 30 mg OC/1300 mg APAP] under fasted conditions). The study included a screening visit and 4 confinement periods of approximately 60 hours each, with a minimum of 7 days between the start of each period, and a minimum 7-day follow-up period.

Blood was collected predose and 15, 30, and 45 minutes and 1, 2, 3, 4, 5, 6, 6.5, 7, 8, 9, 10, 12, 16, 18, 20, 24, 36, and 48 hours after dosing. PK parameters (AUC₀₋ₜ, AUC_{0-inf}, Cₘₐₓ, Tₘₐₓ, t_{lag}, Kₑₗ, and t_{1/2}) for oxycodone and APAP were calculated by noncompartmental methods. Analysis of variance was performed to compare treatment conditions A, B, and C using the dose-normalized (plasma concentration divided by dose) natural log-transformed PK parameters (AUC_{0-inf}, AUC₀₋ₜ, and Cₘₐₓ), and linear mixed models analysis compared the same PK parameters for treatments C and D. Dose normalization was utilized to compare concentration across different dosage strengths. A 90% confidence interval of the geometric least-squares means ratio fully contained within 80% to 125% for AUC_{0-inf}, AUC₀₋ₜ, and Cₘₐₓ indicated no difference between treatments. Dose-normalized AUC and Cₘₐₓ for oxycodone and APAP were used for comparisons.

Pharmacokinetic parameter estimates for OC are presented in Table 37A, and OC plasma concentration-time profiles are presented in FIG. 21. There was no lag in absorption of OC for the 1 and 2 tablet dosing configurations of the extended release formulation and the commercially-available immediate release tablet under fasted conditions. Plasma concentrations of OC rose rapidly after administration of the extended release formulation in a similar fashion to the commercially-available immediate release tablet, and peak plasma levels of OC were observed (Tₘₐₓ) at 4 and 3 hours for the 1 or 2 tablet dosing configuration of the extended release formulation compared with 7 hours after the initial dose of 1 tablet of the commercially-available immediate release tablet (1 hour after the second dose) and 0.75 hours after the initial dose of 2 tablets of the commercially-available immediate release tablet. Mean plasma concentrations of OC from the extended release formulation were detectable through 36 hours in most subjects following all treatments and t_{1/2} was about 4 to 5 hours across all treatments. The extent of exposure (AUC₀₋ₜ and AUC_{0-inf}) for the 2 tablet dosing configuration of the extended release formulation increased proportionally with dose compared with the 1-tablet dosing configuration of the extended release formulation. Dose-normalized oxycodone AUC₀₋ₜ and AUC_{0-inf} were comparable across treatments A, B, and C, indicating similar bioavailability of oxycodone for ER OC/APAP (1 or 2 tablets once) and IR OC/APAP (1 tablet twice). The dose-normalized oxycodone Cₘₐₓ was comparable for the 1- and 2-tablet doses of ER OC/APAP, indicating dose proportionality of oxycodone between 1 and 2 tablets of ER OC/APAP.

TABLE 37A
Oxycodone Pharmacokinetic Estimates (7.5/325)

| | | | | | Treatment C | | Treatment D | |
|---|---|---|---|---|---|---|---|---|
| | Treatment A | | Treatment B | | IR OC/APAP | | IR OC/APAP | |
| | ER OC/APAP | | ER OC/APAP | | (1 tablet | | (2 tablets | |
| | (1 tablet; | | (2 tablets; | | twice; | | twice; | |
| | 7.5/325 mg) | | 15/650 mg) | | 15/650 mg^{c}) | | 30/1300 mg^{c}) | |
| Parameter | (n = 33) | | (n = 33) | | (n = 33) | | (n = 27^{d}) | |
| AUC₀₋ₜ | 87.43 | (24.59) | 185.98 | (47.64) | 191.15 | (53.43) | 401.23 | (110.56) |
| (ng · h/mL) | | | | | | | | |
| AUC_{0-inf} | 89.85 | (24.73)^{b} | 187.71 | (47.58) | 193.10 | (53.22) | 403.04 | (110.45) |
| (ng · h/mL) | | | | | | | | |
| Cₘₐₓ (ng/mL) | 8.41 | (2.06) | 16.39 | (4.31) | 20.82 | (5.98) | 41.24 | (12.12) |
| Tₘₐₓ (h)^{a} | 4.00 | (0.75-5.92) | 3.00 | (0.75-6.50) | 7.38 | (0.50-10.00) | 0.75 | (0.50-12.00) |
| t_{lag} (h)^{a} | 0.00 | (0.00-0.50) | 0.00 | (0.00-0.52) | 0.00 | (0.00-0.25) | 0.00 | (0.00-0.25) |
| t_{1/2} (h) | 4.50 | (0.78)^{b} | 4.87 | (0.93) | 4.08 | (0.89) | 4.34 | (1.02) |
| Kₑₗ (h⁻¹) | 0.1590 | (0.0307)^{b} | 0.1473 | (0.0274) | 0.1770 | (0.0352) | 0.1688 | (0.0415) |

^{a}Median (minimum-maximum).
^{b}N = 32

No dose-dumping was observed in any subject receiving the ER formulation. The interindividual variability (CV %) for Cₘₐₓ of OC after administration of 1 or 2 tablets of the ER formulation was comparable to 1 tablet of the commercially-available immediate release tablet and less than 29% for all 3 treatments. Similarly the interindividual variability (CV %) for AUC of OC was 28% or less for 1 and 2 tablets of the ER formulation and 1 tablet of the commercially-available immediate release tablet.

Table 37B presents APAP PK parameter estimates and FIG. 22 presents APAP plasma concentration-time profiles. The appearance of plasma concentrations of APAP for all dose configurations of the extended release formulation and the commercially-available immediate release tablet showed no lag. Plasma concentrations of APAP rose rapidly after administration of the extended release formulation, similar to that observed with the commercially-available immediate release tablet. APAP Cₘₐₓ was comparable for 2 tablets of ER OC/APAP and 1 tablet every 6 hours of IR OC/APAP (15 mg OC/650 mg APAP total for both). Peak plasma levels of APAP following administration of the 1 tablet and 2 tablet dosing configurations of the extended release formulation were observed (median Tₘₐₓ) at 0.75 hours after dosing compared with 0.5 hours after the first dose of the commercially-available immediate release tablet (1 and 2 tablets). Mean plasma concentrations of APAP were detectable through 36 hours following all treatments and the mean t₁₁₂ was approximately 4 to 7 hours across treatment groups. The extent of exposure (AUC) to APAP following dosing with 1 and 2 tablets of the extended release formulation increased proportionally with dose. Dose normalized AUC₀₋ₜ, AUC_{0-inf}, and Cₘₐₓ for APAP were comparable across all treatment groups, indicating the bioavailability of APAP was similar with ER OC/APAP (1 or 2 tablets once) to that with IR OC/APAP (1 tablet twice), and also indicating dose proportionality between 1 and 2 tablets for APAP with ER OC/APAP.

TABLE 37B
APAP Pharmacokinetic Estimates (7.5/325)

| | | | | | Treatment C | | Treatment D | |
|---|---|---|---|---|---|---|---|---|
| | Treatment A | | Treatment B | | IR OC/APAP | | IR OC/APAP | |
| | ER OC/APAP | | ER OC/APAP | | (1 tablet | | (2 tablets | |
| | (1 tablet; | | (2 tablets; | | twice; | | twice; | |
| | 7.5/325 mg) | | 15/650 mg) | | 15/650 mg^{c}) | | 30/1300 mg^{c}) | |
| Parameter | (n = 33) | | (n = 33) | | (n = 33) | | (n = 27^{d}) | |
| AUC₀₋ₜ | 15871 | (4841) | 32665 | (10894) | 33040 | (9589) | 69837 | (22945) |
| (ng · h/mL) | | | | | | | | |
| AUC_{0-inf} | 16995 | (5073) | 34836 | (11067)^{b} | 34236 | (10126)^{b} | 71949 | (24234)^{c} |
| (ng · h/mL) | | | | | | | | |
| Cₘₐₓ (ng/mL) | 2632 | (918) | 5230 | (2086) | 4878 | (1545) | 10741 | (4123) |
| Tₘₐₓ (h)^{a} | 0.75 | (0.25-2.02) | 0.75 | (0.25-4.00) | 0.50 | (0.25-9.00) | 0.50 | (0.25-12.00) |
| t_{lag} (h)^{a} | 0.00 | (0.00-0.50) | 0.00 | (0.00-0.25) | 0.00 | (0.00-0.00) | 0.00 | (0.00-0.00) |
| t_{1/2} (h) | 5.33 | (1.53) | 6.88 | (2.15)^{b} | 4.41 | (1.16)^{b} | 5.76 | (1.47)^{c} |
| Kₑₗ (h⁻¹) | 0.1421 | (0.0479) | 0.1103 | (0.0337)^{b} | 0.1669 | (0.0411)^{b} | 0.1291 | (0.0368)^{c} |

^{a}Median (minimum-maximum).
^{b}N = 32
^{c}N = 25

No dose-dumping was observed in any subject receiving the ER formulation. The interindividual variability (CV %) for Cₘₐₓ of APAP was slightly more after administration of 1 and 2 tablets of the ER formulation (35% and 40%, respectively) than for 1 tablet of the commercially-available immediate release tablet (32%). The interindividual variability (CV %) for AUC of APAP was less than 33% for all 3 treatments.

Both OC and APAP were rapidly absorbed under all conditions with no lag in plasma concentrations. Both OC and APAP levels were sufficiently high within 1 hour after administration of the extended release formulation. Peak exposure to OC was 18% to 21% lower for the ER formulation than for the commercially-available immediate release tablet (1 tablet Q6 h). OC levels were sustained over the proposed 12 h dosing interval. By 12 hours after dosing with the extended release formulation, APAP plasma levels were less than 20% of Cₘₐₓ. Total exposure to both OC and APAP from the extended release formulation was equivalent to that of 1 tablet of the commercially-available immediate release tablet.

Adverse events were also monitored throughout the study. Table 38 presents the most frequently occurring treatment-emergent adverse events (TEAEs). Overall, 41 subjects (85%) reported ≧1 TEAE; 44% were considered by the investigator to be mild in intensity and 42% were considered to be moderate in intensity. There were no serious adverse events. The most common TEAEs were nausea, vomiting, somnolence, pruritus, and headache, which are consistent with those associated with opioid therapy. A total of 19 subjects experienced vomiting and were discontinued early from the study, as specified in the protocol. TEAEs were higher after 2-tablet dosing with IR OC/APAP (75.8%) than after 1-tablet or 2-tablet dosing with ER OC/APAP (25.6% and 51.2%, respectively) or 1-tablet dosing with IR OC/APAP (56.4%). Overall, TEAEs for ER OC/APAP were similar to those for IR OC/APAP. Most individual hematology and serum chemistry values were within the normal range. All changes that were noted as abnormal were considered by the investigator to not be clinically significant, except for elevated bilirubin in 1 subject (2%), which was considered by the investigator to be mild and possibly related to study medication.

TABLE 38
Most Frequently Occurring Treatment-Emergent Adverse Events

| | | | IR OC/APAP | IR OC/APAP | |
|---|---|---|---|---|---|
| | ER OC/APAP | ER OC/APAP | (1 tablet | (2 tablets | |
| | (1 tablet; | (2 tablets; | twice; | twice; | |
| | 7.5/325 mg) | 15/650 mg) | 15/650 mg^{a}) | 30/1300 mg^{a}) | Overall |
| TEAE, n (%) | (n = 39) | (n = 41) | (n = 39) | (n = 33^{b}) | (N = 48) |
| Any TEAE | 10 (25.6) | 21 (51.2) | 22 (56.4) | 25 (75.8) | 41 (85.4) |
| Nausea | 4 (10.3) | 12 (29.3) | 12 (30.8) | 17 (51.5) | 33 (68.8) |
| Vomiting | 2 (5.1) | 7 (17.1) | 4 (10.3) | 6 (18.2) | 19 (39.6) |
| Somnolence | 2 (5.1) | 5 (12.2) | 5 (12.8) | 9 (27.3) | 17 (35.4) |
| Pruritus | 0 | 3 (7.3) 5 | 5 (12.8) | 12 (36.4) | 16 (33.3) |
| Headache | 2 (5.1) | 8 (19.5) | 5 (12.8) | 4 (12.1) | 14 (29.2) |
| Dizziness | 1 (2.6) | 4 (9.8) | 4 (10.3) | 7 (21.2) | 13 (27.1) |

^{a}IR OC/APAP was dosed as 1 tablet every 6 hours and counted as a single dose
^{b}Completed all other treatment conditions

This study indicates that ER OC/APAP demonstrates a biphasic delivery of oxycodone, with a rapid rise after dosing as seen with IR OC/APAP, followed by controlled release that peaked at 3 to 4 hours post-dose and extended over 12 hours. APAP concentrations rose rapidly and then tapered off at 7 to 12 hours post-dose. Lower APAP concentrations at the end of the dosing period may reduce APAP accumulation and thus lessen the possibility of the potential hepatotoxic effects of APAP. Bioavailability of OC and APAP throughout the dosing interval were comparable between ER OC/APAP (1 or 2 tablets) and IR OC/APAP (1 tablet twice) under fasted conditions. Dose proportionality with respect to AUC and Cₘₐₓ was observed between the 1-tablet and 2-tablet doses of ER OC/APAP. ER OC/APAP was generally well tolerated, with the most frequently reported TEAEs being nausea, headache, vomiting, and somnolence. These findings demonstrate that ER OC/APAP yields plasma concentrations comparable to those of IR OC/APAP with 12-hour dosing, with a tolerability profile consistent with opioid analgesics.

To further analyze the absorption of OC and APAP from the ER formulation, the plasma concentrations of OC and APAP following administration of 1 tablet of the ER formulation, 2 tablets of the ER formulation, and the commercially-available immediate release tablet were deconvolved using WinNonlin 5.2 (Pharsight). Deconvolution evaluates in vivo drug release and delivery based on data for a known drug input. Depending upon the type of reference input information available, the drug transport evaluated will be either a simple in vivo drug release (e.g., gastro-intestinal release) or a composite form, typically consisting of an in vivo release followed by a drug delivery to the general systemic circulation. It can estimate the cumulative amount and fraction absorbed over time for the subjects, given PK profile data and dose. For a pure immediate release (IR) or an extended release (ER) formulation the cumulative absorption plot shows a monoexponential curve whereas for a bilayer formulation (IR+ER) a biexponential (rapid phase followed by slower phase) absorption curve will be observed. FIG. 23 and FIG. 24 present the deconvolution plots for OC and APAP, respectively. For each, there is an early rapid phase of absorption that is followed by a later slower phase of absorption from the ER formulation.

### Example 11

### Clinical Pharmacokinetic Analysis of an Extended Release Formulation of 7.5 mg Oxycodone/325 mg Acetaminophen—Multiple Doses

An open-label, randomized, 3-period crossover study was performed to evaluate the steady-state PK, bioavailability, and safety of the extended release formulation containing 7.5 mg OC/325 mg APAP in healthy subjects (see selected example from Chart No. 1). The PK and bioavailability of the ER formulation administered as 1 or 2 tablets every 12 hours for 4.5 days (9 doses) was compared to the commercially-available immediate release tablet (immediate release 7.5 mg OC/325 mg APAP) administered as 1 tablet every 6 hours for 4.5 days (18 doses) under fasted conditions (10 hours for the first dose on Days 1 and 5; at least 1 hour for all other doses). This study was conducted in 48 male and female subjects, with equal gender distribution.

The PK behavior of OC on Study Day 1 (see Table 39) was similar to that observed in the single dose study (see Example 10). There was no lag (median t_{lag} 0 hours) in the absorption of OC following administration of the ER formulation (1 or 2 tablets) and the commercially-available immediate release tablet, and no dose-dumping was observed for any subject. Peak plasma levels were observed at 3 hours after administration of 1 and 2 tablets of the ER formulation and at 1 hour after the second dose of the commercially-available immediate release tablet (FIG. 25). On Day 1, interindividual variability (% CV) in the Cₘₐₓ for OC was slightly higher for 1 tablet (29%) than for 2 tablets (23%) of the ER formulation or the commercially-available immediate release tablet (up to 22%). The variability in the AUC₀₋₁₂ₕᵣ for OC was comparable between all 3 treatments (21% to 23%). Minimum (trough) plasma concentrations (Cmin) of OC achieved steady-state levels by Day 4 for 1 tablet of the ER formulation and the commercially-available immediate release tablet and by Day 3 for 2 tablets of the ER formulation. Trough levels of OC on Days 2 through 5 for 2 tablets of the ER formulation were comparable to those observed for the commercially-available immediate release tablet.

TABLE 39
Oxycodone Pharmacokinetic Estimates - Day 1

| | | | | | Treatment C | |
|---|---|---|---|---|---|---|
| | | | | | Commercially- | |
| | | | | | available | |
| | Treatment A | | Treatment B | | immediate | |
| | ER Formulation | | ER Formulation | | release tablet | |
| | (1 Tablet Q12h) | | (2 Tablets Q12h) | | (1 Tablet Q6h) | |
| | Mean (SD) | | Mean (SD) | | Mean (SD) | |
| Parameter | (N = 33) | | (N = 33) | | (N = 33) | |
| AUC_{0-12 h} | 66.93 | (15.14) | 135.89 | (30.81) | 141.73 | (29.78) |
| (ng · h/mL) | | | | | | |
| Cₘₐₓ (ng/mL) | 8.34 | (2.37) | 17.05 | (3.97) | 21.93 | (4.80) |
| Tₘₐₓ (h)^{a} | 3.00 | (0.75-7.00) | 3.00 | (0.50-5.92) | 7.00 | (0.50-8.00) |
| t_{lag} (h)^{a} | 0.00 | (0.00-0.50) | 0.00 | (0.00-0.32) | 0.00 | (0.00-0.25) |

^{a}Median (minimum-maximum).

On Day 5 (see Table 40), steady state was achieved and the median Tₘₐₓ^{ss} was observed at 2 hours following 1 tablet or 2 tablets of the ER formulation and at 30 min following the second daily dose of the commercially-available immediate release tablet. Maximum observed plasma concentrations at steady-state (Cₘₐₓ^{ss}) for OC for the 1 and 2 tablet dosing configurations of the ER formulation were not equivalent to the commercially-available immediate release tablet. On Day 5, interindividual variability (% CV) in Cₘₐₓ^{ss} and AUC₀₋₁₂ₕ^{ss} for OC was comparable between all 3 treatments (up to 29%). The degree of fluctuation (DFL) in and the swing of plasma concentrations for the ER formulation over the last 12 hour dosing interval on Day 5 were 15% to 22% less than that observed for the commercially-available immediate release tablet.

TABLE 40
Oxycodone Pharmacokinetic Estimates - Day 5

| | | | | | Treatment C | |
|---|---|---|---|---|---|---|
| | | | | | Commercially- | |
| | | | | | available | |
| | Treatment A | | Treatment B | | immediate | |
| | ER Formulation | | ER Formulation | | release tablet | |
| | (1 Tablet Q12h) | | (2 Tablets Q12h) | | (1 Tablet Q6h) | |
| | Mean (SD) | | Mean (SD) | | Mean (SD) | |
| Parameter | (N = 33) | | (N = 33) | | (N = 33) | |
| AUC_{0-12 h}^{ss} | 102.36 | (29.30) | 208.59 | (59.28) | 208.93 | (57.30) |
| (ng · h/mL) | | | | | | |
| Cₐᵥ^{ss} (ng/mL) | 8.53 | (2.44) | 17.38 | (4.94) | 17.41 | (4.78) |
| Cₘₐₓ^{ss} (ng/mL) | 12.67 | (3.48) | 25.67 | (7.49) | 30.50 | (8.91) |
| Cₘᵢₙ^{ss} (ng/mL) | 4.06 | (1.40) | 8.98 | (3.52) | 8.78 | (3.17) |
| DFL (%) | 101.72 | (14.14) | 97.17 | (18.80) | 126.83 | (27.93) |
| Swing | 2.23 | (0.64) | 2.03 | (0.70) | 2.67 | (0.92) |
| Tₘₐₓ^{ss} (h)^{a} | 2.00 | (0.50-10.00) | 2.00 | (0.50-7.00) | 6.50 | (0.50-8.02) |
| t_{1/2} (h)^{c} | 5.46 | (1.24) | 6.11 | (1.46) | 5.47 | (1.70)^{b} |
| Kₑₗ (1/h)^{c} | 0.1326 | (0.0269) | 0.1199 | (0.0291) | 0.1387 | (0.0418)^{b} |

^{a}Median (minimum-maximum).
^{b}N = 32
^{c}Days 5 to 7.

The PK behavior of APAP on Study Day 1 (see Table 41) was similar to that observed in the single dose study (see Example 10). Acetaminophen was rapidly absorbed following a single dose of 1 or 2 tablets of the ER formulation and in a similar fashion to the commercially-available immediate release tablet. (FIG. 26). There was no lag in plasma concentrations following any of the 3 dosing regimens (median t_{lag} 0 hours), and no dose-dumping was observed for any subject. Peak APAP plasma concentrations were observed 30 to 45 minutes after administration of 1 or 2 tablets of the ER formulation and at 30 minutes after the first dose of the commercially-available immediate release tablet on Day 1. The Cₘₐₓ for APAP occurred following the first 325 mg dose of the commercially-available immediate release tablet, rather than after the second dose. Dose proportionality for Cₘₐₓ and AUC₀₋₁₂ₕ was observed over the range of 325 mg to 650 mg APAP after a single administration of 1 or 2 tablets of the ER formulation. The Cₘᵢₙ of APAP achieved steady-state levels by Day 4 for 1 tablet and by Day 2 for 2 tablets of the ER formulation and for the commercially-available immediate release tablet. Trough levels of APAP on Days 2 through 5 for 2 tablets of the ER formulation were comparable to those observed for the commercially-available immediate release tablet. On Day 1, interindividual variability (% CV) in Cₘₐₓ and AUC₀₋₁₂ₕᵣ for APAP was comparable between all 3 treatments (31% or less).

TABLE 41
APAP Pharmacokinetic Estimates - Day 1

| | | | | | Treatment C | |
|---|---|---|---|---|---|---|
| | | | | | Commercially- | |
| | | | | | available | |
| | Treatment A | | Treatment B | | immediate | |
| | ER Formulation | | ER Formulation | | release tablet | |
| | (1 Tablet Q12h) | | (2 Tablets Q12h) | | (1 Tablet Q6h) | |
| | Mean (SD) | | Mean (SD) | | Mean (SD) | |
| Parameter | (N = 33) | | (N = 33) | | (N = 33) | |
| AUC_{0-12 h} | 12192 | (3331) | 24141 | (6436) | 24884 | (6656) |
| (ng · h/mL) | | | | | | |
| Cₘₐₓ (ng/mL) | 2631 | (815) | 5245 | (1473) | 5146 | (1553) |
| Tₘₐₓ (h)^{a} | 0.55 | (0.25-3.00) | 0.75 | (0.25-2.00) | 0.50 | (0.25-8.00) |
| t_{lag} (h)^{a} | 0.00 | (0.00-0.25) | 0.00 | (0.00-0.25) | 0.00 | (0.00-0.00) |

^{a}Median (minimum-maximum).

On Day 5 of the study, median Tₘₐₓ^{ss} for APAP was observed at 30 minutes following 1 or 2 tablets of the ER formulation and at 30 minutes following the first daily dose of the commercially-available immediate release tablet (see Table 42). Acetaminophen concentrations following administration of 325 mg or 650 mg APAP (1 or 2 tablets) Q12 h were proportional to dose. The DFL in and swing of plasma APAP levels for the ER formulation were equivalent to the commercially-available immediate release tablet. On Day 5, interindividual variability (% CV) in Cₘₐₓ^{ss} for APAP was slightly higher following administration of 2 tablets of the ER formulation (33%) than the % CV seen for 1 tablet of the ER formulation and the commercially-available immediate release tablet (˜27%). Interindividual variability in AUC₀₋₁₂ₕ^{ss} for APAP was comparable between all 3 treatments (up to 27%).

TABLE 42
APAP Pharmacokinetic Estimates - Day 5

| | | | | | Treatment C | |
|---|---|---|---|---|---|---|
| | | | | | Commercially- | |
| | | | | | available | |
| | Treatment A | | Treatment B | | immediate | |
| | ER Formulation | | ER Formulation | | release tablet | |
| | (1 Tablet Q12h) | | (2 Tablets Q12h) | | (1 Tablet Q6h) | |
| | Mean (SD) | | Mean (SD) | | Mean (SD) | |
| Parameter | (N = 33) | | (N = 33) | | (N = 33) | |
| AUC_{0-12 h}^{ss} | 15307 | (4092) | 28512 | (7714) | 28719 | (7023) |
| (ng · h/mL) | | | | | | |
| Cₐᵥ^{ss} (ng/mL) | 1276 | (341) | 2376 | (643) | 2393 | (585) |
| Cₘₐₓ^{ss} (ng/mL) | 3117 | (840) | 5872 | (1932) | 5968 | (1639) |
| Cₘᵢₙ^{ss} (ng/mL) | 474.67 | (163) | 870.42 | (336) | 922.58 | (321) |
| DFL (%) | 212.08 | (52.29) | 218.06 | (81.14) | 213.79 | (50.53) |
| Swing | 5.95 | (2.04) | 6.63 | (3.61) | 5.94 | (2.24) |
| Tₘₐₓ^{ss} (h)^{a} | 0.50 | (0.25-3.00) | 0.50 | (0.25-3.02) | 0.50 | (0.25-8.02) |
| t_{1/2} (h)^{c} | 5.60 | (1.35)^{b} | 7.47 | (2.89) | 5.74 | (2.98)^{b} |
| Kₑₗ (1/h)^{c} | 0.1308 | (0.0317)^{b} | 0.1026 | (0.0292) | 0.1416 | (0.0515)^{b} |

^{a}Median (minimum-maximum).
^{b}N = 31
^{c}Days 5 to 7.

Both OC and APAP were rapidly absorbed under all conditions with no lag in plasma concentrations. Both OC and APAP levels were sufficiently high within 1 hour after administration of the ER formulation as a single dose and at steady-state. OC levels were sustained over the proposed 12 h dosing interval. Plasma APAP concentrations decreased to below 1,000 ng/mL between doses of the ER formulation, thus minimizing the chances of its accumulation and the possibility of hepatotoxicity. Total exposure to both OC and APAP from the ER formulation was equivalent to that of the commercially-available immediate release tablet.

### Example 12

### Clinical Evaluation of the Safety and Analgesic Efficacy of an Extended Release Formulation of Oxycodone and Acetaminophen for Acute Pain

Pain relief for acute post-surgical pain requires immediate-release (IR) compounds acting within 1 hour of administration. These IR compounds, however, have a short half-life and require frequent administration; this is inconvenient to patients and leads to poor compliance. Such patients may benefit from an extended-release (ER) oral formulation of oxycodone hydrochloride (OC) and acetaminophen (APAP) that is designed to (1) provide the immediate-release of each drug to attain rapid therapeutic levels (within 1 hour of dosing) and (2) provide continuous release of each drug to maintain the plasma levels of each drug within therapeutic windows for sustained analgesia (up to 12 hours). Furthermore, combining analgesics with distinct mechanisms of action provides maximum efficacy while reducing the toxicity of each agent, as the amount of OC and APAP can remain within the lower, safer end of their therapeutic windows. This ER formulation may provide the advantages of both immediate and prolonged pain relief from two analgesic compounds, potentially offering greater convenience to patients and greater dosing compliance.

Accordingly, a randomized, double-blind, placebo-controlled, phase 3 study was conducted to demonstrate the efficacy of repeated doses of 15 mg OC/650 mg APAP (see selected example from Chart No. 1, which may be referred to in this example as the “study medication”) versus placebo, and to determine the safety and tolerability of multiple oral doses of the OC/APAP formulation administered to subjects with acute postoperative, moderate to severe pain.

In this clinical trial, ER OC/APAP was studied in an established acute pain model in patients undergoing a first metatarsal bunionectomy. Medication effects were evaluated 48 hours post-procedure (double-blind) and continued throughout a voluntary open-label treatment period (up to 14 days). Patients aged 18 to 75 years undergoing unilateral, first metatarsal bunionectomy who reported at least moderate or severe pain intensity and numeric rating scale score of ≧4 (out of 10) between the hours of 4:00 AM and 12:00 PM (after cessation of intravenous popliteal nerve block) on the first postoperative day were eligible for the study.

FIG. 93 presents a summary of the study design. The study was conducted in the following phases: 1) pre-treatment phase consisting of a) screening, b) surgery, and c) recovery/qualification periods; 2) double-blind phase consisting of a single-dose period followed by a multiple-dose period which begins with either (i) the request of the second dose of study medication, or (ii) 12 hours after the first dose of study medication; and 3) a voluntary open-label extension (“OLE”) phase.

The subjects consisted of males and females who were of good general health, and had undergone a primary unilateral first metatarsal bunionectomy. Each subject participated in the double-blind study for up to 40 days, including a screening period of up to 30 days, a surgical period of 1 day, a blinded dosing phase of 2 days, and, for those not entering the OLE phase, a follow-up period of about 7 days. The OLE phase lasted up to 14 days, bringing the total participation time to approximately 54 days.

The single-dose period of the double-blind phase evaluated the onset and duration of analgesia of a single dose of extended release 15 mg OC/650 mg APAP (as two 7.5/325 tablets) versus placebo. The time from the initial dose of study medication to the onset of perceptible pain relief and to the onset of meaningful pain relief was measured. The subjects provided additional pain assessments (e.g., pain intensity scores measured using the 11 point NPRS scale at regular intervals). The use of supplemental analgesia was permitted (i.e., ibuprofen 400 mg up to 6 times per day [2400 mg/d]) during the double-blind and open-label phases of the study.

The multiple-dose period of the double-blind phase evaluated the analgesic effects of multiple doses of extended release 15 mg OC/650 mg APAP versus placebo with subjects dosed regularly every 12 hours for 48 hours. The multiple dose period began either upon administration of the second dose after the subject's request for additional pain relief or 12 hours after the first dose of study medication. Pain relief and intensity will be among the data measured in this arm of the study.

After completion of study evaluations 48 hours after the second dose of study medication, subjects were encouraged to enter the open-label extension phase of the study. During this time they were provided with doses of 15 mg OC/650 mg APAP to be taken every 12 hours until the study medication was no longer needed, for up to 14 days. The open-label extension phase (starting 48 hours after the second dose) evaluated the safety profile as determined by adverse events (AE) and evaluated subject satisfaction with analgesic effects. Eligibility criteria for the open-label extension phase of the study included completing the double-blind phase of the study; having a pain intensity score at completion of the double-blind phase of the study, but no later than 52 hours after receiving the first dose of study drug; signing an open-label extension consent form prior to surgery; and agreeing to participate in the open-label extension phase of the study. The open-label phase lasted up to 14 days, with clinic visits at days 7 and 14 (±1 day), followed by a telephone call 7 days (±2 days) after the last dose.

Exclusion criteria included any medical condition that might decrease study compliance or alter the absorption, distribution, metabolism, or excretion of the study drug (e.g., severe chronic diarrhea, chronic constipation, irritable bowel syndrome, or unexplained weight loss); gastric bypass surgery or gastric band; history of intolerance to short-term opioid use; and treatment with study drug or bunionectomy in previous 3 months.

Safety and tolerability assessments were conducted throughout the open-label phase of the study and included physical examinations, measurement of vital signs (e.g., sitting blood pressure, pulse rate, and temperature), and clinical laboratory tests (i.e., chemistry, hematology, and urinalysis). Adverse events were collected at each visit and the 7-day follow-up phone call. Global assessment of patient satisfaction was evaluated at 48 hours or early termination for the blinded-dosing phase and at every clinic visit for the open-label phase. The study assessed the patient's satisfaction with treatment across 5 dimensions, such as ease of administration and level of pain relief, on a categorical scale (i.e., very satisfied, satisfied, neither satisfied nor dissatisfied, dissatisfied, or very dissatisfied).

Descriptive statistics were summarized for baseline characteristics and global assessment of satisfaction. Medication adherence and treatment-emergent adverse events (TEAEs) were summarized using frequencies and percentages. Summary statistics for actual values and changes from baseline were calculated for the physical examination findings, laboratory test results, vital signs, and pulse oximetry, and a shift analysis examined categorical changes from baseline to various time points.

Pain intensity was rated with an 11-item numerical rating scale (0=no pain; 10=the worst pain imaginable). The primary outcome measure was the summed pain intensity difference over the first 48 hours (SPID48). SPID48 was calculated as the sum of time-weighted pain intensity difference (PID) scores over the first 48 hours (PID=[baseline pain intensity score]−[pain intensity score at time point of interest]). Secondary outcome measures included pain intensity scores, PID associated with each pain intensity score, and SPID at multiple time points over 48 hours; total pain relief (TOTPAR) at multiple time points over the first 48 hours; and the time to perceptible, meaningful, and confirmed pain relief. To reduce the confounding effects of censored (unusable) pain scores due to use of supplemental analgesia (i.e., ibuprofen), PID was estimated using multiple imputation techniques and 6-hour censoring, respectively. The double stopwatch method was used to determine time to onset of pain relief. Global assessment of subject satisfaction was conducted during the study. Safety and tolerability assessments were conducted throughout the double-blind and open-label phases of the study; adverse events were assessed at follow-up, and any significant measures were followed-up as medically indicated.

A total of 329 patients were enrolled and received dose of study drug in the blinded-dosing phase of the study 166 patients received ER OC/APAP; 163 received placebo. 293 patients (89.1%) completed the double-blind phase of the study. 146 patients (49.8%; prior ER OC/APAP, n=77; prior placebo, n=69) who completed the double-blind phase of the study entered the open-label phase of the study, with 129 patients (88.4%) completing the open-label extension. 145 patients attended the 1-week follow-up visit, and 36 patients attended the 2-week follow-up visit. Demographic characteristics of the open-label safety population were generally similar between groups. During the open-label dosing phase, 120 patients (82.2%) received ±20% of the expected doses.

Efficacy analyses were performed on a modified intent-to-treat (mITT) population (N=303) randomized to treatment with either ER OC/APAP (n=150) or placebo (n=153). Demographic and baseline characteristics of the mITT population were generally similar between groups. The mean baseline pain intensity score for the mITT population was 6.2 (SD=1.7) for the ER OC/APAP treatment population and 6.0 (SD=1.5) for the placebo population.

The superior efficacy of the study medication compared to the placebo in the treatment of acute pain after bunionectomy was demonstrated consistently across a variety of validated pain measures. The primary endpoint, the sum of the pain intensity difference over the 48-hour blinded period, was statistically significantly greater in the study medication group compared to the placebo group. Indeed, the subjects treated with the study medication had less pain than the placebo-treated subjects. The multiple imputation mean summed pain intensity difference over the first 48 hours (“SPID₄₈”) was significantly greater for the study medication group than the placebo group, namely, 114.9 versus 66.9, with a treatment difference of 48.0, which was statistically significant (P<0.001).

Decrease in pain intensity scores over time are shown in FIG. 94 (for hours 0 to 2) and FIG. 95 (for hours 0 to 48). Mean PID for ER OC/APAP was numerically superior beginning at the earliest time point measured (15 min); statistical significance was reached 30 minutes after the first dose of study drug (P<0.02). Mean SPID over 0-4 (6.5), 0-12 (13.0), 0-24 (27.7), and 0-36 hours (39.7) were all statistically significant for ER OC/APAP versus placebo (P<0.001 for all comparisons). Furthermore, as shown in Table 100, mean TOTPAR over 0-4, 0-12, 0-24, 0-36, and 0-48 hours were all significantly greater for ER OC/APAP versus placebo.

TABLE 100
Total Pain Relief Over 0-4, 0-12, 0-24, 0-36, and 0-48 Hours

| | | | Treatment | | |
|---|---|---|---|---|---|
| TOTPAR | ER OC/APAP | Placebo | Difference | | P |
| Interval | (n = 150)^{a} | (n = 153)^{a} | (95% CI) | | Value |
| 0-4 h | 6.8 (0.4) | 3.4 (0.4) | 3.4 | (2.4-4.4) | <0.001 |
| 0-12 h | 16.5 (0.9) | 11.2 (0.8) | 5.3 | (2.9-7.7) | <0.001 |
| 0-24 h | 38.4 (1.7) | 26.8 (1.6) | 11.6 | (7.1-16.2) | <0.001 |
| 0-36 h | 64.2 (2.5) | 47.5 (2.5) | 16.8 | (9.8-23.8) | <0.001 |
| 0-48 h | 91.3 (3.5) | 70.9 (3.4) | 20.5 | (11.0-30.0) | <0.001 |

^{a}Mean (SE)
CI = confidence interval

The proportion of patients with ≧30% reduction in pain intensity score at different times during the first 2 hours of treatment is shown in FIG. 96. The proportion of 30% responders was significantly higher with ER OC/APAP than with placebo from as early as 30 minutes after the first dose, and the difference increased over the subsequent 90 minutes. This fast onset of action was further demonstrated by the mean TOTPAR values over the first 4 hours (Table 100), as well as by the times to perceptible and confirmed perceptible pain relief.

More patients receiving ER OC/APAP experienced perceptible, meaningful, and confirmed perceptible pain relief. As shown in Table 101, median time to pain relief was significantly shorter for ER OC/APAP compared with placebo.

TABLE 101
Median Time to Onset of Perceptible,
Confirmed Perceptible, and Meaningful Pain Relief

| | ER OC/ | | |
|---|---|---|---|
| | APAP | Placebo | |
| Time, min | (n = 150) | (n = 153) | P Value |
| To perceptible pain relief | 33.56 | 43.63 | <0.001 |
| To confirmed perceptible pain relief | 47.95 | NE | <0.001 |
| To meaningful pain relief | 92.25 | NE | <0.001 |

NE = could not be estimated due to less than half the subjects experiencing confirmed or meaningful pain relief.

At the end of the double-blind phase, more patients indicated they were either “satisfied” or “very satisfied” with time taken for medication to work and level of pain relief by pain medication for ER OC/APAP compared with placebo. FIG. 97 presents the percentage of patients “satisfied” or “very satisfied” on items of the global assessment of satisfaction at 48 hours. As would be expected, there were no differences between groups on ease of taking, frequency of taking, or amount of medication taken.

Overall, during the blinded-dosing phase of the study, 37.7% of patients in the safety population (124/329) experienced a treatment-emergent adverse event (TEAE). The most common TEAEs reported during the blinded-dosing phase of the study are summarized in Table 102. As expected for this class of medication, a greater percentage of patients receiving ER OC/APAP reported nausea (30.7% vs 5.5%), dizziness (13.3% vs 1.2%), headache (9.6% vs 4.9%), skin and subcutaneous disorders (9.0% vs 4.3%), vomiting (9.0% vs 0%), and somnolence (3.6% vs 0.6%) compared with patients receiving placebo, respectively. Constipation was reported by a small percentage of patients receiving either ER OC/APAP or placebo (4.2% vs 3.1%, respectively). One patient in the group receiving ER OC/APAP reported a severe TEAE (headache), and no serious adverse events were reported during the blinded-dosing phase of the study.

TABLE 102
Summary of Treatment-Emergent Adverse
Events Occurring in >3% of Patients

| Treatment-Emergent | ER OC/APAP | Placebo | All Patients |
|---|---|---|---|
| Adverse Event, n (%) | (n = 166) | (n = 163) | (N = 329) |
| Any TEAE | 89 (53.6) | 35 (21.5) | 124 (37.7) |
| Nausea | 51 (30.7) | 9 (5.5) | 60 (18.2) |
| Dizziness | 22 (13.3) | 2 (1.2) | 24 (7.3) |
| Headache | 16 (9.6) | 8 (4.9) | 24 (7.3) |
| Skin and subcutaneous | 15 (9.0) | 7 (4.3) | 22 (6.7) |
| tissue disorders | | | |
| Vomiting | 15 (9.0) | 0 | 15 (4.6) |
| Constipation | 7 (4.2) | 5 (3.1) | 12 (3.6) |
| Somnolence | 6 (3.6) | 1 (0.6) | 7 (2.1) |

Overall, the subjects that were administered the study medication during the blinded dosing phase had less pain, greater pain relief, less need for rescue medication, and rated the study medication more highly as a pain reliever. The result of this study therefore demonstrated that the study medication was shown to provide rapid, significant, and consistent analgesic efficacy over the 12-hour dosing interval, with pain relief satisfaction statistically significantly better than the placebo over the 48-hour blinded treatment period. The majority of subjects were also very satisfied in their global assessment of therapy during the OLE phase. The safety findings indicate that study medication is well tolerated, with a safety profile desirable from a low-dose opioid/APAP treatment.

Table 103 presents a summary of the treatment-emergent adverse events (TEAEs) occurring during the open label phase. A total of 64 patients (43.8%) experiences ≧1 TEAE. The most frequently reported TEAEs were primarily gastrointestinal related (nausea, vomiting, constipation) and central nervous system-related (somnolence, headache, dizziness).

TABLE 103
Treatment-Emergent Adverse Events
Occurring During the Open-Label Phase

| | | Prior | |
|---|---|---|---|
| | Prior Double-Blind | Double-Blind | All |
| Treatment-Emergent | ER OC/APAP | Placebo | Patients |
| Adverse Event, n (%) | (n = 77) | (n = 69) | (N = 146) |
| Any TEAE | 25 (32.5) | 39 (56.5) | 64 (43.8) |
| Nausea | 8 (10.4) | 18 (26.1) | 26 (17.8) |
| Vomiting | 3 (3.9) | 8 (11.6) | 11 (7.5) |
| Constipation | 4 (5.2) | 5 (7.2) | 9 (6.2) |
| Somnolence | 1 (1.3) | 6 (8.7) | 7 (4.8) |
| Headache | 4 (5.2) | 2 (2.9) | 6 (4.1) |
| Dizziness | 2 (2.6) | 4 (5.8) | 6 (4.1) |
| Peripheral edema | 3 (3.9) | 1 (1.4) | 4 (2.7) |
| Pruritus | 1 (1.3) | 3 (4.3) | 4 (2.7) |
| Infection | 1 (1.3) | 3 (4.3) | 4 (2.7) |

One patient reported 3 severe TEAEs, and 1 patient reported a serious adverse event (i.e., deep vein thrombosis determined by the investigator to be unrelated to treatment with the study drug). Changes from baseline in laboratory values (i.e., hematology, serum chemistry, and urinalysis) were generally small and were similar between treatment groups during double-blind periods and similar between the double-blind and open-label periods. Six patients (4.1%) had alanine aminotransferase and/or aspartate aminotransferase times the upper limit of normal values at least once during the study. Total bilirubin remained within the normal reference range in all 6 cases. None met Hy's Law criteria.

Table 104 presents the vital sign measures and changes from baseline after 7 days of open-label treatment. Vital signs during the open-label phase were normal in >90% of patients at any visit. During the open-label phase, ≦1.4% of patients had shifts from normal to abnormal oxygen saturation at any time point.

TABLE 104
Vital Sign Measures and Changes From Baseline
After 7 Days of Open-Label Treatment

| | Baseline | OL Visit | Change |
|---|---|---|---|
| | Value | Day 7 | From |
| Vital Sign | (n = 146) | (n = 145) | Baseline |
| Systolic blood | | | |
| pressure, mmHg | | | |
| Mean (SD) | 117.3 (14.12) | 120.6 (14.4) | 3.4 (12.34) |
| Median | 116.0 | 120.0 | 3.0 |
| Diastolic blood | | | |
| pressure, mmHg | | | |
| Mean (SD) | 73.2 (9.31) | 75.2 (9.07) | 2.1 (8.45) |
| Median | 72.0 | 74.0 | 2.0 |
| Heart rate, beats/min | | | |
| Mean (SD) | 73.9 (10.81) | 74.1 (11.62) | 0.2 (10.99) |
| Median | 74.0 | 74.0 | −1.0 |
| Respiratory rate, | | | |
| breaths/min | | | |
| Mean (SD) | 16.3 (1.94) | 15.8 (1.68) | −0.5 (2.52) |
| Median | 16.0 | 16.0 | 0.0 |
| Body temperature, | | | |
| ° C. | | | |
| Mean (SD) | 36.66 (0.50) | 36.40 (0.54) | −0.26 (0.58) |
| Median | 36.70 | 36.50 | −0.20 |
| Oxygen saturation, % | | | |
| Mean (SD) | 97.6 (1.69) | 98.0 (1.60) | 0.4 (1.76) |
| Median | 98.0 | 98.0 | 0.0 |

* By Day 14, very few patients required pain medications (n = 36); therefore, data from n = 145 completing day 7 are shown above.

FIG. 98 presents the proportion of patients “satisfied” or “very satisfied” with ER OC/APAP after 7 or 14 days of open-label phase treatment, according to five different measures. At the 7-day follow-up, more than 88% (of 144 patients) indicated they were “very satisfied” or “satisfied” on all measures. At the 14-day follow-up, more than 83% (of 36 patients) indicated they were “very satisfied” or “satisfied” on all measures.

The results of this study demonstrate that multiple-dose administration of ER OC/APAP was generally well tolerated. The most frequently reported adverse events were consistent with those seen with other opioids in general, and specifically, oxycodone. Shifts in laboratory test results, vital signs, and oxygen saturation were generally small and not clinically significant. All changes in clinical laboratory tests and vital signs that were outside of the defined reference range(s) were not clinically significant according to the investigator. More than 80% of patients were very satisfied or satisfied with every measure of treatment assessed, including 94.4% for ease of administration, 86.1% for time for the medication to work, and 83.3% for level of pain relief.

### Example 13

### Clinical Evaluation of the Safety and Efficacy of an Extended Release Formulation of Oxycodone and Acetaminophen for Chronic Pain

A multi-center, phase 3, open label safety study of doses of extended release 15 mg OC/650 mg APAP (see selected example from Chart No. 1) administered at 12 hour intervals for up to 35 days in a patient population having pain associated with osteoarthritis (OA) of the knee or hip or chronic low back pain (CLBP) were conducted. The primary objective of the study was to determine the safety and tolerability of doses of extended release 15 mg OC/650 mg APAP for up to 35 days of use. Secondary objectives such as pain relief, changes in pain intensity, and pain-related quality of life were also assessed.

Participants in the study were adults with clinical diagnoses of osteoarthritis of the knee or hip, with moderate to severe pain intensity despite chronic use of stable doses of nonopioid or opioid analgesics; or chronic low back pain that was moderate to severe in intensity and present for several hours a day for months. Patients transitioned from nonopioid analgesics to opioid combination therapy. A 3-day washout period was required for all patients taking analgesic medications.

FIG. 99 presents a summary of the study design. The study included a screening period of up to two weeks followed by a 3-day washout period. ER OC/APAP was administered as 2 tablets every 12 hours for up to 35 days. Weekly clinic visits were used to assess safety and tolerability, as well as efficacy. Assessments occurred at >2 hours after dosing.

Subjects enrolled in the study were treated with 2 tablets of extended release 7.5 mg OC/325 mg APAP every 12 hours (Q12 h) for between 10 days and 35 days. Subjects initially took 1 tablet of 7.5 mg OC/325 mg APAP under clinic supervision. Subjects were observed for opioid tolerability symptoms. Subjects who experience opioid tolerability symptoms, or moderate to severe adverse events, were discontinued from the study. Subjects who did not experience opioid tolerability symptoms, or moderate to severe adverse events, were given a second tablet of 7.5 mg OC/325 mg APAP under clinic supervision. If subjects still did not experience opioid tolerability symptoms, or moderate to severe adverse events, they were sent home with supplies for dosing with 2 tablets of 7.5 mg OC/325 mg APAP Q12 h for one week. If subjects did experience opioid tolerability symptoms, or moderate to severe adverse events, they were sent home with supplies for dosing with 1 tablet of 7.5 mg OC/325 mg APAP Q12 h for one week. Supplemental analgesia, consisting of 400 mg ibuprofen (two 200 mg tablets), could be taken as needed every 4 to 6 hours to manage breakthrough pain (2400 mg maximum daily).

Subjects that continued in the study beyond one week took 2 tablets Q12 h for up to a total of 35 days, during which time they returned to the clinic for subsequent assessments of safety and efficacy. After the Day 36 visit, subjects were instructed to return to their pre-study medication. Subjects whose pain subsided prior to the Day 36 visit, or who discontinued the study medication for other reasons were instructed to return any remaining study medication.

The following safety assessments were conducted at baseline and weekly throughout the study period: treatment adherence, treatment-emergent adverse events (TEAEs), vital signs, pulse oximetry, and liver function tests. Additional laboratory tests (chemistry, hematology, and urinalysis) were performed at screening and the final visit. The following efficacy assessments were conducted for patients in the study: Brief Pain Inventory (BPI) for all patients; Western Ontario and McMaster Universities Arthritis Index (WOMAC) questionnaire for patients with osteoarthritis; Roland-Morris Disability Questionnaire (RMDQ) for patients with chronic low back pain.

Summary statistics, frequency counts, and percentages were calculated for baseline demographics, duration of exposure, and TEAEs. Shift analysis examining changes from baseline to end of treatment were performed for physical examination findings and laboratory test results. For vital signs and pulse oximetry, actual measurements at baseline and at end of treatment (taken at least 2 hours post-dose), along with change from baseline, were summarized. Summary statistics for actual values and changes from baseline were calculated for the secondary efficacy assessments.

FIG. 100 provides a summary of the patient disposition in the study. Of the 376 patients enrolled, 75.8% completed the study. The most common reason for discontinuation was TEAE. Per protocol, any patient who experienced emesis or moderate or severe nausea within 4 hours of dosing was discontinued. Mean duration of ER OC/APAP exposure was 29.2 days, with duration of exposure ≧10 days for 82.4% of patients. 94.1% of patients received ±20% of the expected doses.

Overall, 62.5% of patients experienced ≧1 TEAE. TEAEs that occurred in ≧5% of patients were nausea, vomiting, dizziness, somnolence, constipation, pruritus, and headache. Table 105 presents a summary of the TEAE events. Most TEAEs were rated by the investigator to be mild or moderate in intensity; 17 patients reported a total of 22 severe TEAEs, of whom 10 patients experienced ≧1 severe gastrointestinal event (nausea, n=6; vomiting, n=5; and constipation, n=1). A total of 4 patients experienced ≧1 severe adverse event (as rated by the investigator).

TABLE 105
Treatment-Emergent Adverse Events Occurring
in ≧5% of Patients, Safety Population

| Treatment-Emergent | OA, Hip | OA, Knee | CLBP | Total |
|---|---|---|---|---|
| Adverse Event, n (%) | (n = 12) | (n = 129) | (n = 235) | (N = 376) |
| Nausea | 2 (16.7) | 29 (22.5) | 56 (23.8) | 87 (23.1) |
| Vomiting | 2 (16.7) | 17 (13.2) | 38 (16.2) | 57 (15.2) |
| Dizziness | 3 (25.0) | 20 (15.5) | 33 (14.0) | 56 (14.9) |
| Somnolence | 1 (8.3) | 11 (8.5) | 31 (13.2) | 43 (11.4) |
| Constipation | 1 (8.3) | 14 (10.9) | 27 (11.5) | 42 (11.2) |
| Pruritus | 0 (0) | 8 (6.2) | 19 (8.1) | 27 (7.2) |
| Headache | 0 (0) | 6 (4.7) | 13 (5.5) | 19 (5.1) |

CLBP = chronic low back pain;
OA = osteoarthritis.

The majority of patients (>85% on 31 of 35 measures) had normal hematology, chemistry, and urinalysis values at end of treatment; few subjects shifted from normal to abnormal values. Table 106 presents a summary of the changes in vital signs or pulse oximetry. Changes in physical examination findings, vital signs, and oxygen saturation were not clinically significant. One patient (0.2%) was found to have hypopnea that was considered related to study medication and resulted in discontinuation.

TABLE 106
Changes in Vital Signs and Pulse Oximetry, All Participants

| | | End of | |
|---|---|---|---|
| | Baseline | Treatment | |
| Measure, Mean (SD) | (N = 376) | (n = 367) | Change |
| Systolic blood pressure, | 125.9 (13.7) | 124.9 (13.0) | −0.9 (13.0) |
| mmHg | | | |
| Diastolic blood pressure, | 78.0 (9.1) | 77.5 (8.8) | −0.5 (9.0) |
| mmHg | | | |
| Heart rate, beats/min | 74.5 (9.7) | 73.2 (10.4) | −1.2 (9.6) |
| Respiratory rate, breaths/ | 16.0 (2.0) | 16.1 (2.0) | 0.1 (1.6) |
| min | | | |
| Body temperature, C.° | 36.7 (0.4) | 36.6 (0.4) | −0.03 (0.4) |
| Oxygen saturation, % | 97.1 (1.6) | 97.1 (1.7) | −0.04 (1.6) |

Shifts in liver function test results from normal at baseline to elevated at the end of treatment were observed in a small number of patients. 25 (6.9%) patients had shifts from normal to elevated ALT (alanine aminotransferase); 18 (5.0%) experienced a shift from normal to elevated AST (aspartate aminotransferase); and 2 (0.6%) patients had shifts from normal to elevated bilirubin. 4 (1.1%) patients had either ALT and/or AST values >5 times the upper limit of normal (ULN) at some point during the study; none met Hy's Law criteria (ALT>3 times ULN or AST>3 times ULN, associated with total bilirubin >2 times ULN and serum alkaline phosphatase <2 times ULN). 10 (2.7%) patients had liver function test results that the investigator deemed clinically significant; 5 (1.4%) patients discontinued due to these adverse events, and all resolved or were resolving after discontinuation.

The extended release 7.5 mg OC/325 mg APAP tablets that were provided to the subjects who participated in this study, delivered effective pain management. Several measures of pain control and relief were demonstrated in subjects with either OA or CLBP. Scores for (i) worst pain in the last 24 hours, (ii) least pain in the last 24 hours, (iii) average pain in the last 24 hours, and (iv) current pain all decreased from baseline to the end of treatment, by 47%, 57%, 52%, and 60% (mean score change), respectively. Further, the greatest improvement occurred at Day 36. FIG. 101 presents the brief pain inventory (changes in pain intensity from baseline to end of treatment). Substantial decreases in pain intensity (worst pain, average pain, and current pain) were observed early in treatment and continued throughout the study. In addition, patients' mean score for pain interference questions (0=no interference; 10=completely interferes) decreased from 5.4 at baseline to 2.2 at end of treatment (change from baseline, −3.2; P<0.0001).

The percent of pain relief also increased steadily from baseline through Day 36 (mean improvement of 55%). Pain-related quality of life, as measured by the modified Brief Pain Inventory short form (“mBPI-sf”) pain interference score, continually improved at each visit. FIGS. 102-105 present the changes from baseline on domains of the WOMAC (pain, stiffness, physical function, and total) in patients with osteoarthritis. Statistically significant improvements from baseline to end of treatment were seen across all domains of the WOMAC for patients with osteoarthritis. In addition, statistically significant improvements in function/disability from baseline to end of treatment were reported by patients with chronic low back pain; RMDQ scores improved from 11.0 at baseline to 6.1 at endpoint, a reduction of 4.9 points (P<0.001). The WOMAC pain scores and RMDQ scores both improved from baseline to end of treatment, by 46% (mean total score, OA) and 45% (mean score, CLBP), respectively.

Moreover, the 7.5 mg OC/325 mg APAP tablet used in this study was an immediate release/extended release, opioid/nonopioid combination product using gastric retentive technology that demonstrated a safety profile consistent with expectations for a low dose opioid/APAP combination product. This study further established that the safety profile of extended dosing (up to 35 days) of the 7.5 mg OC/325 mg APAP tablet and suggests that the 7.5 mg OC/325 mg APAP tablet effectively controls pain management in patients with OA or CLBP. Overall, no apparent clinically significant treatment-related trends were observed in most clinical laboratory test results, vital signs, pulse oximetry measurements, or physical examination findings. All changes in hematology, chemistry, urinalysis, vital signs, and oxygen saturation that were outside of the defined reference range(s) were not clinically significant according to the investigator. 10 (2.7%) patients had liver function test values assessed as clinically significant, although none met Hy's Law criteria. The safety/tolerability profile of ER OC/APAP was consistent with expectations for an opioid/APAP combination product. The most frequently reported adverse events were nausea, vomiting, dizziness, somnolence, and constipation. Statistically significant analgesia and improvements in function were observed in patients with osteoarthritis and chronic low back pain, as measured by BPI, WOMAC, and RMDQ. The safety, tolerability, and effectiveness findings in this study support the administration of the 7.5 mg OC/325 mg APAP tablet disclosed herein every 12 hours to patients for the management of moderate to severe acute pain.

### Example 14

### Partial Areas Under the Curve for Oxycodone and Acetaminophen

Partial AUCs were calculated for a bilayer extended release tablet disclosed herein containing acetaminophen and oxycodone, and an immediate release acetaminophen and oxycodone tablet. Specifically, Partial AUCs were calculated for the acetaminophen and oxycodone tablets of (1) Treatment B of Example 10, (2) Treatment C of Example 9, and (3) Treatment D of Example 10. These results are summarized in Tables 43-46.

TABLE 43
Mean (SD) Parameter Estimates for Partial AUCs for
Acetaminophen.

| | AUC_{0-1.7 h} | AUC_{1.7-48 h} | AUC₀₋ₜ |
|---|---|---|---|
| Study | (ng · h/mL) | (ng · h/mL) | (ng · h/mL) |
| Treatment B (Ex. 10) | 6029 | 28435 | 32644 |
| Treatment C (Ex. 9) | 5854 | 25539 | 29741 |

TABLE 44
Additional Mean (SD) Parameter Estimates for Partial AUCs for Acetaminophen.

| | AUC_{0-12 h} | AUC_{0-12 h} | AUC_{12-36 h} | AUC_{8-12 h} | AUC₀₋ₜ |
|---|---|---|---|---|---|
| Study | (ng · h/mL) | (ng · h/mL) | (ng · h/mL) | (ng · h/mL) | (ng · h/mL) |
| Treatment B (Ex. 10) | 25912 | 22615 | 7978 | 4401 | 32644 |
| Treatment C (Ex. 9) | 24102 | 20875 | 6854 | 3910 | 29741 |

TABLE 45
Percent of AUC₀₋ₜ Acetaminophen

| | | AUC_{1-12 h} | AUC_{12-36 h} | |
|---|---|---|---|---|
| | AUC₀₋₁₂ₕ | (Tₘₐₓ to end of | (end of dosing | |
| | (dosing | dosing | interval to last | |
| Study | interval) | interval) | concentration) | AUC_{8-12 h} |
| Treatment B | 79% | 69% | 24% | 13% |
| (Ex. 10) | | | | |
| Treatment C | 81% | 70% | 23% | 13% |
| (Ex. 9) | | | | |

TABLE 46
Mean(SD) Parameter Estimates for Partial AUCs for Oxycodone.

| | AUC_{0-2.8 h} | AUC_{2.8-48 h} | AUC₀₋ₜ |
|---|---|---|---|
| Study | (ng · h/mL) | (ng · h/mL) | (ng · h/mL) |
| Treatment B | 28.75 | 158.49 | 185.93 |
| (Ex. 10) | | | |
| Treatment C | 27.89 | 164.27 | 190.66 |
| (Ex. 9) | | | |

The bioequivalence determinations between two tablets of a pharmaceutical composition described herein, each containing 7.5 mg oxycodone and 325 mg acetaminophen and an immediate release tablet comprising 7.5 mg oxycodone and 325 mg acetaminophen can be found in Tables 47 and 48.

TABLE 47
Bioequivalence Determination for Acetaminophen

| | |
|---|---|
| LSM | 90% CI |

| Parameter | Ratio | Lower | Upper |
|---|---|---|---|
| Ln (AUC_{0-1.7 h}) | 101.97 | 82.90 | 125.43 |
| Ln (AUC_{1.7-48 h}) | 91.15 | 80.58 | 103.11 |
| Ln (AUC₀₋ₜ) | 93.14 | 82.40 | 105.28 |

TABLE 48
Bioequivalence Determination for Oxycodone

| | |
|---|---|
| LSM | 90% CI |

| Parameter | Ratio | Lower | Upper |
|---|---|---|---|
| Ln (AUC_{0-2.8 h}) | 99.04 | 87.83 | 111.68 |
| Ln (AUC_{2.8-48 h}) | 103.21 | 92.57 | 115.06 |
| Ln (AUC₀₋ₜ) | 102.19 | 92.34 | 113.09 |

The results demonstrate that the plasma concentrations of both oxycodone and acetaminophen rose rapidly with no lag time for a pharmaceutical composition of the present invention and an immediate release tablet comprising 7.5 mg oxycodone and 325 mg acetaminophen. See FIG. 29. Further, 30 minutes after administration of a dose of a pharmaceutical composition of the present invention (i.e., 2 tablets of 7.5 oxycodone/325 acetaminophen), oxycodone levels were within the therapeutic range (>5 ng/mL). Thus, an analgesic effect will be seen in opioid naïve patients. In addition, a pharmaceutical composition of the present invention was able to maintain oxycodone levels above 5 ng/mL for up to 12 hours after dosing, suggesting that the analgesic effect may extend to the next dosing cycle.

Concentrations of acetaminophen resulting from a dose of a pharmaceutical composition of the present invention (i.e., 2 tablets of 7.5 oxycodone/325 acetaminophen), decreased to less than 900 ng/mL (>17% of Cmax) by 12 hours after administration. This decreased concentration of acetaminophen at the end of the dosing cycle allows for sufficient acetaminophen or “APAP time off” between doses.

Oxycodone and acetaminophen levels from a pharmaceutical composition of the present invention (i.e., 2 tablets of 7.5 oxycodone/325 acetaminophen) declined at a similar rate to an immediate release tablet comprising 7.5 mg oxycodone and 325 mg acetaminophen, with a terminal elimination half-life of approximately 4 to 5 hours.

### Example 15

### Partial Areas Under the Curve for Oxycodone and Acetaminophen Administered with Food

Partial AUCs were calculated for a bilayer extended release tablet disclosed herein containing acetaminophen and oxycodone, and an immediate release acetaminophen and oxycodone tablet. Specifically, Partial AUCs were calculated for the acetaminophen and oxycodone tablets of (1) Treatment A of Example 4, (2) Treatment A of Example 6 (one tablet), and (3) Treatment C of Example 4. These results are summarized in Tables 49-50.

TABLE 49
Mean (SD) Parameter Estimates for Partial AUCs for
Acetaminophen.

| | AUC_{0-3.2 h} | AUC_{3.2-48 h} | AUC₀₋ₜ |
|---|---|---|---|
| Study | (ng · h/mL) | (ng · h/mL) | (ng · h/mL) |
| Treatment A (Ex. 4) | 8042 | 23810 | 30245 |
| Treatment A (Ex. 6) | 9145 | 23319 | 31478 |
| (one tablet) | | | |

TABLE 50
Mean (SD) Parameter Estimates for Partial AUCs for Oxycodone.

| | AUC_{0-4.3 h} | AUC_{4.3-48 h} | AUC₀₋ₜ |
|---|---|---|---|
| Study | (ng · h/mL) | (ng · h/mL) | (ng · h/mL) |
| Treatment A (Ex. 4) | 48.62 | 152.57 | 199.43 |
| | (15.99) | (49.86) | (59.47) |
| Treatment A (Ex. 6) | 53.29 | 167.50 | 219.20 |
| (one tablet) | (17.12) | (51.83) | (55.99) |

The bioequivalence determinations between the pharmaceutical composition described herein, containing 15 mg oxycodone and 650 mg acetaminophen and an immediate release product comprising 15 mg oxycodone and 650 mg acetaminophen can be found in Tables 51 and 52.

TABLE 51
Bioequivalence Determination for Acetaminophen

| | |
|---|---|
| LSM | 90% CI |

| Parameter | Ratio | Lower | Upper |
|---|---|---|---|
| Ln(AUC_{0-3.2 h}) | 114.46 | 96.21 | 136.16 |
| Ln (AUC_{3.2-48 h}) | 94.62 | 83.31 | 107.47 |
| Ln (AUC₀₋ₜ) | 101.32 | 90.00 | 114.07 |

TABLE 52
Bioequivalence Determination for Oxycodone

| | |
|---|---|
| LSM | 90% CI |

| Parameter | Ratio | Lower | Upper |
|---|---|---|---|
| Ln (AUC_{0-4.3 h}) | 109.87 | 94.98 | 127.08 |
| Ln (AUC_{4.3-48 h}) | 109.75 | 94.48 | 127.48 |
| Ln (AUC₀₋ₜ) | 110.53 | 97.39 | 125.44 |

Exposure to oxycodone and acetaminophen was comparable between Treatment A of Example 4 and Treatment A of Example 6 (one tablet). Thus, these results indicate that the release of oxycodone and acetaminophen is consistent across studies. Plasma concentration-time profiles are presented in FIGS. 30A and 30B.

The initial exposure to oxycodone (AUC_{0-4.3h}) was slightly outside the bioequivalence parameters established by the FDA (upper 90% CI 127%). The initial exposure to acetaminophen (AUC_{0-3.2h}) was outside of the FDA's bioequivalence parameters (upper 90% CI 136%).

The extended (sustained) exposure to oxycodone (AUC_{4.3-48h}) was slightly outside the FDA's limit for bioequivalence (upper 90% CI 127%). However, the extended exposure to acetaminophen (AUC_{3.2-48h}) and total exposure (AUC₀₋ₜ) for both oxycodone and acetaminophen was equivalent between studies.

### Example 16

### Mechanical Crushing into Powder Form

Drug abusers often tamper with extended release opioid-containing formulations by crushing the dosage form. This process generally serves several functions, including destroying the extended release properties of the dosage form and enabling the dosage form to be processed for unintended methods of administration, such as snorting or intravenous injection. Accordingly, comparative tamper resistance experiments were performed on an extended release tablet dosage form of the pharmaceutical composition of the present invention containing 7.5 mg oxycodone HCl and 325 mg acetaminophen (see Chart 1) (the “product”) and a commercially available immediate-release tablet containing 7.5 mg oxycodone/325 mg acetaminophen (the “comparator”).

The product and comparator tablets were subjected to standard mechanical crushing by the following means: a hammer, a pill crusher, a mortar and pestle, a knife, two spoons, a utility knife, a blender, a coffee mill, and a coffee grinder. The success or failure of the particle size reduction was then visually assessed. In some cases, a sieving analysis was also utilized to quantitatively measure if significant particle size reduction occurred. Generally, drug abusers desire to crush pharmaceutical formulations into a fine powder, as this form is convenient for processing the tablet into a snortable or injectable form.

The results demonstrated that in most instances, the comparator was easily broken down into smaller pieces by each of the mechanical means listed above. Accordingly, in most instances, the comparator offered little tamper resistance as it could easily be mechanically crushed into a suitable powder. In contrast, the physical properties of the product tablet prevented the product tablet from being crushed into a fine powder. Indeed, in relation to the comparator, the product tablet was more difficult to break down using the methods listed above. Specifically, all of the mechanical methods described above were ineffective at producing a suitable powder from the product tablets except grinding in a mortar and pestle. Consequently, the product tablets offer improved protection from the mechanical crushing methods employed by drug abusers.

### Example 17

### Abuse Resistance Properties of Product Powders Produced by Grinding Using a Mortar and Pestle

An in vitro dissolution test with human abuse liability (“HAL”) predictions was conducted to determine the cumulative amount of drug released from intact and crushed tablets of the extended release pharmaceutical compositions disclosed herein and a commercially-available immediate release oxycodone and acetaminophen tablet.

Comparator tablets (the “comparator”) containing a total of 7.5 mg of oxycodone HCl and a total of 325 mg acetaminophen were obtained. Six comparator tablets were ground with a mortar and pestle and placed into capsules, while six tablets were used as is (i.e., kept intact, but placed into capsules). Dissolution profiles for the intact and crushed tablets were determined in a USP type II apparatus. Six intact tablets and six crushed tablets were weighed, placed in a sinker, and dropped into an equilibrated dissolution bath vessel containing 900 mL of (helium sparged) 0.1 N HCl heated to 37° C.±0.5° C. The mixture was stirred at 100±4 rpm, and the temperature was maintained at 37° C.±0.5° C. for 12 hr. The bath vessel was covered with a low evaporation vessel cover. Samples (5 mL) were removed at 5 min, 10 min, 20 min, 30 min, and 60 min. Each sample was filtered through a 0.45 μm filter and analyzed by HPLC using standard procedures. The release profile of oxycodone HCl from intact and crushed comparator is shown in FIG. 31.

Bilayer formulations described herein were prepared, each containing a total of 7.5 mg of oxycodone HCl, a total of 325 mg of acetaminophen, and an extended release polymer. Six product tablets (as defined in Example 16) were ground with a mortar and pestle and placed into capsules, while twelve product tablets were used as is. The same dissolution method as described for the intact and crushed comparator above was used to obtain release profiles for intact and crushed product tablets. However, six of the intact product tablets (labeled as “Intact”) were sampled (5 mL) at 0.25 hr, 0.5 hr, 1 hr, 2 hr, 4 hr, 6 hr, 8 hr, and 12 hr. The release profiles of acetaminophen and oxycodone HCl from the intact and crushed product tablets are shown in FIGS. 32A and 32B (for acetaminophen) and 33A and 33B (for oxycodone). In these figures, “intact” refers to the intact product tablets sampled at 0.25 hr, 0.5 hr, 1 hr, 2 hr, 4 hr, 6 hr, 8 hr, and 12 hr. “HAL_Intact” refers to the intact product tablets sampled at the same time intervals as the crushed tablets, namely, 5 min, 10 min, 20 min, 30 min, 1 hr, 2 hr, 4 hr, 6 hr, 8 hr, 12 hr.

These results show that for release of oxycodone HCl from the comparator tablets, there is no substantial difference in the release profiles for crushed and intact tablets for abuse purposes. In each case, almost all of the oxycodone HCl was released in as little as ten minutes. In stark contrast, there are substantial differences in the release profiles for crushed and intact product tablets. The intact product tablets surprisingly exhibited a higher release rate of both active ingredients than the crushed product tablets in the first hour. This suggests that upon grinding the product tablets, the active ingredients in the immediate release portion are incorporated into the extended release portion, and the product tablet loses its immediate release characteristics. This feature may effectively negate a drug abuser's purpose for crushing the product tablet in the first place—to obtain an early onset of analgesia.

Predicted pharmacokinetic parameters were obtained for these in vitro release profiles for the crushed and intact products and comparator tablets by using in vitro in vivo correlation (“IVIVC”) technique. These results, which are summarized in Table 53, demonstrate that the abuse quotients for the crushed and intact comparator tablets are orders of magnitude higher than the abuse quotients for the crushed and intact product tablets. This is consistent with the experimentally determined pharmacokinetic parameters from Example 10.

TABLE 53
Predicted pharmacokinetic parameters and abuse quotient for intact
and crushed product and comparator tablets.

| | | | |
|---|---|---|---|
| | | | Abuse Quotient |
| Product | Cₘₐₓ (ng/mL) | Tₘₐₓ (hr) | (ng/mL · hr) |

Predicted

| | | | |
|---|---|---|---|
| Comparator (intact) | 32.5 | 0.16 | 203.1 |
| Comparator (crushed) | 30.8 | 0.17 | 181.2 |
| Product (intact) | 17.5 | 6 | 2.9 |
| Product (crushed) | 20.6 | 4 | 5.2 |

Experimental - see Example 10

| | | | |
|---|---|---|---|
| Comparator (intact) | 41.6 | 0.7 | 59.4 |
| Product (intact) | 16.4 | 3.2 | 5.1 |

### Example 18

### Preconditioning the Tablets by Crisping

Drug abusers often precondition the tablet by a process known as crisping. This procedure is intended to remove some of the tablet fillers, making the drug easier to crush and insufflate or inject. Accordingly, an experiment was performed to determine a drug abuser's ability to crisp a tablet dosage form of the pharmaceutical composition of the present invention containing 7.5 mg oxycodone HCl and 325 mg acetaminophen (see Chart 1) (the “product”) as compared to a commercially available immediate-release tablet containing 7.5 mg oxycodone/325 mg acetaminophen (the “comparator”).

First, the product and comparator tablet were crushed into a powder and placed in a spoon. The spoon was then heated from underneath with an open flame. Once the powder began to caramelize and smoke, the heat was removed and the powder was mixed using a metal spatula. The spoon was again heated until the powder began to caramelize further. The heat was once again and removed, and the powder was allowed to cool. The resulting powders were then removed from the spoon and placed in a mortar and pestle for subsequent crushing. The comparator tablet resulted in a powder that could be easily crushed into a fine powder. Unlike the comparator tablet, the product tablet resulted in a sticky composition, rendering the product tablet unsuitable for grinding into a fine powder after the crisping process.

### Example 19

### Separation Studies

To determine the ease at which the immediate release (IR) and extended release (ER) layers of a bilayer form of the pharmaceutical composition disclosed herein could be tampered with, several attempts were made at separating the immediate release (IR) and extended release (ER) layers of the product (as defined in Example 18). Initially, a tablet dosage form of the pharmaceutical composition of the present invention was positioned with the inscribed side facing up and cut completely through vertically. Upon slicing the tablet, observations revealed no visual distinction between the IR and ER layers. The tablet was then re-oriented and sliced from several additional angles. However, no demarcation line was observed between the IR and ER layers. Consequently, a drug abuser could not visually distinguish the IR and ER layers of the pharmaceutical composition disclosed herein by simply cutting the dosage form.

### Example 20

### Injectability Studies

An injectability study was conducted to determine the extent to which crushed and dissolved tablets of the pharmaceutical composition disclosed herein containing 7.5 mg oxycodone/325 mg acetaminophen (the “product”) could be drawn into a syringe for intravenous administration as compared to a commercially available immediate-release tablet containing 7.5 mg oxycodone/325 mg acetaminophen that had been crushed and dissolved (the “comparator”). Intravenous administration is a common practice used by drug abusers as a means to potentiate their drugs by administering the drug as one large bolus instead of a steady release over time. Two measurable entities were evaluated: the amount of useable fluid that was harvested through the process and the concentration of oxycodone in these aliquots. This study employed a standard 1 mL insulin syringe equipped with 22-, 26-, and 30-gauge needles, which are the typical sizes of needles used by intravenous drug users.

An intact product and comparator tablet were each ground in a mortar and pestle to yield a fine powder. The powder was then placed onto a tablespoon secured to a laboratory ring stand. 3 mL of deionized water was added to the spoon and was mixed into a slurry in an attempt to dissolve the active ingredient. To enhance solubility of the drug, a butane lighter was used to uniformly heat the bottom of the spoon. When the solution began to boil slightly, heat was removed and any liquid lost was replenished. A traditional insulin syringe (1 mL) with a makeshift cotton ball filter and the various gauge needles was used to extract the resulting liquid into the syringe.

Three types of cotton filters were evaluated for use in this procedure. The first filter was a small cotton plug placed between the needle hub and barrel of the syringe. This filter clogged for all three gauges when attempts were made to draw liquid into the syringe. The second filter was formed by inserting the tip of the syringe needle into the end of a Q-tip. This second filter also prevented an appreciable amount of fluid to be drawn into the syringe. The third filter was a small piece of cotton attached to the end of the needle. The third filter was chosen for further study because it was the only filter evaluated in which liquid could be drawn into the syringe for all three gauges without clogging the filter. The drawn liquid was collected, measured and quantified by LC/MS/MS analysis.

When water was mixed with the ground product tablet, the solid did not completely dissolve upon heating. Instead, a pasty material was produced that did not readily disperse when mixed. The product required almost constant mixing of the crushed powder and water with constant heating to produce a removable liquid. It was difficult to generate a homogeneous mixture of liquid that could be drawn into a syringe because the combined volume of the crushed product tablet and the 3 mL of water essentially filled the spoon to capacity. Additionally, with heating, it was necessary to replenish the evaporated water to maintain a constant slurry level in the spoon. Liquid samples were drawn from the bottom of the spoon with a 1 mL syringe with the cotton plug on the tip. This study demonstrated that only about 1 mL of liquid could consistently be drawn into the syringe, independent of needle size. The resulting liquid in the syringe was murky and not transparent due to particulate matter.

In contrast, a large portion of the comparator readily dissolved when mixed and heated in the tablespoon. The resulting liquid in the syringe therefore contained much less particulate matter than the liquid resulting from the product tablet.

These results indicate that injection is not a preferred form of drug diversion for the product tablets. When adding water to the ground tablets, the user may recover only a small portion of that liquid for use in a syringe. The product tablet tended to produce a semi-solid paste that interfered with liquid recovery through the syringe. The overall results indicate a recovery of less than 20% of the oxycodone in the product tablet.

### Example 21

### Snorting Studies

Another method of tampering and diversion is to grind a tablet into a fine powder and insufflate (snort) the powder. The inhaled powder is deposited inside the nasal passage, and the oxycodone is absorbed through the mucous membranes of the nasal passage. In order for the procedure to work efficiently, the powder must deposit as a thin layer onto the nasal tissue in the sinus cavity. A study was performed to estimate the effectiveness of this process using the pharmaceutical composition disclosed herein containing 7.5 mg oxycodone/325 mg acetaminophen (the “product”) and a commercially available immediate-release tablet containing 7.5 mg oxycodone/325 mg acetaminophen (the “comparator”).

Product tablet and a comparator tablet were ground in a mortar and pestle. 1 mL of water was added to each ground tablet, and the resulting combination was mixed in an attempt to produce a thin slurry, which mimics the interface between the nasal passage and the absorptive tissue. The product tablet formed a paste that tended to clump. The comparator produced a more fluid consistency. Consequently, the comparator produced a more effective coating for absorption of insufflated oxycodone in the nasal cavity than the product disclosed herein.

### Example 22

### Dose Dumping Studies

Dose dumping is the process of releasing the active ingredient(s) of an extended release pharmaceutical formulation in a short period of time in a manner in which the entire dosage, or a significant portion of the dosage, becomes available for absorption in the body. This is often achieved by ingesting tablets along with alcoholic beverages to enhance drug delivery. The alcohol serves as a means to act on either the coating of a tablet to help release the active ingredients or to promote greater absorption within the body. This method is employed by drug abusers as an attempt to potentiate analgesic drugs. Release of elevated quantities of drug can lead to increased euphoric effects but can also cause adverse effects, some of which may be fatal.

Two dissolution experiments were performed in a dose dumping study. The dissolutions were designed to examine the differences between intact pharmaceutical compositions disclosed herein containing 7.5 mg oxycodone/325 mg acetaminophen (the “product”) and a commercially available immediate-release tablet containing 7.5 mg oxycodone/325 mg acetaminophen (the “comparator”) when exposed to simulated gastric fluid dissolution media (“SGF”). The first dissolution was performed in 75 mL of SGF in the absence of vodka. The second dissolution was performed in 75 mL of a 50:50 mixture of SGF and 80-proof vodka. This was designed to measure the extent that the product and comparator may be abused by the simultaneous intake of alcohol. Both dissolutions were performed at room temperature and were mixed on a stir plate. Aliquots were removed at 0.25, 0.50, 1, 2 and 4 hours for quantification by LC/MS/MS, a summary of which is contained in Table 54 below.

TABLE 54
Mean percent recovery of oxycodone in (i) simulated gastrointestinal
fluid and (ii) a solution containing 50% simulated gastric fluid and
50% 80-proof vodka.

| | | Mean Percent Recovery at time = t | | | | |
|---|---|---|---|---|---|---|
| Fluid | Intact Tablet | 0.25 hr | 0.5 hr | 1 hr | 2 hr | 4 hr |
| SGF | Product | 15% | 30% | 43% | 57% | 80% |
| SGF | Comparator | 104% | 102% | 105% | 102% | 100% |
| SGF:EtOH | Product | 12% | 23% | 35% | 46% | 62% |
| SGF:EtOH | Comparator | 101% | 101% | 103% | 100% | 102% |

At the end of the four hour dissolution, the product tablets were still visible but had lost their outer coating in SGF both in the presence and absence of vodka. Addition of ethanol to the SGF produced a slight decrease in the dissolution rate of the product tablet. Comparator tablets were dissolved in SGF both in the presence and absence of vodka after five minutes. Consequently, the product tablets were resistant to dose dumping when compared to the comparator tablets.

### Example 23

### Varying Polyox Grades Comprising 25% by Weight of the Extended Release Portion of Bilayer Formulations

Single layer tablet formulations containing only the extended release portion were prepared, each tablet containing a total of 9 mg of oxycodone HCl and a total of 250 mg of acetaminophen. Since these tablets contained only the extended release portion, they contained 50% of the total acetaminophen for the bilayer tablet and 60% of the total oxycodone HCl for the bilayer tablet. In a first formulation, POLYOX® 205 was employed as the extended release component in an amount of 25% by weight of the ER portion, and therefore, the tablet weight. In a second formulation, POLYOX® 1105 was employed as the extended release component in an amount of 25% by weight of the tablet of ER portion. In a third formulation, POLYOX® N-60K was employed as the extended release component in an amount of 25% by weight of the tablet or ER portion.

Dissolution profiles for the three above-described compositions were determined in USP Type II apparatus. Six tablets of each composition were weighed, placed in a sinker, and dropped into an equilibrated dissolution bath vessel containing 900 mL of (helium sparged) 0.1 N HCl heated to 37° C.±0.5° C. The mixture was stirred at 150±6 rpm, and the temperature was maintained at 37° C.±0.5° C. through 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. The final time point for the Polyox 205 was 17 hrs; the final time point for the Polyox 1105 was 15 hrs; and the final time point for the Polyox N60k was 18 hrs and 40 minutes. Each sample was filtered through a 0.45 μm filter and analyzed by HPLC using standard procedures.

The cumulative release profiles of acetaminophen and oxycodone from these compositions are shown in FIGS. 34 and 35, respectively. This data represents dissolution for the extended release portion with the immediate release data theoretically added. These figures demonstrate that as the average molecular weight of the POLYOX® extended release component increases, the rate of dissolution at each time point decreases. For example, the formulations containing POLYOX® 205, 1105, and N-60K had released about 59%, about 56%, and about 55% acetaminophen after 15 minutes, respectively; about 63%, about 59%, and about 57% acetaminophen after 30 minutes, respectively; about 69%, about 64%, and about 61% acetaminophen after 1 hr, respectively; about 78%, about 73%, and about 67% acetaminophen after 2 hr, respectively; about 91%, about 87%, and about 76% acetaminophen after 4 hr, respectively; about 97%, about 95%, and about 84% acetaminophen after 6 hr, respectively; and about 98%, about 97%, and about 90% acetaminophen after 8 hr, respectively.

The same general trend of a decreased release rate with a higher molecular weight POLYOX® grade was also observed for the oxycodone. For example, the formulations containing POLYOX® 205, 1105, and N-60K had released about 53%, about 50%, and about 48% oxycodone after 15 minutes, respectively; about 60%, about 56%, and about 53% oxycodone after 30 minutes, respectively; about 68%, about 63%, and about 59% oxycodone after 1 hr, respectively; about 80%, about 75%, and about 67% oxycodone after 2 hr, respectively; about 94%, about 91%, and about 80% oxycodone after 4 hr, respectively; about 100%, about 98%, and about 89% oxycodone after 6 hr, respectively; and about 100%, about 99%, and about 95% oxycodone after 8 hr, respectively.

### Example 24

### Varying Polyox Grades Comprising 45% by Weight of the Extended Release Portion of Bilayer Formulations

Single layer formulations containing only the extended release portion described herein were prepared, each tablet containing a total of 9 mg of oxycodone HCl and a total of 250 mg of acetaminophen. Since these tablets contained only the extended release portion, they contained 50% of the total acetaminophen for a bilayer tablet and 60% of the total oxycodone HCl for a bilayer tablet. In a first formulation, POLYOX® 205 was employed as the extended release component in an amount of 45% by weight of the tablet or ER portion. In a second formulation, POLYOX® 1105 was employed as the extended release component in an amount of 45% by weight of the tablet or ER portion. In a third formulation, POLYOX® N-60K was employed as the extended release component in an amount of 45% by weight of the tablet or ER portion. The other excipients in the extended release portion were microcrystalline cellulose, spress B825, citric acid anhydrous, EDTA, hydroxypropyl cellulose, silicon dioxide, and magnesium stearate.

Dissolution profiles for the three above-described formulations were determined in USP Type II apparatus. Six tablets of each formulation were weighed, placed in a sinker, and dropped into an equilibrated dissolution bath vessel containing 900 mL of (helium sparged) 0.1 N HCl heated to 37° C.±0.5° C. The mixture was stirred at 150±6 rpm, and the temperature was maintained at 37° C.±0.5° C. through 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. The final time point for the Polyox 205 was 17 hours; the final time point for Polyox 1105 was 17.5 hours; and the final time point for Polyox N60k was 23.5 hours. Each sample was filtered through a 0.45 μm filter and analyzed by HPLC using standard procedures.

The cumulative release profiles of acetaminophen and oxycodone from these compositions are shown in FIGS. 36 and 37, respectively. This data represents dissolution for the extended release portion with the immediate release data theoretically added. Consistent with the results of Example 23, the rate of dissolution at each time point decreases as the molecular weight of POLYOX® increases. For example, the formulations containing POLYOX® 205, 1105, and N-60K had released about 53%, about 53%, and about 53% acetaminophen after 15 minutes, respectively; about 56%, about 55%, and about 54% acetaminophen after 30 minutes, respectively; about 61%, about 60%, and about 57% acetaminophen after 1 hr, respectively; about 70%, about 67%, and about 63% acetaminophen after 2 hr, respectively; about 85%, about 81%, and about 71% acetaminophen after 4 hr, respectively; about 95%, about 90%, and about 79% acetaminophen after 6 hr, respectively; about 99%, about 95%, and about 85% acetaminophen after 8 hr, respectively; and about 99%, about 96% and about 93% acetaminophen after 12 hr.

The formulations containing POLYOX® 205, 1105, and N-60K also released about 47%, about 47%, and about 46% oxycodone after 15 minutes, respectively; about 51%, about 50%, and about 49% after 30 minutes, respectively; about 59%, about 56%, and about 53% oxycodone after 1 hr, respectively; about 70%, about 67%, and about 62% oxycodone after 2 hr, respectively; about 88%, about 83%, and about 74% oxycodone after 4 hr, respectively; about 99%, about 93%, and about 83% oxycodone after 6 hr, respectively; and about 100%, about 97%, and about 90% oxycodone after 8 hr, respectively.

### Example 25

### Varying the Concentrations of a Specific Polyox Grade in the Extended Release Portion of Bilayer Formulations

The data from Examples 23 and 24 indicate that an increase in the amount of POLYOX® in the pharmaceutical composition retards the release of oxycodone and acetaminophen from the pharmaceutical composition. To confirm this observation, single layer extended release formulations described herein were prepared, each containing a total of 9 mg of oxycodone HCl and a total of 250 mg of acetaminophen. Since these tablets contained only the extended release portion, they contained 50% of the total acetaminophen for the bilayer tablet and 60% of the total oxycodone for the bilayer tablet. In a first formulation, POLYOX® 1105 was employed as the extended release component in an amount of 25% by weight of the tablet or ER portion. In a second formulation, POLYOX™ 1105 was employed as the extended release component in an amount of 35% by weight of the tablet or ER portion. In a third formulation, POLYOX™ 1105 was employed as the extended release component in an amount of 45% by weight of the tablet or ER portion. In a fourth formulation, POLYOX® 1105 was employed as the extended release component in an amount of 55% by weight of the tablet or ER portion. The amount of the microcrystalline cellulose in the four formulations was adjusted to account for the differing amounts of POLYOX® 1105 in each formulation. The other excipients in the extended release portion were B825, citric acid anhydrous, EDTA, hydroxypropyl cellulose, silicon dioxide, and magnesium stearate. However, the percentages for all the other excipients remained the same for each formulation, and were consistent with the percentages used in Example 24.

Dissolution profiles for the above-described formulations were determined in USP Type II apparatus. Six tablets of each formulation were weighed, placed in a sinker, and dropped into an equilibrated dissolution bath vessel containing 900 mL of (helium sparged) 0.1 N HCl heated to 37° C.±0.5° C. The mixture was stirred at 150±6 rpm, and the temperature was maintained at 37° C.±0.5° C. through 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. The final time point for the 25%, 35%, 45%, and 55% formulations was 15 hr, 15 hr, 17.5 hr, and 17.5 hr, respectively. Each sample was filtered through a 0.45 μm filter and analyzed by HPLC using standard procedures.

The cumulative release profiles of acetaminophen and oxycodone from these compositions are shown in FIGS. 38 and 39, respectively. These profiles confirm that as the amount of POLYOX® 1105 used in the pharmaceutical formulations increase, the release rate of the acetaminophen and oxycodone generally decreases. For example, the formulations containing 25%, 45%, and 55% POLYOX® 1105 had released about 56%, about 53%, and about 53% acetaminophen after 15 minutes, respectively; about 59%, about 56%, about 55%, and about 55% acetaminophen after 30 minutes, respectively; about 64%, about 61%, about 60%, and about 59% acetaminophen after 1 hr, respectively; about 73%, about 70%, about 67%, and about 66% acetaminophen after 2 hr, respectively; about 87%, about 84%, about 81%, and about 79% acetaminophen after 4 hr, respectively; about 95%, about 93%, about 90%, and about 89% acetaminophen after 6 hr, respectively; about 97%, about 97%, about 95%, and about 95% acetaminophen after 8 hr, respectively; and about 97%, about 97%, about 96%, and about 98% acetaminophen after 12 hr, respectively.

Similar trends were observed for the cumulative release of oxycodone. However, there was no observable difference in the release of oxycodone from the formulations containing 45% and 55% POLYOX® 1105. For example, the formulations containing 25%, 45%, and 55% POLYOX® 1105 had released about 50%, about 47%, and about 45% oxycodone after 15 minutes, respectively; about 56%, about 51%, about 50%, and about 50% oxycodone after 30 minutes, respectively; about 63%, about 58%, about 56%, and about 56% oxycodone after 1 hr, respectively; about 75%, about 70%, about 67%, and about 66% oxycodone after 2 hr, respectively; about 91%, about 87%, about 83%, and about 82% oxycodone after 4 hr, respectively; about 98%, about 96%, about 93%, and about 93% oxycodone after 6 hr, respectively; about 99%, about 99%, about 97%, and about 98% oxycodone after 8 hr, respectively; and about 99%, about 100%, about 97%, and about 100% oxycodone after 12 hr, respectively.

### Example 26

### In vitro Dissolution of Controlled-Release Bilayer Tablets Containing 7.5 mg Oxycodone and 325 mg Acetaminophen Performed at a 100 rpm Paddle Speed

Three batches of bilayer formulations described herein were prepared, each containing a total of 7.5 mg of oxycodone HCl and a total of 325 mg of acetaminophen. 50% of the acetaminophen was contained in the immediate release portion, and the other 50% was contained in the ER layer. 25% of the oxycodone HCl was contained in the immediate release portion of the formulation, and the other 75% was contained in the ER layer. POLYOX™ 1105 was employed as the extended release component in an amount of 45% by weight of the ER portion.

Dissolution profiles for the formulations of each batch were determined in a USP Type II apparatus. Twelve tablets from each batch were weighed, placed in a sinker, and dropped into an equilibrated dissolution bath vessel containing 900 mL of (helium sparged) 0.1 N HCl heated to 37° C.±0.5° C. The mixture was stirred at 100±4 rpm, and the temperature was maintained at 37° C.±0.5° C. for 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. Each sample was filtered through a 0.45 μm filter and analyzed by HPLC using standard procedures.

The cumulative percent release of acetaminophen and oxycodone from each batch are described in Table 55.

TABLE 55
Release rate data of bilayer tablets (7.5 mg oxycodone HCl; 325 mg
acetaminophen) using a 100 rpm dissolution method.

| | Oxycodone HCl | | | | Acetaminophen | | | |
|---|---|---|---|---|---|---|---|---|
| Time | | | | Max | Mean | | | Max |
| (Hours) | Mean (%) | RSD | Min (%) | (%) | (%) | RSD | Min (%) | (%) |

Batch 1

| | | | | | | | | |
|---|---|---|---|---|---|---|---|---|
| 0.25 | 31.7 | 2.1 | 30.6 | 32.5 | 51.8 | 1.4 | 50.9 | 53.1 |
| 0.5 | 37.1 | 1.3 | 36.3 | 37.8 | 54.3 | 1.3 | 53.5 | 55.6 |
| 1.0 | 45.4 | 1.0 | 44.9 | 46.0 | 58.6 | 1.2 | 57.7 | 60.1 |
| 2.0 | 58.5 | 1.3 | 57.4 | 59.7 | 66.0 | 1.2 | 64.8 | 67.7 |
| 4.0 | 78.6 | 1.7 | 76.8 | 80.5 | 78.5 | 1.5 | 77.0 | 80.6 |
| 6.0 | 92.2 | 1.8 | 90.0 | 94.7 | 88.0 | 1.6 | 86.0 | 90.3 |
| 8.0 | 99.5 | 1.8 | 97.4 | 102.7 | 93.8 | 1.5 | 91.8 | 96.3 |
| 12.0 | 101.7 | 1.4 | 99.7 | 104.3 | 96.1 | 1.0 | 94.9 | 98.2 |

Batch 2

| | | | | | | | | |
|---|---|---|---|---|---|---|---|---|
| 0.25 | 31.6 | 3.5 | 29.6 | 34.0 | 52.1 | 4.0 | 48.8 | 55.8 |
| 0.5 | 37.2 | 3.2 | 34.9 | 39.9 | 54.5 | 3.8 | 51.4 | 58.3 |
| 1.0 | 45.4 | 3.3 | 42.4 | 48.3 | 59.1 | 3.5 | 56.0 | 63.1 |
| 2.0 | 58.9 | 1.7 | 57.3 | 61.1 | 66.4 | 3.0 | 63.6 | 70.0 |
| 4.0 | 79.1 | 1.5 | 77.7 | 81.5 | 78.7 | 2.5 | 75.4 | 81.8 |
| 6.0 | 93.1 | 1.3 | 91.5 | 95.8 | 87.7 | 2.2 | 84.4 | 90.7 |
| 8.0 | 100.2 | 1.2 | 98.7 | 102.3 | 93.5 | 1.9 | 90.4 | 96.2 |
| 12.0 | 102.7 | 1.3 | 100.4 | 104.4 | 95.6 | 2.0 | 92.6 | 98.4 |

Batch 3

| | | | | | | | | |
|---|---|---|---|---|---|---|---|---|
| 0.25 | 30.4 | 1.6 | 29.3 | 31.0 | 52.2 | 2.3 | 49.6 | 54.2 |
| 0.5 | 35.7 | 1.6 | 34.2 | 36.7 | 54.6 | 2.3 | 52.0 | 56.6 |
| 1.0 | 43.5 | 1.8 | 42.0 | 45.1 | 58.6 | 2.2 | 56.0 | 60.8 |
| 2.0 | 56.1 | 1.9 | 54.4 | 58.0 | 65.5 | 2.1 | 63.1 | 68.0 |
| 4.0 | 75.4 | 1.8 | 73.3 | 77.6 | 77.3 | 2.0 | 74.8 | 80.0 |
| 6.0 | 88.9 | 1.7 | 86.1 | 91.4 | 86.5 | 2.2 | 83.7 | 90.1 |
| 8.0 | 97.0 | 1.5 | 94.7 | 99.8 | 93.0 | 2.1 | 90.1 | 96.8 |
| 12.0 | 100.4 | 1.1 | 98.7 | 102.4 | 96.5 | 1.6 | 93.2 | 98.3 |

### Example 27

### In vitro Dissolution of Controlled-Release Bilayer Tablets Containing 15 mg Oxycodone and 650 mg Acetaminophen Performed at a 150 rpm Paddle Speed

Bilayer formulations described herein were prepared, each containing a total of 15 mg of oxycodone HCl and a total of 650 mg of acetaminophen. 50% of the acetaminophen was contained in the immediate release portion, and the other 50% was contained in the ER layer. 25% of the oxycodone HCl was contained in the immediate release portion of the formulation, and the other 75% was contained in the ER layer. POLYOX™ 1105 was employed as the extended release component in an amount of 45% by weight of the ER portion.

Dissolution profiles for the formulations were determined in a USP Type II apparatus. Six tablets were weighed, placed in a sinker, and dropped into an equilibrated dissolution bath vessel containing 900 mL of (helium sparged) 0.1 N HCl heated to 37° C.±0.55° C. The mixture was stirred at 150±6 rpm, and the temperature was maintained at 37° C.±0.5° C. for 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. Each sample was filtered through a 0.45 μm filter and analyzed by HPLC using standard procedures.

The cumulative percent release of acetaminophen and oxycodone from each batch are described in Table 56.

TABLE 56
Release rate data of bilayer tablets (15 mg oxycodone HCl; 325 mg
acetaminophen) using a 150 rpm dissolution method.

| Time (hr) | Oxycodone HCl (%) | Acetaminophen (%) |
|---|---|---|
| 0.25 | 33.7 | 54.4 |
| 0.50 | 39.0 | 56.5 |
| 1 | 47.4 | 60.6 |
| 2 | 61.4 | 68.1 |
| 4 | 81.7 | 81.1 |
| 6 | 95.2 | 90.8 |
| 8 | 101.2 | 96.0 |
| 12 | 102.3 | 97.6 |

### Example 28

### Ethanol Release Testing at a 100 rpm Paddle Speed

The ethanol release studies discussed above in Example 8 were repeated, except that the solutions were stirred at a paddle speed of 100 rpm and additional aliquots were sampled at 240 min and 480 min. Tables 57, 58, 59, 60, and 61 present the percent release of OC and APAP in the presence of 0%, 5%, 10%, 20%, and 40% ethanol, respectively. FIG. 40 presents dissolution profiles for OC and FIG. 41 presents dissolution profiles for APAP in the presence of 0%, 5%, 20%, and 40% ethanol. Like the results at a paddle speed of 150 rpm, these data reveal that, for both OC and APAP, the dissolution in 5%, 20%, or 40% ethanol was either comparable or slower than the dissolution in 0% ethanol, indicating no dose dumping for this formulation.

TABLE 57
Percent Release in 0% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 32.5 | 3.7 | 31.5 | 36.0 | 52.2 | 1.6 | 50.7 | 53.4 |
| 30 | 37.6 | 2.5 | 36.6 | 39.9 | 54.6 | 1.4 | 53.2 | 55.7 |
| 45 | 42.1 | 2.7 | 40.9 | 44.8 | 56.8 | 1.4 | 55.3 | 57.9 |
| 60 | 45.8 | 2.1 | 44.6 | 48.1 | 58.8 | 1.4 | 57.4 | 59.8 |
| 75 | 49.6 | 2.3 | 48.2 | 52.2 | 60.8 | 1.4 | 59.2 | 61.8 |
| 90 | 53.1 | 2.4 | 51.7 | 55.8 | 62.6 | 1.4 | 60.9 | 63.8 |
| 105 | 56.3 | 2.4 | 54.8 | 59.3 | 64.3 | 1.4 | 62.6 | 65.6 |
| 120 | 59.5 | 2.5 | 57.6 | 63.0 | 66.0 | 1.4 | 64.2 | 67.3 |
| 240 | 80.3 | 2.5 | 77.3 | 84.9 | 78.6 | 1.8 | 76.3 | 80.6 |
| 480 | 102.4 | 1.8 | 100.5 | 107.2 | 95.5 | 1.6 | 92.6 | 97.7 |

TABLE 58
Percent Release in 5% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 31.5 | 2.5 | 30.0 | 32.9 | 52.6 | 2.1 | 51.4 | 55.1 |
| 30 | 36.8 | 2.4 | 35.6 | 38.5 | 55.1 | 2.0 | 53.8 | 57.6 |
| 45 | 40.9 | 2.8 | 38.9 | 43.5 | 57.1 | 2.0 | 55.8 | 59.6 |
| 60 | 44.6 | 3.7 | 42.1 | 48.4 | 58.9 | 2.0 | 57.6 | 61.4 |
| 75 | 48.0 | 3.6 | 46.0 | 52.6 | 60.7 | 1.9 | 59.4 | 63.2 |
| 90 | 51.0 | 3.1 | 49.3 | 55.3 | 62.3 | 1.9 | 61.0 | 64.7 |
| 105 | 54.3 | 3.2 | 51.8 | 58.6 | 63.9 | 2.0 | 62.6 | 66.4 |
| 120 | 57.1 | 3.2 | 54.6 | 61.7 | 65.5 | 1.9 | 64.1 | 67.8 |
| 240 | 76.6 | 3.2 | 73.8 | 83.0 | 77.2 | 2.1 | 75.5 | 80.6 |
| 480 | 99.9 | 2.7 | 95.8 | 106.8 | 94.4 | 1.7 | 92.6 | 98.1 |

TABLE 59
Percent Release in 10% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 30.3 | 3.1 | 28.9 | 32.1 | 51.7 | 1.8 | 50.1 | 53.4 |
| 30 | 35.6 | 3.3 | 33.7 | 37.3 | 54.1 | 1.9 | 52.4 | 55.8 |
| 45 | 39.6 | 2.6 | 37.6 | 40.9 | 56.0 | 1.9 | 54.3 | 57.8 |
| 60 | 43.1 | 2.6 | 41.2 | 44.7 | 57.8 | 1.9 | 56.1 | 59.5 |
| 75 | 46.2 | 2.3 | 44.1 | 47.5 | 59.5 | 1.8 | 57.7 | 61.1 |
| 90 | 49.3 | 2.1 | 47.3 | 50.6 | 61.1 | 1.8 | 59.3 | 62.8 |
| 105 | 52.2 | 2.2 | 50.1 | 53.6 | 62.6 | 1.8 | 60.9 | 64.2 |
| 120 | 54.8 | 2.3 | 52.8 | 56.4 | 64.1 | 1.8 | 62.3 | 65.6 |
| 240 | 73.8 | 2.2 | 70.8 | 76.1 | 75.5 | 1.7 | 73.4 | 77.4 |
| 480 | 98.4 | 2.1 | 94.7 | 101.1 | 93.5 | 1.6 | 91.0 | 95.9 |

TABLE 60
Percent Release in 20% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 28.0 | 6.0 | 23.9 | 30.3 | 50.2 | 5.1 | 43.0 | 53.0 |
| 30 | 33.6 | 4.5 | 30.7 | 35.6 | 53.4 | 3.1 | 49.5 | 55.9 |
| 45 | 37.9 | 2.9 | 35.7 | 39.6 | 55.5 | 2.6 | 52.6 | 57.9 |
| 60 | 41.2 | 3.1 | 39.2 | 43.2 | 57.3 | 2.3 | 55.1 | 59.8 |
| 75 | 44.1 | 2.9 | 42.3 | 46.6 | 59.0 | 2.2 | 57.0 | 61.4 |
| 90 | 46.5 | 3.5 | 42.7 | 49.1 | 60.5 | 2.1 | 58.6 | 62.9 |
| 105 | 49.8 | 2.9 | 48.0 | 52.8 | 61.9 | 2.1 | 60.2 | 64.4 |
| 120 | 52.2 | 2.8 | 49.9 | 54.8 | 63.3 | 2.0 | 61.7 | 65.9 |
| 240 | 72.2 | 2.1 | 69.4 | 74.7 | 76.0 | 1.7 | 74.1 | 78.4 |
| 480 | 95.7 | 2.3 | 91.7 | 98.7 | 91.9 | 1.7 | 89.3 | 94.6 |

TABLE 61
Percent Release in 40% Ethanol

| Time | OC | | | | APAP | | | |
|---|---|---|---|---|---|---|---|---|
| (Min) | Mean | RSD | Minimum | Maximum | Mean | RSD | Minimum | Maximum |
| 15 | 11.9 | 13.9 | 10.0 | 15.1 | 16.7 | 23.2 | 12.3 | 22.9 |
| 30 | 21.1 | 15.4 | 17.3 | 26.2 | 30.4 | 22.3 | 21.7 | 40.7 |
| 45 | 26.8 | 11.6 | 22.4 | 30.3 | 38.5 | 15.3 | 29.6 | 44.8 |
| 60 | 30.8 | 7.0 | 26.8 | 34.0 | 43.1 | 9.2 | 35.9 | 47.1 |
| 75 | 34.2 | 5.0 | 31.5 | 36.8 | 46.1 | 5.3 | 41.1 | 49.2 |
| 90 | 36.9 | 3.2 | 35.1 | 38.8 | 48.3 | 3.3 | 44.6 | 50.2 |
| 105 | 39.6 | 3.3 | 37.3 | 41.2 | 49.8 | 2.4 | 47.3 | 51.3 |
| 120 | 41.9 | 3.3 | 39.4 | 44.2 | 51.1 | 2.3 | 48.3 | 52.7 |
| 240 | 57.0 | 1.8 | 55.7 | 58.9 | 60.8 | 2.0 | 58.9 | 63.6 |
| 480 | 80.6 | 1.6 | 78.4 | 83.7 | 77.2 | 1.3 | 75.7 | 78.7 |

### Example 29

### Clinical Evaluation of the Relative Abuse Potential of an Extended Release Formulation of Oxycodone and Acetaminophen

A randomized, double-blind, double-dummy, active- and placebo-controlled study was conducted to assess the relative abuse potential of a bilayer tablet comprising an immediate release portion and an extended release portion disclosed herein containing 7.5 mg oxycodone/325 mg acetaminophen (see Chart One) versus an immediate release oxycodone HCl/acetaminophen tablet in non-dependent, recreational opioid users. The study consisted of a screening period, an in-clinic period, and a follow-up period, and was completed by 55 male and female subjects.

After the subjects were screened, in-clinic tests were performed to ensure that the subjects were not physically dependent on opioids, and that they could discriminate between the effects of oxycodone versus the placebo. Upon completion, the study medications were randomly administered as a single oral dose to each subject, and were as follows:

Group A: two tablets disclosed herein containing 7.5 mg oxycodone HCl and 325 mg acetaminophen each plus two placebo tablets disclosed herein plus eight placebo immediate release capsules.

Group B: four tablets disclosed herein containing 7.5 mg oxycodone HCl and 325 mg acetaminophen each plus eight placebo immediate release capsules.

Group C: two immediate release capsules containing 7.5 mg oxycodone HCl and 325 mg acetaminophen each plus six placebo immediate release capsules plus four placebo tablets disclosed herein.

Group D: four immediate release capsules containing 7.5 mg oxycodone HCl and 325 mg acetaminophen each plus four placebo immediate release capsules plus four placebo tablets disclosed herein.

Group E: four crushed tablets disclosed herein containing 7.5 mg oxycodone HCl and 325 mg acetaminophen each placed in eight capsules plus four placebo tablets disclosed herein.

Group F: four crushed immediate release tablets containing 7.5 mg oxycodone HCl and 325 mg acetaminophen each placed in four capsules plus four placebo immediate release capsules plus four placebo tablets disclosed herein.

Group G: four placebo tablets disclosed herein plus eight placebo immediate release capsules.

The study consisted of seven treatment periods, each of which involved a single treatment of one of the study medications followed by a wash-out period. All subjects received each of the seven treatments according to their treatment sequence. Only subjects who completed all 7 arms of the study are included in the results set forth below.

The mean pharmacokinetic parameters for oxycodone are presented in Table 62, and the oxycodone plasma concentration versus time profiles are presented in FIG. 42. Further, the mean pharmacokinetic parameters for acetaminophen are presented in Table 63, and the acetaminophen plasma concentration versus time profiles are presented in FIG. 43.

TABLE 62
Mean pharmacokinetic parameters for oxycodone

| PK | Group | Group | Group | Group | | |
|---|---|---|---|---|---|---|
| Parameters | A | B | C | D | Group E | Group F |
| AUC0-1 | 6.30 | 14.68 | 16.95 | 37.51 | 2.79 | 25.55 |
| (ng · h/mL) | | | | | | |
| AUC0-2 | 18.18 | 38.83 | 43.82 | 85.04 | 20.23 | 67.82 |
| (ng · h/mL) | | | | | | |
| AUC0-8 | 88.05 | 181.16 | 129.57 | 257.73 | 168.05 | 224.68 |
| (ng · h/mL) | | | | | | |
| AUC0-12 | 116.43 | 239.95 | 148.94 | 305.18 | 224.03 | 266.58 |
| (ng · h/mL) | | | | | | |
| AUC0-inf | 167.76 | 339.83 | 166.35 | 349.09 | 286.54 | 303.84 |
| (ng · h/mL) | | | | | | |
| AUC0-t | 153.04 | 313.31 | 163.72 | 343.00 | 276.71 | 298.80 |
| (ng · h/mL) | | | | | | |
| Cmax | 14.42 | 31.36 | 34.09 | 66.15 | 32.22 | 55.11 |
| (ng/mL) | | | | | | |
| Tmax (h)a | 3.02 | 2.65 | 1.25 | 1.26 | 3.54 | 1.49 |
| t½ (h) | 6.46 | 6.32 | 3.92 | 3.85 | 4.18 | 3.83 |

aMedian

TABLE 63
Mean pharmacokinetic parameters for acetaminophen

| PK | | | | | | |
|---|---|---|---|---|---|---|
| Parameters | Group A | Group B | Group C | Group D | Group E | Group F |
| AUC0-1 | 2666.81 | 5650.73 | 4887.49 | 9859.41 | 1042.11 | 7864.78 |
| (ng · h/mL) | | | | | | |
| AUC0-2 | 6024.60 | 12560.98 | 10489.16 | 20372.68 | 4760.07 | 17585.04 |
| (ng · h/mL) | | | | | | |
| AUC0-8 | 17943.23 | 37825.20 | 24864.17 | 50711.32 | 27822.46 | 45789.11 |
| (ng · h/mL) | | | | | | |
| AUC0-12 | 21271.16 | 44611.06 | 27236.97 | 55818.54 | 34594.15 | 50463.26 |
| (ng · h/mL) | | | | | | |
| AUC0-t | 25782.36 | 53465.93 | 29082.51 | 60576.35 | 41504.81 | 54797.92 |
| (ng · h/mL) | | | | | | |
| AUC0-inf | 28522.23 | 57229.79 | 30327.22 | 62661.73 | 43677.32 | 56582.84 |
| (ng · h/mL) | | | | | | |
| Cmax | 4085.26 | 8735.00 | 7822.63 | 15367.59 | 5451.72 | 13688.28 |
| (ng/mL) | | | | | | |
| Tmax (h)a | 0.94 | 0.97 | 0.86 | 0.78 | 2.95 | 1.05 |
| t½ (h) | 7.03 | 6.81 | 4.98 | 6.08 | 5.39 | 5.98 |

aMedian

As evidenced in Tables 62 and 63, the 7.5 mg oxycodone HCl and 325 mg acetaminophen tablets disclosed herein, when crushed, produced lower AUCs and Cmax values and longer Tmax values than crushed immediate release 7.5 mg oxycodone HCl and 325 mg acetaminophen tablets. The subjects also rated the immediate release 7.5 mg oxycodone HCl and 325 mg acetaminophen tablets (Group D) higher than the 7.5 mg oxycodone HCl and 325 mg acetaminophen tablets disclosed herein (Group B) on drug liking, drug high, and good drug effects. See Table 64; see also FIGS. 44-46.

TABLE 64
Analysis of Variance for LS Mean Difference in Emax* - VAS Scores
for Drug Liking, Drug High, and Good Drug Effects

| Parameter | Group B | Group D |
|---|---|---|
| Emax for Drug Liking (points) | 26.373 | 35.558 |
| LS mean | (22.755, 29.992) | (31.940, 39.177) |
| 95% CI | | |
| LS mean difference from | −9.19 | — |
| IR-OC/APAP 30/1300 mg | (−13.140, −5.231) | |
| 95% CI of difference | | |
| Unadjusted p-value | <.001 | |
| Adjusted p-value | <.001 | |
| Emax for Drug High (points) | 47.889 | 75.959 |
| LS mean | (41.395, 54.384) | (69.464, 82.454) |
| 95% CI | | |
| LS mean difference from | −28.07 | — |
| IR-OC/APAP 30/1300 mg | (−34.924, −21.215) | |
| 95% CI of difference | | |
| Unadjusted p-value | <.001 | |
| Adjusted p-value | <.001 | |
| Emax for Good Drug Effects | 55.358 | 75.317 |
| (points) | (47.904, 62.812) | (67.863, 82.772) |
| LS mean | | |
| 95% CI | | |
| LS mean difference from | −19.96 | — |
| IR-OC/APAP 30/1300 mg | (−27.868, −12.050) | |
| 95% CI of difference | | |
| Unadjusted p-value | <.001 | |
| Adjusted p-value | <.001 | |

*maximum effect the drug can cause

Further, it was surprisingly discovered that all the AUC measurements for oxycodone and acetaminophen were lower for treatment Group E (four crushed tablets disclosed herein containing 7.5 mg oxycodone HCl and 325 mg acetaminophen placed in eight capsules for a total dose of 30 mg oxycodone HCl and 1300 mg) than for treatment Group B (four intact tablets disclosed herein containing 7.5 mg oxycodone HCl and 325 mg acetaminophen for a total dose of 30 mg oxycodone HCl and 1300 mg). The Tmax for oxycodone and for acetaminophen was longer for treatment Group E than for treatment Group B. In addition, the Cmax of acetaminophen was lower for treatment Group E than for treatment Group B.

As shown in Table 65, the results also demonstrated that the subjects liked the 7.5 mg oxycodone HCl and 325 mg acetaminophen tablets disclosed herein more when they were intact versus when they were crushed. Thus, the subjects were more likely to take the 7.5 mg oxycodone HCl and 325 mg acetaminophen tablets disclosed herein in intact form than in a crushed form. See Table 65.

TABLE 65*
Summary of Take Drug Again Assessment, ARCI Scores,
and Overall Drug Liking Scores

| | | | | | | | |
|---|---|---|---|---|---|---|---|
| Timepoint | Group A | Group B | Group C | Group D | Group E | Group F | Group G |

Take Drug Again Assessment (“TDAA”)

| | | | | | | | |
|---|---|---|---|---|---|---|---|
| 1 h | 59.79 | 70.98 | 76.43 | 80.17 | 55.34 | 77.59 | 52.19 |
| 4 h | 59.84 | 74.41 | 71.50 | 74.07 | 68.81 | 78.07 | 49.83 |
| 8 h | 58.69 | 72.18 | 69.29 | 72.68 | 65.00 | 73.72 | 50.12 |
| 24 h | 58.22 | 71.34 | 68.38 | 71.68 | 66.45 | 74.47 | 49.38 |

ARCI/AMBG Scores

| | | | | | | | |
|---|---|---|---|---|---|---|---|
| Amphetamine | | | | | | | |
| Group Score | | | | | | | |
| 0.5 h | 11.72 | 12.96 | 12.33 | 13.78 | 10.45 | 12.83 | 10.53 |
| 1 h | 13.12 | 16.44 | 17.03 | 18.46 | 12.48 | 17.72 | 10.59 |
| 2 h | 12.46 | 15.80 | 14.48 | 15.90 | 14.91 | 16.66 | 10.60 |
| 4 h | 10.91 | 13.84 | 12.17 | 13.58 | 14.00 | 13.97 | 9.83 |
| Morphine | | | | | | | |
| Benzedrine | | | | | | | |
| Group Score | | | | | | | |
| 0.5 h | 14.90 | 17.43 | 16.19 | 19.44 | 12.81 | 16.78 | 12.41 |
| 1 h | 17.66 | 24.62 | 26.33 | 29.14 | 15.64 | 25.50 | 12.64 |
| 2 h | 17.25 | 23.61 | 23.03 | 24.73 | 20.88 | 25.55 | 13.05 |
| 4 h | 14.28 | 19.82 | 17.40 | 20.10 | 19.91 | 20.78 | 11.48 |

Global Assessment Overall Drug Liking

| | | | | | | | |
|---|---|---|---|---|---|---|---|
| 8 h | 60.14 | 72.78 | 68.36 | 73.66 | 67.52 | 74.07 | 49.65 |
| 24 h | 58.38 | 70.50 | 68.03 | 70.83 | 67.59 | 73.95 | 50.90 |

*Mean data from the mITT Population as data was unavailable for all 55 subjects.

Further, the pupils of the subjects who completed the study were measured prior to administration of any of the study medications, and prior to each pharmacokinetic sample collection timepoint for up to 12 hours to determine the effect of the oxycodone on their pupils. The percentage of the subjects pupils' constriction is presented in Table 66 and FIG. 47.

TABLE 66
Summary of Pupillometry Pharmacodynamic Response from the
Pharmacokinetic/Pharmacodynamic Model of Oxycodone
Concentration and Effect on Pupil Diameter (Completers Population) --
% Constriction

| | | | | | Planar 95% |
|---|---|---|---|---|---|
| | Model | | Standard | | CI |
| Treatment | Parameter | Estimate | Error | CV % | Lower-Upper |
| Group A | Imax (%) | 21.87 | 1.41 | 6.4 | 18.99-24.75 |
| | Ke0 (1/hr) | 1.66 | 0.38 | 22.7 | 0.89-2.42 |
| | IC50 | 5.10 | 0.61 | 12.0 | 3.85-6.35 |
| | (ng/mL) | | | | |
| | Gamma | 3.79 | 1.30 | 34.3 | 1.13-6.45 |
| Group B | Imax (%) | 36.42 | 1.31 | 3.6 | 33.73-39.10 |
| | Ke0 (1/hr) | 1.79 | 0.25 | 14.2 | 1.27-2.31 |
| | IC50 | 11.36 | 0.73 | 6.5 | 9.87-12.86 |
| | (ng/mL) | | | | |
| | Gamma | 4.21 | 0.81 | 19.2 | 2.56-5.86 |
| Group C | Imax (%) | 59.78 | 20.76 | 34.7 | 17.38-102.18 |
| | Ke0 (1/hr) | 1.38 | 0.12 | 8.8 | 1.13-1.63 |
| | IC50 | 17.68 | 6.51 | 36.8 | 4.39-30.97 |
| | (ng/mL) | | | | |
| | Gamma | 2.04 | 0.58 | 28.5 | 0.85-3.23 |
| Group D | Imax (%) | 47.86 | 2.65 | 5.5 | 42.45-53.28 |
| | Ke0 (1/hr) | 0.92 | 0.11 | 12.0 | 0.70-1.15 |
| | IC50 | 18.50 | 1.83 | 9.9 | 14.76-22.23 |
| | (ng/mL) | | | | |
| | Gamma | 3.72 | 0.48 | 12.8 | 2.74-4.69 |
| Group E | Imax (%) | 40.76 | 3.11 | 7.6 | 34.41-47.10 |
| | Ke0 (1/hr) | 1.38 | 0.20 | 14.6 | 0.97-1.80 |
| | IC50 | 11.76 | 1.09 | 9.3 | 9.53-13.99 |
| | (ng/mL) | | | | |
| | Gamma | 2.66 | 0.51 | 19.2 | 1.62-3.70 |
| Group F | Imax (%) | 48.14 | 3.44 | 7.1 | 41.11-55.16 |
| | Ke0 (1/hr) | 1.09 | 0.11 | 10.2 | 0.86-1.32 |
| | IC50 | 17.32 | 1.72 | 9.9 | 13.81-20.83 |
| | (ng/mL) | | | | |
| | Gamma | 2.91 | 0.39 | 13.4 | 2.12-3.71 |

### Example 30

### Single Dose Pharmacokinetic Analysis of an Extended Release Formulation of Oxycodone and Acetaminophen Administered Under Fasted Conditions

An open-label, randomized, single-dose, four-period crossover study to evaluate the pharmacokinetics, bioavailability and safety of a bilayer tablet formulation comprising an immediate release portion and an extended release portion containing 7.5 mg oxycodone and 325 mg acetaminophen (see selected example from Chart No. 1) (“Treatment A”) with either a commercially-available tablet containing 15 mg oxycodone (“Treatment B”) or a commercially-available tablet containing 37.5 mg tramadol and 325 mg acetaminophen (“Treatment C”) and a commercially-available immediate release tablet containing 7.5 mg oxycodone and 325 mg acetaminophen (“Treatment D”), was conducted in healthy subjects in a fasted state.

The subjects were randomly assigned to 1 of 4 treatment sequences: A/D/B/C, B/A/C/D, C/B/D/A, or D/C/A/B. As mentioned above, Treatments A, B, C, and D were as follows:
Treatment A: 2 tablets of a bilayer tablet formulation containing 7.5 mg oxycodone and 325 mg acetaminophen administered orally under fasted conditions, 1 tablet at a time, at Hour 0 on Day 1 of the period;
Treatment B: 1 tablet of a commercially-available tablet containing 15 mg oxycodone administered orally under fasted conditions at Hour 0 and Hour 6 on Day 1 of the period;
Treatment C: 1 tablet of a commercially-available tablet containing 37.5 mg tramadol and 325 mg acetaminophen administered orally under fasted conditions at Hour 0 and Hour 6 on Day 1 of the period; and
Treatment D: 1 tablet of a commercially-available immediate release tablet containing 7.5 mg oxycodone and 325 mg acetaminophen administered orally under fasted conditions at Hour 0 and Hour 6 on Day 1 of the period.

Each period started at the check-in and ended at the check-in of the subsequent period. There was a minimum 7-day interval between the start of each period. Subjects received their assigned study drug treatment beginning on Hour 0 of Day 1. In all 4 treatment periods, the subjects fasted for at least 10 hours before administration of the study drug at Hour 0. Further, for Treatments B, C, and D, the subjects also fasted for at least 1 hour before the Hour 6 study drug administration.

During each period, blood samples were collected at designated times before dosing and up to 36 hours after dosing for a pharmacokinetic analysis of oxycodone and acetaminophen. The pharmacokinetic parameters for oxycodone are presented in Table 67, and the oxycodone plasma concentration versus time profiles are presented in FIG. 48. Further, the pharmacokinetic parameters for acetaminophen are presented in Table 68, and the acetaminophen plasma concentration versus time profiles are presented in FIG. 49.

TABLE 67
Pharmacokinetic Parameters for Oxycodone by Treatment
(2 hours after second dose)

| | N = 29 | | AUCt | AUCinf | | | | |
|---|---|---|---|---|---|---|---|---|
| | F = 7 | Cmax | (ng · h/ | (ng · h/ | Tmax | Tlag | Kel | t½ |
| Treatment | M = 22 | (ng/mL) | mL) | mL) | (h) | (h) | (1/h) | (h) |
| A | Mean | 14.28 | 167.93 | 169.38 | | | | |
| | Median | 14.9 | 166.33 | 167.26 | 4 | 0 | 0.1579 | 4.39 |
| | % CV | 20.6 | 21.9 | 21.9 | 69.3 | 199.3 | 15.4 | 14.5 |
| B | Mean | 31.27 | 334.77 | 336.46 | | | | |
| | Median | 30.8 | 338.91 | 341.14 | 8 | 0 | 0.1793 | 3.87 |
| | % CV | 26.1 | 18.7 | 18.7 | 74.8 | 254.4 | 8.5 | 8.5 |
| D | Mean | 19.42 | 169.91 | 171.59 | | | | |
| | Median | 19.4 | 166.17 | 167.09 | 8 | 0 | 0.1721 | 4.03 |
| | % CV | 23.8 | 20.2 | 19.9 | 61.4 | 199.3 | 13.7 | 12.9 |

TABLE 68
Pharmacokinetic Parameters for Acetaminophen by Treatment

| | N = 29 | | | | | | | |
|---|---|---|---|---|---|---|---|---|
| | F = 7 | Cmax | AUCt | AUCinf | Tmax | Tlag | Kel | t½ |
| Treatment | M = 22 | (ng/mL) | (ng · h/mL) | (ng · h/mL) | (h) | (h) | (1/h) | (h) |
| A | Mean | 4653.79 | 29105.98 | 30897.99 | | | | |
| | Median | 4420 | 28862.75 | 29979.62 | 0.75 | 0 | 0.1364 | 5.08 |
| | % CV | 29.2 | 23.6 | 23.1 | 50.2 | 299.6 | 30.9 | 41.6 |
| C | Mean | 4255.52 | 29953.73 | 31051.84 | | | | |
| | Median | 4320 | 29639 | 30402.51 | 2 | 0 | 0.1783 | 3.89 |
| | % CV | 23.6 | 21.9 | 21.6 | 96.1 | 160.3 | 24.8 | 31.5 |
| D | Mean | 4387.24 | 29191.1 | 30316.61 | | | | |
| | Median | 4430 | 28834.25 | 29858.94 | 0.75 | 0 | 0.1694 | 4.09 |
| | % CV | 30.2 | 23.6 | 23.6 | 125.3 | 373.9 | 26.6 | 28.1 |

As evidenced in Tables 67 and 68 above, there was no lag in absorption of either oxycodone or acetaminophen for any of the Treatments. The dose-normalized pharmacokinetic parameters of oxycodone following single and multiple doses of the extended release formulation disclosed herein (Treatment A) were equivalent to the commercially-available oxycodone tablet (Treatment B) with respect to Cmax, AUC0-t, and AUC0-inf (single dose). There was also no significant difference in the median Tmax of oxycodone between Treatment A and Treatment B. In addition, there was no lag (median tlag=0) in the plasma oxycodone levels for any of the treatments.

Similarly, dose-normalized pharmacokinetic parameters of acetaminophen following a single dose of the extended release formulation disclosed herein (Treatment A) were equivalent to the commercially-available tramadol/acetaminophen tablet (Treatment C) with respect to Cₘₐₓ, AUC₀₋ₜ, and AUC_{0-inf}. The median Tₘₐₓ of acetaminophen from Treatment A occurred significantly earlier than the median Tₘₐₓ for Treatment C. Further, the plasma concentrations of acetaminophen in subjects who were administered Treatment A were less than 1000 ng/mL 12 hours post-dose. There was also no lag (median t_{lag}=0) in the plasma acetaminophen levels for any of the treatments.

Consequently, the comparable PK findings between treatment groups in this study support the use of the extended release formulation containing 7.5 mg oxycodone and 325 mg acetaminophen disclosed herein (Treatment A) for acute pain over the proposed 12-hour dosing interval.

### Example 31

### Multiple Dose Pharmacokinetic Analysis of an Extended Release Formulation of Oxycodone and Acetaminophen Administered Under Fasted Conditions

An open-label, randomized, multiple-dose, four-period crossover study to evaluate the pharmacokinetics, bioavailability and safety a bilayer tablet formulation comprising an immediate release portion and an extended release portion containing 7.5 mg oxycodone and 325 mg acetaminophen (see selected example from Chart No. 1) with either a commercially-available tablet containing 15 mg oxycodone or a commercially-available tablet containing 37.5 mg tramadol and 325 mg acetaminophen was conducted in subjects in a fasted state. All treatments were then compared against a commercially-available immediate release tablet containing 7.5 mg oxycodone and 325 mg acetaminophen.

The subjects were randomly assigned to 1 of 4 treatment sequences: A/D/B/C, B/A/C/D, C/B/D/A, or D/C/A/B. Treatments A, B, C, and D were as follows:
Treatment A: 2 tablets of a bilayer tablet formulation disclosed herein containing 7.5 mg oxycodone and 325 mg acetaminophen administered orally, 1 tablet at a time, Q12 h for 4.5 days (9 doses).
Treatment B: 1 tablet of a commercially-available tablet containing 15 mg oxycodone administered orally Q6 h for 4.5 days (18 doses).
Treatment C: 1 tablet of a commercially-available tablet containing 37.5 mg tramadol and 325 mg acetaminophen administered orally Q6 h for 4.5 days (18 doses).
Treatment D: 1 tablet of a commercially-available immediate release tablet containing 7.5 mg oxycodone and 325 mg acetaminophen administered orally Q6 h for 4.5 days (18 doses).

For Treatment A, at Hour 0 on Day 1 and Hour 96 on Day 5, subjects received 2 tablets of the extended release tablet formulation disclosed herein containing 7.5 mg oxycodone and 325 mg acetaminophen after an overnight fast of at least 10 hours. Subjects received subsequent doses of Treatment A administered Q12 h at Hours 12, 24, 36, 48, 60, 72, and 84 after a fast of at least 1 hour.

For Treatments B, C, and D, at Hour 0 on Day 1 and Hour 96 on Day 5, subjects received 1 tablet of either a commercially-available tablet containing 15 mg oxycodone, a commercially-available tablet containing 37.5 mg tramadol and 325 mg acetaminophen, or a commercially-available immediate release tablet containing 7.5 mg oxycodone and 325 mg acetaminophen after an overnight fast of at least 10 hours. Subjects received subsequent doses of Treatments B, C, or D administered Q6 h at Hours 6, 12, 18, 24, 30, 36, 42, 48, 54, 60, 66, 72, 78, 84, 90, and 102 after a fast of at least 1 hour.

During each period, serial blood samples for pharmacokinetic analysis of oxycodone and acetaminophen were collected before dosing and up to 12 hours after dosing. For steady-state analysis, blood samples were collected on Days 2 through 4 before the morning dose of study drug and on Day 5 before dosing and through 132 hours (Day 6).

The mean pharmacokinetic parameters for oxycodone at steady states are presented in Table 69, and the mean oxycodone plasma concentration versus time profiles are presented in FIG. 50. As evidenced in Table 69, there was no lag in absorption of oxycodone for Treatments A, B, and D. The median Tmaxss for oxycodone was significantly shorter for Treatment A than for Treatment D. Moreover, the total systemic and peak exposure of oxycodone for Treatment A at steady state was equivalent to Treatment B when measured by Cmaxss, Cminss, and AUC0-12 hss on Day 5. A patient's exposure to oxycodone (AUC0-12 hss) and (Cmaxss) of Treatment A over the dosing interval at steady state was bioequivalent to Treatment B. The Cmaxss, Cminss, Cavgss, and AUC0-12 hss of oxycodone for Treatment A was also equivalent to Treatment D.

TABLE 69
Mean Pharmacokinetic Parameters for Oxycodone at Steady State

| | Treatment A | | Treatment B | | Treatment D | |
|---|---|---|---|---|---|---|
| | (2 tablets Q12h) | | (1 tablet Q6h) | | (1 tablet Q6h) | |
| Parameter | (n = 24) | | (n = 24) | | (n = 24) | |
| AUC_{0-12 h}^{ss} (ng · h/mL) | 208.34 | (45.34) | 376.88 | (83.90) | 191.54 | (42.81) |
| C_{avg}^{ss} (ng/mL) | 17.36 | (3.78) | 31.41 | (6.99) | 15.96 | (3.57) |
| Cₘₐₓ^{ss} (ng/mL) | 24.00 | (5.38) | 45.15 | (10.54) | 26.32 | (6.18) |
| Cₘᵢₙ^{ss} (ng/mL) | 9.31 | (2.39) | 19.91 | (4.93) | 8.81 | (2.40) |
| DFL (%) | 83.89 | (17.58) | 79.94 | (19.83) | 110.90 | (33.39) |
| Swing | 1.65 | (0.58) | 1.32 | (0.50) | 2.13 | (0.94) |
| Tₘₐₓ^{ss} (h)^{a} | 3.00 | (1.00, 5.92) | 3.00 | (1.00, 12.00) | 7.25 | (0.50, 8.13) |
| Days 5 through 6 | | | | | | |
| Kₑₗ (1/h) | 0.1318 | (0.0223) | 0.1525 | (0.0206) | 0.1517 | (0.0205) |
| t_{1/2} (h) | 5.40 | (0.87) | 4.62 | (0.59) | 4.65 | (0.62) |

^{a}For Tₘₐₓ and t_{lag}, the median (minimum, maximum) values are presented.

The mean pharmacokinetic parameters for acetaminophen at steady state are presented in Table 70, and the mean acetaminophen plasma concentration versus time profiles are presented in FIG. 51. As evidenced in Table 70, no significant difference in Tmaxss for acetaminophen was observed between Treatments A, C, and D. Further, the dose-normalized pharmacokinetic parameters of acetaminophen for the extended release formulation disclosed herein (Treatment A) were equivalent to the commercially-available tramadol/acetaminophen tablet (Treatment C) with respect to Cmaxss, Cavgss, and AUC0-12 hss. The dose-normalized Cminss of acetaminophen for Treatment A was approximately 21% lower than Cminss of acetaminophen for Treatment C, and 22% lower than Cₘᵢₙ^{ss} of acetaminophen for Treatment D. In contrast, the dose-normalized Cₘᵢₙ^{ss} of acetaminophen for Treatment C was equivalent to the Cₘᵢₙ^{ss} of acetaminophen for Treatment D.

The degree of fluctuation of and swing in the plasma concentrations of acetaminophen were also comparable between Treatments A, C, and D. In addition, the plasma concentrations of acetaminophen in subjects who were administered Treatment A decreased below 1000 ng/mL between doses.

TABLE 70
Mean Pharmacokinetic Parameters for Acetaminophen at Steady
State

| | Treatment A | | Treatment C | | Treatment D | |
|---|---|---|---|---|---|---|
| | (2 tablets Q12h) | | (1 tablet Q6h) | | (1 tablet Q6h) | |
| Parameter | (n = 24) | | (n = 24) | | (n = 24) | |
| AUC_{0-12 h}^{ss} | 28160.40 | (5807.09) | 29711.92 | (5427.37) | 29284.22 | (5477.73) |
| (ng · h/mL) | | | | | | |
| C_{avg}^{ss} (ng/mL) | 2346.70 | (483.92) | 2475.99 | (452.28) | 2440.35 | (456.48) |
| Cₘₐₓ^{ss} (ng/mL) | 4792.50 | (1132.40) | 5078.33 | (1189.70) | 4876.67 | (1383.08) |
| Cₘᵢₙ^{ss} (ng/mL) | 852.75 | (273.25) | 1070.92 | (367.35) | 1069.13 | (291.83) |
| DFL (%) | 169.13 | (39.83) | 163.90 | (47.17) | 155.25 | (38.77) |
| swing | 5.08 | (2.07) | 4.22 | (2.14) | 3.81 | (1.63) |
| Tₘₐₓ^{ss} (h)^{a} | 1.00 | (0.50, 4.00) | 0.88 | (0.25, 8.00) | 0.75 | (0.25, 8.00) |
| Days 5 through 6 | | | | | | |
| Kₑₗ (1/h) | 0.1072 | (0.0285) | 0.1355 | (0.0279) | 0.1201 | (0.0338) |
| t_{1/2} (h) | 6.90 | (1.76) | 5.32 | (1.10) | 6.21 | (1.79) |

^{a}For Tₘₐₓ and t_{lag}, the median (minimum, maximum) values are presented.

Thus, the comparable PK findings between treatment groups in this study support the use of the extended release formulation containing 7.5 mg oxycodone and 325 mg acetaminophen disclosed herein (Treatment A) for acute pain over the proposed 12-hour dosing interval.

### Example 32

### Clinical Half-Value Duration Analysis for Oxycodone After Single and Multiple Doses of Controlled-Release Oxycodone/Acetaminophen (CR OC/APAP) Tablets

Controlled-release (CR) formulations are designed to reduce peak-to-trough fluctuations in plasma concentrations of medications, which may result in lower peak concentrations and potential reduction in the incidence and/or intensity of adverse events. Conventional pharmacokinetic (PK) measures, such as maximum plasma concentration (Cₘₐₓ) and time to Cₘₐₓ (Tₘₐₓ), alone may not sufficiently describe the characteristics of CR formulations. Half-value duration (HVD) does not provide direct information about a medication's duration of action; however, it has been considered a clinical correlate of the in vivo duration of action of a CR medication and as a complementary measure to traditional PK measures to more fully describe the performance characteristics of CR formulations. HVD is defined as the period of time during a dosing cycle that plasma concentrations are equal to or above the half of the maximum concentration (≧50% Cₘₐₓ).

A study analyzing the PK data from two randomized, open-label, crossover studies (1 single-dose and 1 multiple-dose study) was conducted. PK data were analyzed for 29 subjects in the single-dose study and 24 subjects in the multiple-dose study. Demographics and baseline characteristics of subjects were similar in the 2 studies. Participants meeting any of the following criteria were excluded from the study: current recreational drug use; history of abuse/addiction or recent illicit drug use (within 2 years) or nicotine use (within 6 months); history of any condition that may interfere with the absorption, distribution, metabolism, or excretion of study medication; or history of gastric bypass or gastric band surgery.

The following treatments were administered under fasted conditions:
Oral doses of CR OC/APAP (7.5 mg OC/325 mg APAP) (see selected example from Chart No. 1) administered as 2 tablets (total dose: 15 mg/650 mg) taken once (single-dose study) or 2 tablets taken twice daily every 12 hours over 4.5 days for 9 doses (multiple-dose study)
Oral doses of commercially available IR OC/APAP 7.5 mg/325 mg (see selected example from Chart No. 1), administered during a separate trial period as 1 tablet every 6 hours for 2 doses (single-dose study) or 1 tablet every 6 hours over 4.5 days for 18 doses (multiple-dose study)

Blood samples for PK analysis were collected up to 36 hours (single-dose study) or up to 132 hours (after the hour-0 dose; multiple-dose study). Plasma oxycodone concentrations were determined using a validated liquid chromatography/tandem mass spectrometry (LC-MS/MS) method. HVD, degree of fluctuation, Cₘₐₓ, Tₘₐₓ, and area under the concentration-time curve (AUC) were calculated for oxycodone. Safety and tolerability were monitored throughout each study.

Individual plasma concentration versus actual time data were used to estimate the PK parameters of oxycodone by standard noncompartmental methods. In both the single-dose and multiple-dose studies, analysis of variance was performed to compare treatments using the natural log-transformed, dose-normalized (amount absorbed corrected by dose administered) PK parameters (Cₘₐₓ and AUC) or natural log-transformed PK parameters (degree of fluctuation) as the dependent variables, with sequence, treatment, and period as fixed effects and subjects nested within sequences as random effect. HVD of oxycodone after CR OC/APAP (single- or multiple-dose) was compared with that after IR OC/APAP using paired 2-tailed t tests. Descriptive statistics and paired t tests were calculated for the percentage difference in HVD relative to the IR product (% RDHVD). The % RDHVD was calculated as the average percent difference in HVD for CR OC/APAP relative to IR OC/APAP for individual subjects.

FIG. 52 presents the plasma oxycodone concentration over time during the single-dose study. FIG. 52 further presents the HVD for the CR and IR formulations administered. Moreover, Table 71 provides a summary of the PK measures for oxycodone in the initial 12 hours after dosing, for both the single-dose study and the multi-dose study. In the single-dose study, the HVD of oxycodone was significantly greater for CR OC/APAP compared with IR OC/APAP (9.65 h vs 5.94 h, difference of 3.71 h, P<0.0001). There was a significant increase in HVD by 77.5% for CR OC/APAP versus IR OC/APAP (P<0.0001). AUC values for oxycodone after administration of CR OC/APAP and IR OC/APAP (dose-normalized) were comparable. The Cₘₐₓ of oxycodone (normalized for dose) was 27% lower for CR OC/APAP compared with IR OC/APAP during the initial 12 hours after dosing. Oxycodone Tₘₐₓ was observed at 4 hours for CR OC/APAP and 8 hours (2 hours after the second dose) for IR OC/APAP.

TABLE 71
Pharmacokinetic Measures for Oxycodone, Initial 12 Hours After Dosing

| | | | | | Multiple-Dose | | | |
|---|---|---|---|---|---|---|---|---|
| Parameter, | Single-Dose Study | | | | Study, Day 1 | | | |
| mean (SD) | CR OC/APAP | | IR OC/APAP | | CR OC/APAP | | IR OC/APAP | |
| Cₘₐₓ, ng/mL | 14.28 | (2.9) | 19.42 | (4.6) | 16.04 | (3.6) | 19.83 | (5.1) |
| Tₘₐₓ, h^{a} | 4.00 | (0.75-12.0) | 8.00 | (0.5-12.0) | 3.00 | (0.5-8.0) | 8.00 | (0.5-10.0) |
| AUC, ng · h/mL | 169.34 | (37.0)^{b} | 171.53 | (34.1)^{b} | 136.14 | (23.7)^{c} | 132.45 | (22.8)^{c} |
| HVD, h | 9.65 | (2.8) | 5.94 | (2.2) | 7.9 | (1.7) | 5.54 | (2.5) |

^{a}Median (range)
^{b}AUC_{0-inf}
^{c}AUC_{0-12 h}

FIG. 53 presents the plasma oxycodone concentration versus time during the first day of the multi-dose study. FIG. 53 further presents the HVD for the CR and IR formulations administered. Moreover, as explained above, Table 71 provides a summary of the PK measures for oxycodone in the initial 12 hours after dosing, for both the single-dose study and the multi-dose study. In the multi-dose study, the HVD for oxycodone over the dosing interval on day 1 was significantly greater for CR OC/APAP compared with IR OC/APAP (7.90 h vs 5.54 h, difference of 2.35 h, P<0.0001). There was a significant increase in HVD by 70.5% for CR OC/APAP versus IR OC/APAP (P=0.0002). After initial dosing (day 1) in the multiple-dose study, the Cₘₐₓ of oxycodone was lower for CR OC/APAP compared with IR OC/APAP. AUC was similar between treatments.

FIG. 54 presents the steady-state plasma oxycodone concentration versus time during day 5 of the multi-dose study. FIG. 54 further presents the HVD for the CR and IR formulations administered. Furthermore, Table 72 presents a summary of the PK measures for oxycodone for day 5 of the multi-dose study. HVD at steady state (day 5) was significantly greater for CR OC/APAP compared with IR OC/APAP (7.85 h vs 5.79 h, difference of 2.06 h, P=0.0008). There was a significant increase in HVD by 65.6% for CR OC/APAP versus IR OC/APAP (P=0.0002). Cₘₐₓ and AUC for oxycodone were similar after steady-state dosing of CR OC/APAP and IR OC/APAP. Degree of fluctuation at steady state was 23% lower for CR OC/APAP compared with IR OC/APAP (84% vs 111%).

TABLE 72
Steady-State Pharmacokinetic Measures for Oxycodone (day 5)

| Parameter | CR OC/APAP | | IR OC/APAP | |
|---|---|---|---|---|
| Cₘₐₓ, ng/mL^{a} | 24.00 | (5.4) | 26.32 | (6.2) |
| Tₘₐₓ, h^{b} | 3.00 | (1.0-5.9) | 7.25 | (0.5-8.13) |
| AUC_{0-12 h}, ng · h/mL^{a,c} | 208.34 | (45.3) | 191.54 | (42.8) |
| Degree of fluctuation, % | 83.89 | (17.6) | 110.9 | (33.4) |
| HVD, h^{a} | 7.85 | (1.4) | 5.79 | (2.8) |

^{a}Mean (SD)
^{b}Median (range)
^{c}Mean exposure within 0-12 hours after the hour-96 dose (day 5, at steady state).

Table 73 summarizes the most frequently occurring treatment-emergent adverse events (TEAEs) reported during the single-dose and multi-dose studies. The most frequently reported TEAEs following administration of CR OC/APAP were nausea, vomiting, pruritus, dizziness, and headache.

TABLE 73
Most Frequently Occurring Treatment-Emergent
Adverse Events (≧5% with CR OC/APAP)

| | Single-Dose Study | | Multiple-Dose Study | |
|---|---|---|---|---|
| Treatment-Emergent | CR | IR | CR | IR |
| Adverse Event, n | OC/APAP | OC/APAP | OC/APAP | OC/APAP |
| (%) | (n = 39) | (n = 40) | (n = 33) | (n = 31) |
| Any TEAE | 9 (23.1) | 15 (37.5) | 15 (45.5) | 20 (64.5) |
| Nausea | 5 (12.8) | 9 (22.5) | 8 (24.2) | 9 (29.0) |
| Dizziness | 3 (7.7) | 4 (10.0) | 4 (12.1) | 4 (12.9) |
| Vomiting | 1 (2.6) | 1 (2.5) | 7 (21.2) | 5 (16.1) |
| Headache | 1 (2.6) | 3 (7.5) | 5 (15.2) | 3 (9.7) |
| Somnolence | 3 (7.7) | 3 (7.5) | 1 (3.0) | 1 (3.2) |
| Pruritus | 1 (2.6) | 1 (2.5) | 7 (21.2) | 2 (6.5) |
| Feeling hot | 1 (2.6) | 2 (5.0) | 2 (6.1) | 2 (6.5) |
| Abdominal pain | 1 (2.6) | 0 (0.0) | 2 (6.1) | 3 (9.7) |

The results of this study demonstrate that administration of CR OC/APAP resulted in a greater time above 50% of the Cₘₐₓ for oxycodone compared with administration of IR OC/APAP. At steady state, CR OC/APAP was associated with significantly less fluctuation in plasma oxycodone concentrations compared with IR OC/APAP. Traditional PK measures indicate equivalent or lower Cₘₐₓ and equivalent AUC for oxycodone after administration of CR OC/APAP compared with IR OC/APAP. CR OC/APAP was generally well tolerated, with TEAEs that were consistent with those associated with opioid therapy. The results of this PK analysis support the administration of CR OC/APAP every 12 hours for the management of moderate to severe acute pain.

### Example 33

### Dose Proportionality and Linearity of Acetaminophen after Single or Multiple Oral Doses of Controlled-Release Oxycodone/Acetaminophen (CR OC/APAP) Tablets

Data were pooled from randomized, crossover, single- and multiple-dose studies conducted in healthy adults (aged 18-55 years) to evaluate the dose proportionality and dose linearity of the APAP component of CR OC/APAP when administered as 1, 2, or 4 tablets (325 mg, 650 mg, or 1300 mg APAP, respectively). CR OC/APAP tablets employ a dual-layer biphasic delivery mechanism that, when administered as 2 tablets, include an immediate-release component delivering 3.75 mg OC/325 mg APAP and an extended-release component delivering 11.25 mg OC/325 mg APAP. (See selected example from Chart No. 1)

The following treatments were administered under fasted conditions:
Single-dose studies: 1, 2, or 4 tablets of CR OC/APAP (325 mg, 650 mg, or 1300 mg APAP, respectively) (see selected example from Chart No. 1) administered orally once
Multiple-dose studies: 1 or 2 tablets of CR OC/APAP (325 mg or 650 mg APAP, respectively) (see selected example from Chart No. 1) administered orally every 12 hours for 4.5 days (9 doses)

One single-dose study (highest dosage) enrolled healthy recreational drug users; all other studies enrolled healthy subjects with specific exclusions for drug use.

Blood samples for plasma analysis of APAP were collected up to 48 hours after dosing in single-dose studies and up to 144 hours after the hour-0 dose in multiple-dose studies. Area under the plasma APAP concentration-time curve from time 0 extrapolated to infinity (AUC_{0-inf}) and maximum plasma concentration (Cₘₐₓ)) were compared across dose levels of CR OC/APAP. Dose linearity for CR OC/APAP was assessed; linearity indicates that drug disposition (absorption, distribution, metabolism, and excretion) are constant whatever the dose. For a linear PK system, the measures of exposure, such as maximal blood concentration (Cₘₐₓ) or area under the curve from 0 to infinity (AUC) on the y-axis, are linearly (i.e., by a straight line) related to dose on the x-axis. Dose proportionality for CR OC/APAP was assessed; dose proportionality occurs when increases in the administered dose are accompanied by proportional increases in a measure of exposure, such as AUC or Cₘₐₓ.

Individual plasma concentration versus actual time data were used to estimate the PK parameters of APAP by standard non-compartmental methods. Non-transformed and log-transformed data for AUC_{0-inf} and Cₘₐₓ were evaluated by analysis of variance (ANOVA) using the SAS mixed procedure to verify no effect of study; data were tested for normality. AUC_{0-inf} and Cₘₐₓ were divided by dose (dose-normalized) for dose proportionality analyses. Dose normalization (calculated as plasma concentration divided by dose) was utilized to compare concentration across different dosage strengths. Dose linearity and proportionality were determined using linear regression of non-transformed data, including 95% confidence intervals (CIs) and 95% prediction limits. Slope, y-intercept, and R² were calculated. The slope and the intercept together define the linear relationship between dose and AUC, or dose and Cₘₐₓ. The coefficient of determination, R², represents the proportion of variability in a data set that is accounted for by a statistical model.

FIG. 55 presents the plasma APAP concentration versus time following single-dose administration of CR OC/APAP. FIG. 56 presents the plasma APAP concentration versus time following multi-dose administration of CR OC/APAP. FIG. 57 presents the steady-state plasma APAP concentration versus time following multi-dose administration of CR OC/APAP (hours 96 to 144 on day 5, relative to the start of dosing at Hour-0). For both the single-dose and multiple-dose studies, plasma APAP concentrations rose rapidly and in a dose-dependent manner. In multiple-dose studies, steady-state APAP levels were reached by day 2 (24 hours after first dose) with 2-tablet dosing and day 4 with 1-tablet dosing.

Table 74 summarizes the linearity of APAP PK parameters in the single-dose and multi-dose studies. As shown in Table 74, after both single and multiple doses of CR OC/APAP, APAP AUC_{0-inf} and Cₘₐₓ were linear with respect to dose.

TABLE 74
Linearity of APAP Pharmacokinetic Parameters

| Parameter | Single-Dose | Multiple-Dose |
|---|---|---|
| (95% CI) | (N = 119) | (N = 57) |
| AUC_{0-inf} | | |
| Slope | 41.021 | 40.177 |
| | (36.915, 45.126) | (32.089, 48.265) |
| y-Intercept | 4024.292 | 2249.134 |
| | (627.210, 7421.373) | (−2227.061, 6725.329) |
| R² | 0.6657 | 0.5255 |
| Cₘₐₓ | | |
| Slope | 6.336 | 7.079 |
| | (5.637, 7.035) | (5.125, 9.032) |
| y-Intercept | 467.019 | 816.140 |
| | (−123.280, 1057.318) | (−265.031, 1897.312) |
| R² | 0.6080 | 0.3708 |

Table 75 presents the proportionality of APAP PK parameters (dose normalized). Dose-normalized APAP AUC_{0-inf} and Cₘₐₓ values after single and multiple doses of CR OC/APAP were proportional to dose as indicated by linear regression with slopes approximately equal to zero. Tests for dose proportionality showed that the slope of dose-adjusted AUC_{0-inf} and Cₘₐₓ were approximately equal to zero for both single doses (−0.007 and −0.001, respectively) and at steady state (−0.011 and −0.004, respectively).

TABLE 75
Proportionality of APAP Pharmacokinetic Parameters
(Dose Normalized)

| Parameter | Single-Dose | | Multiple-Dose | |
|---|---|---|---|---|
| (95% CI) | (N = 119) | | (N = 57) | |
| AUC_{0-inf} | | | | |
| Slope | −0.007 | (−0.013, −0.002) | −0.011 | (−0.026, 0.005) |
| y-Intercept | 52.943 | (48.255, 57.631) | 50.557 | (42.124, 58.990) |
| R² | 0.0321 | | 0.0214 | |
| Cₘₐₓ | | | | |
| Slope | −0.001 | (−0.002, 0.000) | −0.004 | (−0.007, 0.000) |
| y-Intercept | 7.877 | (7.088, 8.665) | 10.845 | (8.905, 12.786) |

| | | |
|---|---|---|
| R² | 0.0219 | 0.0517 |

Safety and tolerability in each study was assessed using standard measures, including adverse event monitoring and clinical laboratory testing. Tables 76 and 77 present the most common treatment-emergent adverse events (TEAEs) in the single-dose and multi-dose studies, respectively. TEAEs in the single-dose studies (Table 76) and the multiple-dose studies (Table 77) were consistent with those expected with opioid analgesic therapy. The most common TEAEs at the dose levels tested were:
1 tablet (7.5 mg OC/325 mg APAP): nausea (single-dose studies) and headache (multiple-dose studies)
2 tablets (15 mg OC/650 mg APAP): nausea (single- and multiple-dose studies)
4 tablets (30 mg OC/1300 mg APAP): pruritus (single-dose study in healthy recreational drug users)

TABLE 76
Most Common Treatment-Emergent
Adverse Events (≧5% of patients), Single-Dose Studies

| | CR | | | | CR |
|---|---|---|---|---|---|
| | OC/APAP | | | | OC/APAP |
| | 1 tablet | CR OC/APAP | | | 4 tablets |
| | (7.5/325 | 2 tablets | | | (30/1300 |
| | mg) | (15/650 mg) | | | mg) |
| TEAE, n (%) | Study 1 | Study 1 | Study 2 | Study 3* | Study 3* |
| Dizziness | 1 (2.6) | 4 (9.8) | 3 (7.7) | 0 | 0 |
| Euphoric | 1 (2.6) | 3 (7.3) | 1 (2.6) | 0 | 0 |
| mood | | | | | |
| Headache | 1 (2.6) | 8 (19.5) | 1 (2.6) | 4 (6.9) | 3 (5.4) |
| Nausea | 4 (10.3) | 12 (29.3) | 5 (12.9) | 0 | 6 (10.7) |
| Pruritus | 0 | 3 (7.3) | 1 (2.6) | 2 (3.4) | 8 (14.3) |
| Vomiting | 2 (5.1) | 7 (17.1) | 1 (2.6) | 1 (1.7) | 2 (3.6) |
| Somnolence | 2 (5.1) | 5 (12.2) | 3 (7.7) | 0 | 4 (7.1) |

*Subjects enrolled in Study 3 were healthy recreational drug users.

TABLE 77
Most Common Treatment-Emergent
Adverse Events (≧5% of patients), Multiple-Dose Studies

| | |
|---|---|
| CR OC/APAP | CR OC/APAP |
| 1 tablet | 2 tablets |
| (7.5/325 mg) | (15/650 mg) |

| TEAE, n (%) | Study 1 | Study 1 | Study 2 |
|---|---|---|---|
| Abdominal pain | 0 | 3 (7.3) | 0 |
| Dizziness | 5 (12.5) | 5 (12.2) | 4 (12.1) |
| Headache | 8 (20.0) | 0 | 5 (15.2) |
| Hiccups | 0 | 4 (9.8) | 0 |
| Infrequent bowel | 2 (5) | 5 (12.2) | 0 |
| movements | | | |
| Nausea | 5 (12.5) | 12 (29.3) | 8 (24.2) |
| Pruritus | 5 (12.5) | 10 (24.4) | 7 (21.2) |
| Somnolence | 5 (12.5) | 5 (12.2) | 0 |
| Vomiting | 1 (2.5) | 5 (12.2) | 7 (21.2) |

Most changes in serum chemistry and hematology were not considered by the investigator to be clinically meaningful, and none affected subject safety. One subject (0.8%) in a single-dose study had abnormal elevated bilirubin, which was considered by the investigator to be mild and possibly related to study medication. One subject (2%) in a multiple-dose study had anemia, which was not considered related to study medication.

The results of this study demonstrate that APAP plasma concentrations following single- and multiple-dose administration of CR OC/APAP (across a range of 1, 2, and 4 tablets) were linear and proportional with respect to dose. All doses were generally well tolerated, with the most frequently occurring TEAEs varying with dose. TEAEs were consistent with those expected with opioid analgesic therapy. These findings demonstrate dose proportionality and dose linearity of the APAP component of CR OC/APAP up to 30 mg/1300 mg.

### Example 34

### Dose Proportionality and Linearity of Oxycodone after Single or Multiple Oral Doses of Controlled-Release Oxycodone/Acetaminophen (CR OC/APAP) Tablets

Data were pooled from randomized, crossover, single- and multiple-dose studies conducted in healthy adults (aged 18-55 years) to evaluate the dose proportionality and dose linearity of the oxycodone component of CR OC/APAP when administered as 1, 2, or 4 tablets (7.5 mg, 15 mg, or 30 mg oxycodone, respectively). CR OC/APAP tablets employ a dual-layer biphasic delivery mechanism that, when administered as 2 tablets, include an immediate-release component delivering 3.75 mg OC/325 mg APAP and an extended-release component delivering 11.25 mg OC/325 mg APAP. (See selected example from Chart No. 1)

The following treatments were administered under fasted conditions:
Single-dose studies: 1, 2, or 4 tablets of CR OC/APAP (7.5 mg, 15 mg, or 30 mg oxycodone, respectively) (see selected example from Chart No. 1) administered orally once
Multiple-dose studies: 1 or 2 tablets of CR OC/APAP (7.5 mg or 15 mg oxycodone, respectively) (see selected example from Chart No. 1) administered every 12 hours for 4.5 days (9 doses)

One single-dose study (highest dosage) enrolled healthy recreational drug users; all other studies enrolled healthy subjects with specific exclusions for drug use.

Blood samples for plasma analysis of oxycodone were collected up to 48 hours after dosing in single-dose studies and up to 144 hours after the hour-0 dose in multiple-dose studies. Area under the plasma oxycodone concentration-time curve from time 0 extrapolated to infinity (AUC_{0-inf}) and maximum plasma concentration (Cₘₐₓ)) were compared across dose levels of CR OC/APAP. Dose linearity for CR OC/APAP was assessed; linearity indicates that drug disposition processes (absorption, distribution, metabolism, and excretion) are constant whatever the dose. For a linear PK system, the measures of exposure, such as maximal blood concentration (Cₘₐₓ) or area under the curve from 0 to infinity (AUC) on the y-axis, are linearly (i.e., by a straight line) related to dose on the x-axis. Dose proportionality for CR OC/APAP was assessed; dose proportionality occurs when increases in the administered dose are accompanied by proportional increases in a measure of exposure, such as AUC or Cₘₐₓ.

Individual plasma concentration versus actual time data were used to estimate the PK parameters of oxycodone by standard non-compartmental methods. Non-transformed and log-transformed data for AUC_{0-inf} and Cₘₐₓ were evaluated by analysis of variance (ANOVA) using the SAS mixed procedure to verify no effect of study; data were tested for normality. AUC_{0-inf} and Cₘₐₓ were divided by dose (dose-normalized) for dose proportionality analyses. Dose normalization (calculated as plasma concentration divided by dose) was utilized to compare concentration across different dosage strengths. Dose linearity and proportionality were determined using linear regression of non-transformed data, including 95% confidence intervals (CIs) and 95% prediction limits. Slope, y-intercept, and R² were calculated. The slope and the intercept together define the linear relationship between dose and AUC, or dose and Cₘₐₓ. The coefficient of determination, R², represents the proportion of variability in a data set that is accounted for by a statistical model.

FIG. 58 presents the plasma oxycodone concentration versus time following single-dose administration of CR OC/APAP. FIG. 59 presents the plasma oxycodone concentration versus time following multi-dose administration of CR OC/APAP. FIG. 60 presents the steady-state plasma oxycodone concentration versus time following multi-dose administration of CR OC/APAP (hours 96 to 144 on day 5, relative to the start of dosing at Hour-0). For both the single-dose and multiple-dose studies, plasma oxycodone concentrations rose rapidly and were sustained in a dose-dependent manner. In multiple-dose studies, steady-state oxycodone levels were reached by day 2 and 3 (24-48 hours after first dose) with 2-tablet dosing and day 4 with 1-tablet dosing.

Table 78 summarizes the linearity of oxycodone PK parameters in the single-dose and multi-dose studies. As shown in Table 78, after both single and multiple doses of CR OC/APAP, oxycodone AUC_{0-inf} and Cₘₐₓ were linear with respect to dose.

TABLE 78
Linearity of Oxycodone Pharmacokinetic Parameters

| Parameter | Single-Dose | | Multiple-Dose | |
|---|---|---|---|---|
| (95% CI) | (N = 119) | | (N = 57) | |
| AUC_{0-inf} | | | | |
| Slope | 11.093 | (10.022, 12.165) | 14.151 | (11.477, 16.824) |
| y-Intercept | 7.202 | (−13.583, 27.987) | −3.774 | (−37.919, 30.371) |
| R² | 0.6767 | | 0.5570 | |
| Cₘₐₓ | | | | |
| Slope | 1.050 | (0.964, 1.136) | 1.640 | (1.308, 1.972) |
| y-Intercept | −0.417 | (−2.090, 1.256) | 0.362 | (−3.877, 4.602) |

| | | |
|---|---|---|
| R² | 0.7384 | 0.5229 |

Table 79 presents the proportionality of oxycodone PK parameters (dose normalized). Dose-normalized oxycodone AUC_{0-inf} and Cₘₐₓ values after single and multiple doses of CR OC/APAP were proportional to dose as indicated by linear regression with slopes approximately equal to zero. Tests for dose proportionality showed that the slope of dose-adjusted AUC_{0-inf} and Cₘₐₓ were approximately equal to zero for both single doses (−0.024 and −0.001, respectively) and at steady state (0.033 and −0.003, respectively).

TABLE 79
Proportionality of Oxycodone Pharmacokinetic Parameters
(Dose Normalized)

| Parameter | Single-Dose | | Multiple-Dose | |
|---|---|---|---|---|
| (95% CI) | (N = 119) | | (N = 57) | |
| AUC_{0-inf} | | | | |
| Slope | −0.024 | (−0.079, 0.030) | 0.033 | (−0.180, 0.247) |
| y-Intercept | 12.015 | (10.950, 13.081) | 13.397 | (10.665, 16.129) |
| R² | 0.0038 | | 0.0011 | |
| Cₘₐₓ | | | | |
| Slope | −0.001 | (−0.005, 0.004) | −0.003 | (−0.029, 0.023) |
| y-Intercept | 1.041 | (0.953, 1.129) | 1.712 | (1.377, 2.047) |

| | | |
|---|---|---|
| R² | 0.0004 | 0.0006 |

Safety and tolerability in each study was assessed using standard measures, including adverse event monitoring and clinical laboratory testing. Tables 80 and 81 present the most common treatment-emergent adverse events (TEAEs) in the single-dose and multi-dose studies, respectively. TEAEs in the single-dose studies (Table 80) and the multiple-dose studies (Table 81) were consistent with those expected with opioid analgesic therapy. The most common TEAEs at the dose levels tested were: for 1 tablet (7.5 mg OC/325 mg APAP)—nausea (single-dose studies) and headache (multiple-dose studies); for 2 tablets (15 mg OC/650 mg APAP)—nausea (single- and multiple-dose studies); and for 4 tablets (30 mg OC/1300 mg APAP)—pruritus (single-dose study in healthy recreational drug users).

TABLE 80
Most Common Treatment-Emergent
Adverse Events (≧5% of patients), Single-Dose Studies

| | CR | | | | CR |
|---|---|---|---|---|---|
| | OC/APAP | CR OC/APAP | | | OC/APAP |
| Treatment-Emergent | 1 tablet | 2 tablets | | | 4 tablets |
| Adverse | (7.5/325 mg) | (15/650 mg) | | | (30/1300 mg) |
| Event, n (%) | Study 1 | Study 1 | Study 2 | Study 3* | Study 3* |
| Dizziness | 1 (2.6) | 4 (9.8) | 3 (7.7) | 0 | 0 |
| Euphoric mood | 1 (2.6) | 3 (7.3) | 1 (2.6) | 0 | 0 |
| Headache | 1 (2.6) | 8 (19.5) | 1 (2.6) | 4 (6.9) | 3 (5.4) |
| Nausea | 4 (10.3) | 12 (29.3) | 5 (12.9) | 0 | 6 (10.7) |
| Pruritus | 0 | 3 (7.3) | 1 (2.6) | 2 (3.4) | 8 (14.3) |
| Vomiting | 2 (5.1) | 7 (17.1) | 1 (2.6) | 1 (1.7) | 2 (3.6) |
| Somnolence | 2 (5.1) | 5 (12.2) | 3 (7.7) | 0 | 4 (7.1) |

*Subjects enrolled in Study 3 were healthy recreational drug users.

TABLE 81
Most Common Treatment-Emergent
Adverse Events (≧5% of patients), Multiple-Dose Studies

| | CR OC/APAP | CR OC/APAP | |
|---|---|---|---|
| | 1 tablet | 2 tablets | |
| Treatment-Emergent | (7.5 mg/325 mg) | (15 mg/650 mg) | |
| Adverse Event, n (%) | Study 1 | Study 1 | Study 2 |
| Abdominal pain | 0 | 3 (7.3) | 0 |
| Dizziness | 5 (12.5) | 5 (12.2) | 4 (12.1) |
| Headache | 8 (20.0) | 0 | 5 (15.2) |
| Hiccups | 0 | 4 (9.8) | 0 |
| Infrequent bowel | 2 (5) | 5 (12.2) | 0 |
| movements | | | |
| Nausea | 5 (12.5) | 12 (29.3) | 8 (24.2) |
| Pruritus | 5 (12.5) | 10 (24.4) | 7 (21.2) |
| Somnolence | 5 (12.5) | 5 (12.2) | 0 |
| Vomiting | 1 (2.5) | 5 (12.2) | 7 (21.2) |

The results of this study demonstrate that oxycodone plasma concentrations following single- and multiple-dose administration of CR OC/APAP (across a range of 1, 2, and 4 tablets) were linear and proportional with respect to dose. All doses were generally well tolerated and TEAEs were consistent with those expected with opioid analgesic therapy. These findings demonstrate dose proportionality and dose linearity of the oxycodone component of CR OC/APAP up to 30 mg/1300 mg.

### Example 35

### Comparison of Subjective Drug Effects of Orally Administered Controlled-Release Oxycodone/Acetaminophen (CR OC/APAP) Tablets Versus Immediate-Release Oxycodone/Acetaminophen Tablets in Recreational Users of Prescription Opioids

A single-center, randomized, double-blind, double-dummy, active- and placebo-controlled, crossover study was conducted to compare the extent to which CR OC/APAP intact, CR OC/APAP crushed, and commercially-available IR OC/APAP formulations produce certain subjective effects that have been associated with drug abuse in recreational opioid users, such as drug liking, drug high, and good drug effects. Pharmacodynamic (PD) parameters were assessed after administration of intact and crushed CR OC/APAP and IR OC/APAP to recreational opioid users. Participants included healthy adult male and female nondependent, recreational opioid users who reported occasions of recreational opioid use over the past year, including occasion within the past 12 weeks.

Participants initially underwent a naloxone challenge test to confirm a lack of physical dependence on opioids and a drug discrimination test to determine that they could detect the subjective effects of oxycodone. During the drug discrimination test, all participants received single doses of IR OC/APAP (15 mg/650 mg) and placebo and reported on the subjective effects. Participants who could not discriminate between active drug and placebo or tolerate this single dose were excluded from enrollment. A total of 107 participants entered the study, were checked into the clinical facility, and completed the study inclusion phase. Safety analyses were conducted in 61 participants who passed the drug discrimination phase and entered the treatment phase. PK and PD analyses were performed with data collected from the 55 participants who completed the assessment phase. Participants had a mean age of 26 years and approximately 75% were male. 95% of participants had a history of alcohol abuse and 67% had a history of tobacco use.

The treatment phase included a total of 7 assessment periods, with a 72-hour washout period between doses. During the treatment phase, because of the history of prescription opioid abuse in this population, IR OC/APAP was encapsulated for proper blinding. To ensure blinding of all study treatments, participants received matching placebos for each possible treatment configuration so dose administration in each treatment period consisted of 8 capsules and 4 tablets. For administration of the CR OC/APAP, controlled-release tablets containing 7.5 mg OC/325 mg APAP were used. (See selected example from Chart No. 1). During each assessment period, participants received a single dose of one of the 7 study treatments:

CR OC/APAP intact

High dose: 30 mg OC/1300 mg APAP (4 tablets)

Low dose: 15 mg OC/650 mg APAP (2 tablets)

IR OC/APAP intact

High dose: 30 mg OC/1300 mg APAP (4 tablets, over-encapsulated)

Low dose: 15 mg OC/650 mg APAP (2 tablets, over-encapsulated)

CR OC/APAP crushed (encapsulated)

High dose: 30 mg OC/1300 mg APAP (8 capsules)

IR OC/APAP crushed (encapsulated)

High dose: 30 mg OC/1300 mg APAP (4 capsules)

Placebo

The primary outcome measures were participant-reported visual analog scale (VAS) scores for: drug liking—assessed on a 100-mm bipolar VAS (0 mm=Strong disliking; 50 mm=Neither like or dislike; 100 mm=Strong liking); drug high—assessed on a 100-mm unipolar VAS (0 mm=None; 100 mm=Extremely); and good drug effects—assessed on a 100-mm unipolar VAS (0 mm=None; 100 mm=Extremely). Secondary outcome measures included pupillometry and other subjective measures, such as Take Drug Again Assessment (TDAA) bipolar 0- to 100-point VAS, Global Assessment of Overall Drug Liking bipolar 0- to 100-point VAS, and Addiction Research Center Inventory/Amphetamine Morphine Benzedrine Group (ARCI/AMBG) 4-point scale.

Peak drug effects (Eₘₐₓ) was a primary pharmacodynamic outcome analyzed. Higher Eₘₐₓ values indicate greater maximum perceived subjective effects. The time to Eₘₐₓ (TEₘₐₓ) was also analyzed. Lower TEmax values indicate shorter time to maximum perceived subjective effects. The total area under the effective curve (AUE), which measures the total subjective effect over a specific time periods, with lower values indicating lesser effect, was assessed at multiple time points from 0 to 12 hours. Analyses of pharmacodynamic outcomes were performed using least-squares mean (LSM) scores with 95% confidence intervals (CIs), which were compared using a linear, mixed-model analysis of variance (ANOVA) model with fixed effects for sequence, period, and treatment, and a random effect for participants nested in the sequence. Data were adjusted for multiple comparisons.

FIGS. 61-64 present the mean drug liking VAS scores over 12 hours. FIGS. 65-68 present the mean drug high VAS scores over 12 hours. As shown in FIGS. 61-68, all CR OC/APAP formulations produced more delayed and lower mean peak positive effects according to VAS scores for drug liking, drug high, and good drug effects compared with IR OC/APAP. VAS scores for good drug effects (results not shown in FIGS. 61-68) were similar to those for drug high. FIG. 69 presents the least-squares mean Eₘₐₓ for drug liking for high-dose intact CR OC/APAP vs. IR OC/APAP. FIG. 70 presents the least-squares mean Eₘₐₓ for drug high and good drug effects for high-dose intact CR OC/APAP vs. IR OC/APAP. Eₘₐₓ values for these outcomes after the administration of high-dose intact CR OC/APAP were significantly lower (P<0.001) than for high-dose intact IR OC/APAP. (See FIGS. 69 and 70). FIG. 71 presents the least-squares mean TEₘₐₓ for drug liking, drug high, and good drug effects for high-dose intact CR OC/APAP vs. IR OC/APAP. TEₘₐₓ was delayed for high-dose intact CR OC/APAP compared with IR OC/APAP, but only the difference for drug liking (P<0.05) reached statistical significance. (See FIG. 71).

AUE values at early timepoints (i.e., 0-1 h and 0-2 h) for these VAS ratings were also significantly lower for high-dose intact CR OC/APAP compared with high-dose intact IR OC/APAP (P<0.01 for each comparison). Comparisons between low-dose intact CR OC/APAP and IR OC/APAP and high-dose crushed CR OC/APAP and IR OC/APAP also demonstrated that the CR OC/APAP formulations produced significantly lower Eₘₐₓ scores for drug liking, drug high, and good drug effects (P<0.001 for each comparison). As shown in Table 82, Crushing CR OC/APAP did not increase these effects and produced similar or less drug liking, drug high, and good drug effects than the same dose of intact CR OC/APAP or IR OC/APAP. Crushed CR OC/APAP had a significantly longer TEₘₐₓ (P<0.02) and significantly lower AUE₀₋₁ₕ, AUE₀₋₂ₕ, and AUE₀₋₄ₕ (P<0.02) for drug liking, drug high, and good drug effects than the same dose of intact CR OC/APAP. Moreover, secondary subjective measures (i.e., TDAA, global assessment of overall drug liking, and ARCI/AMBG) produced results that were consistent with the primary subjective measures.

TABLE 82
Pharmacodynamic Comparisons: Crushed vs Intact

| | CR OC/APAP High-Dose | | | CR OC/APAP High-Dose | | |
|---|---|---|---|---|---|---|
| | Crushed vs IR OC/APAP | | | Intact vs High-Dose | | |
| | High-Dose Intact | | | Crushed | | |
| | Drug | Drug | Good Drug | Drug | Drug | Good Drug |
| Variable | Liking | High | Effects | Liking | High | Effects |
| Eₘₐₓ | | | | | | |
| LSM difference | 12.3 | 33.4 | 28.5 | 3.1 | 5.3 | 8.5 |
| Adjusted P | <0.001 | <0.001 | <0.001 | 0.127 | 0.130 | 0.035 |
| TEₘₐₓ | | | | | | |
| LSM difference | −1.1 | −1.5 | −1.4 | −0.6 | −1.0 | −0.9 |
| Unadjusted P | <0.001 | <0.001 | <0.001 | 0.012 | <0.001 | <0.001 |
| AUE_{0-1 h} | | | | | | |
| LSM difference | 10.2 | 22.4 | 23.0 | 5.6 | 9.8 | 12.6 |
| Unadjusted P | 0.004 | <0.001 | <0.001 | <0.001 | <0.001 | <0.001 |
| AUE_{0-2 h} | | | | | | |
| LSM difference | 23.1 | 65.6 | 59.0 | 14.4 | 27.5 | 31.1 |
| Unadjusted P | <0.001 | <0.001 | <0.001 | <0.001 | <0.001 | <0.001 |
| AUE_{0-4 h} | | | | | | |
| LSM difference | 21.7 | 78.2 | 69.3 | 14.9 | 26.2 | 34.1 |
| Unadjusted P | <0.001 | <0.001 | <0.001 | 0.016 | 0.004 | 0.001 |
| AUE_{0-8 h} | | | | | | |
| LSM difference | 13.6 | 69.0 | 58.9 | 15.0 | 19.6 | 31.4 |
| Unadjusted P | 0.184 | <0.001 | <0.001 | 0.142 | 0.134 | 0.060 |
| AUE_{0-12 h} | | | | | | |
| LSM difference | 9.9 | 63.7 | 51.2 | 15.9 | 22.0 | 36.5 |
| Unadjusted P | 0.493 | <0.001 | 0.014 | 0.214 | 0.153 | 0.077 |

*Drug liking measured using a 100-mm bipolar VAS (0 = Strong disliking; 50 = Neither like nor dislike; 100 = Strong liking).
**Drug high and good drug effects measured using a 100-mm unipoler (0 = None; 100 = Extremely).

FIG. 72 presents the mean pupillometry scores over 12 hours. Pupil size followed a similar time course as the subjective effects. Notably, the delay in effects associated with crushing CR OC/APAP was demonstrated in this physiologic effect.

The occurrence of adverse events (AEs) after administration of study drug was also assessed. The most frequently observed AEs were consistent with oxycodone-containing medications. AEs included nausea, vomiting, and pruritus and were more common with IR OC/APAP compared with CR OC/APAP.

The results of this study demonstrated that, within the sample population of recreational prescription opioid users, CR OC/APAP produced less drug liking, drug high, and good drug effects compared with IR OC/APAP, a finding that may be associated with a lower potential for abuse. In addition, crushing CR OC/APAP lowered and delayed the effects compared with intact CR OC/APAP and IR OC/APAP, an effect that is opposite to the intent of drug abusers. This study showed that intact and crushed CR OC/APAP administered at both low and high doses produced less drug liking, drug high, and good drug effects compared with IR OC/APAP at the same doses. The CR OC/APAP tablet technology includes a dual-layer, biphasic delivery profile with an IR component and an ER component; when the CR OC/APAP tablet is crushed, these 2 layers become mixed, delaying the onset of the medication.

### Example 36

### Steady-State Pharmacokinetics of 1 and 2 Tablets a Controlled-Release Oxycodone and Acetaminophen (CR OC/APAP) Combination, Compared With Immediate-Release Oxycodone and Acetaminophen

A single-center, open-label, randomized, phase 1, multiple-dose, 3-period, 6-sequence, crossover study was conducted to evaluate the steady-state PK and bioavailability of both the oxycodone and APAP components of 1- or 2-tablet dosages of CR OC/APAP administered every 12 hours over 4.5 days compared with the 1-tablet dosing of IR OC/APAP administered every 6 hours over 4.5 days. The study further assessed the dose proportionality of 1- and 2-tablet dosages of CR OC/APAP at steady state, and evaluated the relative safety of CR OC/APAP compared with IR OC/APAP.

Participants in the study were normal healthy male or non-lactating, non-pregnant female subjects aged 18 to 55 years with body mass index of ≧9 to ≦30 kg/m and a minimum weight of 130 lbs. Exclusion criteria included smoking or use of nicotine containing products in the previous 6 months; history of drug or alcohol use or positive urine test for drugs of abuse; use of prescription or over-the-counter drugs within 14 days of study check-in; history of drug allergy, hypersensitivity, or intolerance of opioid drug products (including oxycodone) or APAP; history of any condition that may interfere with the absorption, distribution, metabolism, or excretion of study drug; or previous gastric bypass or gastric band surgery. A total of 48 adults enrolled in the studies. 33 (68.8%) completed all 3 treatment periods. Subjects were randomized to receive the following treatments in a crossover design under fasted conditions:
Treatment A: CR OC/APAP, 1 tablet (7.5 mg OC/325 mg APAP) (see selected example from Chart No. 1) taken every 12 hours for 4.5 days (9 tablets total)
Treatment B: CR OC/APAP, 2 tablets (total, 15 mg OC/650 mg APAP) (see selected example from Chart No. 1) taken every 12 hours for 4.5 days (18 tablets total)
Treatment C: commercially-available IR OC/APAP, 1 tablet (7.5 mg OC/325 mg APAP) taken every 6 hours for 4.5 days (18 tablets total)

The study included a screening visit and 3 confinement periods of approximately 7 days each, with a minimum of 14 days between the start of each period, and a telephone follow-up period of at least 7 days after study completion. Plasma samples were collected at the following times up to 144 hours after dosing:
Day 1: pre-dose, and 15, 30, and 45 minutes and 1, 2, 3, 4, 6, 7, 8, 10, and 12 hours post-dose, with additional samples collected at 15, 30, and 45 minutes after the 6-hour dose in treatment group C
Days 2-4: before the morning dose at times 24, 48, and 72 hours
Days 5-7: just prior to the 96-hour dose (day 5) and at 15, 30, and 45 minutes after the dose at hour 96, and at hours 97, 98, 99, 100, 102, 103, 104, 106, 108, 112, 120 (day 6), 132, and 144 (day 7); additional samples were collected at 15, 30, and 45 minutes after the dose at 102 hours for treatment group C

FIG. 73 provides a simulation of plasma concentrations over 6 days (144 h) with multiple dosing of CR OC/APAP (administered q12 h) and IR OC/APAP (administered q6 h).

PK parameters for oxycodone and APAP were calculated using standard non-compartmental methods. Adverse events were monitored throughout the study. Analysis of variance (ANOVA) was performed to compare treatment conditions A, B, and C using the natural log-transformed, dose-normalized (plasma concentration divided by dose) PK parameters at steady state (Cₘₐₓₛₛ, Cₘᵢₙₛₛ, Cₐₛₛ, and AUC₀₋₁₂ₕₛₛ [mean exposure within 0-12 h after the hour 96 dose on day 5, at steady state]) or natural log-transformed PK parameters (degree of fluctuation around the average plasma concentration at steady state, defined as [Cₘₐₓₛₛ−Cₘᵢₙₛₛ]/Cₐᵥₛₛ). Geometric least-squares (LS) means, percent ratio of the geometric LS means, and the corresponding 90% confidence intervals (CI) for the ratios of the geometric LS means were calculated for treatment comparisons. A 90% CI of the geometric LS means ratio fully contained within 80% to 125% indicated no difference between treatments. ANOVA was performed to analyze the untransformed PK parameter (Kₑₗ and t_{1/2}). Wilcoxon signed-rank test was utilized to compare untransformed time with maximum observed plasma concentration at steady state (Tₘₐₓₛₛ) between treatments. P≦0.05 was considered a significant difference between treatments.

FIG. 74 presents the mean steady-state plasma concentration of oxycodone following the last dose of CR OC/APAP. Steady state for oxycodone with CR OC/APAP (2 tablets) was reached by day 3; the oxycodone Cₘᵢₙ on days 2 to 4 was >10 ng/mL for the 2-tablet dosage. The estimated oxycodone PK parameters on day 5 while at steady state are presented in Table 83. Steady-state results for oxycodone with CR OC/APAP (2 tablets) and IR OC/APAP indicated comparable AUC₀₋₁₂ₕₛₛ, Cₐᵥₛₛ, and Cₘᵢₙₛₛ; however, Cₘₐₓₛₛ, swing, and degree of fluctuation were 16%, 24%, and 23% lower for CR OC/APAP, respectively. Bioavailability of CR OC/APAP (2 tablets) was similar to that for IR OC/APAP as demonstrated by the similar AUC values; however, CR OC/APAP exhibited less fluctuation in plasma concentrations over time. Steady-state PK assessments for oxycodone (dose-normalized) showed no difference relative to AUC₀₋₁₂ₕₛₛ, Cₘₐₓₛₛ, Cₘᵢₙₛₛ, Cₐᵥₛₛ, and degree of fluctuation between 1 and 2 tablets of CR OC/APAP. Dose proportionality with respect to oxycodone AUC (LS means ratio of the comparison=97.97%) and Cₘₐₓ (LS means ratio of the comparison=98.99%) was seen between 1 and 2 tablets of CR OC/APAP; the 90% CIs of the LS means ratios for 1 versus 2 tablets were fully contained within the predefined no-difference range of 80% to 125% for oxycodone.

TABLE 83
Steady-State (Day 5) Pharmacokinetic Estimates for Oxycodone (n = 33)

| | CR OC/APAP | | CR OC/APAP | | IR OC/APAP | |
|---|---|---|---|---|---|---|
| | (1 tablet q12h; | | (2 tablets q12h; | | (1 tablet q6h; | |
| Parameter, mean (SD) | 7.5 mg/325 mg) | | 15 mg/650 mg) | | 15 mg/650 mg) | |
| AUC₀₋₁₂ₕₛₛ, ng · h/mL^{a} | 102.36 | (29.30) | 208.59 | (59.28) | 208.93 | (57.30) |
| Cₐᵥₛₛ, ng/mL | 8.53 | (2.44) | 17.38 | (4.94) | 17.41 | (4.78) |
| Cₘₐₓₛₛ, ng/mL | 12.67 | (3.48) | 25.67 | (7.49) | 30.50 | (8.91) |
| Cₘᵢₙₛₛ, ng/mL | 4.06 | (1.40) | 8.98 | (3.52) | 8.78 | (3.17) |
| Degree of fluctuation, % | 101.72 | (14.14) | 97.17 | (18.80) | 126.83 | (27.93) |
| Swing | 2.23 | (0.64) | 2.03 | (0.70) | 2.67 | (0.92) |
| Tₘₐₓₛₛ, h^{b} | 2.00 | (0.50-10.00) | 2.00 | (0.50-7.00) | 6.50 | (0.50-8.02) |
| t_{1/2}, h | 5.46 | (1.24) | 6.11 | (1.46) | 5.47 | (1.70) |
| Kₑₗ, L/h | 0.1326 | (0.0269) | 0.1199 | (0.0291) | 0.1387 | (0.0418) |

^{a}Mean exposure within 0 to 12 hours after the hour 96 dose (day 5, at steady state)
^{b}Median (range)

FIG. 75 presents the mean steady-state plasma concentration of APAP following the last dose of CR OC/APAP. The estimated PK parameters for APAP on day 5 while at steady state are presented in Table 84. Steady state with respect to APAP was reached on day 2 (2 tablets) and day 4 (1 tablet) of CR OC/APAP. On day 5, 7 to 12 hours after the last dose of CR OC/APAP (2 tablets), APAP plasma levels tapered off to levels below those observed after an equivalent dose of IR OC/APAP administered every 6 hours. Steady-state PK assessments for APAP (dose normalized) showed no difference between any of the 3 treatment arms for AUC₀₋₁₂ₕₛₛ, Cₘₐₓₛₛ, Cₘᵢₙₛₛ, Cₐᵥₛₛ, and degree of fluctuation. These results demonstrate that bioavailability of CR OC/APAP is similar to that for IR OC/APAP. Furthermore, these results demonstrate dose proportionality with respect to APAP with CR OC/APAP (both 1 and 2 tablets).

TABLE 84
Steady-State (Day 5) Pharmacokinetic Estimates for Acetaminophen (n = 33)

| | CR OC/APAP | | CR OC/APAP | | IR OC/APAP | |
|---|---|---|---|---|---|---|
| | (1 tablet q12h; | | (2 tablets q12h; | | (1 tablet q6h; | |
| Parameter, mean (SD) | 7.5 mg/325 mg) | | 15 mg/650 mg) | | 15 mg/650 mg) | |
| AUC₀₋₁₂ₕₛₛ, ng · h/mL^{a} | 15307 | (4092) | 28512 | (7714) | 28719 | (7023) |
| Cₐᵥₛₛ, ng/mL | 1276 | (341) | 2376 | (643) | 2393 | (585) |
| Cₘₐₓₛₛ, ng/mL | 3117 | (840) | 5872 | (1932) | 5968 | (1639) |
| Cₘᵢₙₛₛ, ng/mL | 474.67 | (163) | 870.42 | (336) | 922.58 | (321) |
| Degree of fluctuation, % | 212.08 | (52.29) | 218.06 | (81.14) | 213.79 | (50.53) |
| Swing | 5.95 | (2.04) | 6.63 | (3.61) | 5.94 | (2.24) |
| Tₘₐₓₛₛ, h^{b} | 0.50 | (0.25-3.00) | 0.50 | (0.25-3.02) | 0.50 | (0.25-8.02) |
| t_{1/2}, h | 5.60 | (1.35) | 7.47 | (2.89) | 5.74 | (2.98) |
| Kₑₗ, L/h | 0.1308 | (0.0317) | 0.1026 | (0.0292) | 0.1416 | (0.0515) |

^{a}Mean exposure within 0 to 12 hours after the hour 96 dose (day 5, at steady state)
^{b}Median (range)

Table 85 presents a summary of the most frequently occurring treatment-emergent adverse events (TEAEs). Overall, 42 subjects (87.5%) reported ≧1 TEAE; 67% were considered by the investigator to be mild in intensity and 21% were considered to be moderate in intensity. There were no serious TEAEs. The most frequently reported TEAEs overall were nausea, pruritus, headache, and dizziness. Fewer subjects receiving 1 tablet of CR OC/APAP (47.5%) experienced TEAEs than subjects receiving 2 tablets of CR OC/APAP (70.7%) and IR OC/APAP (73.2%). Ten participants discontinued because of vomiting (1 tablet of CR OC/APAP, n=1; 2 tablets of CR OC/APAP, n=5; and 1 tablet of IR OC/APAP, n=4) per protocol specification. Most individual hematology and serum chemistry values were within the normal range. All changes outside the reference range were considered by the investigator to not be clinically significant except for the hematology results from 1 subject (2%) indicating anemia (not considered related to study medication). There were no significant differences in tolerability between the 2-tablet treatment with CR OC/APAP and IR OC/APAP.

TABLE 85
Summary of Most Frequently Occurring Treatment Emergent Adverse Events

| | CR OC/APAP | CR OC/APAP | IR OC/APAP | |
|---|---|---|---|---|
| Treatment-Emergent | (1 tablet q12h; | (2 tablets q12h; | (1 tablet q6h; | |
| Adverse Event, | 7.5 mg/325 mg) | 15 mg/650 mg) | 15 mg/650 mg) | Overall |
| n (%) | (n = 40) | (n = 41) | (n = 41) | (N = 48) |
| Any TEAE | 19 (47.5) | 29 (70.7) | 30 (73.2) | 42 (87.5) |
| Nausea | 5 (12.5) | 12 (29.3) | 13 (31.7) | 22 (45.8) |
| Pruritus | 5 (12.5) | 10 (24.4) | 10 (24.4) | 18 (37.5) |
| Headache | 9 (22.5) | 4 (9.8) | 7 (17.1) | 16 (33.3) |
| Dizziness | 5 (12.5) | 6 (14.6) | 7 (17.1) | 15 (31.3) |
| Vomiting | 1 (2.5) | 5 (12.2) | 4 (9.8) | 10 (20.8) |
| Infrequent bowel | 4 (10.0) | 5 (12.2) | 3 (7.3) | 10 (20.8) |
| movements | | | | |
| Somnolence | 5 (12.5) | 5 (12.2) | 1 (2.4) | 8 (16.7) |

The results of this study demonstrate that both oxycodone and APAP plasma concentrations rose quickly (Tₘₐₓ at 2 hours and 30 minutes, respectively) after administration of CR OC/APAP, and then APAP concentrations tapered off more rapidly than oxycodone concentrations over the 12-hour dosing interval. At steady state, the PK profile of CR OC/APAP administered every 12 hours demonstrated: lower peak concentrations and 23% lower degree of fluctuation in the concentration of oxycodone than with IR OC/APAP; dose proportionality was demonstrated between the 1- and 2-tablet dosages of CR OC/APAP; comparable bioavailability of oxycodone and APAP with that of IR OC/APAP administered every 6 hours. CR OC/APAP was generally well tolerated. TEAEs for CR OC/APAP were typical for those of a low-dose opioid, and similar to those of IR OC/APAP at equivalent doses. The consistent PK profile and safety findings support a 12-hour dosing interval for CR OC/APAP for at least 4.5 days.

### Example 37

### Comparison of the Pharmacokinetic Profile of Oral, Controlled-Release Formulation of Oxycodone/Acetaminophen (CR OC/APAP) Analgesic at Steady State Versus Marketed Immediate-Release Tablets

A single-center, randomized, open-label, multiple comparator, multiple-dose study was conducted to evaluate the steady-state PK characteristics as well as safety and tolerability of CR OC/APAP (administered as 2 tablets every 12 hours) compared with those of commercially-available IR oxycodone, IR tramadol/APAP, and IR OC/APAP (administered every 6 hours) in healthy participants. Participants in the study were healthy adults aged 18 to 55 years, with weight ≧130 lb and body mass index of ≧19 to <30 kg/m². Current recreational drug users were excluded, as were subjects with a history of abuse/addiction or recent illicit drug use (within 2 years) or nicotine use (within 6 months). Other exclusion criteria included a history of any condition that might interfere with the absorption, distribution, metabolism, or excretion of study drug; or history of gastric bypass or gastric band surgery. A total of 48 adults were enrolled, and 24 (50.0%) completed all treatment periods and were included in the PK analysis.

Participants were randomly assigned to receive each study medication in 1 of 4 treatment sequences (4 periods, ˜7 days each) separated by a minimum of 13 days. The following treatments were administered under fasted conditions.
CR OC/APAP 7.5 mg/325 mg (see selected example from Chart No. 1); 2 tablets (total dose, 15 mg/650 mg) every 12 hours for 4.5 days
IR oxycodone (commercially-available) 15 mg; 1 tablet every 6 hours for 4.5 days
IR tramadol/APAP (commercially-available) 37.5 mg/325 mg; 1 tablet every 6 hours for 4.5 days
IR OC/APAP (commercially-available) 7.5 mg/325 mg; 1 tablet every 6 hours for 4.5 days

For participants who completed all treatment periods, the PK of oxycodone was compared among CR OC/APAP, IR oxycodone, and IR OC/APAP, and the PK of APAP was compared among CR OC/APAP, IR tramadol/APAP, and IR OC/APAP. Blood samples for bioanalysis of oxycodone and APAP were collected predose and at 30 minutes and 1, 2, 3, 4, 6, 6.5, 8, and 12 hours after dosing on day 1; before the morning dose on days 2 through 4; predose on day 5 (96 hours); and at 96.5, 97, 98, 99, 100, 102, 102.5, 104, 108, 120 (day 6), and 132 hours.

Oxycodone and APAP concentrations were determined using liquid chromatography/tandem mass spectrometry. Plasma concentration versus time data were used to estimate the PK parameters of oxycodone and APAP, calculated using standard non-compartmental methods. For steady-state data (day 5), analysis of variance was performed to compare treatments using the natural log-transformed, dose-normalized (plasma concentration divided by dose administered) PK parameters (Cₘₐₓₛₛ, Cₘᵢₙₛₛ, C_{avgss}, and AUC₀₋₁₂ₛₛ) or natural log-transformed PK parameters (degree of fluctuation of the plasma concentration [100·(Cₘₐₓₛₛ−Cₘᵢₙₛₛ)/C_{avgss}] and swing of plasma concentrations, defined as [(Cₘₐₓₛ−Cₘᵢₙₛₛ)/Cₘᵢₙₛₛ]) as the dependent variable, with sequence, treatment, and period as fixed effects and subject nested within sequences as random effect. Dose normalization was utilized to compare concentration across different dosage strengths. Geometric least-squares (LS) means, percent ratio of the geometric LS means, and the corresponding 90% confidence interval (CI) of the geometric LS means were calculated for treatment comparisons. 90% CI of the geometric LS means ratio fully contained within 80% to 125% concluded no difference between treatments. Wilcoxon signed-rank test was utilized to compare untransformed time to maximum observed plasma concentration at steady state (Tₘₐₓₛₛ) between treatments. P≦0.05 was considered a significant difference between treatments. Summary statistics were compiled for treatment-emergent adverse events (TEAEs).

FIG. 76 presents the steady-state (day 5) mean oxycodone plasma concentration over time for the study completers (n=24). Steady state for oxycodone was attained at day 2 (24 h) after initial CR OC/APAP administration, and the oxycodone Cₘᵢₙ on days 2 to 4 was >11 ng/mL. Mean plasma oxycodone concentrations increased rapidly after CR OC/APAP administration and were approximately 37% of the peak plasma concentration 12 hours after dosing. The estimated oxycodone PK parameters on day 5 (steady state) are presented in Table 86. Dose-normalized AUC₀₋₁₂ₕₛₛ, C_{avgss}, Cₘₐₓₛₛ, and Cₘᵢₙₛₛ of oxycodone were comparable across groups (90% CIs for the treatment comparisons were fully contained within the predefined no-difference range of 80%-125%). Dose-normalized mean degree of fluctuation of oxycodone was 84% for CR OC/APAP during the dosing interval (q12 h) and was 111% for IR OC/APAP (q6 h); CR OC/APAP demonstrated a 23% lower degree of fluctuation than IR OC/APAP. Dose-normalized swing of oxycodone from CR OC/APAP was 1.65, 31% higher than that from IR oxycodone (1.32) but 20% lower than that from IR OC/APAP (2.13). The elimination half-life (t_{1/2}) for oxycodone from CR OC/APAP was approximately 1 hour longer than IR oxycodone and IR OC/APAP.

TABLE 86
Steady-State (Day 5) Pharmacokinetic Estimates for Oxycodone (n = 24)

| Parameter, mean (SD) | CR OC/APAP | | IR OC/APAP | | IR Oxycodone | |
|---|---|---|---|---|---|---|
| AUC₀₋₁₂ₕₛₛ, ng · h/mL | 208.34 | (45.34) | 191.54 | (42.81) | 376.88 | (83.90) |
| Cₐᵥₛₛ, ng/mL | 17.36 | (3.78) | 15.96 | (3.57) | 31.41 | (6.99) |
| Cₘₐₓₛₛ, ng/mL | 24.00 | (5.38) | 26.32 | (6.18) | 45.15 | (10.54) |
| Cₘᵢₙₛₛ, ng/mL | 9.31 | (2.39) | 8.81 | (2.40) | 19.91 | (4.93) |
| Degree of fluctuation, % | 83.89 | (17.58) | 110.90 | (33.39) | 79.94 | (19.83) |
| Swing | 1.65 | (0.58) | 2.13 | (0.94) | 1.32 | (0.50) |
| Tₘₐₓₛₛ, h^{a} | 3.00 | (1.00-5.92) | 7.25 | (0.50-8.13) | 3.00 | (1.00-12.00) |
| Kₑₗ, L/h | 0.1318 | (0.0223) | 0.1517 | (0.0205) | 0.1525 | (0.0206) |
| t_{1/2}, h | 5.40 | (0.87) | 4.65 | (0.62) | 4.62 | (0.59) |

^{a}Median (range)

FIG. 77 presents the steady-state (day 5) mean APAP plasma concentration over time for the study completers (n=24). The estimated PK parameters for APAP on day 5 while at steady state are presented in Table 87. Steady state for APAP was attained by day 2 (24 h) after initial CR OC/APAP administration. On day 5, mean plasma concentrations of APAP increased rapidly after administration of CR OC/APAP, but declined to 17% of peak concentration (Cₘₐₓₛₛ) by 12 hours after dosing. Mean plasma concentrations of APAP from CR OC/APAP were less than those from IR tramadol/APAP and IR OC/APAP approximately 7 hours after dosing on day 5. Dose-normalized AUC₀₋₁₂ₕₛₛ, C_{avgss}, Cₘₐₓₛₛ, and degree of fluctuation were comparable across treatments. Dose-normalized Cₘᵢₙₛₛ after CR OC/APAP was 21% lower than IR tramadol/APAP and 22% lower than IR OC/APAP. The t_{1/2} of APAP from CR OC/APAP (LS mean=8.3 h) was similar to IR OC/APAP (LS mean=7.8 h), and approximately 2.31 hours longer compared with IR tramadol/APAP (LS mean=5.98 h).

TABLE 87
Steady-State (Day 5) Pharmacokinetic Estimates for APAP (n = 24)

| Parameter, | | | | | | |
|---|---|---|---|---|---|---|
| mean (SD) | CR OC/APAP | | IR OC/APAP | | IR Tramadol/APAP | |
| AUC₀₋₁₂ₕₛₛ, ng · h/mL | 28160.40 | (5807.09) | 29284.22 | (5477.73) | 29711.92 | (5427.37) |
| Cₐᵥₛₛ, ng/mL | 2346.70 | (483.92) | 2440.35 | (456.48) | 2475.99 | (452.28) |
| Cₘₐₓₛₛ, ng/mL | 4792.50 | (1132.40) | 4876.67 | (1383.08) | 5078.33 | (1189.70) |
| Cₘᵢₙₛₛ, ng/mL | 852.75 | (273.25) | 1069.13 | (291.83) | 1070.92 | (367.35) |
| Degree of | 169.13 | (39.83) | 155.25 | (38.77) | 163.90 | (47.17) |
| fluctuation, % | | | | | | |
| Swing | 5.08 | (2.07) | 3.81 | (1.63) | 4.22 | (2.14) |
| Tₘₐₓₛₛ, h^{a} | 1.00 | (0.50-4.00) | 0.75 | (0.25-8.00) | 0.88 | (0.25-8.00) |
| Kₑₗ, L/h | 0.1072 | (0.0285) | 0.1201 | (0.0338) | 0.1355 | (0.0279) |
| t_{1/2}, h | 6.90 | (1.76) | 6.21 | (1.79) | 5.32 | (1.10) |

^{a}Median (range)

Standard safety assessments including adverse event monitoring and clinical laboratory tests were performed throughout the study. Overall, 44 of 48 (91.7%) enrolled participants reported ≧1 TEAE; all were rated by the investigator to be mild or moderate in severity; there were no serious or severe TEAEs. As shown in Table 88, the most frequently occurring TEAEs were nausea, vomiting, dizziness, pruritus, and headache. More subjects reported TEAEs with IR oxycodone administration (82.4%) than with IR OC/APAP (64.5%), CR OC/APAP (45.5%), or IR tramadol/APAP (42.9%). Gastrointestinal TEAEs were reported for 50.0%, 35.5%, 30.3%, and 14.3% during IR oxycodone, IR OC/APAP, CR OC/APAP, and IR tramadol/APAP administration, respectively. 22 (45.8%) subjects were withdrawn from the study due to vomiting (as required by the protocol); 7 (21.2%) during CR OC/APAP treatment. No apparent clinically significant treatment-related trends were observed in clinical laboratory assessments or physical examination findings.

TABLE 88
Most Frequently Occurring (>20% in the Overall Group) TEAEs

| | | IR | IR | | |
|---|---|---|---|---|---|
| | CR | Oxy- | Tramadol/ | IR | |
| TEAE, | OC/APAP | codone | APA | OC/APAP | Overall |
| n (%) | (n = 33) | (n = 34) | P (n = 28) | (n = 31) | (N = 48) |
| Any TEAE | 15 (45.5) | 28 (82.4) | 12 (42.9) | 20 (64.5) | 44 (91.7) |
| Nausea | 8 (24.2) | 13 (38.2) | 2 (7.1) | 9 (29.0) | 26 (54.2) |
| Vomiting | 7 (21.2) | 8 (23.5) | 2 (7.1) | 5 (16.1) | 22 (45.8) |
| Dizziness | 4 (12.1) | 13 (38.2) | 2 (7.1) | 4 (12.9) | 19 (39.6) |
| Pruritus | 7 (21.2) | 13 (38.2) | 5 (17.9) | 2 (6.5) | 19 (39.6) |
| Headache | 5 (15.2) | 5 (14.7) | 3 (10.7) | 3 (9.7) | 14 (29.2) |

The results of this study demonstrate that steady state was achieved by day 2 (24 h) of CR OC/APAP initiation for both oxycodone and APAP. Both oxycodone and APAP plasma concentrations rose quickly after administration of CR OC/APAP, with a shorter Tₘₐₓ for APAP (1 h) than for oxycodone (3 h). At steady state, CR OC/APAP (2 tablets every 12 hours) produced a comparable PK profile to IR products dosed every 6 hours, with less fluctuation in oxycodone concentrations compared with IR OC/APAP and lower trough plasma concentrations of APAP compared with both IR comparators prior to subsequent dosing. CR OC/APAP was generally well tolerated; the most frequently reported TEAEs were nausea, vomiting, and pruritus. TEAEs were similar to IR products containing oxycodone. These findings support the safe and appropriate administration of CR OC/APAP during a dosing interval of every 12 hours.

### Example 38

### Comparison of the Pharmacokinetic Profile of a Single Dose of a Controlled-Release Oxycodone and Acetaminophen Combination Tablet (CR OC/APAP) and Marketed Immediate-Release Opioids and Opioid/Acetaminophen Combination Tablets

A single-center, randomized, open-label, single-dose study was conducted to evaluate the single-dose pharmacokinetic (PK) characteristics, safety, and tolerability of CR OC/APAP every 12 hours for a single dose (administered once) compared with those of commercially-available forms of IR oxycodone, IR tramadol/APAP, and IR OC/APAP every 6 hours for 2 doses in healthy participants. Participants in the study were healthy adults aged 18 to 55 years, with a weight ≧130 lb and a body mass index of ≧19 to <30 kg/m². Current recreational drug users were excluded, as were subjects with a history of abuse/addiction or recent illicit drug use (within 2 years) or nicotine use (within 6 months). Other exclusion criteria included a history of any condition that might interfere with the absorption, distribution, metabolism, or excretion of study drug; or history of gastric bypass or gastric band surgery. A total of 48 adults were enrolled; 30 (62.5%) completed all treatment periods. Data from 29 subjects who completed all 4 study periods were included in the PK analyses; data from 1 completer were excluded due to a protocol violation (consumed prohibited medications).

Participants were randomly assigned to receive each study medication in 1 of 4 treatment periods (˜48 h each) separated by a minimum of 7 days. The following treatments were administered under fasted conditions:
CR OC/APAP (OC/APAP) 7.5 mg/325 mg (see selected example from Chart No. 1); 2 tablets (total dose, 15 mg/650 mg) administered once

IR oxycodone (commercially-available) 15 mg; 1 tablet every 6 hours for 2 doses
IR tramadol/APAP (commercially-available) 37.5 mg/325 mg; 1 tablet every 6 hours for 2 doses
IR OC/APAP (commercially-available) 7.5 mg/325 mg; 1 tablet every 6 hours for 2 doses

Subjects who completed all 4 treatment periods of the study were included in PK analyses. The PK of oxycodone was compared among CR OC/APAP, IR oxycodone, and IR OC/APAP. The PK of APAP was compared among CR OC/APAP, IR tramadol/APAP, and IR OC/APAP. Blood samples for bioanalysis of oxycodone and APAP were collected predose (<1 h prior to dosing), and at 15, 30, and 45 minutes and 1, 2, 3, 4, 6, 6.5, 7, 8, 9, 10, 12, 16, 18, 20, 24, and 36 hours after dosing. Oxycodone and APAP concentrations were determined using liquid chromatography/tandem mass spectrometry. Plasma concentration versus time data were used to estimate the PK parameters of oxycodone and APAP, calculated using standard non-compartmental methods. An analysis of variance was performed to compare treatments using the natural log-transformed dose-normalized (plasma concentration divided by dose) PK parameters (AUC₀₋ₜ, AUC_{0-inf}, and Cₘₐₓ) or the nontransformed PK parameters (Kₑₗ and t_{1/2}) as the dependent variables with sequence, treatment, and period as fixed effects and subjects nested within sequences as random effect. Geometric least-squares (LS) means, ratio of geometric LS means, the corresponding 90% confidence interval (CI) for the LS means ratio, intrasubject variability, and P values for testing the fixed effects were summarized. A 90% CI of the geometric LS means ratio fully contained within 80% to 125% concluded no difference between treatments. Wilcoxon signed-rank test was performed to determine the statistical significance of the median difference for Tₘₐₓ and lag time (t_{lag}). P≦0.05 was considered a significant difference between treatments. Summary statistics were compiled for treatment-emergent adverse events (TEAEs).

FIG. 78 presents the mean plasma concentrations of oxycodone versus time for the three treatments containing oxycodone. Table 89 presents a summary of the plasma PK parameters for oxycodone. As shown in FIG. 78 and Table 89, mean plasma concentrations of oxycodone increased rapidly after CR OC/APAP administration. Lag time (t_{lag}) was 0; median Tₘₐₓ was 4 hours. Oxycodone was eliminated slowly (mean plasma oxycodone concentrations from CR OC/APAP were approximately 45% of peak 12 hours after dosing). The total dose-normalized systemic exposure to oxycodone from a single dose of CR OC/APAP was comparable to that from 2 doses (q6 h) of the comparators (IR oxycodone and IR OC/APAP). Peak concentrations of oxycodone from CR OC/APAP were achieved at 4 hours; time to Cₘₐₓ for IR oxycodone (8 h; P=0.065) and IR OC/APAP (8 h; P=0.004) occurred approximately 2 hours after the second dose. Oxycodone Cₘₐₓ with CR OC/APAP (dose-normalized) was equivalent to Cₘₐₓ achieved with IR oxycodone, but 27% lower than that with IR OC/APAP.

TABLE 89
Plasma Pharmacokinetic Parameters for Oxycodone (n = 29)

| Parameter, mean (SD) | CR OC/APAP^{a} | | IR OC/APAP^{a} | | IR Oxycodone^{b} | |
|---|---|---|---|---|---|---|
| AUC₀₋ₜ, ng · h/mL | 167.90 | (36.83) | 169.85 | (34.23) | 334.61 | (62.46) |
| AUC_{0-inf}, ng · h/mL | 169.34 | (37.03) | 171.53 | (34.05) | 336.30 | (62.77) |
| Cₘₐₓ, ng/mL | 14.28 | (2.94) | 19.42 | (4.62) | 31.27 | (8.17) |
| Tₘₐₓ, h^{c} | 4.00 | (0.75-12.00) | 8.00^{d} | (0.50-12.00) | 8.00^{d} | (0.75-12.00) |
| T_{lag}, h^{c} | 0.00 | (0.00-0.25) | 0.00 | (0.00-0.25) | 0.00 | (0.00-0.27) |
| Kₑₗ, h⁻¹ | 0.1577 | (0.0223) | 0.1764 | (0.0226) | 0.1796 | (0.0141) |
| t_{1/2}, h | 4.47 | (0.58) | 3.99 | (0.48) | 3.88 | (0.31) |

^{a}15 mg oxycodone
^{b}30 mg oxycodone
^{c}Median (range)
^{d}2 hours after second dose

FIG. 79 presents the mean plasma concentrations of APAP versus time for the three treatments containing APAP. Table 90 presents a summary of the plasma PK parameters for APAP. As shown in FIG. 79 and Table 90, mean plasma concentrations of APAP increased rapidly after CR OC/APAP administration. Mean plasma APAP levels from CR OC/APAP were only 18% of peak by 12 hours after dosing. APAP plasma concentrations for CR OC/APAP fell below the levels for the comparators by 8 hours after dosing (2 hours after the second dose of the comparator). The total dose-normalized systemic exposure (AUC₀₋ₜ and AUC_{0-inf}) to APAP from a single dose of CR OC/APAP was comparable to that from 2 doses (q6 h) of the comparators (IR tramadol/APAP and IR OC/APAP). The 90% CIs of the ratios of geometric LS means for AUC₀₋ₜ and AUC_{0-inf} were fully contained within the predefined no-difference range of 80% to 125%. Dose-normalized peak concentrations of APAP were equivalent, but were achieved more rapidly with CR OC/APAP than with IR tramadol/APAP (P<0.001).

TABLE 90
Plasma Pharmacokinetic Parameters for APAP (n = 29)

| Parameter, | | | | | | |
|---|---|---|---|---|---|---|
| mean (SD) | CR OC/APAP^{a} | | IR OC/APAP^{a} | | IR Tramadol/APAP^{a} | |
| AUC₀₋ₜ, ng · h/mL | 29064.91 | (6851.20) | 29192.56 | (6892.03) | 29934.67 | (6577.90) |
| AUC₀₋ₜ, ng · h/mL | 30759.04 | (7000.49) | 30367.98 | (7290.82) | 30989.26 | (6759.41) |
| Cₘₐₓ, ng/mL | 4653.79 | (1360.28) | 4387.24 | (1326.26) | 4255.52 | (1004.33) |
| Tₘₐₓ, h^{b} | 0.75 | (0.50-2.00) | 0.75 | (0.25-12.00) | 2.00 | (0.50-9.00) |
| T_{lag}, h^{b} | 0.00 | (0.00-0.25) | 0.00 | (0.00-0.25) | 0.00 | (0.00-0.50) |
| Kₑₗ, h⁻¹ | 0.1328 | (0.0375) | 0.1684 | (0.0435) | 0.1780 | (0.0399) |
| t_{1/2}, h | 5.75 | (2.07) | 4.41 | (1.24) | 4.12 | (1.08) |

^{a}Total dose of 650 mg APAP
^{b}Median (range)

Standard safety assessments including adverse event monitoring and clinical laboratory tests (bilirubin and other liver function tests, and serum chemistry, hematology, and urinalysis measures) were performed throughout the study. Table 91 presents a summary of the most frequently occurring TEAEs. Overall, 29 of 48 (60.4%) enrolled participants reported TEAE. The most common TEAEs after CR OC/APAP administration were nausea, dizziness, and somnolence. More subjects reported TEAEs after receiving IR oxycodone (58.1%) than after receiving IR OC/APAP (37.5%), CR OC/APAP (23.1%), or IR tramadol/APAP (22.2%). All TEAEs were rated by the investigator as either mild or moderate in severity; there were no serious or severe TEAEs. 13 subjects (27.1%) were withdrawn from the study due to vomiting (as required by the protocol); 10 subjects (23.3%) during IR oxycodone treatment and 1 each during treatment with CR OC/APAP (2.6%), IR tramadol/APAP (2.8%), and IR OC/APAP (2.5%). No clinically significant treatment-related trends were observed in clinical laboratory assessments (including bilirubin and other liver function tests) or physical examination findings.

TABLE 91
Most Frequently Occurring (>10% in the Overall Group) TEAEs

| | CR | IR | IR | IR | |
|---|---|---|---|---|---|
| TEAE, | OC/APAP^{a} | Oxycodone^{b} | Tramadol/APAP^{c} | OC/APAP^{d} | Overall |
| n (%) | (n = 39) | (n = 43) | (n = 36) | (n = 40) | (N = 48) |
| Any TEAE | 9 (23.1) | 25 (58.1) | 8 (22.2) | 15 (37.5) | 29 (60.4) |
| Nausea | 5 (12.8) | 15 (34.9) | 3 (8.3) | 9 (22.5) | 21 (43.8) |
| Dizziness | 3 (7.7) | 10 (23.3) | 2 (5.6) | 4 (10.0) | 16 (33.3) |
| Vomiting | 1 (2.6) | 10 (23.3) | 1 (2.8) | 1 (2.5) | 13 (27.1) |
| Headache | 1 (2.6) | 5 (11.6) | 3 (8.3) | 3 (7.5) | 10 (20.8) |
| Somnolence | 3 (7.7) | 2 (4.7) | 0 (0.0) | 3 (7.5) | 5 (10.4) |
| Feeling hot | 1 (2.6) | 2 (4.7) | 0 (0.0) | 2 (5.0) | 5 (10.4) |
| Pruritus | 1 (2.6) | 5 (11.6) | 0 (0.0) | 1 (2.5) | 5 (10.4) |

^{a}2 tablets once; 15 mg OC/650 mg APAP total
^{b}1 tablet q6h; 30 mg OC total
^{c}1 tablet q6h; 75 mg tramadol/650 mg APAP total
^{d}1 tablet q6h; 15 mg OC/650 mg APAP total

The results of this study demonstrate that plasma oxycodone and APAP levels rose rapidly after a single dose of CR OC/APAP. Plasma oxycodone concentrations were sustained throughout the proposed dosing interval (12 hours); however, APAP concentrations slowly declined to 18% of the peak at 12 hours. The low APAP plasma concentrations at 12 hours suggest little accumulation of APAP after repeated dosing of CR OC/APAP. The similarity of the relevant PK parameters to the IR marketed compounds supports a dosing interval of CR OC/APAP as used in this study. CR OC/APAP was generally well tolerated; the most frequently reported TEAEs were nausea, dizziness, and somnolence. These findings support a 12-hour dosing interval of CR OC/APAP for patients with moderate to severe acute pain.

### Example 39

### Half-Value Duration Analysis for Acetaminophen after Single and Multiple Doses of Oral Controlled-Release Oxycodone/Acetaminophen (CR OC/APAP) Tablets

Post hoc analysis of PK data from two randomized, open-label, crossover studies (1 single dose and 1 multiple dose) was performed to evaluate the half-value duration (HVD) for APAP after single and multiple doses (administered q12 h) of CR OC/APAP (2 tablets; total, 15 mg OC/650 mg APAP) (see selected example from Chart No. 1) compared with commercially-available IR OC/APAP (1 tablet; 7.5 mg/325 mg) administered every 6 hours. Participants were healthy adults aged 18 to 55 years, with weight 130 lb and a body mass index of 19 to <30 kg/m². Exclusion criteria included: current recreational drug use; history of abuse/addiction or recent illicit drug use (within 2 years) or nicotine use (within 6 months); history of any condition that may interfere with the absorption, distribution, metabolism, or excretion of study medication; or history of gastric bypass or gastric band surgery. PK data were analyzed for 29 subjects in the single-dose study and 24 subjects in the multiple-dose study; data from 1 participant in the single-dose study were excluded due to the use of prohibited medication (APAP).

The following treatments were administered under fasted conditions:
Oral doses of CR OC/APAP (7.5 mg OC/325 mg APAP) (see selected example from Chart No. 1) administered as 2 tablets (total dose, 15 mg/650 mg) taken once (single-dose study) or 2 tablets taken twice daily every 12 hours over 4.5 days for 9 doses (multiple-dose study)
Oral doses of IR OC/APAP (commercially available) 7.5 mg/325 mg administered during a separate trial period as 1 tablet every 6 hours for 2 doses (single-dose study) or 1 tablet every 6 hours over 4.5 days for 18 doses (multiple-dose study)

Blood samples for bioanalysis of APAP were collected up to 36 hours after dosing in the single-dose study and up to 132 hours after the hour-0 dose in the multiple-dose study. HVD, degree of fluctuation (100·[Cₘₐₓₛₛ−Cₘᵢₙₛₛ]/C_{avgss}), Cₘₐₓ, Tₘₐₓ, and area under the concentration-time curve (AUC) were calculated for APAP. Descriptive statistics were used to report demographics and baseline characteristics. For the multiple-dose study, analyses were performed for both the initial dose period (day 1, 0-12 h) and at steady state (day 5, 0- to 12-h dosing interval; i.e., 96-108 h). PK analyses included subjects who completed each study. Mean concentration-time profiles were presented on a linear scale. Individual plasma concentration versus actual time data were used to estimate the PK parameters of APAP. HVD of APAP after CR OC/APAP (single or multiple dose) was compared with that after IR OC/APAP using paired 2-tailed t tests. Descriptive statistics and paired t tests were calculated for the percentage difference in HVD relative to the IR product (% RDHVD); % RDHVD was calculated as the average percentage difference in HVD for CR OC/APAP relative to IR OC/APAP for individual subjects.

FIGS. 80 and 81 present the plasma APAP concentration over time and HVD for APAP in the initial 12 hours after dosing for the single-dose and multi-dose studies, respectively. FIG. 80 presents the single-dose study results. FIG. 81 presents the multi-dose study results. Table 92 presents the PK measures for APAP in the initial 12 hours after dosing for both the single-dose and multi-dose studies. As shown in FIGS. 80 and 81 and in Table 92, HVD of APAP was not significantly different for CR OC/APAP and IR OC/APAP after the first administration (difference of 0.73 h [P=0.133] in the single-dose study, and 0.27 h [P=0.520] on day 1 in the multiple-dose study). AUC and Cₘₐₓ for APAP were similar after administration of CR OC/APAP and IR OC/APAP.

TABLE 92
Pharmacokinetic Measures for APAP, Initial 12 Hours After Dosing

| Parameter, | Single-Dose Study | | | | Multiple-Dose Study, Day 1 | | | |
|---|---|---|---|---|---|---|---|---|
| mean (SD) | CR OC/APAP | | IR OC/APAP | | CR OC/APAP | | IR OC/APAP | |
| Cₘₐₓ, ng/mL | 4653.79 | (1360.3) | 4387.24 | (1326.3) | 4857.5 | (1066.5) | 4317.92 | (1006.3) |
| Tₘₐₓ, h^{a} | 0.75 | (0.5-2.0) | 0.75 | (0.25-12.0) | 1.00 | (0.5-4.0) | 0.53 | (0.5-8.0) |
| AUC, ng · h/mL | 30759.0 | (7000.5)^{b} | 30368.0 | (7290.8)^{b} | 24924.3 | (5667.5)^{c} | 25093.7 | (5085.0)^{c} |
| HVD, h | 3.68 | (1.8) | 4.41 | (2.3) | 3.33 | (1.5) | 3.60 | (2.0) |

^{a}Median (range)
^{b}AUC_{0-inf}
^{c}AUC_{0-12 h}

FIG. 82 presents the steady-state plasma APAP concentration over time and HVD for APAP during day 5 of the multi-dose study. Table 93 presents the steady-state (day 5) PK measures for APAP. Steady state APAP concentrations were reached within 2 days (24 h after first dose) for CR OC/APAP and at 1 day (12 h) for IR OC/APAP. At day 5 under steady-state conditions, the HVD of APAP after administration of CR OC/APAP was significantly greater than that after administration of IR OC/APAP (difference of 1.13 h [P=0.024]). HVD was 106% greater for CR OC/APAP versus IR OC/APAP. There was no significant difference in degree of fluctuation between CR OC/APAP and IR OC/APAP. AUC during the first 12 hours after dosing on day 5 (at steady state), Cₘₐₓ, and Tₘₐₓ for APAP were similar for CR OC/APAP and IR OC/APAP.

TABLE 93
Steady-State Pharmacokinetic Measures for APAP (Day 5)

| Parameter | CR OC/APAP | | IR OC/APAP | |
|---|---|---|---|---|
| Cₘₐₓₛₛ, ng/mL^{a} | 4792.50 | (1132.4) | 4876.67 | (1383.1) |
| Tₘₐₓₛₛ, h^{b} | 1.00 | (0.5-4.0) | 0.75 | (0.25-8.0) |
| AUC₀₋₁₂ₕₛₛ, ng · h/mL^{a} | 28160 | (5807) | 29284 | (5478) |
| Degree of fluctuation, % | 169.13 | (39.8) | 155.25 | (38.8) |
| HVDₛₛ, h^{a} | 4.24 | (1.4) | 3.11 | (1.8) |

^{a}Mean (SD)
^{b}Median (range)

Safety and tolerability were monitored throughout each study. Table 94 presents the most frequently occurring treatment-emergent adverse events (TEAEs). The most frequently reported TEAEs following administration of CR OC/APAP were nausea, vomiting, pruritus, dizziness, and headache.

TABLE 94
Most Frequently Occurring Treatment-Emergent
Adverse Events (≧5% with CR OC/APAP)

| | Single-Dose Study | | Multiple-Dose Study | |
|---|---|---|---|---|
| | | IR | CR | IR |
| | CR OC/APAP | OC/APAP | OC/APAP | OC/APAP |
| TEAE, n (%) | (n = 39) | (n = 40) | (n = 33) | (n = 31) |
| Any TEAE | 9 (23.1) | 15 (37.5) | 15 (45.5) | 20 (64.5) |
| Nausea | 5 (12.8) | 9 (22.5) | 8 (24.2) | 9 (29.0) |
| Dizziness | 3 (7.7) | 4 (10.0) | 4 (12.1) | 4 (12.9) |
| Vomiting | 1 (2.6) | 1 (2.5) | 7 (21.2) | 5 (16.1) |
| Headache | 1 (2.6) | 3 (7.5) | 5 (15.2) | 3 (9.7) |
| Somnolence | 3 (7.7) | 3 (7.5) | 1 (3.0) | 1 (3.2) |
| Pruritus | 1 (2.6) | 1 (2.5) | 7 (21.2) | 2 (6.5) |
| Feeling hot | 1 (2.6) | 2 (5.0) | 2 (6.1) | 2 (6.5) |
| Abdominal | 1 (2.6) | 0 (0.0) | 2 (6.1) | 3 (9.7) |
| pain | | | | |

The results of this study demonstrate that Cₘₐₓ, Tₘₐₓ, and AUC of APAP were similar after single-dose administration as well as after dosing at steady state. HVD for APAP at steady state was significantly greater for CR OC/APAP compared with IR OC/APAP. The HVD after initial dosing was similar between treatments. CR OC/APAP was generally well tolerated, with TEAEs that were consistent with those associated with opioid therapy. The results of this PK analysis support the administration of CR OC/APAP every 12 hours for the management of moderate to severe acute pain.

### Example 40

### Relationship Between Oxycodone Pharmacokinetics and Subjective Drug Effects Following Oral Administration of an Immediate-Release Combination of Oxycodone and Acetaminophen and Controlled-Release Oxycodone/Acetaminophen (CR OC/APAP) Tablets

A single-center, randomized, double-blind, double-dummy, active- and placebo-controlled, crossover study was conducted to examine the relationship between oxycodone PK parameters and the pharmacodynamic (PD) parameters of participant-reported drug liking, drug high, and good drug effects, which have been associated with abuse. PK and PD parameters were assessed after administration of intact and crushed CR OC/APAP (see selected example from Chart No. 1) and commercially-available IR OC/APAP to recreational opioid users. Participants included healthy adult (18-55 y) male and female nondependent, recreational opioid users who reported occasions of recreational opioid use over the past year, including ≧1 occasion within the past 12 weeks.

Participants initially underwent a naloxone challenge test to confirm a lack of physical dependence on opioids and a drug discrimination test to determine that they could detect the subjective effects of oxycodone. During the drug discrimination test, all participants received single doses of IR OC/APAP (15 mg/650 mg total; two tablets of 7.5 mg/325 mg each) (see selected example from Chart No. 1) and placebo and reported on the subjective effects. Participants who could not discriminate between active drug and placebo or tolerate this single dose were excluded from enrollment. The treatment phase included a total of 7 assessment periods, with a 72-hour washout period between doses. Out of 107 participants who entered the study, were checked into the clinical facility, and completed the study inclusion phase, 61 participants met inclusion criteria and entered the treatment phase; of these, 55 completed all 7 assessment periods and were included in the analysis population. Participants had a mean age of 26 years and approximately 73% were male. Most participants (95%) had a history of alcohol use, and the majority (67%) had a history of tobacco use.

Because of the history of prescription opioid abuse in this population, IR OC/APAP was encapsulated for proper blinding. To ensure blinding of all study treatments, participants received matching placebos for each possible treatment configuration so dose administration in each treatment period consisted of 8 capsules and 4 tablets. During each assessment period, participants received a single dose of one of the 7 study treatments:

CR OC/APAP intact

High dose: 30 mg/1300 mg (4 tablets)

Low dose: 15 mg/650 mg (2 tablets)

IR OC/APAP intact

High dose: 30 mg/1300 mg (4 tablets, over-encapsulated)

Low dose: 15 mg/650 mg (2 tablets, over-encapsulated)

CR OC/APAP crushed (encapsulated)

High dose: 30 mg/1300 mg (8 capsules)

IR OC/APAP crushed (encapsulated)

High dose: 30 mg/1300 mg (4 capsules)

Placebo

Plasma was extracted from whole blood samples collected before dosing and up to 24 hours after each dose. Plasma oxycodone and APAP concentrations were determined using liquid chromatography with tandem mass spectrometry detection. Oxycodone and APAP PK parameters, including Cₘₐₓ, Tₘₐₓ, and area under the concentration-time curve (AUC) for 0-1 h, 0-2 h, 0-4 h, 0-8 h, 0-12 h, and from hour 0 extrapolated to infinity, were analyzed by standard non-compartmental methods using WinNonlin®, version 6.1 or higher (Pharsight, Cary, N.C.). Pharmacodynamic (PD) outcome measures included patient-reported visual analog scale (VAS) scores for: drug liking—assessed on a 100-mm bipolar VAS (0 mm=Strong disliking; 50 mm=Neither like or dislike; 100 mm=Strong liking); drug high—assessed on a 100-mm unipolar VAS (0 mm=None; 100 mm=Extremely); and good drug effects—assessed on a 100-mm unipolar VAS (0 mm=None; 100 mm=Extremely). PD measures included peak drug effects (Eₘₐₓ), time to Eₘₐₓ (TEₘₐₓ), and area under the drug effect curve (AUE) assessed at multiple time points from 0-12 hours. The relationship between oxycodone PK and PD outcomes was evaluated by calculating correlation coefficients between these parameters (e.g., Cₘₐₓ vs Eₘₐₓ and AUC₀₋ₓ vs AUE₀₋ₓ) using SAS® version 9.1 or higher (SAS Institute Inc., Cary, N.C.).

Table 95 presents a summary of the oxycodone PK parameters from this study. Oxycodone Cₘₐₓ following the administration of high- and low-dose intact CR OC/APAP was approximately half the value observed with the same doses of IR OC/APAP. Median oxycodone Tₘₐₓ was significantly longer for intact CR OC/APAP compared with the same dose of intact IR OC/APAP, representing increases of 185% and 96%, respectively, for low and high doses (P<0.001). Oxycodone AUC values for the first 4 hours after dosing were also lower for intact CR OC/APAP than IR OC/APAP; however, overall oxycodone exposure (AUC₀₋ₜ and AUC_{0-inf}) was equivalent between formulations. High-dose CR OC/APAP produced oxycodone Cₘₐₓ levels that were similar for the intact and crushed formulation. Crushing CR OC/APAP significantly reduced the IR characteristic. Median Tₘₐₓ for crushed CR OC/APAP was delayed by 1.5 hours versus intact CR OC/APAP (73% increase). High-dose intact CR OC/APAP produced oxycodone levels soon after dosing (e.g., AUC₀₋₂ₕ) that were considerably greater than those for crushed CR OC/APAP (36.7 ng·h/mL vs 17.3 ng·h/mL).

TABLE 95
Oxycodone Pharmacokinetic Parameters

| | Low-Dose | | High-Dose | | High-Dose | |
|---|---|---|---|---|---|---|
| | OC/APAP | | OC/APAP | | OC/APAP | |
| | (15 mg/650 mg) | | (30 mg/1300 mg) | | (30 mg/1300 mg) | |
| PK | Intact | | Intact | | Crushed | |
| Parameters | CR | IR | CR | IR | CR | IR |
| Cₘₐₓ, ng/mL^{a} | 14.4 | 34.1 | 31.4 | 66.2 | 32.2 | 55.1 |
| | (3.6) | (9.7) | (7.6) | (24.5) | (8.5) | (18.7) |
| Tₘₐₓ, h^{b} | 3.08 | 1.08 | 2.08 | 1.06 | 3.59 | 1.08 |
| | (0.58-6.08) | (0.42-4.18) | (0.55-6.12) | (0.52-8.15) | (1.10-6.10) | (0.52-5.17) |

^{a}Mean (SD)
^{b}Median (range)

FIGS. 83 to 88 present the 12-hour oxycodone plasma concentrations and PD outcomes for drug liking and drug high. In general, drug liking, drug high, and good drug effects were greater for all IR OC/APAP formulations compared with CR OC/APAP. In addition, crushing CR OC/APAP delayed and did not increase positive subjective effects and produced similar or less drug liking, drug high, and good drug effects than the same dose of intact CR OC/APAP or IR OC/APAP. Overall, the formulations that produced higher oxycodone Cₘₐₓ and AUC also produced greater drug liking, drug high, and good drug effects than those that produced lower oxycodone concentrations (see FIGS. 83 to 88). Pharmacokinetic and pharmacodynamic results for good drug effects (results not shown) were similar to those for drug high. As shown in Table 96, strong correlations were observed between oxycodone Cₘₐₓ and AUC₀₋ₓ and Eₘₐₓ and AUE₀₋ₓ, respectively, for drug liking, drug high, and good drug effects (R²=0.711-0.997). Specifically, larger oxycodone Cₘₐₓ values were correlated with a higher Eₘₐₓ for all PD assessments. Analyses also showed strong correlations for AUE₀₋₄ₕ and AUE₀₋₈ₕ with Cₘₐₓ (R²=0.801-0.947). (See Table 96). The correlations between AUE and oxycodone Cₘₐₓ and Tₘₐₓ were not as strong as the comparisons of Eₘₐₓ and Cₘₐₓ (see Table 96; see also FIGS. 89 to 92 presenting correlation plots for PK and PD outcomes). The assessed PK parameters showed slightly stronger relationship to the PD parameters for drug high compared with drug liking and good drug effects (see FIGS. 89 to 92; data for good drug effects not shown).

TABLE 96
Correlation Coefficients for Comparisons of
Pharmacodynamic and Pharmacokinetic Parameters

| | AUE | Correlation Coefficient | | |
|---|---|---|---|---|
| PD/PK Parameters | Range | Liking | High | Good Drug Effects |
| Eₘₐₓ vs Cₘₐₓ | — | 0.9012 | 0.9364 | 0.8901 |
| AUE vs Cₘₐₓ | 0-1 h | 0.2945 | 0.4234 | 0.3497 |
| | 0-2 h | 0.5806 | 0.7098 | 0.6542 |
| | 0-4 h | 0.8349 | 0.9472 | 0.9101 |
| | 0-8 h | 0.8010 | 0.9339 | 0.8877 |
| | 0-12 h | 0.7609 | 0.8932 | 0.8310 |
| AUC_{0-1 h} | 0-1 h | 0.9656 | 0.9973 | 0.9831 |
| AUC_{0-2 h} | 0-2 h | 0.9126 | 0.9759 | 0.9550 |
| AUC_{0-4 h} | 0-4 h | 0.8703 | 0.9667 | 0.9420 |
| AUC_{0-8 h} | 0-8 h | 0.7976 | 0.9403 | 0.9105 |
| AUC_{0-12 h} | 0-12 h | 0.7105 | 0.8949 | 0.8432 |
| AUE vs Tₘₐₓ | 0-1 h | 0.6692 | 0.7359 | 0.6842 |
| | 0-2 h | 0.7731 | 0.7971 | 0.7926 |
| | 0-4 h | 0.5438 | 0.5447 | 0.5424 |
| | 0-8 h | 0.2785 | 0.3101 | 0.2811 |
| | 0-12 h | 0.2473 | 0.2385 | 0.2131 |

The results of these analyses demonstrate that intact high- and low-dose CR OC/APAP have PK profiles that produce lower Cₘₐₓ and longer Tₘₐₓ for OC than IR OC/APAP, which are positively correlated with the PD outcomes of lower drug liking, drug high, and good drug effects. Crushing CR OC/APAP further slowed the rate of oxycodone release and produced corresponding decreases in drug liking, drug high, and good drug effects relative to those produced by comparable doses of intact CR OC/APAP and intact and crushed IR OC/APAP. Previous analyses have demonstrated a strong correlation between oxycodone PK and self-reported drug effects. In addition, abuse potential studies of other prescription opioids that also conducted PK assessments have shown that lower and more prolonged increases in opioid concentrations are associated with lower drug liking, drug high, and good drug effects, although specific correlation analyses were not conducted. In total, the results of these analyses demonstrate that the participant-reported drug effects resulting from CR OC/APAP administration were strongly correlated with the oxycodone PK profile. In particular, CR OC/APAP, with its PK profile of decreased oxycodone Cₘₐₓ and relatively lower early oxycodone exposure, produced lower degrees of drug liking, drug high, and good drug effects than those of IR OC/APAP, which produced higher oxycodone Cₘₐₓ and greater early oxycodone exposure.

All references cited herein are hereby incorporated by reference. The foregoing is offered primarily for purposes of illustration. It will be readily apparent to those skilled in the art that further drugs can be included, and that the shapes, components, additives, proportions, methods of formulation, and other parameters described herein can be modified further or substituted in various ways without departing from the spirit and scope of the invention.

What is claimed:

1. A solid oral dosage form comprising:
(a) at least one immediate release portion comprising acetaminophen and oxycodone or a pharmaceutically acceptable salt thereof; and
(b) at least one extended release portion comprising acetaminophen, oxycodone or a pharmaceutically acceptable salt thereof, and an extended release component;
wherein the total amount of acetaminophen in the dosage form is about 325 mg to about 650 mg, and the total amount of oxycodone or its pharmaceutically acceptable salt in the dosage form is about 5 mg to about 15 mg; and
wherein upon oral administration of the dosage form to a subject, the dosage form provides a higher AUC for oxycodone when the dosage form is administered to the subject in an intact state versus when the dosage form is administered to the subject in a crushed or ground state.

2. The solid oral dosage form of claim 1, wherein the extended release component is an extended release polymer.

3. The solid oral dosage form of claim 2, wherein the extended release portion comprises, by weight of the extended release portion, from about 30% to about 50% of the extended release polymer.

4. The solid oral dosage form of claim 2, wherein the extended release polymer is polyethylene oxide.

5. The solid oral dosage form of claim 4, wherein the polyethylene oxide has a molecular weight of about 900,000 Daltons to about 7,000,000 Daltons.

6. The solid oral dosage form of claim 1, wherein upon oral administration of the dosage form to a subject, the dosage form provides a longer Tₘₐₓ for oxycodone when the dosage form is administered to the subject in a crushed or ground state versus when the dosage form is administered to the subject in an intact state.

7. The solid oral dosage form of claim 6, wherein administration of the dosage form to a subject produces a mean Tₘₐₓ for oxycodone that is at least about 30 minutes greater when the dosage form is administered in a crushed or ground state versus when the dosage form is administered in an intact state.

8. The solid oral dosage form of claim 6, wherein administration of the dosage form to a subject produces a mean Tₘₐₓ for oxycodone that is at least about 45 minutes greater when the dosage form is administered in a crushed or ground state versus when the dosage form is administered in an intact state.

9. The solid oral dosage form of claim 6, wherein administration of the dosage form to a subject produces a mean Tₘₐₓ for oxycodone that is at least about 60 minutes greater when the dosage form is administered in a crushed or ground state versus when the dosage form is administered in an intact state.

10. The solid oral dosage form of claim 6, wherein administration of the dosage form to a subject produces a mean Tₘₐₓ for oxycodone that is at least about 75 minutes greater when the dosage form is administered in a crushed or ground state versus when the dosage form is administered in an intact state.

11. The solid oral dosage form of claim 6, wherein administration of the dosage form to a subject produces a mean Tₘₐₓ for oxycodone that is at least about 90 minutes greater when the dosage form is administered in a crushed or ground state versus when the dosage form is administered in an intact state.

12. The solid oral dosage form of claim 6, wherein administration of the dosage form to a subject produces a mean Tₘₐₓ for oxycodone that is at least about 105 minutes greater when the dosage form is administered in a crushed or ground state versus when the dosage form is administered in an intact state.

13. The solid oral dosage form of claim 6, wherein administration of the dosage form to a subject produces a mean Tₘₐₓ for oxycodone that is at least about 120 minutes greater when the dosage form is administered in a crushed or ground state versus when the dosage form is administered in an intact state.

14. The solid oral dosage form of claim 1, wherein upon oral administration of the dosage form to a subject, the dosage form provides a AUC(0-1 hr) for oxycodone that is about 50% to about 1000% higher when the dosage form is administered to the subject in an intact state versus when the dosage form is administered to the subject in a crushed or ground state.

15. The solid oral dosage form of claim 14, wherein upon oral administration of the dosage form to a subject, the dosage form provides a AUC(0-1 hr) for oxycodone that is about 100% to about 900% higher when the dosage form is administered to the subject in an intact state versus when the dosage form is administered to the subject in a crushed or ground state.

16. The solid oral dosage form of claim 14, wherein upon oral administration of the dosage form to a subject, the dosage form provides a AUC(0-1 hr) for oxycodone that is about 200% to about 800% higher when the dosage form is administered to the subject in an intact state versus when the dosage form is administered to the subject in a crushed or ground state.

17. The solid oral dosage form of claim 14, wherein upon oral administration of the dosage form to a subject, the dosage form provides a AUC(0-1 hr) for oxycodone that is about 300% to about 700% higher when the dosage form is administered to the subject in an intact state versus when the dosage form is administered to the subject in a crushed or ground state.

18. The solid oral dosage form of claim 1, wherein upon oral administration of the dosage form to a subject, the dosage form provides a AUC(0-2 hr) for oxycodone that is about 50% to about 500% higher when the dosage form is administered to the subject in an intact state versus when the dosage form is administered to the subject in a crushed or ground state.

19. The solid oral dosage form of claim 18, wherein upon oral administration of the dosage form to a subject, the dosage form provides a AUC(0-2 hr) for oxycodone that is about 100% to about 400% higher when the dosage form is administered to the subject in an intact state versus when the dosage form is administered to the subject in a crushed or ground state.

20. The solid oral dosage form of claim 18, wherein upon oral administration of the dosage form to a subject, the dosage form provides a AUC(0-2 hr) for oxycodone that is about 150% to about 300% higher when the dosage form is administered to the subject in an intact state versus when the dosage form is administered to the subject in a crushed or ground state.

21. The solid oral dosage form of claim 18, wherein upon oral administration of the dosage form to a subject, the dosage form provides a AUC(0-2 hr) for oxycodone that is about 50% to about 250% higher when the dosage form is administered to the subject in an intact state versus when the dosage form is administered to the subject in a crushed or ground state.

22. The solid dosage form of claim 1, wherein upon oral administration of the dosage form to a subject, the dosage form provides a Tmax for oxycodone that decreases by about 5% to about 70% when the dosage form is administered in an intact state versus when the dosage form is administered in a crushed or ground state.

23. The solid dosage form of claim 22, wherein the Tmax for oxycodone is decreased by about 5% to about 50% when the dosage form is administered in an intact state versus when the dosage form is administered in a crushed or ground state.

24. The solid dosage form of claim 22, wherein the Tmax for oxycodone is decreased by about 5% to about 40% when the dosage form is administered in an intact state versus when the dosage form is administered in a crushed or ground state.

25. The solid dosage form of claim 22, wherein the Tmax for oxycodone is decreased by about 5% to about 30% when the dosage form is administered in an intact state versus when the dosage form is administered in a crushed or ground state.

26. The solid dosage form of claim 22, wherein the Tmax for oxycodone is decreased by about 5% to about 20% when the dosage form is administered in an intact state versus when the dosage form is administered in a crushed or ground state.

27. The solid dosage form of claim 22, wherein the Tmax for oxycodone is decreased by about 10% to about 40% when the dosage form is administered in an intact state versus when the dosage form is administered in a crushed or ground state.

28. The solid dosage form of claim 22, wherein the Tmax for oxycodone is decreased by about 20% to about 60% when the dosage form is administered in an intact state versus when the dosage form is administered in a crushed or ground state.

29. The solid dosage form of claim 6, wherein the solid dosage form contains a total of about 325 mg of acetaminophen and about 5 mg of oxycodone or its pharmaceutically acceptable salt.

30. The solid dosage form of claim 6, wherein the solid dosage form contains a total of about 325 mg of acetaminophen and about 7.5 mg of oxycodone or its pharmaceutically acceptable salt.

31. The solid dosage form of claim 6, wherein the solid dosage form contains a total of about 325 mg of acetaminophen and about 10 mg of oxycodone or its pharmaceutically acceptable salt.

32. The solid dosage form of claim 6, wherein the solid dosage form contains a total of about 325 mg of acetaminophen and about 15 mg of oxycodone or its pharmaceutically acceptable salt.

33. The solid dosage form of claim 14, wherein the solid dosage form contains a total of about 325 mg of acetaminophen and about 5 mg of oxycodone or its pharmaceutically acceptable salt.

34. The solid dosage form of claim 14, wherein the solid dosage form contains a total of about 325 mg of acetaminophen and about 7.5 mg of oxycodone or its pharmaceutically acceptable salt.

35. The solid dosage form of claim 14, wherein the solid dosage form contains a total of about 325 mg of acetaminophen and about 10 mg of oxycodone or its pharmaceutically acceptable salt.

36. The solid dosage form of claim 14, wherein the solid dosage form contains a total of about 325 mg of acetaminophen and about 15 mg of oxycodone or its pharmaceutically acceptable salt.

37. The solid dosage form of claim 18, wherein the solid dosage form contains a total of about 325 mg of acetaminophen and about 5 mg of oxycodone or its pharmaceutically acceptable salt.

38. The solid dosage form of claim 18, wherein the solid dosage form contains a total of about 325 mg of acetaminophen and about 7.5 mg of oxycodone or its pharmaceutically acceptable salt.

39. The solid dosage form of claim 18, wherein the solid dosage form contains a total of about 325 mg of acetaminophen and about 10 mg of oxycodone or its pharmaceutically acceptable salt.

40. The solid dosage form of claim 18, wherein the solid dosage form contains a total of about 325 mg of acetaminophen and about 15 mg of oxycodone or its pharmaceutically acceptable salt.

41. The solid oral dosage form of claim 1, wherein upon oral administration of the dosage form to a subject, the dosage form provides a longer Tₘₐₓ for acetaminophen when the dosage form is administered to the subject in a crushed or ground state versus when the dosage form is administered to the subject in an intact state.

42. The solid oral dosage form of claim 41, wherein administration of the dosage form to a subject produces a mean Tₘₐₓ for acetaminophen that is at least about one hour greater when the dosage form is administered in a crushed or ground state as compared to an intact state.

43. The solid oral dosage form of claim 1, wherein upon oral administration of the dosage form to a subject, the dosage form provides a higher Cₘₐₓ for acetaminophen when the dosage form is administered to the subject in an intact state versus when the dosage form is administered to the subject in a crushed or ground state.

44. The solid oral dosage form of claim 1, wherein the total amount of acetaminophen in the composition is about 325 mg and the total amount of oxycodone or its pharmaceutically acceptable salt in the dosage form is about 7.5 mg.

45. The solid oral dosage form of claim 1, wherein the total amount of acetaminophen in the dosage form is about 325 mg and the total amount of oxycodone or its pharmaceutically acceptable salt in the dosage form is about 5 mg.

46. The solid oral dosage form of claim 1, wherein the total amount of acetaminophen in the dosage form is about 325 mg and the total amount of oxycodone or its pharmaceutically acceptable salt in the dosage form is about 10 mg.

47. The solid oral dosage form of claim 1, wherein the total amount of acetaminophen in the dosage form is about 325 mg and the total amount of oxycodone or its pharmaceutically acceptable salt in the dosage form is about 15 mg.

48. A solid oral dosage form comprising:
(a) at least one immediate release portion comprising acetaminophen and oxycodone or a pharmaceutically acceptable salt thereof; and
(b) at least one extended release portion comprising acetaminophen, oxycodone or a pharmaceutically acceptable salt thereof, and an extended release component;
wherein the total amount of acetaminophen in the dosage form is about 325 mg to about 650 mg, and the total amount of oxycodone or its pharmaceutically acceptable salt in the dosage form is about 5 mg to about 15 mg; and
wherein upon oral administration of the dosage form to a subject, the dosage form provides an abuse quotient for oxycodone that is higher when the dosage form is administered to the subject in an intact state versus when the dosage form is administered to the subject in a crushed or ground state.

49. The solid oral dosage form of claim 48, wherein the abuse quotient for oxycodone is decreased by about 5% to about 90% when the dosage form is administered in a crushed or ground state versus when the dosage form is administered in an intact state.

50. The solid oral dosage form of claim 49, wherein the abuse quotient for oxycodone is decreased by about 10% to about 80% when the dosage form is administered in a crushed or ground state versus when the dosage form is administered in an intact state.

51. The solid oral dosage form of claim 49, wherein the abuse quotient for oxycodone is decreased by about 15% to about 70% when the dosage form is administered in a crushed or ground state versus when the dosage form is administered in an intact state.

52. The solid oral dosage form of claim 49, wherein the abuse quotient for oxycodone is decreased by about 20% to about 60% when the dosage form is administered in a crushed or ground state versus when the dosage form is administered in an intact state.

53. The solid oral dosage form of claim 48, wherein the solid dosage form contains a total of about 325 mg of acetaminophen and about 5 mg of oxycodone or its pharmaceutically acceptable salt.

54. The solid oral dosage form of claim 48, wherein the solid dosage form contains a total of about 325 mg of acetaminophen and about 7.5 mg of oxycodone or its pharmaceutically acceptable salt.

55. The solid oral dosage form of claim 48, wherein the solid dosage form contains a total of about 325 mg of acetaminophen and about 10 mg of oxycodone or its pharmaceutically acceptable salt.

56. The solid oral dosage form of claim 48, wherein the solid dosage form contains a total of about 325 mg of acetaminophen and about 15 mg of oxycodone or its pharmaceutically acceptable salt.

57. The solid oral dosage form of claim 48, wherein the extended release component is an extended release polymer.

58. The solid oral dosage form of claim 57, wherein the extended release portion comprises, by weight of the extended release portion, from about 30% to about 50% of the extended release polymer.

59. The solid oral dosage form of claim 57, wherein the extended release polymer is polyethylene oxide.

60. The solid oral dosage form of claim 59, wherein the polyethylene oxide has a molecular weight of about 900,000 Daltons to about 7,000,000 Daltons.

61. The solid oral dosage form of claim 48, wherein administration of the dosage form to a subject produces a mean Tₘₐₓ for oxycodone that is at least about 30 minutes greater when the dosage form is administered in a crushed or ground state versus when the dosage form is administered in an intact state.

62. The solid oral dosage form of claim 48, wherein administration of the dosage form to a subject produces a mean Tₘₐₓ for oxycodone that is at least about 45 minutes greater when the dosage form is administered in a crushed or ground state versus when the dosage form is administered in an intact state.

63. The solid oral dosage form of claim 48, wherein administration of the dosage form to a subject produces a mean Tₘₐₓ for oxycodone that is at least about 60 minutes greater when the dosage form is administered in a crushed or ground state versus when the dosage form is administered in an intact state.

64. The solid oral dosage form of claim 48, wherein administration of the dosage form to a subject produces a mean Tₘₐₓ for oxycodone that is at least about 75 minutes greater when the dosage form is administered in a crushed or ground state versus when the dosage form is administered in an intact state.

65. The solid oral dosage form of claim 48, wherein administration of the dosage form to a subject produces a mean Tₘₐₓ for oxycodone that is at least about 90 minutes greater when the dosage form is administered in a crushed or ground state versus when the dosage form is administered in an intact state.

66. A solid oral dosage form comprising:
(a) at least one immediate release portion comprising acetaminophen and oxycodone or a pharmaceutically acceptable salt thereof; and
(b) at least one extended release portion comprising acetaminophen, oxycodone or a pharmaceutically acceptable salt thereof, and an extended release component;
wherein the total amount of acetaminophen in the dosage form is about 325 mg to about 650 mg, and the total amount of oxycodone or its pharmaceutically acceptable salt in the dosage form is about 5 mg to about 15 mg; and
wherein administration of the dosage form to a subject produces a mean Tₘₐₓ for oxycodone that is at least about 30 minutes greater when the dosage form is administered in a crushed or ground state versus when the dosage form is administered in an intact state.

67. The solid oral dosage form of claim 66, wherein administration of the dosage form to a subject produces a mean Tₘₐₓ for oxycodone that is at least about 60 minutes greater when the dosage form is administered in a crushed or ground state versus when the dosage form is administered in an intact state.

68. The solid oral dosage form of claim 66, wherein administration of the dosage form to a subject produces a mean Tₘₐₓ for oxycodone that is at least about 75 minutes greater when the dosage form is administered in a crushed or ground state versus when the dosage form is administered in an intact state.

69. The solid oral dosage form of claim 66, wherein administration of the dosage form to a subject produces a mean Tₘₐₓ for oxycodone that is at least about 90 minutes greater when the dosage form is administered in a crushed or ground state versus when the dosage form is administered in an intact state.

70. The solid oral dosage form of claim 66, wherein the extended release portion comprises, by weight of the extended release portion, from about 30% to about 50% of an extended release polymer comprising polyethylene oxide having a molecular weight of about 900,000 Daltons to about 7,000,000 Daltons.

71. The solid oral dosage form of claim 66, wherein the solid dosage form contains a total of about 325 mg of acetaminophen and about 5 mg of oxycodone or its pharmaceutically acceptable salt.

72. The solid oral dosage form of claim 66, wherein the solid dosage form contains a total of about 325 mg of acetaminophen and about 7.5 mg of oxycodone or its pharmaceutically acceptable salt.

73. The solid oral dosage form of claim 66, wherein the solid dosage form contains a total of about 325 mg of acetaminophen and about 10 mg of oxycodone or its pharmaceutically acceptable salt.

74. The solid oral dosage form of claim 66, wherein the solid dosage form contains a total of about 325 mg of acetaminophen and about 15 mg of oxycodone or its pharmaceutically acceptable salt.

75. The solid oral dosage form of claim 70, wherein upon oral administration of the dosage form to a subject, the dosage form provides a AUC(0-1 hr) for oxycodone that is about 50% to about 1000% higher when the dosage form is administered to the subject in an intact state versus when the dosage form is administered to the subject in a crushed or ground state.
